SEQUENCE LISTING (409 sequences)

SEQ ID NO: 1 - DNA, 62 bp, synthetic construct
  source 1..62: mol_type = other DNA
  modified_base 60..61: mod_base = OTHER; note = phosphorothioated base
atctcatccctgcgtgtctccgactgcacagctcgaatgcgatcaaaaaacatgagcagcat

SEQ ID NO: 2 - DNA, 64 bp, synthetic construct
  source 1..64: mol_type = other DNA
  modified_base 62..63: mod_base = OTHER; note = phosphorothioated base
atctcatccctgcgtgtctccgactgcacatcacacatgaatgatcaaaaaacatgagcagcat

SEQ ID NO: 3 - DNA, 62 bp, synthetic construct
  source 1..62: mol_type = other DNA
  modified_base 60..61: mod_base = OTHER; note = phosphorothioated base
atctcatccctgcgtgtctccgactgcactgtgtaggcatgatcaaaaaacatgagcagcat

SEQ ID NO: 4 - DNA, 62 bp, synthetic construct
  source 1..62: mol_type = other DNA
  modified_base 60..61: mod_base = OTHER; note = phosphorothioated base
atctcatccctgcgtgtctccgactgcacatgtatcctctgatcaaaaaacatgagcagcat

SEQ ID NO: 5 - DNA, 62 bp, synthetic construct
  source 1..62: mol_type = other DNA
  modified_base 60..61: mod_base = OTHER; note = phosphorothioated base
atctcatccctgcgtgtctccgactgcacatatagcctatgatcaaaaaacatgagcagcat

SEQ ID NO: 6 - DNA, 62 bp, synthetic construct
  source 1..62: mol_type = other DNA
  modified_base 60..61: mod_base = OTHER; note = phosphorothioated base
atctcatccctgcgtgtctccgactgcacgattcatgctcgatcaaaaaacatgagcagcat

SEQ ID NO: 7 - DNA, 64 bp, synthetic construct
  source 1..64: mol_type = other DNA
  modified_base 62..63: mod_base = OTHER; note = phosphorothioated base
atctcatccctgcgtgtctccgactgcacacatcctgcatgtgatcaaaaaacatgagcagcat

SEQ ID NO: 8 - DNA, 62 bp, synthetic construct
  source 1..62: mol_type = other DNA
  modified_base 60..61: mod_base = OTHER; note = phosphorothioated base
atctcatccctgcgtgtctccgactgcacactgcacgaatgatcaaaaaacatgagcagcat

SEQ ID NO: 9 - DNA, 62 bp, synthetic construct
  source 1..62: mol_type = other DNA
  modified_base 60..61: mod_base = OTHER; note = phosphorothioated base
atctcatccctgcgtgtctccgactgcactgccatagcacgatcaaaaaacatgagcagcat

SEQ ID NO: 10 - DNA, 62 bp, synthetic construct
  source 1..62: mol_type = other DNA
  modified_base 60..61: mod_base = OTHER; note = phosphorothioated base
atctcatccctgcgtgtctccgactgcacagttgtgctgtgatcaaaaaacatgagcagcat

SEQ ID NO: 11 - DNA, 62 bp, synthetic construct
  source 1..62: mol_type = other DNA
  modified_base 60..61: mod_base = OTHER; note = phosphorothioated base
atctcatccctgcgtgtctccgactgcacttagatatcatgatcaaaaaacatgagcagcat

SEQ ID NO: 12 - DNA, 64 bp, synthetic construct
  source 1..64: mol_type = other DNA
  modified_base 62..63: mod_base = OTHER; note = phosphorothioated base
atctcatccctgcgtgtctccgactgcacatcctgtgcgcatgatcaaaaaacatgagcagcat

SEQ ID NO: 13 - DNA, 62 bp, synthetic construct
  source 1..62: mol_type = other DNA
  modified_base 60..61: mod_base = OTHER; note = phosphorothioated base
atctcatccctgcgtgtctccgactgcacatgcgtcctgtgatcaaaaaacatgagcagcat

SEQ ID NO: 14 - DNA, 64 bp, synthetic construct
  source 1..64: mol_type = other DNA
  modified_base 62..63: mod_base = OTHER; note = phosphorothioated base
atctcatccctgcgtgtctccgactgcacaatgctctgcatagatcaaaaaacatgagcagcat

SEQ ID NO: 15 - DNA, 62 bp, synthetic construct
  source 1..62: mol_type = other DNA
  modified_base 60..61: mod_base = OTHER; note = phosphorothioated base
atctcatccctgcgtgtctccgactgcactacattgcacagatcaaaaaacatgagcagcat

SEQ ID NO: 16 - DNA, 62 bp, synthetic construct
  source 1..62: mol_type = other DNA
  modified_base 60..61: mod_base = OTHER; note = phosphorothioated base
atctcatccctgcgtgtctccgactgcacatagcgagccagatcaaaaaacatgagcagcat

SEQ ID NO: 17 - DNA, 62 bp, synthetic construct
  source 1..62: mol_type = other DNA
  modified_base 60..61: mod_base = OTHER; note = phosphorothioated base
atctcatccctgcgtgtctccgactgcactctgtattgcagatcaaaaaacatgagcagcat

SEQ ID NO: 18 - DNA, 62 bp, synthetic construct
  source 1..62: mol_type = other DNA
  modified_base 60..61: mod_base = OTHER; note = phosphorothioated base
atctcatccctgcgtgtctccgactgcacatcatcgattcgatcaaaaaacatgagcagcat

SEQ ID NO: 19 - DNA, 62 bp, synthetic construct
  source 1..62: mol_type = other DNA
  modified_base 60..61: mod_base = OTHER; note = phosphorothioated base
atctcatccctgcgtgtctccgactgcactgtgaatatgcgatcaaaaaacatgagcagcat

SEQ ID NO: 20 - DNA, 64 bp, synthetic construct
  source 1..64: mol_type = other DNA
  modified_base 62..63: mod_base = OTHER; note = phosphorothioated base
atctcatccctgcgtgtctccgactgcacagaagctgcatgagatcaaaaaacatgagcagcat

SEQ ID NO: 21 - DNA, 62 bp, synthetic construct
  source 1..62: mol_type = other DNA
  modified_base 60..61: mod_base = OTHER; note = phosphorothioated base
atctcatccctgcgtgtctccgactgcacgcatcctcacagatcaaaaaacatgagcagcat

SEQ ID NO: 22 - DNA, 62 bp, synthetic construct
  source 1..62: mol_type = other DNA
  modified_base 60..61: mod_base = OTHER; note = phosphorothioated base
atctcatccctgcgtgtctccgactgcacacaacatatcagatcaaaaaacatgagcagcat

SEQ ID NO: 23 - DNA, 64 bp, synthetic construct
  source 1..64: mol_type = other DNA
  modified_base 62..63: mod_base = OTHER; note = phosphorothioated base
atctcatccctgcgtgtctccgactgcacacatcagctcaatgatcaaaaaacatgagcagcat

SEQ ID NO: 24 - DNA, 64 bp, synthetic construct
  source 1..64: mol_type = other DNA
  modified_base 62..63: mod_base = OTHER; note = phosphorothioated base
atctcatccctgcgtgtctccgactgcacatgcatataatgcgatcaaaaaacatgagcagcat

SEQ ID NO: 25 - DNA, 64 bp, synthetic construct
  source 1..64: mol_type = other DNA
  modified_base 62..63: mod_base = OTHER; note = phosphorothioated base
atctcatccctgcgtgtctccgactgcactcaatgcatcagcgatcaaaaaacatgagcagcat

SEQ ID NO: 26 - DNA, 62 bp, synthetic construct
  source 1..62: mol_type = other DNA
  modified_base 60..61: mod_base = OTHER; note = phosphorothioated base
atctcatccctgcgtgtctccgactgcacaatgtgtgctagatcaaaaaacatgagcagcat

SEQ ID NO: 27 - DNA, 64 bp, synthetic construct
  source 1..64: mol_type = other DNA
  modified_base 62..63: mod_base = OTHER; note = phosphorothioated base
atctcatccctgcgtgtctccgactgcactctcgcatgcaatgatcaaaaaacatgagcagcat

SEQ ID NO: 28 - DNA, 62 bp, synthetic construct
  source 1..62: mol_type = other DNA
  modified_base 60..61: mod_base = OTHER; note = phosphorothioated base
atctcatccctgcgtgtctccgactgcactagcagccagcgatcaaaaaacatgagcagcat

SEQ ID NO: 29 - DNA, 62 bp, synthetic construct
  source 1..62: mol_type = other DNA
  modified_base 60..61: mod_base = OTHER; note = phosphorothioated base
atctcatccctgcgtgtctccgactgcactgccagactgtgatcaaaaaacatgagcagcat

SEQ ID NO: 30 - DNA, 64 bp, synthetic construct
  source 1..64: mol_type = other DNA
  modified_base 62..63: mod_base = OTHER; note = phosphorothioated base
atctcatccctgcgtgtctccgactgcacacatggcagcacagatcaaaaaacatgagcagcat

SEQ ID NO: 31 - DNA, 62 bp, synthetic construct
  source 1..62: mol_type = other DNA
  modified_base 60..61: mod_base = OTHER; note = phosphorothioated base
atctcatccctgcgtgtctccgactgcacacttgcagatcgatcaaaaaacatgagcagcat

SEQ ID NO: 32 - DNA, 64 bp, synthetic construct
  source 1..64: mol_type = other DNA
  modified_base 62..63: mod_base = OTHER; note = phosphorothioated base
atctcatccctgcgtgtctccgactgcactatgccacagcatgatcaaaaaacatgagcagcat

SEQ ID NO: 33 - DNA, 64 bp, synthetic construct
  source 1..64: mol_type = other DNA
  modified_base 62..63: mod_base = OTHER; note = phosphorothioated base
atctcatccctgcgtgtctccgactgcacaacatcagcatgagatcaaaaaacatgagcagcat

SEQ ID NO: 34 - DNA, 64 bp, synthetic construct
  source 1..64: mol_type = other DNA
  modified_base 62..63: mod_base = OTHER; note = phosphorothioated base
atctcatccctgcgtgtctccgactgcactgcagtgattcatgatcaaaaaacatgagcagcat

SEQ ID NO: 35 - DNA, 64 bp, synthetic construct
  source 1..64: mol_type = other DNA
  modified_base 62..63: mod_base = OTHER; note = phosphorothioated base
atctcatccctgcgtgtctccgactgcacagcacctgcatcagatcaaaaaacatgagcagcat

SEQ ID NO: 36 - DNA, 62 bp, synthetic construct
  source 1..62: mol_type = other DNA
  modified_base 60..61: mod_base = OTHER; note = phosphorothioated base
atctcatccctgcgtgtctccgactgcacttatgctatcagatcaaaaaacatgagcagcat

SEQ ID NO: 37 - DNA, 64 bp, synthetic construct
  source 1..64: mol_type = other DNA
  modified_base 62..63: mod_base = OTHER; note = phosphorothioated base
atctcatccctgcgtgtctccgactgcacatctcagtgcaatgatcaaaaaacatgagcagcat

SEQ ID NO: 38 - DNA, 62 bp, synthetic construct
  source 1..62: mol_type = other DNA
  modified_base 60..61: mod_base = OTHER; note = phosphorothioated base
atctcatccctgcgtgtctccgactgcacacagtcaatgtgatcaaaaaacatgagcagcat

SEQ ID NO: 39 - DNA, 62 bp, synthetic construct
  source 1..62: mol_type = other DNA
  modified_base 60..61: mod_base = OTHER; note = phosphorothioated base
atctcatccctgcgtgtctccgactgcacatagagcctcagatcaaaaaacatgagcagcat

SEQ ID NO: 40 - DNA, 62 bp, synthetic construct
  source 1..62: mol_type = other DNA
  modified_base 60..61: mod_base = OTHER; note = phosphorothioated base
atctcatccctgcgtgtctccgactgcacttgtgtcatgagatcaaaaaacatgagcagcat

SEQ ID NO: 41 - DNA, 62 bp, synthetic construct
  source 1..62: mol_type = other DNA
  modified_base 60..61: mod_base = OTHER; note = phosphorothioated base
atctcatccctgcgtgtctccgactgcacaatgcactcgagatcaaaaaacatgagcagcat

SEQ ID NO: 42 - DNA, 62 bp, synthetic construct
  source 1..62: mol_type = other DNA
  modified_base 60..61: mod_base = OTHER; note = phosphorothioated base
atctcatccctgcgtgtctccgactgcacgtcattgcacagatcaaaaaacatgagcagcat

SEQ ID NO: 43 - DNA, 62 bp, synthetic construct
  source 1..62: mol_type = other DNA
  modified_base 60..61: mod_base = OTHER; note = phosphorothioated base
atctcatccctgcgtgtctccgactgcacatcactgcaacgatcaaaaaacatgagcagcat

SEQ ID NO: 44 - DNA, 64 bp, synthetic construct
  source 1..64: mol_type = other DNA
  modified_base 62..63: mod_base = OTHER; note = phosphorothioated base
atctcatccctgcgtgtctccgactgcacttgcatgcgatgcgatcaaaaaacatgagcagcat

SEQ ID NO: 45 - DNA, 62 bp, synthetic construct
  source 1..62: mol_type = other DNA
  modified_base 60..61: mod_base = OTHER; note = phosphorothioated base
atctcatccctgcgtgtctccgactgcacgtgcgcaagcagatcaaaaaacatgagcagcat

SEQ ID NO: 46 - DNA, 62 bp, synthetic construct
  source 1..62: mol_type = other DNA
  modified_base 60..61: mod_base = OTHER; note = phosphorothioated base
atctcatccctgcgtgtctccgactgcactatctcataatgatcaaaaaacatgagcagcat

SEQ ID NO: 47 - DNA, 64 bp, synthetic construct
  source 1..64: mol_type = other DNA
  modified_base 62..63: mod_base = OTHER; note = phosphorothioated base
atctcatccctgcgtgtctccgactgcacatggctatgcactgatcaaaaaacatgagcagcat

SEQ ID NO: 48 - DNA, 62 bp, synthetic construct
  source 1..62: mol_type = other DNA
  modified_base 60..61: mod_base = OTHER; note = phosphorothioated base
atctcatccctgcgtgtctccgactgcactatgaatgagcgatcaaaaaacatgagcagcat

SEQ ID NO: 49 - DNA, 62 bp, synthetic construct
  source 1..62: mol_type = other DNA
  modified_base 60..61: mod_base = OTHER; note = phosphorothioated base
atctcatccctgcgtgtctccgactgcactatgcaccatcgatcaaaaaacatgagcagcat

SEQ ID NO: 50 - DNA, 62 bp, synthetic construct
  source 1..62: mol_type = other DNA
  modified_base 60..61: mod_base = OTHER; note = phosphorothioated base
atctcatccctgcgtgtctccgactgcacacaatgtgcgcgatcaaaaaacatgagcagcat

SEQ ID NO: 51 - DNA, 62 bp, synthetic construct
  source 1..62: mol_type = other DNA
  modified_base 60..61: mod_base = OTHER; note = phosphorothioated base
atctcatccctgcgtgtctccgactgcacaatgactatctgatcaaaaaacatgagcagcat

SEQ ID NO: 52 - DNA, 62 bp, synthetic construct
  source 1..62: mol_type = other DNA
  modified_base 60..61: mod_base = OTHER; note = phosphorothioated base
atctcatccctgcgtgtctccgactgcacagcctcagcgtgatcaaaaaacatgagcagcat

SEQ ID NO: 53 - DNA, 64 bp, synthetic construct
  source 1..64: mol_type = other DNA
  modified_base 62..63: mod_base = OTHER; note = phosphorothioated base
atctcatccctgcgtgtctccgactgcactctgctgtgcaatgatcaaaaaacatgagcagcat

SEQ ID NO: 54 - DNA, 64 bp, synthetic construct
  source 1..64: mol_type = other DNA
  modified_base 62..63: mod_base = OTHER; note = phosphorothioated base
atctcatccctgcgtgtctccgactgcactgtgcatctgcctgatcaaaaaacatgagcagcat

SEQ ID NO: 55 - DNA, 62 bp, synthetic construct
  source 1..62: mol_type = other DNA
  modified_base 60..61: mod_base = OTHER; note = phosphorothioated base
atctcatccctgcgtgtctccgactgcacaactatctgcagatcaaaaaacatgagcagcat

SEQ ID NO: 56 - DNA, 64 bp, synthetic construct
  source 1..64: mol_type = other DNA
  modified_base 62..63: mod_base = OTHER; note = phosphorothioated base
atctcatccctgcgtgtctccgactgcacagatctcatgaatgatcaaaaaacatgagcagcat

SEQ ID NO: 57 - DNA, 62 bp, synthetic construct
  source 1..62: mol_type = other DNA
  modified_base 60..61: mod_base = OTHER; note = phosphorothioated base
atctcatccctgcgtgtctccgactgcactatcatccagtgatcaaaaaacatgagcagcat

SEQ ID NO: 58 - DNA, 62 bp, synthetic construct
  source 1..62: mol_type = other DNA
  modified_base 60..61: mod_base = OTHER; note = phosphorothioated base
atctcatccctgcgtgtctccgactgcactgctacaagcagatcaaaaaacatgagcagcat

SEQ ID NO: 59 - DNA, 62 bp, synthetic construct
  source 1..62: mol_type = other DNA
  modified_base 60..61: mod_base = OTHER; note = phosphorothioated base
atctcatccctgcgtgtctccgactgcacaatatgcacgtgatcaaaaaacatgagcagcat

SEQ ID NO: 60 - DNA, 62 bp, synthetic construct
  source 1..62: mol_type = other DNA
  modified_base 60..61: mod_base = OTHER; note = phosphorothioated base
atctcatccctgcgtgtctccgactgcacacttctgcgatgatcaaaaaacatgagcagcat

SEQ ID NO: 61 - DNA, 62 bp, synthetic construct
  source 1..62: mol_type = other DNA
  modified_base 60..61: mod_base = OTHER; note = phosphorothioated base
atctcatccctgcgtgtctccgactgcacaagcatatctagatcaaaaaacatgagcagcat

SEQ ID NO: 62 - DNA, 62 bp, synthetic construct
  source 1..62: mol_type = other DNA
  modified_base 60..61: mod_base = OTHER; note = phosphorothioated base
atctcatccctgcgtgtctccgactgcacaatgacagctcgatcaaaaaacatgagcagcat

SEQ ID NO: 63 - DNA, 62 bp, synthetic construct
  source 1..62: mol_type = other DNA
  modified_base 60..61: mod_base = OTHER; note = phosphorothioated base
atctcatccctgcgtgtctccgactgcacatatgacctgagatcaaaaaacatgagcagcat

SEQ ID NO: 64 - DNA, 62 bp, synthetic construct
  source 1..62: mol_type = other DNA
  modified_base 60..61: mod_base = OTHER; note = phosphorothioated base
atctcatccctgcgtgtctccgactgcacagccgatatcagatcaaaaaacatgagcagcat

SEQ ID NO: 65 - DNA, 62 bp, synthetic construct
  source 1..62: mol_type = other DNA
  modified_base 60..61: mod_base = OTHER; note = phosphorothioated base
atctcatccctgcgtgtctccgactgcacagtcagttgcagatcaaaaaacatgagcagcat

SEQ ID NO: 66 - DNA, 64 bp, synthetic construct
  source 1..64: mol_type = other DNA
  modified_base 62..63: mod_base = OTHER; note = phosphorothioated base
atctcatccctgcgtgtctccgactgcactcatctgcgcaatgatcaaaaaacatgagcagcat

SEQ ID NO: 67 - DNA, 64 bp, synthetic construct
  source 1..64: mol_type = other DNA
  modified_base 62..63: mod_base = OTHER; note = phosphorothioated base
atctcatccctgcgtgtctccgactgcactattgcatgctctgatcaaaaaacatgagcagcat

SEQ ID NO: 68 - DNA, 62 bp, synthetic construct
  source 1..62: mol_type = other DNA
  modified_base 60..61: mod_base = OTHER; note = phosphorothioated base
atctcatccctgcgtgtctccgactgcacatataatcacagatcaaaaaacatgagcagcat

SEQ ID NO: 69 - DNA, 62 bp, synthetic construct
  source 1..62: mol_type = other DNA
  modified_base 60..61: mod_base = OTHER; note = phosphorothioated base
atctcatccctgcgtgtctccgactgcactgagaatgtgtgatcaaaaaacatgagcagcat

SEQ ID NO: 70 - DNA, 62 bp, synthetic construct
  source 1..62: mol_type = other DNA
  modified_base 60..61: mod_base = OTHER; note = phosphorothioated base
atctcatccctgcgtgtctccgactgcacatgcatggtacgatcaaaaaacatgagcagcat

SEQ ID NO: 71 - DNA, 64 bp, synthetic construct
  source 1..64: mol_type = other DNA
  modified_base 62..63: mod_base = OTHER; note = phosphorothioated base
atctcatccctgcgtgtctccgactgcacatgcatgcgaggagatcaaaaaacatgagcagcat

SEQ ID NO: 72 - DNA, 62 bp, synthetic construct
  source 1..62: mol_type = other DNA
  modified_base 60..61: mod_base = OTHER; note = phosphorothioated base
atctcatccctgcgtgtctccgactgcacaatctgcatacgatcaaaaaacatgagcagcat

SEQ ID NO: 73 - DNA, 64 bp, synthetic construct
  source 1..64: mol_type = other DNA
  modified_base 62..63: mod_base = OTHER; note = phosphorothioated base
atctcatccctgcgtgtctccgactgcacagcatgagcgccagatcaaaaaacatgagcagcat

SEQ ID NO: 74 - DNA, 62 bp, synthetic construct
  source 1..62: mol_type = other DNA
  modified_base 60..61: mod_base = OTHER; note = phosphorothioated base
atctcatccctgcgtgtctccgactgcacttatctgatctgatcaaaaaacatgagcagcat

SEQ ID NO: 75 - DNA, 64 bp, synthetic construct
  source 1..64: mol_type = other DNA
  modified_base 62..63: mod_base = OTHER; note = phosphorothioated base
atctcatccctgcgtgtctccgactgcacatccagcgcatgtgatcaaaaaacatgagcagcat

SEQ ID NO: 76 - DNA, 62 bp, synthetic construct
  source 1..62: mol_type = other DNA
  modified_base 60..61: mod_base = OTHER; note = phosphorothioated base
atctcatccctgcgtgtctccgactgcacagttcatctgtgatcaaaaaacatgagcagcat

SEQ ID NO: 77 - DNA, 62 bp, synthetic construct
  source 1..62: mol_type = other DNA
  modified_base 60..61: mod_base = OTHER; note = phosphorothioated base
atctcatccctgcgtgtctccgactgcacaacatacatcagatcaaaaaacatgagcagcat

SEQ ID NO: 78 - DNA, 62 bp, synthetic construct
  source 1..62: mol_type = other DNA
  modified_base 60..61: mod_base = OTHER; note = phosphorothioated base
atctcatccctgcgtgtctccgactgcacggctagatgcagatcaaaaaacatgagcagcat

SEQ ID NO: 79 - DNA, 64 bp, synthetic construct
  source 1..64: mol_type = other DNA
  modified_base 62..63: mod_base = OTHER; note = phosphorothioated base
atctcatccctgcgtgtctccgactgcactgccgagcagcatgatcaaaaaacatgagcagcat

SEQ ID NO: 80 - DNA, 64 bp, synthetic construct
  source 1..64: mol_type = other DNA
  modified_base 62..63: mod_base = OTHER; note = phosphorothioated base
atctcatccctgcgtgtctccgactgcactgcctcagatcatgatcaaaaaacatgagcagcat

SEQ ID NO: 81 - DNA, 62 bp, synthetic construct
  source 1..62: mol_type = other DNA
  modified_base 60..61: mod_base = OTHER; note = phosphorothioated base
atctcatccctgcgtgtctccgactgcactgatcagtggcgatcaaaaaacatgagcagcat

SEQ ID NO: 82 - DNA, 64 bp, synthetic construct
  source 1..64: mol_type = other DNA
  modified_base 62..63: mod_base = OTHER; note = phosphorothioated base
atctcatccctgcgtgtctccgactgcacacatatggcatatgatcaaaaaacatgagcagcat

SEQ ID NO: 83 - DNA, 62 bp, synthetic construct
  source 1..62: mol_type = other DNA
  modified_base 60..61: mod_base = OTHER; note = phosphorothioated base
atctcatccctgcgtgtctccgactgcacagatcgccacagatcaaaaaacatgagcagcat

SEQ ID NO: 84 - DNA, 62 bp, synthetic construct
  source 1..62: mol_type = other DNA
  modified_base 60..61: mod_base = OTHER; note = phosphorothioated base
atctcatccctgcgtgtctccgactgcacttgatgatagtgatcaaaaaacatgagcagcat

SEQ ID NO: 85 - DNA, 64 bp, synthetic construct
  source 1..64: mol_type = other DNA
  modified_base 62..63: mod_base = OTHER; note = phosphorothioated base
atctcatccctgcgtgtctccgactgcacatctctggctgcagatcaaaaaacatgagcagcat

SEQ ID NO: 86 - DNA, 64 bp, synthetic construct
  source 1..64: mol_type = other DNA
  modified_base 62..63: mod_base = OTHER; note = phosphorothioated base
atctcatccctgcgtgtctccgactgcacatctggtgcatgtgatcaaaaaacatgagcagcat

SEQ ID NO: 87 - DNA, 62 bp, synthetic construct
  source 1..62: mol_type = other DNA
  modified_base 60..61: mod_base = OTHER; note = phosphorothioated base
atctcatccctgcgtgtctccgactgcacagcagcttcgcgatcaaaaaacatgagcagcat

SEQ ID NO: 88 - DNA, 64 bp, synthetic construct
  source 1..64: mol_type = other DNA
  modified_base 62..63: mod_base = OTHER; note = phosphorothioated base
atctcatccctgcgtgtctccgactgcacgcatatggcagcagatcaaaaaacatgagcagcat

SEQ ID NO: 89 - DNA, 64 bp, synthetic construct
  source 1..64: mol_type = other DNA
  modified_base 62..63: mod_base = OTHER; note = phosphorothioated base
atctcatccctgcgtgtctccgactgcacatgcagatggcgagatcaaaaaacatgagcagcat

SEQ ID NO: 90 - DNA, 64 bp, synthetic construct
  source 1..64: mol_type = other DNA
  modified_base 62..63: mod_base = OTHER; note = phosphorothioated base
atctcatccctgcgtgtctccgactgcacttcatgcatctcagatcaaaaaacatgagcagcat

SEQ ID NO: 91 - DNA, 64 bp, synthetic construct
  source 1..64: mol_type = other DNA
  modified_base 62..63: mod_base = OTHER; note = phosphorothioated base
atctcatccctgcgtgtctccgactgcacatgcaagtgtgatgatcaaaaaacatgagcagcat

SEQ ID NO: 92 - DNA, 62 bp, synthetic construct
  source 1..62: mol_type = other DNA
  modified_base 60..61: mod_base = OTHER; note = phosphorothioated base
atctcatccctgcgtgtctccgactgcactgttcgctgcagatcaaaaaacatgagcagcat

SEQ ID NO: 93 - DNA, 64 bp, synthetic construct
  source 1..64: mol_type = other DNA
  modified_base 62..63: mod_base = OTHER; note = phosphorothioated base
atctcatccctgcgtgtctccgactgcactcagatcctgcatgatcaaaaaacatgagcagcat

SEQ ID NO: 94 - DNA, 62 bp, synthetic construct
  source 1..62: mol_type = other DNA
  modified_base 60..61: mod_base = OTHER; note = phosphorothioated base
atctcatccctgcgtgtctccgactgcacacacagataatgatcaaaaaacatgagcagcat

SEQ ID NO: 95 - DNA, 62 bp, synthetic construct
  source 1..62: mol_type = other DNA
  modified_base 60..61: mod_base = OTHER; note = phosphorothioated base
atctcatccctgcgtgtctccgactgcacgatgctctggcgatcaaaaaacatgagcagcat

SEQ ID NO: 96 - DNA, 64 bp, synthetic construct
  source 1..64: mol_type = other DNA
  modified_base 62..63: mod_base = OTHER; note = phosphorothioated base
atctcatccctgcgtgtctccgactgcacagatccatcatctgatcaaaaaacatgagcagcat

SEQ ID NO: 97 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgcattcgagctgtgcagtcggagacacgcagggatgaganggn

SEQ ID NO: 98 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcattcatgtgtgatgtgcagtcggagacacgcagggatgaganggn

SEQ ID NO: 99 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcatgcctacacagtgcagtcggagacacgcagggatgaganggn

SEQ ID NO: 100 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcagaggatacatgtgcagtcggagacacgcagggatgaganggn

SEQ ID NO: 101 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcataggctatatgtgcagtcggagacacgcagggatgaganggn

SEQ ID NO: 102 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgagcatgaatcgtgcagtcggagacacgcagggatgaganggn

SEQ ID NO: 103 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcacatgcaggatgtgtgcagtcggagacacgcagggatgaganggn

SEQ ID NO: 104 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcattcgtgcagtgtgcagtcggagacacgcagggatgaganggn

SEQ ID NO: 105 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgtgctatggcagtgcagtcggagacacgcagggatgaganggn

SEQ ID NO: 106 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcacagcacaactgtgcagtcggagacacgcagggatgaganggn

SEQ ID NO: 107 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcatgatatctaagtgcagtcggagacacgcagggatgaganggn

SEQ ID NO: 108 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcatgcgcacaggatgtgcagtcggagacacgcagggatgaganggn

SEQ ID NO: 109 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcacaggacgcatgtgcagtcggagacacgcagggatgaganggn

SEQ ID NO: 110 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatctatgcagagcattgtgcagtcggagacacgcagggatgaganggn

SEQ ID NO: 111 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgtgcaatgtagtgcagtcggagacacgcagggatgaganggn

SEQ ID NO: 112 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctggctcgctatgtgcagtcggagacacgcagggatgaganggn

SEQ ID NO: 113 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgcaatacagagtgcagtcggagacacgcagggatgaganggn

SEQ ID NO: 114 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgaatcgatgatgtgcagtcggagacacgcagggatgaganggn

SEQ ID NO: 115 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgcatattcacagtgcagtcggagacacgcagggatgaganggn

SEQ ID NO: 116 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatctcatgcagcttctgtgcagtcggagacacgcagggatgaganggn

SEQ ID NO: 117 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgtgaggatgcgtgcagtcggagacacgcagggatgaganggn

SEQ ID NO: 118 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgatatgttgtgtgcagtcggagacacgcagggatgaganggn

SEQ ID NO: 119 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcattgagctgatgtgtgcagtcggagacacgcagggatgaganggn

SEQ ID NO: 120 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcgcattatatgcatgtgcagtcggagacacgcagggatgaganggn

SEQ ID NO: 121 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcgctgatgcattgagtgcagtcggagacacgcagggatgaganggn

SEQ ID NO: 122 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctagcacacattgtgcagtcggagacacgcagggatgaganggn

SEQ ID NO: 123 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcattgcatgcgagagtgcagtcggagacacgcagggatgaganggn

SEQ ID NO: 124 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgctggctgctagtgcagtcggagacacgcagggatgaganggn

SEQ ID NO: 125 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcacagtctggcagtgcagtcggagacacgcagggatgaganggn

SEQ ID NO: 126 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatctgtgctgccatgtgtgcagtcggagacacgcagggatgaganggn

SEQ ID NO: 127 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgatctgcaagtgtgcagtcggagacacgcagggatgaganggn

SEQ ID NO: 128 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcatgctgtggcatagtgcagtcggagacacgcagggatgaganggn

SEQ ID NO: 129 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatctcatgctgatgttgtgcagtcggagacacgcagggatgaganggn

SEQ ID NO: 130 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcatgaatcactgcagtgcagtcggagacacgcagggatgaganggn

SEQ ID NO: 131 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatctgatgcaggtgctgtgcagtcggagacacgcagggatgaganggn

SEQ ID NO: 132 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgatagcataagtgcagtcggagacacgcagggatgaganggn

SEQ ID NO: 133 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcattgcactgagatgtgcagtcggagacacgcagggatgaganggn

SEQ ID NO: 134 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcacattgactgtgtgcagtcggagacacgcagggatgaganggn

SEQ ID NO: 135 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgaggctctatgtgcagtcggagacacgcagggatgaganggn

SEQ ID NO: 136 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctcatgacacaagtgcagtcggagacacgcagggatgaganggn

SEQ ID NO: 137 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctcgagtgcattgtgcagtcggagacacgcagggatgaganggn

SEQ ID NO: 138 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgtgcaatgacgtgcagtcggagacacgcagggatgaganggn

SEQ ID NO: 139 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgttgcagtgatgtgcagtcggagacacgcagggatgaganggn

SEQ ID NO: 140 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcgcatcgcatgcaagtgcagtcggagacacgcagggatgaganggn

SEQ ID NO: 141 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgcttgcgcacgtgcagtcggagacacgcagggatgaganggn

SEQ ID NO: 142 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcattatgagatagtgcagtcggagacacgcagggatgaganggn

SEQ ID NO: 143 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcagtgcatagccatgtgcagtcggagacacgcagggatgaganggn

SEQ ID NO: 144 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgctcattcatagtgcagtcggagacacgcagggatgaganggn

SEQ ID NO: 145 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgatggtgcatagtgcagtcggagacacgcagggatgaganggn

SEQ ID NO: 146 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgcgcacattgtgtgcagtcggagacacgcagggatgaganggn

SEQ ID NO: 147 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcagatagtcattgtgcagtcggagacacgcagggatgaganggn

SEQ ID NO: 148 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcacgctgaggctgtgcagtcggagacacgcagggatgaganggn

SEQ ID NO: 149 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcattgcacagcagagtgcagtcggagacacgcagggatgaganggn

SEQ ID NO: 150 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcaggcagatgcacagtgcagtcggagacacgcagggatgaganggn

SEQ ID NO: 151 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgcagatagttgtgcagtcggagacacgcagggatgaganggn

SEQ ID NO: 152 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcattcatgagatctgtgcagtcggagacacgcagggatgaganggn

SEQ ID NO: 153 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcactggatgatagtgcagtcggagacacgcagggatgaganggn

SEQ ID NO: 154 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgcttgtagcagtgcagtcggagacacgcagggatgaganggn

SEQ ID NO: 155 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcacgtgcatattgtgcagtcggagacacgcagggatgaganggn

SEQ ID NO: 156 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcatcgcagaagtgtgcagtcggagacacgcagggatgaganggn

SEQ ID NO: 157 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctagatatgcttgtgcagtcggagacacgcagggatgaganggn

SEQ ID NO: 158 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgagctgtcattgtgcagtcggagacacgcagggatgaganggn

SEQ ID NO: 159 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctcaggtcatatgtgcagtcggagacacgcagggatgaganggn

SEQ ID NO: 160 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgatatcggctgtgcagtcggagacacgcagggatgaganggn

SEQ ID NO: 161 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgcaactgactgtgcagtcggagacacgcagggatgaganggn

SEQ ID NO: 162 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcattgcgcagatgagtgcagtcggagacacgcagggatgaganggn

SEQ ID NO: 163 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcagagcatgcaatagtgcagtcggagacacgcagggatgaganggn

SEQ ID NO: 164 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgtgattatatgtgcagtcggagacacgcagggatgaganggn

SEQ ID NO: 165 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcacacattctcagtgcagtcggagacacgcagggatgaganggn

SEQ ID NO: 166 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgtaccatgcatgtgcagtcggagacacgcagggatgaganggn

SEQ ID NO: 167 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatctcctcgcatgcatgtgcagtcggagacacgcagggatgaganggn

SEQ ID NO: 168 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgtatgcagattgtgcagtcggagacacgcagggatgaganggn

SEQ ID NO: 169 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatctggcgctcatgctgtgcagtcggagacacgcagggatgaganggn

SEQ ID NO: 170 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcagatcagataagtgcagtcggagacacgcagggatgaganggn

SEQ ID NO: 171 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcacatgcgctggatgtgcagtcggagacacgcagggatgaganggn

SEQ ID NO: 172 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcacagatgaactgtgcagtcggagacacgcagggatgaganggn

SEQ ID NO: 173 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgatgtatgttgtgcagtcggagacacgcagggatgaganggn

SEQ ID NO: 174 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgcatctagccgtgcagtcggagacacgcagggatgaganggn

SEQ ID NO: 175 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcatgctgctcggcagtgcagtcggagacacgcagggatgaganggn

SEQ ID NO: 176 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcatgatctgaggcagtgcagtcggagacacgcagggatgaganggn

SEQ ID NO: 177 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgccactgatcagtgcagtcggagacacgcagggatgaganggn

SEQ ID NO: 178 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcatatgccatatgtgtgcagtcggagacacgcagggatgaganggn

SEQ ID NO: 179 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgtggcgatctgtgcagtcggagacacgcagggatgaganggn

SEQ ID NO: 180 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcactatcatcaagtgcagtcggagacacgcagggatgaganggn

SEQ ID NO: 181 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatctgcagccagagatgtgcagtcggagacacgcagggatgaganggn

SEQ ID NO: 182 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcacatgcaccagatgtgcagtcggagacacgcagggatgaganggn

SEQ ID NO: 183 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgcgaagctgctgtgcagtcggagacacgcagggatgaganggn

SEQ ID NO: 184 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatctgctgccatatgcgtgcagtcggagacacgcagggatgaganggn

SEQ ID NO: 185 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatctcgccatctgcatgtgcagtcggagacacgcagggatgaganggn

SEQ ID NO: 186 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatctgagatgcatgaagtgcagtcggagacacgcagggatgaganggn

SEQ ID NO: 187 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcatcacacttgcatgtgcagtcggagacacgcagggatgaganggn

SEQ ID NO: 188 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatctgcagcgaacagtgcagtcggagacacgcagggatgaganggn

SEQ ID NO: 189 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcatgcaggatctgagtgcagtcggagacacgcagggatgaganggn

SEQ ID NO: 190 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcattatctgtgtgtgcagtcggagacacgcagggatgaganggn

SEQ ID NO: 191 - DNA, 64 bp, synthetic construct
  source 1..64: mol_type = other DNA
  misc_feature 1: note = 5' phosphorylation
  modified_base 61: mod_base = OTHER; note = deoxyuridine
  modified_base 64: mod_base = OTHER; note = deoxyuridine
  misc_binding 64: bound_moiety = 3' biotin- triethylene glycol (TEG) spacer arm
tgctgctcatgaaaaaagatcgccagagcatcgtgcagtcggagacacgcagggatgaganggn

SEQ ID NO: 192 - DNA, 66 bp, synthetic construct
  source 1..66: mol_type = other DNA
  misc_feature 1: note = 5' phosphorylation
  modified_base 63: mod_base = OTHER; note = deoxyuridine
  modified_base 66: mod_base = OTHER; note = deoxyuridine
  misc_binding 66: bound_moiety = 3' biotin- triethylene glycol (TEG) spacer arm
tgctgctcatgaaaaaagatcagatgatggatctgtgcagtcggagacacgcagggatgaganggn

SEQ ID NO: 193 - DNA, 63 bp, synthetic construct
  source 1..63: mol_type = other DNA
  modified_base 61..62: mod_base = OTHER; note = phosphorothioated base
atctcatccctgcgtgtctccgactgcactgcaccatcatatgatcaaggacatgagcagcat

SEQ ID NO: 194 - DNA, 63 bp, synthetic construct
  source 1..63: mol_type = other DNA
  modified_base 61..62: mod_base = OTHER; note = phosphorothioated base
atctcatccctgcgtgtctccgactgcacatgctatagcaatgatcaaggacatgagcagcat

SEQ ID NO: 195 - DNA, 63 bp, synthetic construct
  source 1..63: mol_type = other DNA
  modified_base 61..62: mod_base = OTHER; note = phosphorothioated base
atctcatccctgcgtgtctccgactgcacatctgcacatggcgatcaaggacatgagcagcat

SEQ ID NO: 196 - DNA, 63 bp, synthetic construct
  source 1..63: mol_type = other DNA
  modified_base 61..62: mod_base = OTHER; note = phosphorothioated base
atctcatccctgcgtgtctccgactgcactattcagatgcatgatcaaggacatgagcagcat

SEQ ID NO: 197 - DNA, 61 bp, synthetic construct
  source 1..61: mol_type = other DNA
  modified_base 59..60: mod_base = OTHER; note = phosphorothioated base
atctcatccctgcgtgtctccgactgcacatgcaacactagatcaaggacatgagcagcat

SEQ ID NO: 198 - DNA, 61 bp, synthetic construct
  source 1..61: mol_type = other DNA
  modified_base 59..60: mod_base = OTHER; note = phosphorothioated base
atctcatccctgcgtgtctccgactgcactattatgatatgatcaaggacatgagcagcat

SEQ ID NO: 199 - DNA, 61 bp, synthetic construct
  source 1..61: mol_type = other DNA
  modified_base 59..60: mod_base = OTHER; note = phosphorothioated base
atctcatccctgcgtgtctccgactgcactcagaagcatcgatcaaggacatgagcagcat

SEQ ID NO: 200 - DNA, 61 bp, synthetic construct
  source 1..61: mol_type = other DNA
  modified_base 59..60: mod_base = OTHER; note = phosphorothioated base
atctcatccctgcgtgtctccgactgcacttcatatctgagatcaaggacatgagcagcat

SEQ ID NO: 201 - DNA, 61 bp, synthetic construct
  source 1..61: mol_type = other DNA
  modified_base 59..60: mod_base = OTHER; note = phosphorothioated base
atctcatccctgcgtgtctccgactgcacaatagctatgagatcaaggacatgagcagcat

SEQ ID NO: 202 - DNA, 63 bp, synthetic construct
  source 1..63: mol_type = other DNA
  modified_base 61..62: mod_base = OTHER; note = phosphorothioated base
atctcatccctgcgtgtctccgactgcacatggatagctgcagatcaaggacatgagcagcat

SEQ ID NO: 203 - DNA, 61 bp, synthetic construct
  source 1..61: mol_type = other DNA
  modified_base 59..60: mod_base = OTHER; note = phosphorothioated base
atctcatccctgcgtgtctccgactgcacataatatctgcgatcaaggacatgagcagcat

SEQ ID NO: 204 - DNA, 61 bp, synthetic construct
  source 1..61: mol_type = other DNA
  modified_base 59..60: mod_base = OTHER; note = phosphorothioated base
atctcatccctgcgtgtctccgactgcacagcacagaggagatcaaggacatgagcagcat

SEQ ID NO: 205 - DNA, 61 bp, synthetic construct
  source 1..61: mol_type = other DNA
  modified_base 59..60: mod_base = OTHER; note = phosphorothioated base
atctcatccctgcgtgtctccgactgcacaactgcgctctgatcaaggacatgagcagcat

SEQ ID NO: 206 - DNA, 61 bp, synthetic construct
  source 1..61: mol_type = other DNA
  modified_base 59..60: mod_base = OTHER; note = phosphorothioated base
atctcatccctgcgtgtctccgactgcacacaagatgacagatcaaggacatgagcagcat

SEQ ID NO: 207 - DNA, 61 bp, synthetic construct
  source 1..61: mol_type = other DNA
  modified_base 59..60: mod_base = OTHER; note = phosphorothioated base
atctcatccctgcgtgtctccgactgcacagatgacattagatcaaggacatgagcagcat

SEQ ID NO: 208 - DNA, 63 bp, synthetic construct
  source 1..63: mol_type = other DNA
  modified_base 61..62: mod_base = OTHER; note = phosphorothioated base
atctcatccctgcgtgtctccgactgcacatggctcacatatgatcaaggacatgagcagcat

SEQ ID NO: 209 - DNA, 63 bp, synthetic construct
  source 1..63: mol_type = other DNA
  modified_base 61..62: mod_base = OTHER; note = phosphorothioated base
atctcatccctgcgtgtctccgactgcacaagagatgcatctgatcaaggacatgagcagcat

SEQ ID NO: 210 - DNA, 61 bp, synthetic construct
  source 1..61: mol_type = other DNA
  modified_base 59..60: mod_base = OTHER; note = phosphorothioated base
atctcatccctgcgtgtctccgactgcacttagctgtgatgatcaaggacatgagcagcat

SEQ ID NO: 211 - DNA, 61 bp, synthetic construct
  source 1..61: mol_type = other DNA
  modified_base 59..60: mod_base = OTHER; note = phosphorothioated base
atctcatccctgcgtgtctccgactgcactcgcaatagatgatcaaggacatgagcagcat

SEQ ID NO: 212 - DNA, 61 bp, synthetic construct
  source 1..61: mol_type = other DNA
  modified_base 59..60: mod_base = OTHER; note = phosphorothioated base
atctcatccctgcgtgtctccgactgcacactctctcaatgatcaaggacatgagcagcat

SEQ ID NO: 213 - DNA, 63 bp, synthetic construct
  source 1..63: mol_type = other DNA
  modified_base 61..62: mod_base = OTHER; note = phosphorothioated base
atctcatccctgcgtgtctccgactgcacactgcctgatgatgatcaaggacatgagcagcat

SEQ ID NO: 214 - DNA, 61 bp, synthetic construct
  source 1..61: mol_type = other DNA
  modified_base 59..60: mod_base = OTHER; note = phosphorothioated base
atctcatccctgcgtgtctccgactgcactctattcatatgatcaaggacatgagcagcat

SEQ ID NO: 215 - DNA, 61 bp, synthetic construct
  source 1..61: mol_type = other DNA
  modified_base 59..60: mod_base = OTHER; note = phosphorothioated base
atctcatccctgcgtgtctccgactgcacttgagcgcactgatcaaggacatgagcagcat

SEQ ID NO: 216 - DNA, 61 bp, synthetic construct
  source 1..61: mol_type = other DNA
  modified_base 59..60: mod_base = OTHER; note = phosphorothioated base
atctcatccctgcgtgtctccgactgcactcaacatatctgatcaaggacatgagcagcat

SEQ ID NO: 217 - DNA, 63 bp, synthetic construct
  source 1..63: mol_type = other DNA
  modified_base 61..62: mod_base = OTHER; note = phosphorothioated base
atctcatccctgcgtgtctccgactgcacatgcggcgcagcagatcaaggacatgagcagcat

SEQ ID NO: 218 - DNA, 61 bp, synthetic construct
  source 1..61: mol_type = other DNA
  modified_base 59..60: mod_base = OTHER; note = phosphorothioated base
atctcatccctgcgtgtctccgactgcacttgctgtgcgcgatcaaggacatgagcagcat

SEQ ID NO: 219 - DNA, 61 bp, synthetic construct
  source 1..61: mol_type = other DNA
  modified_base 59..60: mod_base = OTHER; note = phosphorothioated base
atctcatccctgcgtgtctccgactgcacatagcgttcatgatcaaggacatgagcagcat

SEQ ID NO: 220 - DNA, 63 bp, synthetic construct
  source 1..63: mol_type = other DNA
  modified_base 61..62: mod_base = OTHER; note = phosphorothioated base
atctcatccctgcgtgtctccgactgcacagccatctgctgagatcaaggacatgagcagcat

SEQ ID NO: 221 - DNA, 61 bp, synthetic construct
  source 1..61: mol_type = other DNA
  modified_base 59..60: mod_base = OTHER; note = phosphorothioated base
atctcatccctgcgtgtctccgactgcacactcagcgccagatcaaggacatgagcagcat

SEQ ID NO: 222 - DNA, 61 bp, synthetic construct
  source 1..61: mol_type = other DNA
  modified_base 59..60: mod_base = OTHER; note = phosphorothioated base
atctcatccctgcgtgtctccgactgcacaacatctgatagatcaaggacatgagcagcat

SEQ ID NO: 223 - DNA, 61 bp, synthetic construct
  source 1..61: mol_type = other DNA
  modified_base 59..60: mod_base = OTHER; note = phosphorothioated base
atctcatccctgcgtgtctccgactgcactgctccgcatcgatcaaggacatgagcagcat

SEQ ID NO: 224 - DNA, 61 bp, synthetic construct
  source 1..61: mol_type = other DNA
  modified_base 59..60: mod_base = OTHER; note = phosphorothioated base
atctcatccctgcgtgtctccgactgcactatcgcttgatgatcaaggacatgagcagcat

SEQ ID NO: 225 - DNA, 61 bp, synthetic construct
  source 1..61: mol_type = other DNA
  modified_base 59..60: mod_base = OTHER; note = phosphorothioated base
atctcatccctgcgtgtctccgactgcactcttctcagcagatcaaggacatgagcagcat

SEQ ID NO: 226 - DNA, 61 bp, synthetic construct
  source 1..61: mol_type = other DNA
  modified_base 59..60: mod_base = OTHER; note = phosphorothioated base
atctcatccctgcgtgtctccgactgcacatggtgtgcacgatcaaggacatgagcagcat

SEQ ID NO: 227 - DNA, 61 bp, synthetic construct
  source 1..61: mol_type = other DNA
  modified_base 59..60: mod_base = OTHER; note = phosphorothioated base
atctcatccctgcgtgtctccgactgcacatatcatcttagatcaaggacatgagcagcat

SEQ ID NO: 228 - DNA, 63 bp, synthetic construct
  source 1..63: mol_type = other DNA
  modified_base 61..62: mod_base = OTHER; note = phosphorothioated base
atctcatccctgcgtgtctccgactgcacatgatcacacaatgatcaaggacatgagcagcat

SEQ ID NO: 229 - DNA, 61 bp, synthetic construct
  source 1..61: mol_type = other DNA
  modified_base 59..60: mod_base = OTHER; note = phosphorothioated base
atctcatccctgcgtgtctccgactgcactgcacattgtagatcaaggacatgagcagcat

SEQ ID NO: 230 - DNA, 61 bp, synthetic construct
  source 1..61: mol_type = other DNA
  modified_base 59..60: mod_base = OTHER; note = phosphorothioated base
atctcatccctgcgtgtctccgactgcactcatggtatatgatcaaggacatgagcagcat

SEQ ID NO: 231 - DNA, 63 bp, synthetic construct
  source 1..63: mol_type = other DNA
  modified_base 61..62: mod_base = OTHER; note = phosphorothioated base
atctcatccctgcgtgtctccgactgcactgcatgctggcgtgatcaaggacatgagcagcat

SEQ ID NO: 232 - DNA, 63 bp, synthetic construct
  source 1..63: mol_type = other DNA
  modified_base 61..62: mod_base = OTHER; note = phosphorothioated base
atctcatccctgcgtgtctccgactgcactcttcatgagcatgatcaaggacatgagcagcat

SEQ ID NO: 233 - DNA, 63 bp, synthetic construct
  source 1..63: mol_type = other DNA
  modified_base 61..62: mod_base = OTHER; note = phosphorothioated base
atctcatccctgcgtgtctccgactgcactcatggagcatatgatcaaggacatgagcagcat

SEQ ID NO: 234 - DNA, 61 bp, synthetic construct
  source 1..61: mol_type = other DNA
  modified_base 59..60: mod_base = OTHER; note = phosphorothioated base
atctcatccctgcgtgtctccgactgcacatactgcctatgatcaaggacatgagcagcat

SEQ ID NO: 235 - DNA, 63 bp, synthetic construct
  source 1..63: mol_type = other DNA
  modified_base 61..62: mod_base = OTHER; note = phosphorothioated base
atctcatccctgcgtgtctccgactgcacaatgcatctatatgatcaaggacatgagcagcat

SEQ ID NO: 236 - DNA, 63 bp, synthetic construct
  source 1..63: mol_type = other DNA
  modified_base 61..62: mod_base = OTHER; note = phosphorothioated base
atctcatccctgcgtgtctccgactgcacgagcacaatgcatgatcaaggacatgagcagcat

SEQ ID NO: 237 - DNA, 63 bp, synthetic construct
  source 1..63: mol_type = other DNA
  modified_base 61..62: mod_base = OTHER; note = phosphorothioated base
atctcatccctgcgtgtctccgactgcacaacatgcacatctgatcaaggacatgagcagcat

SEQ ID NO: 238 - DNA, 63 bp, synthetic construct
  source 1..63: mol_type = other DNA
  modified_base 61..62: mod_base = OTHER; note = phosphorothioated base
atctcatccctgcgtgtctccgactgcacgtgcatggcatctgatcaaggacatgagcagcat

SEQ ID NO: 239 - DNA, 61 bp, synthetic construct
  source 1..61: mol_type = other DNA
  modified_base 59..60: mod_base = OTHER; note = phosphorothioated base
atctcatccctgcgtgtctccgactgcactacagcaatgtgatcaaggacatgagcagcat

SEQ ID NO: 240 - DNA, 61 bp, synthetic construct
  source 1..61: mol_type = other DNA
  modified_base 59..60: mod_base = OTHER; note = phosphorothioated base
atctcatccctgcgtgtctccgactgcacatcagtcctgcgatcaaggacatgagcagcat

SEQ ID NO: 241 - DNA, 61 bp, synthetic construct
  source 1..61: mol_type = other DNA
  modified_base 59..60: mod_base = OTHER; note = phosphorothioated base
atctcatccctgcgtgtctccgactgcacaagcgacatgcgatcaaggacatgagcagcat

SEQ ID NO: 242 - DNA, 63 bp, synthetic construct
  source 1..63: mol_type = other DNA
  modified_base 61..62: mod_base = OTHER; note = phosphorothioated base
atctcatccctgcgtgtctccgactgcacatgctcaatatctgatcaaggacatgagcagcat

SEQ ID NO: 243 - DNA, 61 bp, synthetic construct
  source 1..61: mol_type = other DNA
  modified_base 59..60: mod_base = OTHER; note = phosphorothioated base
atctcatccctgcgtgtctccgactgcacaatcgcatcgtgatcaaggacatgagcagcat

SEQ ID NO: 244 - DNA, 61 bp, synthetic construct
  source 1..61: mol_type = other DNA
  modified_base 59..60: mod_base = OTHER; note = phosphorothioated base
atctcatccctgcgtgtctccgactgcacggcactgcagtgatcaaggacatgagcagcat

SEQ ID NO: 245 - DNA, 63 bp, synthetic construct
  source 1..63: mol_type = other DNA
  modified_base 61..62: mod_base = OTHER; note = phosphorothioated base
atctcatccctgcgtgtctccgactgcactattgctctgcatgatcaaggacatgagcagcat

SEQ ID NO: 246 - DNA, 63 bp, synthetic construct
  source 1..63: mol_type = other DNA
  modified_base 61..62: mod_base = OTHER; note = phosphorothioated base
atctcatccctgcgtgtctccgactgcacatctgcggcacatgatcaaggacatgagcagcat

SEQ ID NO: 247 - DNA, 63 bp, synthetic construct
  source 1..63: mol_type = other DNA
  modified_base 61..62: mod_base = OTHER; note = phosphorothioated base
atctcatccctgcgtgtctccgactgcacatcgatgcagaatgatcaaggacatgagcagcat

SEQ ID NO: 248 - DNA, 61 bp, synthetic construct
  source 1..61: mol_type = other DNA
  modified_base 59..60: mod_base = OTHER; note = phosphorothioated base
atctcatccctgcgtgtctccgactgcacacatgaccagcgatcaaggacatgagcagcat

SEQ ID NO: 249 - DNA, 61 bp, synthetic construct
  source 1..61: mol_type = other DNA
  modified_base 59..60: mod_base = OTHER; note = phosphorothioated base
atctcatccctgcgtgtctccgactgcacaatgctgtagtgatcaaggacatgagcagcat

SEQ ID NO: 250 - DNA, 63 bp, synthetic construct
  source 1..63: mol_type = other DNA
  modified_base 61..62: mod_base = OTHER; note = phosphorothioated base
atctcatccctgcgtgtctccgactgcacatcattgatcatcgatcaaggacatgagcagcat

SEQ ID NO: 251 - DNA, 63 bp, synthetic construct
  source 1..63: mol_type = other DNA
  modified_base 61..62: mod_base = OTHER; note = phosphorothioated base
atctcatccctgcgtgtctccgactgcacactcacaatgcatgatcaaggacatgagcagcat

SEQ ID NO: 252 - DNA, 63 bp, synthetic construct
  source 1..63: mol_type = other DNA
  modified_base 61..62: mod_base = OTHER; note = phosphorothioated base
atctcatccctgcgtgtctccgactgcacatgtggctatcatgatcaaggacatgagcagcat

SEQ ID NO: 253 - DNA, 63 bp, synthetic construct
  source 1..63: mol_type = other DNA
  modified_base 61..62: mod_base = OTHER; note = phosphorothioated base
atctcatccctgcgtgtctccgactgcacatgtatgcgcaatgatcaaggacatgagcagcat

SEQ ID NO: 254 - DNA, 61 bp, synthetic construct
  source 1..61: mol_type = other DNA
  modified_base 59..60: mod_base = OTHER; note = phosphorothioated base
atctcatccctgcgtgtctccgactgcacttgcgctctcagatcaaggacatgagcagcat

SEQ ID NO: 255 - DNA, 61 bp, synthetic construct
  source 1..61: mol_type = other DNA
  modified_base 59..60: mod_base = OTHER; note = phosphorothioated base
atctcatccctgcgtgtctccgactgcacgcgcacaatatgatcaaggacatgagcagcat

SEQ ID NO: 256 - DNA, 63 bp, synthetic construct
  source 1..63: mol_type = other DNA
  modified_base 61..62: mod_base = OTHER; note = phosphorothioated base
atctcatccctgcgtgtctccgactgcacagcgcaccagcatgatcaaggacatgagcagcat

SEQ ID NO: 257 - DNA, 61 bp, synthetic construct
  source 1..61: mol_type = other DNA
  modified_base 59..60: mod_base = OTHER; note = phosphorothioated base
atctcatccctgcgtgtctccgactgcactgctatctggtgatcaaggacatgagcagcat

SEQ ID NO: 258 - DNA, 63 bp, synthetic construct
  source 1..63: mol_type = other DNA
  modified_base 61..62: mod_base = OTHER; note = phosphorothioated base
atctcatccctgcgtgtctccgactgcacatgcatgtggatagatcaaggacatgagcagcat

SEQ ID NO: 259 - DNA, 61 bp, synthetic construct
  source 1..61: mol_type = other DNA
  modified_base 59..60: mod_base = OTHER; note = phosphorothioated base
atctcatccctgcgtgtctccgactgcacatcgcaccagagatcaaggacatgagcagcat

SEQ ID NO: 260 - DNA, 63 bp, synthetic construct
  source 1..63: mol_type = other DNA
  modified_base 61..62: mod_base = OTHER; note = phosphorothioated base
atctcatccctgcgtgtctccgactgcacaatctgatatgatgatcaaggacatgagcagcat

SEQ ID NO: 261 - DNA, 61 bp, synthetic construct
  source 1..61: mol_type = other DNA
  modified_base 59..60: mod_base = OTHER; note = phosphorothioated base
atctcatccctgcgtgtctccgactgcacatactgcaggagatcaaggacatgagcagcat

SEQ ID NO: 262 - DNA, 61 bp, synthetic construct
  source 1..61: mol_type = other DNA
  modified_base 59..60: mod_base = OTHER; note = phosphorothioated base
atctcatccctgcgtgtctccgactgcacttcatgacagagatcaaggacatgagcagcat

SEQ ID NO: 263 - DNA, 63 bp, synthetic construct
  source 1..63: mol_type = other DNA
  modified_base 61..62: mod_base = OTHER; note = phosphorothioated base
atctcatccctgcgtgtctccgactgcactacatcatggcatgatcaaggacatgagcagcat

SEQ ID NO: 264 - DNA, 61 bp, synthetic construct
  source 1..61: mol_type = other DNA
  modified_base 59..60: mod_base = OTHER; note = phosphorothioated base
atctcatccctgcgtgtctccgactgcacttcactcatgagatcaaggacatgagcagcat

SEQ ID NO: 265 - DNA, 61 bp, synthetic construct
  source 1..61: mol_type = other DNA
  modified_base 59..60: mod_base = OTHER; note = phosphorothioated base
atctcatccctgcgtgtctccgactgcacatgcgcttacagatcaaggacatgagcagcat

SEQ ID NO: 266 - DNA, 63 bp, synthetic construct
  source 1..63: mol_type = other DNA
  modified_base 61..62: mod_base = OTHER; note = phosphorothioated base
atctcatccctgcgtgtctccgactgcacagcatctctgcctgatcaaggacatgagcagcat

SEQ ID NO: 267 - DNA, 63 bp, synthetic construct
  source 1..63: mol_type = other DNA
  modified_base 61..62: mod_base = OTHER; note = phosphorothioated base
atctcatccctgcgtgtctccgactgcacactgccatatcatgatcaaggacatgagcagcat

SEQ ID NO: 268 - DNA, 61 bp, synthetic construct
  source 1..61: mol_type = other DNA
  modified_base 59..60: mod_base = OTHER; note = phosphorothioated base
atctcatccctgcgtgtctccgactgcactcagctgcggcgatcaaggacatgagcagcat

SEQ ID NO: 269 - DNA, 61 bp, synthetic construct
  source 1..61: mol_type = other DNA
  modified_base 59..60: mod_base = OTHER; note = phosphorothioated base
atctcatccctgcgtgtctccgactgcacaatgctatcgagatcaaggacatgagcagcat

SEQ ID NO: 270 - DNA, 61 bp, synthetic construct
  source 1..61: mol_type = other DNA
  modified_base 59..60: mod_base = OTHER; note = phosphorothioated base
atctcatccctgcgtgtctccgactgcacagataagagcagatcaaggacatgagcagcat

SEQ ID NO: 271 - DNA, 61 bp, synthetic construct
  source 1..61: mol_type = other DNA
  modified_base 59..60: mod_base = OTHER; note = phosphorothioated base
atctcatccctgcgtgtctccgactgcacagagccactcagatcaaggacatgagcagcat

SEQ ID NO: 272 - DNA, 61 bp, synthetic construct
  source 1..61: mol_type = other DNA
  modified_base 59..60: mod_base = OTHER; note = phosphorothioated base
atctcatccctgcgtgtctccgactgcacatgtaatatcagatcaaggacatgagcagcat

SEQ ID NO: 273 - DNA, 61 bp, synthetic construct
  source 1..61: mol_type = other DNA
  modified_base 59..60: mod_base = OTHER; note = phosphorothioated base
atctcatccctgcgtgtctccgactgcacatagatagccagatcaaggacatgagcagcat

SEQ ID NO: 274 - DNA, 61 bp, synthetic construct
  source 1..61: mol_type = other DNA
  modified_base 59..60: mod_base = OTHER; note = phosphorothioated base
atctcatccctgcgtgtctccgactgcacactagcatggcgatcaaggacatgagcagcat

SEQ ID NO: 275 - DNA, 61 bp, synthetic construct
  source 1..61: mol_type = other DNA
  modified_base 59..60: mod_base = OTHER; note = phosphorothioated base
atctcatccctgcgtgtctccgactgcacaatagcatcgagatcaaggacatgagcagcat

SEQ ID NO: 276 - DNA, 61 bp, synthetic construct
  source 1..61: mol_type = other DNA
  modified_base 59..60: mod_base = OTHER; note = phosphorothioated base
atctcatccctgcgtgtctccgactgcacttctgtgagatgatcaaggacatgagcagcat

SEQ ID NO: 277 - DNA, 61 bp, synthetic construct
  source 1..61: mol_type = other DNA
  modified_base 59..60: mod_base = OTHER; note = phosphorothioated base
atctcatccctgcgtgtctccgactgcacacaatagcgatgatcaaggacatgagcagcat

SEQ ID NO: 278 - DNA, 61 bp, synthetic construct
  source 1..61: mol_type = other DNA
  modified_base 59..60: mod_base = OTHER; note = phosphorothioated base
atctcatccctgcgtgtctccgactgcactgtaggcatctgatcaaggacatgagcagcat

SEQ ID NO: 279 - DNA, 61 bp, synthetic construct
  source 1..61: mol_type = other DNA
  modified_base 59..60: mod_base = OTHER; note = phosphorothioated base
atctcatccctgcgtgtctccgactgcacaacagctctctgatcaaggacatgagcagcat

SEQ ID NO: 280 - DNA, 61 bp, synthetic construct
  source 1..61: mol_type = other DNA
  modified_base 59..60: mod_base = OTHER; note = phosphorothioated base
atctcatccctgcgtgtctccgactgcacagacacaatatgatcaaggacatgagcagcat

SEQ ID NO: 281 - DNA, 61 bp, synthetic construct
  source 1..61: mol_type = other DNA
  modified_base 59..60: mod_base = OTHER; note = phosphorothioated base
atctcatccctgcgtgtctccgactgcactagcaagatcagatcaaggacatgagcagcat

SEQ ID NO: 282 - DNA, 61 bp, synthetic construct
  source 1..61: mol_type = other DNA
  modified_base 59..60: mod_base = OTHER; note = phosphorothioated base
atctcatccctgcgtgtctccgactgcacttgagacatgagatcaaggacatgagcagcat

SEQ ID NO: 283 - DNA, 63 bp, synthetic construct
  source 1..63: mol_type = other DNA
  modified_base 61..62: mod_base = OTHER; note = phosphorothioated base
atctcatccctgcgtgtctccgactgcacagccgcatgcacagatcaaggacatgagcagcat

SEQ ID NO: 284 - DNA, 61 bp, synthetic construct
  source 1..61: mol_type = other DNA
  modified_base 59..60: mod_base = OTHER; note = phosphorothioated base
atctcatccctgcgtgtctccgactgcactcgcatgtggagatcaaggacatgagcagcat

SEQ ID NO: 285 - DNA, 63 bp, synthetic construct
  source 1..63: mol_type = other DNA
  modified_base 61..62: mod_base = OTHER; note = phosphorothioated base
atctcatccctgcgtgtctccgactgcacatgcgagcatggtgatcaaggacatgagcagcat

SEQ ID NO: 286 - DNA, 61 bp, synthetic construct
  source 1..61: mol_type = other DNA
  modified_base 59..60: mod_base = OTHER; note = phosphorothioated base
atctcatccctgcgtgtctccgactgcacaatacatgatcgatcaaggacatgagcagcat

SEQ ID NO: 287 - DNA, 63 bp, synthetic construct
  source 1..63: mol_type = other DNA
  modified_base 61..62: mod_base = OTHER; note = phosphorothioated base
atctcatccctgcgtgtctccgactgcacatggacatatgcagatcaaggacatgagcagcat

SEQ ID NO: 288 - DNA, 61 bp, synthetic construct
  source 1..61: mol_type = other DNA
  modified_base 59..60: mod_base = OTHER; note = phosphorothioated base
atctcatccctgcgtgtctccgactgcacatgcgtcacctgatcaaggacatgagcagcat

SEQ ID NO: 289 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatatgatggtgcagtgcagtcggagacacgcagggatgaganggn

SEQ ID NO: 290 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcattgctatagcatgtgcagtcggagacacgcagggatgaganggn

SEQ ID NO: 291 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcgccatgtgcagatgtgcagtcggagacacgcagggatgaganggn

SEQ ID NO: 292 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatgcatctgaatagtgcagtcggagacacgcagggatgaganggn

SEQ ID NO: 293 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctagtgttgcatgtgcagtcggagacacgcagggatgaganggn

SEQ ID NO: 294 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcatatcataatagtgcagtcggagacacgcagggatgaganggn

SEQ ID NO: 295 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcgatgcttctgagtgcagtcggagacacgcagggatgaganggn

SEQ ID NO: 296 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctcagatatgaagtgcagtcggagacacgcagggatgaganggn

SEQ ID NO: 297 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctcatagctattgtgcagtcggagacacgcagggatgaganggn

SEQ ID NO: 298 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatctgcagctatccatgtgcagtcggagacacgcagggatgaganggn

SEQ ID NO: 299 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcgcagatattatgtgcagtcggagacacgcagggatgaganggn

SEQ ID NO: 300 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctcctctgtgctgtgcagtcggagacacgcagggatgaganggn

SEQ ID NO: 301 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcagagcgcagttgtgcagtcggagacacgcagggatgaganggn

SEQ ID NO: 302 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctgtcatcttgtgtgcagtcggagacacgcagggatgaganggn

SEQ ID NO: 303 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctaatgtcatctgtgcagtcggagacacgcagggatgaganggn

SEQ ID NO: 304 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatatgtgagccatgtgcagtcggagacacgcagggatgaganggn

SEQ ID NO: 305 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcagatgcatctcttgtgcagtcggagacacgcagggatgaganggn

SEQ ID NO: 306 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcatcacagctaagtgcagtcggagacacgcagggatgaganggn

SEQ ID NO: 307 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcatctattgcgagtgcagtcggagacacgcagggatgaganggn

SEQ ID NO: 308 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcattgagagagtgtgcagtcggagacacgcagggatgaganggn

SEQ ID NO: 309 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatcatcaggcagtgtgcagtcggagacacgcagggatgaganggn

SEQ ID NO: 310 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcatatgaatagagtgcagtcggagacacgcagggatgaganggn

SEQ ID NO: 311 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcagtgcgctcaagtgcagtcggagacacgcagggatgaganggn

SEQ ID NO: 312 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcagatatgttgagtgcagtcggagacacgcagggatgaganggn

SEQ ID NO: 313 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatctgctgcgccgcatgtgcagtcggagacacgcagggatgaganggn

SEQ ID NO: 314 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcgcgcacagcaagtgcagtcggagacacgcagggatgaganggn

SEQ ID NO: 315 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcatgaacgctatgtgcagtcggagacacgcagggatgaganggn

SEQ ID NO: 316 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatctcagcagatggctgtgcagtcggagacacgcagggatgaganggn

SEQ ID NO: 317 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctggcgctgagtgtgcagtcggagacacgcagggatgaganggn

SEQ ID NO: 318 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctatcagatgttgtgcagtcggagacacgcagggatgaganggn

SEQ ID NO: 319 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcgatgcggagcagtgcagtcggagacacgcagggatgaganggn

SEQ ID NO: 320 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcatcaagcgatagtgcagtcggagacacgcagggatgaganggn

SEQ ID NO: 321 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctgctgagaagagtgcagtcggagacacgcagggatgaganggn

SEQ ID NO: 322 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcgtgcacaccatgtgcagtcggagacacgcagggatgaganggn

SEQ ID NO: 323 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctaagatgatatgtgcagtcggagacacgcagggatgaganggn

SEQ ID NO: 324 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcattgtgtgatcatgtgcagtcggagacacgcagggatgaganggn

SEQ ID NO: 325 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctacaatgtgcagtgcagtcggagacacgcagggatgaganggn

SEQ ID NO: 326 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcatataccatgagtgcagtcggagacacgcagggatgaganggn

SEQ ID NO: 327 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcacgccagcatgcagtgcagtcggagacacgcagggatgaganggn

SEQ ID NO: 328 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatgctcatgaagagtgcagtcggagacacgcagggatgaganggn

SEQ ID NO: 329 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatatgctccatgagtgcagtcggagacacgcagggatgaganggn

SEQ ID NO: 330 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcataggcagtatgtgcagtcggagacacgcagggatgaganggn

SEQ ID NO: 331 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatatagatgcattgtgcagtcggagacacgcagggatgaganggn

SEQ ID NO: 332 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatgcattgtgctcgtgcagtcggagacacgcagggatgaganggn

SEQ ID NO: 333 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcagatgtgcatgttgtgcagtcggagacacgcagggatgaganggn

SEQ ID NO: 334 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcagatgccatgcacgtgcagtcggagacacgcagggatgaganggn

SEQ ID NO: 335 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcacattgctgtagtgcagtcggagacacgcagggatgaganggn

SEQ ID NO: 336 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcgcaggactgatgtgcagtcggagacacgcagggatgaganggn

SEQ ID NO: 337 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcgcatgtcgcttgtgcagtcggagacacgcagggatgaganggn

SEQ ID NO: 338 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcagatattgagcatgtgcagtcggagacacgcagggatgaganggn

SEQ ID NO: 339 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcacgatgcgattgtgcagtcggagacacgcagggatgaganggn

SEQ ID NO: 340 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcactgcagtgccgtgcagtcggagacacgcagggatgaganggn

SEQ ID NO: 341 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatgcagagcaatagtgcagtcggagacacgcagggatgaganggn

SEQ ID NO: 342 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatgtgccgcagatgtgcagtcggagacacgcagggatgaganggn

SEQ ID NO: 343 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcattctgcatcgatgtgcagtcggagacacgcagggatgaganggn

SEQ ID NO: 344 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcgctggtcatgtgtgcagtcggagacacgcagggatgaganggn

SEQ ID NO: 345 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcactacagcattgtgcagtcggagacacgcagggatgaganggn

SEQ ID NO: 346 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcgatgatcaatgatgtgcagtcggagacacgcagggatgaganggn

SEQ ID NO: 347 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatgcattgtgagtgtgcagtcggagacacgcagggatgaganggn

SEQ ID NO: 348 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatgatagccacatgtgcagtcggagacacgcagggatgaganggn

SEQ ID NO: 349 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcattgcgcatacatgtgcagtcggagacacgcagggatgaganggn

SEQ ID NO: 350 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctgagagcgcaagtgcagtcggagacacgcagggatgaganggn

SEQ ID NO: 351 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcatattgtgcgcgtgcagtcggagacacgcagggatgaganggn

SEQ ID NO: 352 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatgctggtgcgctgtgcagtcggagacacgcagggatgaganggn

SEQ ID NO: 353 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcaccagatagcagtgcagtcggagacacgcagggatgaganggn

SEQ ID NO: 354 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatctatccacatgcatgtgcagtcggagacacgcagggatgaganggn

SEQ ID NO: 355 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctctggtgcgatgtgcagtcggagacacgcagggatgaganggn

SEQ ID NO: 356 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatcatatcagattgtgcagtcggagacacgcagggatgaganggn

SEQ ID NO: 357 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctcctgcagtatgtgcagtcggagacacgcagggatgaganggn

SEQ ID NO: 358 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctctgtcatgaagtgcagtcggagacacgcagggatgaganggn

SEQ ID NO: 359 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatgccatgatgtagtgcagtcggagacacgcagggatgaganggn

SEQ ID NO: 360 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctcatgagtgaagtgcagtcggagacacgcagggatgaganggn

SEQ ID NO: 361 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctgtaagcgcatgtgcagtcggagacacgcagggatgaganggn

SEQ ID NO: 362 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcaggcagagatgctgtgcagtcggagacacgcagggatgaganggn

SEQ ID NO: 363 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcatgatatggcagtgtgcagtcggagacacgcagggatgaganggn

SEQ ID NO: 364 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcgccgcagctgagtgcagtcggagacacgcagggatgaganggn

SEQ ID NO: 365 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctcgatagcattgtgcagtcggagacacgcagggatgaganggn

SEQ ID NO: 366 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctgctcttatctgtgcagtcggagacacgcagggatgaganggn

SEQ ID NO: 367 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctgagtggctctgtgcagtcggagacacgcagggatgaganggn

SEQ ID NO: 368 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctgatattacatgtgcagtcggagacacgcagggatgaganggn

SEQ ID NO: 369 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctggctatctatgtgcagtcggagacacgcagggatgaganggn

SEQ ID NO: 370 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcgccatgctagtgtgcagtcggagacacgcagggatgaganggn

SEQ ID NO: 371 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctcgatgctattgtgcagtcggagacacgcagggatgaganggn

SEQ ID NO: 372 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcatctcacagaagtgcagtcggagacacgcagggatgaganggn

SEQ ID NO: 373 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcatcgctattgtgtgcagtcggagacacgcagggatgaganggn

SEQ ID NO: 374 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcagatgcctacagtgcagtcggagacacgcagggatgaganggn

SEQ ID NO: 375 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcagagagctgttgtgcagtcggagacacgcagggatgaganggn

SEQ ID NO: 376 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcatattgtgtctgtgcagtcggagacacgcagggatgaganggn

SEQ ID NO: 377 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctgatcttgctagtgcagtcggagacacgcagggatgaganggn

SEQ ID NO: 378 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctcatgtctcaagtgcagtcggagacacgcagggatgaganggn

SEQ ID NO: 379 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatctgtgcatgcggctgtgcagtcggagacacgcagggatgaganggn

SEQ ID NO: 380 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatctccacatgcgagtgcagtcggagacacgcagggatgaganggn

SEQ ID NO: 381 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatcaccatgctcgcatgtgcagtcggagacacgcagggatgaganggn

SEQ ID NO: 382 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcgatcatgtattgtgcagtcggagacacgcagggatgaganggn

SEQ ID NO: 383 - DNA, 65 bp, synthetic construct
  source 1..65: mol_type = other DNA
  misc_feature 1: note = 5' phosphorylation
  modified_base 62: mod_base = OTHER; note = deoxyuridine
  modified_base 65: mod_base = OTHER; note = deoxyuridine
  misc_binding 65: bound_moiety = 3' biotin- triethylene glycol (TEG) spacer arm
tgctgctcatgtggaagatctgcatatgtccatgtgcagtcggagacacgcagggatgaganggn

SEQ ID NO: 384 - DNA, 63 bp, synthetic construct
  source 1..63: mol_type = other DNA
  misc_feature 1: note = 5' phosphorylation
  modified_base 60: mod_base = OTHER; note = deoxyuridine
  modified_base 63: mod_base = OTHER; note = deoxyuridine
  misc_binding 63: bound_moiety = 3' biotin- triethylene glycol (TEG) spacer arm
tgctgctcatgtggaagatcaggtgacgcatgtgcagtcggagacacgcagggatgaganggn

SEQ ID NO: 385-386 - skipped (2 entries)

SEQ ID NO: 387 - DNA, 29 bp, synthetic construct
  source 1..29: mol_type = other DNA
atctcatccctgcgtgtctccgactgcac

SEQ ID NO: 388 - DNA, 12 bp, synthetic construct
  source 1..12: mol_type = other DNA
catgagcagcat

SEQ ID NO: 389 - DNA, 12 bp, synthetic construct
  source 1..12: mol_type = other DNA
tatggtcatcga

SEQ ID NO: 390 - DNA, 12 bp, synthetic construct
  source 1..12: mol_type = other DNA
tatggtcgtcga

SEQ ID NO: 391 - DNA, 25 bp, synthetic construct
  source 1..25: mol_type = other DNA
atgcatgcatgatgatgatgcatgc

SEQ ID NO: 392 - DNA, 10 bp, synthetic construct
  source 1..10: mol_type = other DNA
cccctagggg

SEQ ID NO: 393 - DNA, 10 bp, synthetic construct
  source 1..10: mol_type = other DNA
  modified_base 5: mod_base = cm
tcgtcatctg

SEQ ID NO: 394 - DNA, 10 bp, synthetic construct
  source 1..10: mol_type = other DNA
tcgtcatctg

SEQ ID NO: 395 - DNA, 10 bp, synthetic construct
  source 1..10: mol_type = other DNA
ttgtcatttg

SEQ ID NO: 396 - skipped

SEQ ID NO: 397 - DNA, 10 bp, synthetic construct
  source 1..10: mol_type = other DNA
ggggatcccc

SEQ ID NO: 398 - skipped

SEQ ID NO: 399 - DNA, 29 bp, synthetic construct
  source 1..29: mol_type = other DNA
atctcatccctgcgtgtctccgactgcac

SEQ ID NO: 400 - DNA, 22 bp, synthetic construct
  source 1..22: mol_type = other DNA
gatcaaaaaacatgagcagcat

SEQ ID NO: 401 - DNA, 65 bp, synthetic construct
  source 1..65: mol_type = other DNA
acatatgtgcgcgaaaaaacatcaccgactgcccatagagagctgagactgccaaggcacacagg

SEQ ID NO: 402 - DNA, 44 bp, synthetic construct
  source 1..44: mol_type = other DNA
agctctctatgggcagtcggtgatgaaaaaacgcgcacatatgt

SEQ ID NO: 403 - DNA, 22 bp, synthetic construct
  source 1..22: mol_type = other DNA
atgctgctcatgaaaaaagatc

SEQ ID NO: 404 - DNA, 32 bp, synthetic construct
  source 1..32: mol_type = other DNA
  modified_base 29: mod_base = OTHER; note = uracil
  modified_base 32: mod_base = OTHER; note = uracil
gtgcagtcggagacacgcagggatgagatggt

SEQ ID NO: 405 - DNA, 28 bp, synthetic construct
  source 1..28: mol_type = other DNA
cctatcccctgtgtgccttggcagtctc

SEQ ID NO: 406 - DNA, 21 bp, synthetic construct
  source 1..21: mol_type = other DNA
gatcaaggacatgagcagcat

SEQ ID NO: 407 - DNA, 64 bp, synthetic construct
  source 1..64: mol_type = other DNA
acatatgtgcgcgaggaacatcaccgactgcccatagagagctgagactgccaaggcacacagg

SEQ ID NO: 408 - DNA, 43 bp, synthetic construct
  source 1..43: mol_type = other DNA
agctctctatgggcagtcggtgatgaaggtcgcgcacatatgt

SEQ ID NO: 409 - DNA, 21 bp, synthetic construct
  source 1..21: mol_type = other DNA
atgctgctcatgtggaagatc

(19) United States
(12) Patent Application Publication
(10) Pub. No.: US 2026/0132458 A1
(43) Pub. Date: May 14, 2026

(54) SYSTEMS AND METHODS FOR NUCLEIC ACID MISMATCH ERROR DETECTION

(71) Applicant: Ultima Genomics, Inc., Fremont, CA (US)

(72) Inventors: Itai RUSINEK, Holon (IL); Zohar SHIPONY, Rehovot (IL); Eti MEIRI, Shoham (IL); Ariel HAIMOVICH, Binyamina (IL); Omer BARAD, Mazkeret Batya (IL); Doron LIPSON, Hasharon (IL); Shay SHILO, Petah Tikva (IL); Daniel MAZUR, San Diego, CA (US)

(21) Appl. No.: 19/278,437

(22) Filed: Jul. 23, 2025

Related U.S. Application Data
(63) Continuation of application No. PCT/US2024/013236, filed on Jan. 26, 2024.
(60) Provisional application No. 63/441,727, filed on Jan. 27, 2023.
(60) Provisional application No. 63/452,696, filed on Mar. 17, 2023.
(60) Provisional application No. 63/465,346, filed on May 10, 2023.

(51) Int. Cl. C12Q 1/6874; C12Q 1/6886

(52) U.S. Cl. CPC C12Q 1/6874; C12Q 1/6886; C12Q 2600/156

(57) ABSTRACT

A double-stranded template nucleic acid molecule may comprise an adaptor, the adaptor comprising a mismatch portion. The mismatch portion may comprise a first mismatch sequence in a first strand and a second mismatch sequence that is not complementary to the first mismatch sequence in a second strand. When the double-stranded template nucleic acid molecule is amplified to generate a cluster of amplified strands, and the cluster sequenced to generate a sequencing read, the portion of the sequencing read corresponding to the mismatch portion may be analyzed to determine whether the cluster of amplified strands has derived from only one or both strands of the double-stranded template nucleic acid molecule. Disagreements at one or more loci in sequencing signals, base calls, or sequencing reads between the two strand derivatives in the cluster may be used to correct for sequencing error and improve sequencing accuracy.

### CROSS-REFERENCE TO RELATED APPLICATIONS

This application is a continuation application of PCT International Application No. PCT/US2024/013236, filed Jan. 26, 2024, which claims the benefit of U.S. Provisional Application No. 63/441,727, filed on Jan. 27, 2023, 63/452,696, filed on Mar. 17, 2023, and 63/465,346, filed on May 10, 2023, each of which is entirely incorporated by reference herein for all purposes.

### REFERENCE TO A SEQUENCE LISTING

This application contains a Sequence Listing, which has been submitted electronically in xml format and is hereby incorporated by reference in its entirety. Said xml copy, created on Jan. 11, 2026, is named SeqList2-368893-40901.xml and is 845,532 bytes in size.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

Despite the advance of sequencing technology, analyzing samples with high throughput and efficiency still requires laborious efforts.

### SUMMARY

A template nucleic acid molecule that is input to a sequencing library may be double-stranded. In other cases, the template nucleic acid molecule may exist in and/or be converted to double-stranded form during various points in time during the sequencing process, such as during one or more operations of library prep, amplification, or enrichment. Prior to sequencing, the template nucleic acid molecule may be subject to amplification, such as to generate a colony of the template nucleic acid molecule in a cluster (e.g., on a support such as a bead or a spot on a surface)—during sequencing, a cumulative signal detected from the cluster of amplified molecules may be significantly stronger than a signal from a single molecule. However, some amplification protocols may amplify only one of the two strands of a double-stranded template nucleic acid molecule, discarding the information (e.g., sequence information) in the other strand. While in some cases the two strands in the double-stranded template nucleic acid molecule may be perfect reverse complements of each other, in which case discarding one of the two strands does not result in loss of information, in some other cases, the two strands may contain site(s) of base mismatch, in which case discarding one of the strands results in loss of valuable information from the template nucleic acid molecule, including a potential alternative base callout at the site(s) in the sequence as well as the fact that there exist site(s) of base mismatch in the first place. A base mismatch, also referred to as mismatch, as used herein may generally refer to the occurrence of a non-complementary base pairing within a double-stranded portion of a nucleic acid molecule. In some examples, PCR-free library DNA may carry site(s) of base mismatch with relatively high frequency. In other amplification protocols, it may be possible to amplify both strands of a double-stranded template nucleic acid molecule to generate a ‘mix’ of strand derivatives in the amplified cluster. However, signals detected from such ‘mixed’ amplified clusters may need to be qualified by knowledge of what percentage of the amplified cluster is derived from one strand over the other. Thus, it may be beneficial to preserve both strands of a template nucleic acid molecule during amplification. Such preservation may be particularly advantageous in detecting or ruling out single nucleotide polymorphisms (SNPs) and improving SNP detection error rates. A SNP is a genetic variant in a subject's DNA that is especially vulnerable to erroneous detection. When both strands in the double-stranded template nucleic acid molecule are preserved for amplification, it is important that a significant amount of both strands have in fact amplified and are represented in the amplified cluster.

Provided herein are systems and methods that address at least the abovementioned concerns. Provided are systems and methods which preserve both strands of a double-stranded template nucleic acid molecule during amplification, as well as systems and methods which can quantitatively measure the fractions of a cluster of amplified molecules which have derived from the two strands. Provided herein are systems and methods for amplifying both strands of a double-stranded template nucleic acid without dissociating the two strands.

In an aspect, provided is a method for high accuracy sequencing, comprising: (a) providing an amplified cluster of a plurality of nucleic acid molecules derived from a double-stranded nucleic acid molecule which comprises a first strand and a second strand, wherein a first subset of the plurality of nucleic acid molecules each comprises a first sequence that is a copy of at least a portion of the first strand sequence and wherein a second subset of the plurality of nucleic acid molecules each comprises a second sequence that is a reverse complement copy of at least a portion of the second strand sequence; (b) collecting sequencing signals from the amplified cluster to determine a disagreement between the first sequence and the second sequence at a locus; and (c) excluding the locus from single nucleotide variant (SNV) or single nucleotide polymorphism (SNP) calling based at least in part on the disagreement.

In some embodiments, the sequencing signals from the amplified cluster are collected in (b) by hybridizing sequencing primers to the plurality of nucleic acid molecules and simultaneously interrogating both the first subset and the second subset with a same set of nucleotide mixtures.

In some embodiments, the sequencing primers hybridized to the first subset and the second subset comprise a same sequence.

In some embodiments, the sequencing primers hybridized to the first subset and the second subset comprise different sequences.

In some embodiments, in (b) a same nucleotide mixture interrogates a same locus between the first sequence and the second sequence.

In some embodiments, the same locus has a length of 1 base position.

In some embodiments, the same locus has a length of at least 2 base positions.

In some embodiments, the same set of nucleotide mixtures interrogates a same locus between the first sequence and the second sequence.

In some embodiments, the same locus has a length of 1 base position.

In some embodiments, the same locus has a length of at least 2 base positions.

In some embodiments, (b) comprises simultaneously generating sequencing data from the first subset and the second subset.

In some embodiments, the sequencing signals from the amplified cluster are collected in (b) by (i) hybridizing a first set of sequencing primers to the first subset of the plurality of nucleic acid molecules and interrogating the first subset with a first set of nucleotide mixtures, and (ii) hybridizing a second set of sequencing primers to the second subset of the plurality of nucleic acid molecules and interrogating the second subset with a second set of nucleotide mixtures that is different from the first set of nucleotide mixtures.

In some embodiments, the first set of sequencing primers and the second set of sequencing primers comprise different sequences.

In some embodiments, the first set of sequencing primers and the second set of sequencing primers have the same sequence.

In some embodiments, (b) comprises generating sequencing data from the first subset and the second subset at different timepoints.

In some embodiments, the method further comprises generating a single sequencing read from the amplified cluster, wherein the single sequencing read is generated by interrogating both the first subset and the second subset of the plurality of nucleic acid molecules.

In some embodiments, the method further comprises generating two sequencing reads from the amplified cluster, a first sequencing read generated by interrogating the first subset of the plurality of nucleic acid molecules and a second sequencing read generated by interrogating the second subset of the plurality of nucleic acid molecules.

In some embodiments, the method further comprises generating at least two candidate base calls for the locus at which the disagreement is determined.

In some embodiments, the method further comprises comparing the at least two candidate base calls to the locus in a reference sequence and selecting one of the at least two candidate base calls or a base at the locus in the reference sequence for a consensus read.

In some embodiments, the method further comprises generating a sequencing read for the amplified cluster using the sequencing signals.

In some embodiments, the method further comprises aligning the sequencing read to the reference sequence.

In some embodiments, the method further comprises calling one or more SNVs or SNPs for a sample or subject that the double-stranded nucleic acid molecule is derived from.

In some embodiments, the method further comprises detecting a minimal residual disease (MRD), tumor fraction, or circulating tumor fraction in the sample or the subject based on the one or more SNVs or SNPs called.

In some embodiments, the first subset each further comprises a first strand recognition element and wherein the second subset each further comprises a second strand recognition element is different from the first strand recognition element.

In some embodiments, the first strand recognition element and the second strand recognition element are different sequences.

In some embodiments, the different sequences are different homopolymer sequences.

In some embodiments, the different sequences are different non-homopolymer sequences.

In some embodiments, the different sequences have different lengths.

In some embodiments, the different sequences have the same length.

In some embodiments, the different sequences each have a single base length.

In some embodiments, the different sequences each have at least 1 base lengths.

In some embodiments, the different sequences each have at least 3 base lengths.

In some embodiments, the different sequences each have at least 5 base lengths.

In some embodiments, the first strand recognition element and the second strand recognition element are not nucleic acid sequences.

In some embodiments, the method further comprises detecting a presence of one or both of the first strand recognition element and the second strand recognition element in the amplified cluster.

In some embodiments, the detecting comprises sequencing the plurality of nucleic acid molecules to identify a sequence of the first strand recognition element, the second strand recognition element, or both.

In some embodiments, the detecting comprises sequencing the plurality of nucleic acid molecules to identify a disagreement in sequence in a common portion of the plurality of nucleic acid molecules that comprises the first strand recognition element or the second strand recognition element.

In some embodiments, the detecting comprises hybridizing labeled oligonucleotide probes to the first strand recognition element, the second strand recognition element, or both, and detecting signals from the labeled oligonucleotide probes.

In some embodiments, the method further comprises determining a ratio of the first subset and the second subset in the plurality of nucleic acid molecules.

In some embodiments, the ratio is determined by processing signal intensities collected from interrogating the first strand recognition element, the second strand recognition element, or both.

In some embodiments, the method further comprises generating a sequencing read for the amplified cluster, wherein the sequencing read is generated based at least in part on the sequencing signals and the ratio.

In some embodiments, the amplified cluster is immobilized to an individually addressable location on a substrate.

In some embodiments, the substrate comprises at least 1,000,000 individually addressable locations.

In some embodiments, the substrate comprises at least 1,000,000,000 individually addressable locations.

In some embodiments, the substrate comprises at least 5,000,000,000 individually addressable locations.

In some embodiments, the substrate comprises at least 10,000,000,000 individually addressable locations.

In some embodiments, the substrate comprises at least 20,000,000,000 individually addressable locations.

In some embodiments, the substrate is substantially planar.

In some embodiments, the substrate is textured or patterned.

In some embodiments, the substrate is unpatterned.

In some embodiments, the substrate comprises a layer of aminosilane that immobilizes the amplified cluster.

In some embodiments, the substrate comprises a layer of surface primers that immobilizes the amplified cluster.

In some embodiments, the plurality of nucleic acid molecules are coupled to a bead, which bead is immobilized to the individually addressable location on the substrate.

In some embodiments, the substrate is rotated during sequencing of the plurality of nucleic acid molecules.

In some embodiments, the plurality of nucleic acid molecules are single-stranded molecules.

In another aspect, provided is a method for high accuracy sequencing, comprising: (a) providing an amplified cluster of a plurality of nucleic acid molecules derived from a double-stranded nucleic acid molecule which comprises a first strand and a second strand, wherein a first subset of the plurality of nucleic acid molecules each comprises a first strand recognition element and a first sequence that is a copy of at least a portion of the first strand sequence, and wherein a second subset of the plurality of nucleic acid molecules each comprises a second strand recognition element and a second sequence that is a reverse complement copy of at least a portion of the second strand sequence, wherein the first strand recognition element and the second strand recognition elements are different; and (b) detecting a presence of the first strand recognition element, the second strand recognition element, or both in the amplified cluster.

In some embodiments, the plurality of nucleic acid molecules are immobilized to a support.

In some embodiments, the method further comprises subjecting the double-stranded nucleic acid molecule to amplification to generate the plurality of nucleic acid molecules.

In some embodiments, the amplification comprises emulsion PCR (ePCR), emulsion recombinase polymerase amplification (eRPA), PCR, RPA, rolling circle amplification (RCA), multiple displacement amplification (MDA), bridge amplification, or a combination thereof.

In some embodiments, the method further comprises, prior to the amplification, ligating a strand recognition adapter comprising a pair of mismatch sequences to the double-stranded nucleic acid molecule.

In some embodiments, double-stranded nucleic acid molecule is amplified by (1) ligating a hairpin adapter to each end to generate a dumbbell molecule, and subjecting the dumbbell molecule to rolling circle amplification (RCA) to generate a first amplification product, (2) cleaving or digesting portions of the first amplification product corresponding to the hairpin adapter to generate a plurality of copy molecules each comprising a copy of the first strand the second strand.

In some embodiments, double-stranded nucleic acid molecule is amplified by (1) ligating a hairpin adapter to each end to generate a dumbbell molecule, and subjecting the dumbbell molecule to rolling circle amplification (RCA) by contacting the dumbbell molecule with a plurality of random primers and dNTPs comprising dUTPs but not dTTPs to generate first amplification products, (2) hybridizing second primers to the first amplification products in the presence of dNTPs comprising dTTPs but not dUTPs to generate second amplification products, (3) degrading the first amplification products based on uracil residues, to isolate second amplification products, and (4) cleaving or digesting portions of the second amplification product corresponding to the hairpin adapter to generate a plurality of copy molecules each comprising a copy of the first strand the second strand.

In some embodiments, double-stranded nucleic acid molecule is amplified by (1) ligating a hairpin adapter to each end to generate a dumbbell molecule, and subjecting the dumbbell molecule to rolling circle amplification (RCA) to generate a first amplification product, (2) hybridizing second primers to the first amplification product under suppressed strand displacement conditions to generate a plurality of second amplification products each comprising a single copy of the first strand and the second strand.

In some embodiments, the method further comprises ligating a strand recognition adapter comprising a pair of mismatch sequences to each of the plurality of copy molecules to generate the plurality of nucleic acid molecules.

In some embodiments, at least one of the hairpin adapter comprises a strand recognition adapter comprising a pair of mismatch sequences, and wherein the plurality of copy molecules each comprises a copy of the pair of mismatch sequences.

In another aspect, provided is a method for detecting amplified strands on a support, comprising: (a) attaching a double-stranded template molecule to a support to generate a template-attached support, wherein the double-stranded template molecule comprises a first strand and a second strand, wherein the double-stranded template molecule comprises an adaptor comprising a mismatch portion, wherein the mismatch portion comprises in the first strand a first mismatch sequence and in the second strand a second mismatch sequence that is not complementary to the first mismatch sequence; (b) subjecting the double-stranded template molecule of the template-attached support to amplification to generate an amplified support comprising a plurality of amplified strands attached thereto; (c) sequencing the amplified support to generate a sequencing read; and (d) based at least in part on a portion of the sequencing read that corresponds to the mismatch portion, determining a percentage of amplified strands in the plurality of amplified strands in the amplified support that derives from the first strand.

In another aspect, provided is a method for detecting amplified strands on a support, comprising: (a) attaching a double-stranded template molecule to a support to generate a template-attached support, wherein the double-stranded template molecule comprises a first strand and a second strand, wherein the double-stranded template molecule comprises an adaptor comprising a mismatch portion, wherein the mismatch portion comprises in the first strand a first homopolymer sequence and in the second strand a second homopolymer sequence that is not complementary to the first homopolymer sequence; (b) subjecting the double-stranded template molecule of the template-attached support to amplification to generate an amplified support comprising a plurality of amplified strands attached thereto; (c) sequencing the amplified support to generate a sequencing read; and (d) based at least in part on a portion of the sequencing read that corresponds to the mismatch portion, determining a percentage of amplified strands in the plurality of amplified strands in the amplified support that derives from the first strand.

In another aspect, provided is a kit, comprising: a double-stranded adaptor comprising a mismatch portion, wherein the double-stranded adaptor comprises a first strand and a second strand, wherein the mismatch portion comprises in the first strand a first mismatch sequence and in the second strand a second mismatch sequence that is not complementary to the first mismatch sequence.

In some embodiments, the kit further comprises a plurality of double-stranded adaptor comprising the mismatch portion, wherein the mismatch portion of the plurality of double-stranded adaptor is identical.

In another aspect, provided is a kit, comprising: a double-stranded adaptor comprising a mismatch portion, wherein the double-stranded adaptor comprises a first strand and a second strand, wherein the mismatch portion comprises in the first strand a first homopolymer sequence and in the second strand a second homopolymer sequence that is not complementary to the first homopolymer sequence.

In some embodiments, the kit further comprises a plurality of double-stranded adaptor comprising the mismatch portion, wherein the mismatch portion of the plurality of double-stranded adaptor is identical.

In another aspect, provided is a composition, comprising: a double-stranded adaptor comprising a mismatch portion, wherein the double-stranded adaptor comprises a first strand and a second strand, wherein the mismatch portion comprises in the first strand a first mismatch sequence and in the second strand a second mismatch sequence that is not complementary to the first mismatch sequence.

In some embodiments, the composition further comprises a plurality of double-stranded adaptors each comprising the mismatch portion, wherein the mismatch portion of the plurality of double-stranded adaptor is identical.

In some embodiments, the composition further comprises a plurality of template molecules, wherein the plurality of template molecules comprises a plurality of double-stranded template insert molecules ligated to the plurality of double-stranded adaptors.

In some embodiments, the composition further comprises a template molecule, wherein the template molecule comprises a double-stranded template insert molecule ligated to the double-stranded adaptor.

In some embodiments, the composition further comprises a support.

In some embodiments, the support is attached to the template molecule.

In some embodiments, the composition further comprises a support.

In another aspect, provided is a composition, comprising: a double-stranded adaptor comprising a mismatch portion, wherein the double-stranded adaptor comprises a first strand and a second strand, wherein the mismatch portion comprises in the first strand a first homopolymer sequence and in the second strand a second homopolymer sequence that is not complementary to the first homopolymer sequence.

In some embodiments, the composition further comprises a plurality of double-stranded adaptors each comprising the mismatch portion, wherein the mismatch portion of the plurality of double-stranded adaptor is identical.

In another aspect, provided is a method for enrichment, comprising: (a) providing a plurality of balanced supports which comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first enrichment molecules to the plurality of amplified strands, wherein the plurality of first enrichment molecules comprises a first capture sequence complementary to the first mismatch sequence, to generate a first set of enriched balanced supports; and (c) contacting a plurality of second enrichment molecules to the first set of enriched balanced supports, wherein the plurality of second enrichment molecules comprises a second capture sequence comprising the second mismatch sequence, to generate a second set of enriched balanced supports.

In another aspect, provided is a method for enrichment, comprising: (a) providing a plurality of balanced supports which comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first enrichment molecules to the plurality of amplified strands, wherein the plurality of first enrichment molecules comprises a first capture sequence comprising the second mismatch sequence, to generate a first set of enriched balanced supports comprising the second mismatch sequence; and (c) contacting a plurality of second enrichment molecules to the first set of enriched balanced supports, wherein the plurality of second enrichment molecules comprises a second capture sequence complementary to the first mismatch sequence, to generate a second set of enriched balanced supports.

In another aspect, provided is a method for enrichment, comprising: (a) providing a plurality of balanced supports which comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first enrichment molecules to the plurality of amplified strands, wherein the plurality of first enrichment molecules comprises a first capture sequence complementary to the first mismatch sequence, to capture a subset of balanced supports; and (c) removing at least a subset of the subset of balanced supports from the plurality of plurality of balanced supports to generate a first set of enriched, balanced supports.

In another aspect, provided is a method for enrichment, comprising: (a) providing a plurality of balanced supports which comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first enrichment molecules to the plurality of amplified strands, wherein the plurality of first enrichment molecules comprises a first capture sequence comprising the second mismatch sequence, to capture a subset of balanced supports; and (c) removing at least a subset of the subset of balanced supports from the plurality of plurality of balanced supports to generate a first set of enriched, balanced supports.

In another aspect, provided is a method for generating an amplified support with a predetermined forward-reverse strand ratio, comprising: contacting (i) a template-attached support, wherein the template-attached support comprise a support attached to a double-stranded template molecule, wherein the double-stranded template molecule comprises a first strand and a second strand, wherein the double-stranded template molecule comprises an adaptor comprising a mismatch portion, wherein the mismatch portion comprises in the first strand a first mismatch sequence and in the second strand a second mismatch sequence that is not complementary to the first mismatch sequence, (ii) a plurality of forward amplification primers at a first predetermined concentration or comprising a first annealing temperature with the first mismatch sequence, wherein each forward amplification primer of the plurality of forward amplification primers comprises a reverse complement of the first mismatch sequence to hybridize to first amplified strands derived from the first strand, and (iii) a plurality of reverse amplification primers at a second predetermined concentration or comprising a second annealing temperature with the reverse complement of the second mismatch sequence, wherein each reverse amplification primer of the plurality of reverse amplification primers comprises the second mismatch sequence to hybridize to second amplified strands derived from the second strand, to generate the amplified support comprising a plurality of amplified strands with the predetermined forward-reverse strand ratio.

In another aspect, provided is a method for detecting strands on a support, comprising: (a) providing a plurality of balanced supports immobilized to a substrate, wherein the plurality of balanced supports comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first type of probes to the plurality of amplified strands, wherein the plurality of first type of probes each comprises a first capture sequence complementary to the first mismatch sequence and a first detectable moiety; and (c) detecting first signals from the first detectable moiety of a subset of the plurality of first type of probes bound to a subset of the plurality of amplified strands.

In some embodiments, the method further comprises: (d) contacting a plurality of second type of probes to the plurality of amplified strands, wherein the plurality of second type of probes each comprises a second capture sequence comprising the second mismatch sequence and a second detectable moiety; and (e) detecting second signals from the second detectable moiety of a subset of the plurality of second type of probes bound to a second subset of the plurality of amplified strands.

In another aspect, provided is a method of amplification, comprising: (a) providing a double-stranded target molecule and a plurality of adaptors, wherein the double-stranded target molecule comprises a first strand and a second strand and at least one adaptor of the plurality of adaptors comprises a hairpin sequence; (b) exposing the double-stranded target molecule and the plurality of adaptors to conditions sufficient to attach an adaptor to each end of the double-stranded target molecule, thereby generating a double-stranded template-adaptor molecule, wherein at least one attached adaptor comprises a hairpin sequence; and (c) subjecting the double-stranded template-adaptor molecule to amplification to generate a plurality of copies of the double-stranded template-adaptor molecule, wherein each copy of the double-stranded template-adaptor molecule comprises a copy of the sequence of the first strand and a copy of the sequence of the second strand.

In some embodiments, the amplification comprises rolling circle amplification (RCA), and the plurality of copies of the double-stranded template-adaptor molecule is attached to each other.

In some embodiments, the amplification comprises PCR.

In some embodiments, the amplification comprises loop-mediated isothermal amplification (LAMP).

In some embodiments, the at least one adaptor further comprises a first region and a second region that do not have sequence complementarity, wherein the first and second regions are distal from the double-stranded template molecule.

In some embodiments, the double-stranded template-adapter molecule is attached to a support, wherein the support comprises a plurality of primers, wherein a first subset of the primers has sequence complementarity to the first region and a second subset of the primers have sequence complementarity to the second region.

In another aspect, provided is a method of sequencing, comprising: (a) providing a double-stranded template molecule comprising, a first strand and a second strand with sequence complementarity to each other and at least one adaptor region comprising a single-stranded hairpin region; (b) annealing a primer to the single-stranded hairpin region; (c) extending the primer to generate a partially single-stranded template molecule, comprising a double-stranded region and a single-stranded region; (d) processing the partially single-stranded template molecule to generate a single-stranded template molecule; and e sequencing the single-stranded template molecule.

In some embodiments: processing (d) the partially single-stranded template molecule comprises filtering based on the sequence of the single-stranded region; and sequencing (e) comprises targeted sequencing.

In some embodiments: processing (d) the partially single-stranded template molecule comprises methylation conversion of the single-stranded region; and sequencing (e) comprises methylation sequencing.

In another aspect, provided is a method for sequencing, comprising: (a) providing a balanced construct comprising a mixture of a forward strand and a reverse strand, wherein (i) the forward strand comprises a first sequence that is identical to or a reverse complement of a first strand of a double-stranded template molecule of a sample, and (ii) the reverse strand comprises a second sequence that is a reverse complement of or identical to a methylation-converted sequence of a second strand of the double-stranded template molecule of the sample, respectively; and (b) sequencing the forward strand and the reverse strand by: i. hybridizing primers to the forward strand and the reverse strand, respectively, ii. extending the primers with nucleotides in nucleotide flows provided according to a repeating flow order, wherein a nucleotide flow comprises nucleotides of a single canonical base type, wherein the repeating flow order comprises a consecutive three flow order of a thymine-base flow, cytosine-base flow, and thymine-base flow, and iii. detecting flow signals indicative of incorporation of nucleotides, or lack thereof, by the primers subsequent to each respective nucleotide flows.

In some embodiments, the method further comprises using the flow signals detected in (b)(iii) to determine a methylation status of the double-stranded template molecule.

In some embodiments, the forward strand comprises a first strand recognition element comprising a first homopolymer sequence and wherein the reverse strand comprises a second strand recognition element comprising a second homopolymer sequence, wherein the first homopolymer sequence and the second homopolymer sequence comprise different bases.

In some embodiments, the method further comprises using a subset of the flow signals detected in (b)(iii) which corresponds to the first strand recognition element and the second strand recognition element to determine a forward-reverse ratio of a number of copies of the forward strand and a number of copies of the reverse strand on the balance construct.

In some embodiments, the method further comprises determining a methylation status of the double-stranded template molecule based at least in part on the forward-reverse ratio.

Another aspect of the present disclosure provides a system comprising one or more computer processors and computer memory coupled thereto. The computer memory comprises machine executable code that, upon execution by the one or more computer processors, implements any of the methods above or elsewhere herein. Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are incorporated by reference herein to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example workflow for processing a sample for sequencing.

FIG. 2 illustrates examples of individually addressable locations distributed on substrates, as described herein.

FIG. 3A, FIG. 3B, FIG. 3C, FIG. 3D, FIG. 3E, FIG. 3F, and FIG. 3G illustrate different examples of cross-sectional surface profiles of a substrate, as described herein.

FIG. 4 shows an example coating of a substrate with a hexagonal lattice of beads, as described herein.

FIGS. 5A-5B illustrate example systems and methods for loading a sample or a reagent onto a substrate, as described herein.

FIG. 6 illustrates a computerized system for sequencing a nucleic acid molecule.

FIGS. 7A-7C illustrate multiplexed stations in a sequencing system.

FIG. 8 illustrates a computer system that is programmed or otherwise configured to implement methods provided herein.

FIG. 9A illustrates a workflow for extension and amplification on a support, in which one of the template strands is not amplified.

FIG. 9B illustrates a workflow for extension and amplification on a support, in which both template strands are amplified.

FIG. 9C illustrates another workflow for extension and amplification on a support, in which both template strands are amplified.

FIGS. 10A-10B illustrate another workflow for extension and amplification on a support, in which both template strands are amplified, and in which an adaptor comprising a mismatch portion is used.

FIGS. 10C-10D illustrate another workflow for extension and amplification on a support, in which both template strands are amplified, and in which an adaptor comprising a mismatch portion is used.

FIG. 11 illustrates frequency distribution charts of beads having different forward-to-reverse strand ratios for different samples.

FIG. 12A shows a chart plotting (#of reads) vs (mean quality of the 20 bp read following cycle skip substitution) for beads having different forward-reverse % for a first sample.

FIG. 12B shows a chart plotting (#of reads) vs (mean quality of the 20 bp read following cycle skip substitution) for beads having different forward-reverse % for a second sample.

FIG. 13 illustrates a two-step enrichment protocol, according to embodiments of the present disclosure.

FIG. 14 illustrates a support comprising a template nucleic acid molecule, pre-amplification, with different example configurations of strand recognition elements.

FIG. 15A, FIGS. 15B, and 15C illustrate a rolling circle amplification (RCA) workflow, where both template strands are amplified (e.g., prior to attachment to a support), according to embodiments of the present disclosure.

FIG. 15D illustrates an additional workflow for amplifying double-stranded template molecules while retaining forward-reverse strand associations (e.g., amplification without losing double-stranded context).

FIG. 15E illustrates a workflow for dumbbell-based amplification using suppressed strand displacement conditions.

FIG. 15F illustrates a workflow for dumbbell-based amplification using random primers.

FIG. 15G illustrates a possible source of error during library preparation when performing blunt end shearing and ligating hairpin adaptors.

FIGS. 16A-16C illustrate alternatives for various stages of the workflow illustrated in FIGS. 15A-15D. FIGS. 16A and 16B illustrate alternative adaptor constructs. FIG. 16C illustrates alternative restriction site locations and resulting product molecules.

FIG. 16D illustrates a sequencing method, in accordance with embodiments of the present disclosure.

FIG. 17 illustrates another workflow for amplification, where both template strands are amplified, according to embodiments of the present disclosure.

FIGS. 18A and 18B illustrate a bridge PCR amplification workflow, where both template strands are amplified, according to embodiments of the present disclosure.

FIG. 19 illustrates a method for forming a partially double-stranded template molecule, in accordance with embodiments of the present disclosure.

FIG. 20 illustrates an example workflow for using unique molecular identifiers in a double-stranded template molecule.

FIG. 21A illustrates an example workflow for generating a duplex molecule comprising sequences corresponding to both strands of a double-stranded insert molecule.

FIG. 21B illustrates an additional example workflow for generating a duplex molecule comprising sequences corresponding to both strands of a double-stranded insert molecule, using a bending protein. Sequence shown in figure: GGGGATCCCC (SEQ ID NO:397).

FIG. 22 illustrates a method for generating double-stranded template molecules which are partially converted for methylation sequencing.

FIG. 23 illustrates different methods for generating balanced constructed using partially converted molecules for methylation sequencing.

FIG. 24 illustrates one example of how preserving information from both strands of a template nucleic acid molecule for sequencing can permit differentiation of a true mutation from an artificial (false) mutation in a sample.

FIG. 25A illustrates an example flow sequencing method that can be used to generate the sequencing data described herein.

FIG. 25B illustrates an example flowgram.

FIG. 25C shows two example sequences (e.g., extended sequencing primer sequences) that differ at least at one locus. Sequence 1 comprises TATGGTCATCGA (SEQ ID NO: 389) and Sequence 2 comprises TATGGTCGTCGA (SEQ ID NO: 390).

FIG. 25D illustrates sequencing data, using one flow order, for two different extended sequences: TATGGTCATCGA (SEQ ID NO: 389) and TATGGTCGTCGA (SEQ ID NO: 390).

FIG. 25E illustrates sequencing data using the T-A-C-G flow order for two different extended sequences: TATGGTCATCGA (SEQ ID NO: 389) and TATGGTCGTCGA (SEQ ID NO: 390).

FIG. 25F illustrates sequencing data using the A-G-C-T flow order for two different extended sequences: TATGGTCATCGA (SEQ ID NO: 389) and TATGGTCGTCGA (SEQ ID NO: 390).

FIG. 26A illustrates a comparison of SNV profiles for patient-matched FF and FFPE samples.

FIG. 26B illustrates SNVQ distributions for standard and ppmSeq cfDNA libraries. The ppmSeq libraries are divided into those where information from both strands was collected (mixed) and those where information was primarily collected for a single strand (non-mixed).

FIG. 26C illustrates the correlation between circulating tumor fraction measured for each of the 8 patient-specific profiles using standard and ppmSeq library preparation methods.

FIG. 26D illustrates the estimated tumor fraction in matched and control cfDNA samples, for standard cfDNA prep (left) and ppmSeq (right). Each column corresponds to one patient SNV profile, with one matched cfDNA sample and 9 negative control samples.

FIG. 27 illustrates error distribution for different mutation types and different assay types.

FIG. 28 illustrates examples of strand recognition adaptor-ligated template molecules. SEQ ID NOs for the sequences shown in the figure are as follows. (A) Upper sequence 5′ to 3′: SEQ ID NO:399-bar code (BC)-SEQ ID NO:400-insert-SEQ ID NO:401. Middle sequence 5′ to 3′: SEQ ID NO: 402-insert-SEQ ID NO:403-BC-SEQ ID NO: 404. Lower sequence: 5′ to 3′: SEQ ID NO:405. (B) Upper sequence 5′ to 3′: SEQ ID NO:399-bar code (BC)-SEQ ID NO:406-insert-SEQ ID NO:407. Middle sequence 5′ to 3′: SEQ ID NO:408-insert-SEQ ID NO:409-BC-SEQ ID NO:404. Lower sequence: 5′ to 3′: SEQ ID NO:405. Additional modifications as shown in the figure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a sample derived from a subject or specimen.

The term “subject,” as used herein, generally refers to an individual from whom a biological sample is obtained. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “analyte,” as used herein, generally refers to an object that is the subject of analysis, or an object, regardless of being the subject of analysis, that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as to have at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule and where the single-stranded portion is at a 3′ or 5′ end of the same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), 10 Mb, 100 Mb, 1 gigabase or more. A nucleic acid can comprise a sequence of four natural nucleotide bases: adenine (A); cytosine (C); guanine (G); and thymine (T) (or uracil (U) instead of thymine (T) when the nucleic acid is RNA). A nucleic acid may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxy acetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The term “sequencing,” as used herein, generally refers to a process for generating or identifying a sequence of a biological molecule, such as a nucleic acid. The sequence may be a nucleic acid sequence which comprises a sequence of nucleic acid bases. As used herein, the term “template nucleic acid” generally refers to the nucleic acid to be sequenced. The template nucleic acid may be an analyte or be associated with an analyte. For example, the analyte can be a mRNA, and the template nucleic acid is the mRNA or a cDNA derived from the mRNA, or other derivative thereof. In another example, the analyte can be a protein, and the template nucleic acid is an oligonucleotide that is conjugated to an antibody that binds to the protein, or derivative thereof. Examples of sequencing include single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., an A-base-containing solution) to a sequencing reaction space at a first time point and (ii) providing a nucleotide-containing reagent (e.g., G-base-containing solution) to the sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of base types (e.g., A, T, G, C; or U), for example 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space:

(e.g., [A T G C A T G C A T G A T G A T G A T G
C A T G C] (SEQ ID NO: 391)).

Such one-dimensional matrix or linear array of bases in the flow order may also be referred to herein as a “flow space.” A flow order may have any number of nucleotide flows. A “flow position,” as used herein, generally refers to the sequential position of a given nucleotide flow entry in the flow space (e.g., an element in the one-dimensional matrix or linear array). A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A T G C], [A A T T G G C C], [A T], [A/T A/G], [A A], [A], [A T G], etc.). A flow cycle may have any number of nucleotide flows. A given flow cycle may be repeated one or more times in the flow order, consecutively or non-consecutively. Accordingly, the term “flow cycle order,” as used herein, generally refers to an ordering of flow cycles within the flow order, and can be expressed in units of flow cycles. For example, where [A T G C] is identified as a 1^{st} flow cycle, and [A T G] is identified as a 2^{nd} flow cycle, the flow order of [A T G C A T G C A T G A T G A T G A T G C A T G C](SEQ ID NO: 391) may be described as having a flow-cycle order of [1^{st} flow cycle; 1^{st} flow cycle; 2^{nd} flow cycle; 2^{nd} flow cycle; 2^{nd} flow cycle; 1^{st} flow cycle; 1^{st} flow cycle]. Alternatively or in addition, the flow cycle order may be described as [cycle 1, cycle, 2, cycle 3, cycle 4, cycle 5, cycle 6], where cycle 1 is the 1^{st} flow cycle, cycle 2 is the 1^{st} flow cycle, cycle 3 is the 2^{nd} flow cycle, etc.

The term “locus” as used herein generally refers to a base position within a sequence. In some cases, a locus can refer to a specific location in a reference sequence, such as in a reference genome, or a native sample or source. A locus may or may not be a base pair. A locus may have any length, such as 1, 2, 3, 4, 5, 6, 7, 8, 9, 10, or more bases or base pairs. A locus may refer to any contiguous sequence region, such as a homopolymer or non-homopolymer portion of a sequence. A same locus described with respect to two or more sequences, strands, and/or molecules may refer to a common specific base position or common specific location with respect to a native sample or a reference sequence.

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. Amplification of a nucleic acid may be linear, exponential, or a combination thereof. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR (ePCR or emPCR), emulsion RPA (eRPA), dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C. C. PNAS, 1989, 86, 4076-4080 and U.S. Pat. Nos. 5,409,811 and 5,674,716, each of which is herein incorporated by reference in its entirety. Useful methods for clonal amplification from single molecules include rolling circle amplification (RCA) (Lizardi et al., Nat. Genet. 19:225-232 (1998), which is incorporated by reference herein in its entirety), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated by reference herein in its entirety), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adaptor libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated by reference herein in its entirety). Amplification products from a nucleic acid may be identical or substantially identical. A nucleic acid colony resulting from amplification may have identical or substantially identical sequences.

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3):443-53); Smith-Waterman (see, e.g., Smith, Temple F.; and Waterman, Michael S., “Identification of Common Molecular Subsequences” (1981) Journal of Molecular Biology 147:195-197); or BLAST (Basic Local Alignment Search Tool; see, e.g., Altschul S F, Gish W, Miller W, Myers E W, Lipman D J, “Basic local alignment search tool” (1990) J Mol Biol 215 (3):403-410), each of which is incorporated by reference herein in its entirety. As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some embodiments, “substantial identity” exists over a region of the sequences being compared. In some embodiments, substantial identity exists over a region of at least 25 residues in length, at least 50 residues in length, at least 100 residues in length, at least 150 residues in length, at least 200 residues in length, or greater than 200 residues in length. In some embodiments, the sequences being compared are substantially identical over the full length of the sequences being compared. Typically, substantially identical nucleic acid or protein sequences include less than 100% nucleotide or amino acid residue identity, and as such sequences would generally be considered “identical.”

The term “detector,” as used herein, generally refers to a device that is capable of detecting a signal, including a signal indicative of the presence or absence of one or more incorporated nucleotides or fluorescent labels. The detector may simultaneously or substantially simultaneously detect multiple signals. The detector may detect the signal in real-time during, substantially during a biological reaction, such as a sequencing reaction (e.g., sequencing during a primer extension reaction), or subsequent to a biological reaction. In some cases, a detector can include optical and/or electronic components that can detect signals. Non-limiting examples of detection methods, for which a detector is used, include optical detection, spectroscopic detection, electrostatic detection, electrochemical detection, acoustic detection, magnetic detection, and the like. Optical detection methods include, but are not limited to, light absorption, ultraviolet-visible (UV-vis) light absorption, infrared light absorption, light scattering, Rayleigh scattering, Raman scattering, surface-enhanced Raman scattering, Mie scattering, fluorescence, luminescence, and phosphorescence. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products. A detector may be a continuous area scanning detector. For example, the detector may comprise an imaging array sensor capable of continuous integration over a scanning area where the scanning is electronically synchronized to the image of an object in relative motion. A continuous area scanning detector may comprise a time delay and integration (TDI) charge coupled device (CCD), Hybrid TDI, complementary metal oxide semiconductor (CMOS) pseudo TDI device, or TDI line-scan camera.

As used herein, the terms ‘tagment,’ ‘tagmentation’, and ‘tagmented’ may refer to the use of a transposase (e.g., Tn5 transposase) to ligate oligonucleotides to a target molecule (e.g., a double-stranded target molecule). Tagmentation may be used to attach adaptors to the target molecule, for instance where the Tn5 transposase is loaded with the desired adaptors. Descriptions of tagmentation methods are provided in, for example, Picelli. 2014. Tn5 transposase and tagmentation procedures for massively scaled sequencing projects. Genome Res. 24(12), 2033-2040. In some cases, tagmentation is an alternative method to ligation for attachment of multiple double-stranded molecules to each other.

### Sample Processing Methods

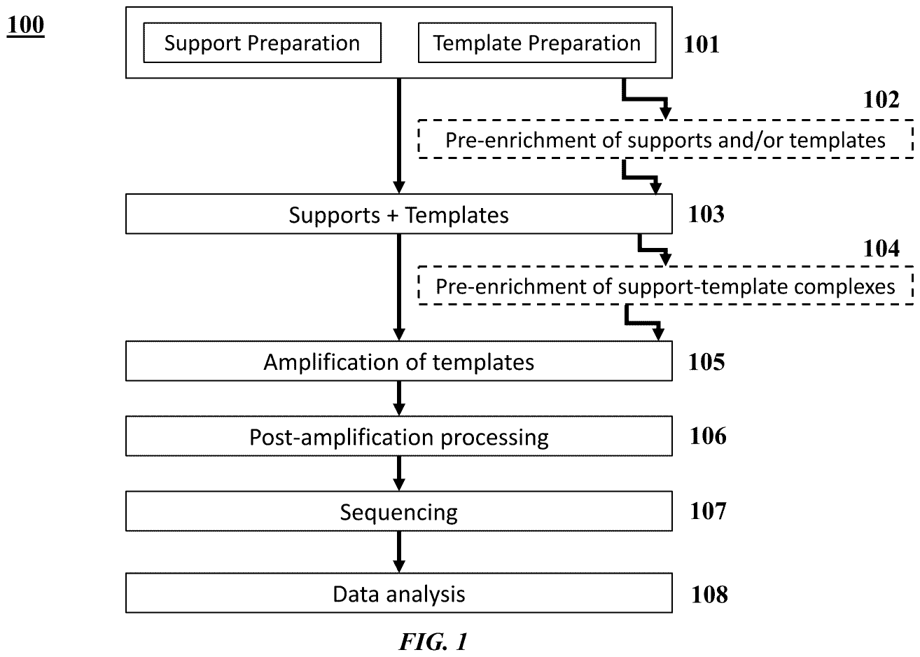

Described herein are devices, systems, methods, compositions, and kits for processing samples, such as to prepare a sample for sequencing, to sequence a sample, and/or to analyze sequencing data. FIG. 1 illustrates an example sequencing workflow 100, according to the devices, systems, methods, compositions, and kits of the present disclosure.

Supports and/or template nucleic acids may be prepared and/or provided (101) to be compatible with downstream sequencing operations (e.g., 107). A support (e.g., bead) may be used to help facilitate sequencing of a template nucleic acid on a substrate. The support may help immobilize a template nucleic acid to a substrate, such as when the template nucleic acid is coupled to the support, and the support is in turn immobilized to the substrate. The support may further function as a binding entity to retain molecules of a colony of the template nucleic acid (e.g., copies comprising identical or substantially identical sequences as the template nucleic acid) together for any downstream processing, such as for sequencing operations. This may be particularly useful in distinguishing a colony from other colonies (e.g., on other supports) and generating amplified sequencing signals for a template nucleic acid sequence.

A support that is prepared and/or provided may comprise an oligonucleotide comprising one or more functional nucleic acid sequences. For example, the support may comprise a capture sequence configured to capture or be coupled to a template nucleic acid (or processed template nucleic acid). For example, the support may comprise the capture sequence, a primer sequence, a barcode sequence, a sample index sequence, a unique molecular identifier (UMI), a flow cell adaptor sequence, an adaptor sequence, a binding sequence for any molecule (e.g., splint, primer, template nucleic acid, capture sequence, etc.), or any other functional sequence useful for a downstream operation, or any combination thereof. The oligonucleotide may be single-stranded, double-stranded, or partially double-stranded.

A support may comprise one or more capture entities, where a capture entity is configured for capture by a capturing entity. A capture entity may be coupled to an oligonucleotide coupled to the support. A capture entity may be coupled to the support. For example, the capturing entity may comprise streptavidin (SA) when the capture moiety comprises biotin. In another example, the capturing entity may comprise a complementary capture sequence when the capture entity comprises a capture sequence (e.g., a capture oligonucleotide that is complementary to the complementary capture sequence). In another example, the capturing entity may comprise an apparatus, system, or device configured to apply a magnetic field when the capture entity comprises a magnetic particle. In another example, the capturing entity may comprise an apparatus, system, or device configured to apply an electrical field when the capture entity comprises a charged particle. In some instances, the capturing entity may comprise one or more other mechanisms configured to capture the capture entity. A capture entity and capturing entity may bind, couple, hybridize, or otherwise associate with each other. The association may comprise formation of a covalent bond, non-covalent bond, and/or releasable bond (e.g., cleavable bond that is cleavable upon application of a stimulus). In some cases, the association may not form any bond. For example, the association may increase a physical proximity (or decrease a physical distance) between the capturing entity and capture entity. In some instances, a single capture entity may be capable of associating with a single capturing entity. Alternatively, a single capture entity may be capable of associating with multiple capturing entities. Alternatively or in addition, a single capturing entity may be capable of associating with multiple capture entities. The capture entity may be capable of linking to a nucleotide. Chemically modified bases comprising biotin, an azide, cyclooctyne, tetrazole, and a thiol, and many others are suitable as capture entities. The capture entity/capturing entity pair may be any combination. The pair may include, but is not limited to, biotin/streptavidin, azide/cyclooctyne, and thiol/maleimide. It will be appreciated that either of the pair may be used as either the capture entity or the capturing entity. In some instances, the capturing entity may comprise a secondary capture entity, for example, for subsequent capture by a secondary capturing entity. The secondary capture entity and secondary capturing entity may comprise any one or more of the capturing mechanisms described elsewhere herein (e.g., biotin and streptavidin, complementary capture sequences, etc.). In some instances, the secondary capture entity can comprise a magnetic particle (e.g., magnetic bead) and the secondary capturing entity can comprise a magnetic system (e.g., magnet, apparatus, system, or device configured to apply a magnetic field, etc.). In some instances, the secondary capture entity can comprise a charged particle (e.g., charged bead carrying an electrical charge) and the secondary capturing entity can comprise an electrical system (e.g., magnet, apparatus, system, or device configured to apply an electric field, etc.).

A support may comprise one or more cleaving moieties. The cleavable moiety may be part of or attached to an oligonucleotide coupled to the support. The cleavable moiety may be coupled to the support. A cleavable moiety may comprise any useful cleavable or excisable moiety that can be used to cleave an oligonucleotide (or portion thereof) from the support. For example, the cleavable moiety may comprise a uracil, a ribonucleotide, or other modified nucleotide that is excisable or cleavable using an enzyme (e.g., UDG, RNAse, endonuclease, exonuclease, etc.). The cleavable moiety may comprise an abasic site or an analog of an abasic site (e.g., dSpacer), a dideoxyribose. The cleavable moiety may comprise a spacer, e.g., C3 spacer, hexanediol, triethylene glycol spacer (e.g., Spacer 9), hexa-ethyleneglycol spacer (e.g., Spacer 18), or combinations or analogs thereof. The cleavable moiety may comprise a photocleavable moiety. The cleavable moiety may comprise a modified nucleotide, e.g., a methylated nucleotide. The modified nucleotide may be recognized specifically by an enzyme (e.g., a methylated nucleotide may be recognized by MspJI). The cleavable moiety may be cleaved enzymatically (e.g., using an enzyme such as UDG, RNAse, APE1, MspJI, etc.). Alternatively, or in addition to, the cleavable moiety may be cleavable using one or more stimuli, e.g., photo-stimulus, chemical stimulus, thermal stimulus, etc.

In some examples, a single support comprises copies of a single species of oligonucleotide, which are identical or substantially identical to each other. In some examples, a single support comprises copies of at least two species of oligonucleotides (e.g., comprising different sequences). For example, a single support may comprise a first subset of oligonucleotides configured to capture a first adaptor sequence of a template nucleic acid and a second subset of oligonucleotides configured to capture a second adaptor sequence of a template nucleic acid.

In some examples, a population of a single species of supports may be prepared and/or provided, where all supports within a species of supports is identical (e.g., has identical oligonucleotide composition (e.g., sequence), etc.). In some examples, a population of multiple species of supports may be prepared and/or provided. For example, a population of supports may be prepared to comprise a plurality of unique support species, where each unique support species comprises a primer sequence unique to said support species. When attaching template nucleic acids to supports, only a template nucleic acid comprising a given adaptor sequence compatible with (e.g., at least partially complementary to) a given primer sequence may be capable of attaching to a given support of a support species comprising the given primer sequence. In another example, a population of supports may be prepared, such that each unique support species comprises a plurality of primer sequences (e.g., a pair of primer sequences) unique to said support species. In some embodiments, the systems and methods disclosed herein can include a population of supports that comprise two, three, four, five, six, seven, eight, nine, ten or more unique support species. Each unique support species can comprise a unique primer sequence that allows selective interactions between the respective support species with an intended binding partner (e.g., a complementary nucleic acid sequence within an adaptor region of a template nucleic acid or an intermediary primer sequence which can subsequently bind to a complementary nucleic acid sequence within an adaptor region of a sample nucleic acid). A population of multiple species of supports may be prepared by first preparing distinct populations of a single species of supports, all different, and mixing such distinct populations of single species of supports to result in the final population of multiple species of supports. A concentration of the different support species within the final mixture may be adjusted accordingly. Devices, systems, methods, compositions, and kits for preparing and using support species are described in further detail in U.S. Patent Pub. No. 20220042072A1 and International Patent Pub. No. WO2022040557A2, each of which is entirely incorporated by reference herein for all purposes.

A template nucleic acid may include an insert sequence sourced from a biological sample. In some cases, the insert sequence may be derived from a larger nucleic acid in the biological sample (e.g., an endogenous nucleic acid), or reverse complement thereof, for example by fragmenting, transposing, and/or replicating from the larger nucleic acid. The template nucleic acid may be derived from any nucleic acid of the biological sample and result from any number of nucleic acid processing operations, such as but not limited to fragmentation, degradation or digestion, transposition, ligation, reverse transcription, extension, etc. A template nucleic acid that is prepared and/or provided may comprise one or more functional nucleic acid sequences. In some cases, the one or more functional nucleic acid sequences may be disposed at one end of the insert sequence. In some cases, the one or more functional nucleic acid sequences may be separated and disposed at both ends of an insert sequence, such as to sandwich the insert sequence. In some cases, a nucleic acid molecule comprising the insert sequence, or complement thereof, may be ligated to one or more adaptor oligonucleotides that comprise such functional nucleic acid sequence(s). In some cases, a nucleic acid molecule comprising the insert sequence, or complement thereof, may be hybridized to a primer comprising such functional nucleic acid sequence(s) and extended to generate a template nucleic acid comprising such functional nucleic acid sequence(s). In some cases, a nucleic acid molecule comprising the insert sequence, or complement thereof, may be hybridized to a primer comprising one or more functional nucleic acid sequence(s) and extended to generate an intermediary molecule, and the intermediary molecule hybridized to a primer comprising additional functional nucleic acid sequence(s) and extended, and so on for any number of extension reactions, to generate a template nucleic acid comprising one or more functional nucleic acid sequence(s). For example, the template nucleic acid may comprise an adaptor sequence configured to be captured by a capture sequence on an oligonucleotide coupled to a support. For example, the template nucleic acid may comprise a capture sequence, a primer sequence, a barcode sequence, a sample index sequence, a unique molecular identifier (UMI), a flow cell adaptor sequence, the adaptor sequence, a binding sequence for any molecule (e.g., splint, primer, template nucleic acid, capture sequence, etc.), or any other functional sequence useful for a downstream operation, or any combination thereof. The template nucleic acid may be single-stranded, double-stranded, or partially double-stranded.

A template nucleic acid may comprise one or more capture entities that are described elsewhere herein. In some cases, in the workflow, only the supports comprise capture entities and the template nucleic acids do not comprise capture entities. In other cases, in the workflow, only the template nucleic acids comprise capture entities and the supports do not comprise capture entities. In other cases, both the template nucleic acids and the supports comprise capture entities. In other cases, neither the supports nor the template nucleic acids comprises capture entities.

A template nucleic acid may comprise one or more cleaving moieties that are described elsewhere herein. In some cases, in the workflow, only the supports comprise cleavable moieties and the template nucleic acids do not comprise cleavable moieties. In other cases, in the workflow, only the template nucleic acids comprise cleavable moieties and the supports do not comprise cleavable moieties. In other cases, both the template nucleic acids and the supports comprise cleavable moieties. In other cases, neither the supports nor the template nucleic acids comprise cleavable moieties. A cleavable moiety may be strategically placed based on a desired downstream amplification workflow, for example.

In some examples, a library of insert sequences are processed to provide a population of template sequences with identical configurations, such as with identical sequences and/or locations of one or more functional sequences. For example, a population of template sequences may comprise a plurality of nucleic acid molecules each comprising an identical first adaptor sequence ligated to a same end. In some examples, a library of insert sequences are processed to provide a population of template sequences with varying configurations, such as with varying sequences and/or locations of one or more functional sequences. For example, a population of template sequences may comprise a first subset of nucleic acid molecules each comprising an identical first adaptor sequence at a first end, and a second subset of nucleic acid molecules each comprising an identical second adaptor sequence at the second end, where the second adaptor sequence is different form the first adaptor sequence. In some instances, a population of template sequences with varying configurations (e.g., varying adaptor sequences) may be used in conjunction with a population of multiple species of supports, such as to reduce polyclonality problems during downstream amplification. A population of multiple configurations of template nucleic acids may be prepared by first preparing distinct populations of a single configuration of template nucleic acids, all different, and mixing such distinct populations of single configurations of template nucleic acids to result in the final population of multiple configurations of template nucleic acids. A concentration of the different configurations of template nucleic acids within the final mixture may be adjusted accordingly.

Optionally, the supports and/or template nucleic acids may be pre-enriched (102). For example, a support comprising a distinct oligonucleotide sequence is isolated from a mixture comprising support(s) that do not have the distinct oligonucleotide sequence. Alternatively, a support population may be provided to comprise substantially uniform supports, where each support comprises an identical surface primer molecule immobilized thereto. For example, template nucleic acids comprising a distinct configuration (e.g., comprising a particular adaptor sequence) is isolated from a mixture comprising template nucleic acids that do not have the distinct configuration. Alternatively, a template nucleic acid population may be provided to comprise substantially uniform configurations. In some cases, the capture entit(ies) on the supports and/or template nucleic acids are used for pre-enrichment.

Subsequent to preparation of the supports and template nucleic acids, the two may be attached (103). A template nucleic acid may be coupled to a support via any method(s) that results in a stable association between the template nucleic acid and the support. For example, the template nucleic acid may hybridize to an oligonucleotide on the support. In another example, the template nucleic acid may hybridize to one or more intermediary molecules, such as a splint, bridge, and/or primer molecule, which hybridizes to an oligonucleotide on the support. Alternatively or in addition, a template nucleic acid may be ligated to one or more nucleic acids on or coupled to the support. Alternatively or in addition, a template nucleic acid may be tagmented with nucleic acids, either in solution or coupled to the support. Alternatively or in addition, a template nucleic acid may be hybridized to an oligonucleotide on a support, which oligonucleotide comprises a primer sequence, and subsequent extension form the primer sequence is performed. Once attached, a plurality of support-template complexes may be generated.

Optionally, support-template complexes may be pre-enriched (104), wherein a support-template complex is isolated from a mixture comprising support(s) and/or template nucleic acid(s) that are not attached to each other. In some cases, the capture entit(ies) on the supports and/or template nucleic acids are used for pre-enrichment.

Subsequent to attachment of the template nucleic acid molecule to the support, the template nucleic acids may be subjected to amplification reactions (105) to generate a plurality of amplification products immobilized to the support. For example, such amplification reactions may comprise performing polymerase chain reaction (PCR) or any other amplification methods described herein, including but not limited to emulsion PCR (ePCR or emPCR), isothermal amplification (e.g., recombinase polymerase amplification (RPA), emulsion RPA (eRPA)), bridge amplification, template walking, etc. In some cases, amplification reactions can occur while the support is immobilized to a substrate. In other cases, amplification reactions can occur off the substrate, such as in solution, or on a different surface or platform. In some cases, amplification reactions can occur in isolated reaction volumes, such as within multiple droplets in an emulsion during emulsion PCR (ePCR or emPCR) or emulsion RPA (eRPA), or in wells. Emulsion PCR methods are described in further detail in U.S. Patent Pub. No. 20220042072A1 and International Patent Pub. No. WO2022040557A2, each of which is entirely incorporated by reference herein for all purposes. Emulsion RPA may comprise performing RPA in compartmentalized droplets in an emulsion. For example, in eRPA, a droplet may comprise a bead (e.g., a single bead), a template (which may or may not be pre-attached to the beads) (e.g., a single template), and RPA reagents (e.g., recombinase, single-stranded binding (SSB) protein, polymerase, primers, dNTPs, etc.).

Subsequent to amplification, the supports (e.g., comprising the template nucleic acids) may be subjected to post-amplification processing (106). Often, subsequent to amplification, a resulting mixture may comprise a mix of positive supports (e.g., those comprising a template nucleic acid molecule) and negative supports (e.g., those not attached to template nucleic acid molecules). Enrichment procedure(s) may isolate positive supports from the mixtures. Example methods of enrichment of amplified supports are described in U.S. Pat. Nos. 10,900,078 and U.S. Patent Pub. No. 20210079464A1 and International Patent Pub. No. WO2022040557A2, each of which is entirely incorporated by reference herein. For example, an on-substrate enrichment procedure may immobilize only the positive supports onto the substrate surface to isolate the positive supports. In some instances, the positive supports may be immobilized to desired locations on the substrate surface (e.g., individually addressable locations), as distinguished from undesired locations (e.g., spacers between the individually addressable locations). In some instances, positive supports and/or negative supports may be processed to selectively remove unamplified surface primers (on the support(s)), such that a resulting positive support retains the template nucleic acid molecule, and a resulting negative support is stripped of the unamplified surface primers. Subsequently, the template nucleic acid(s) on the positive supports may be used to enrich for the positive supports, e.g., by capturing the template nucleic acids.

Subsequent to post-amplification processing, the template nucleic acids may be subject to sequencing (107). The template nucleic acid(s) may be sequenced while attached to the support. Alternatively, the template nucleic acid molecules may be free of the support when sequenced and/or analyzed. In some instances, the template nucleic acids may be sequenced while attached to the support which is immobilized to a substrate. Examples of substrate-based sample processing systems are described elsewhere herein. Any sequencing method described elsewhere herein may be used. In some cases, sequencing by synthesis (SBS) is performed.

In one example (Example A), an SBS method comprises flowing nucleotide reagents according to a flow order comprising a repeat of one 4-base flow (e.g., [A/T/G/C]), where each nucleotide is reversibly terminated (e.g., dideoxynucleotide), and where each base is labeled with a different dye (yielding different optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of each base can be detected by interrogating the different dyes in 4 channels. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example B), an SBS method comprises flowing nucleotide reagents according to a flow order comprising a repeat of a flow cycle of 4 single base flows (e.g., [A T G C]), where each nucleotide is reversibly terminated, and where each base is labeled with a same dye (yielding same frequency optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of the particular base in that flow can be detected by interrogating the wavelength of the dye. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example C), an SBS method comprises flowing nucleotide reagents according to a flow order comprising a repeat of a flow cycle of 4 single base flows (e.g., [A T G C]), where each nucleotide is not terminated, and where each base is labeled with a same dye (yielding same frequency optical signals). With each flow, other sequencing reagents, e.g., sequencing primer, polymerase, buffer, etc. are present to provide sufficient conditions for incorporation of the labeled nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example D), an SBS method comprises flowing nucleotide reagents according to a flow order comprising a repeat of a flow cycle of 4 single base flows (e.g., [A T G C]), where each nucleotide is not terminated, and where only a fraction of the bases in each flow (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals). With each flow, other sequencing reagents, e.g., sequencing primer, polymerase, buffer, etc. are present to provide sufficient conditions for incorporation of the nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example E), an SBS method comprises flowing nucleotide reagents according to a flow order comprising a repeat of a flow cycle of 8 single base flows, with each of the 4 canonical base types flowed twice consecutively within the flow cycle, (e.g., [A A T T G G C C]), where each nucleotide is not terminated, and where only a fraction of the bases in every other flow in the flow cycle (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals) and the nucleotides in the alternating other flow is unlabeled. With each flow, other sequencing reagents, e.g., sequencing primer, polymerase, buffer, etc. are present to provide sufficient conditions for incorporation of the nucleotide into a growing strand hybridized to a template nucleic acid. After one or both of the flows for each canonical base type, an incorporation event or lack thereof of the particular base in that flow can be detected by interrogating the wavelength of the dye. Because the nucleotides are not terminated, if the growing strand is extending through a homopolymer region (e.g., polyT region) of the template nucleic acid, multiple nucleotides may be incorporated during one flow. A first flow of a canonical base type (e.g., A) followed by a second flow of the same canonical base type (e.g., A) may help facilitate completion of incorporation reactions across each growing strand such as to reduce phasing problems. After each or one or more detection events, the labels may be removed (e.g., dyes are cleaved) to reduce signal noise for the next detection.

Labeled nucleotides may comprise a dye, fluorophore, or quantum dot.

It will be appreciated that the combinations of termination states on the nucleotides, label types (e.g., types of dye or other detectable moiety), fraction of labeled nucleotides within a flow, type of nucleotide bases in each flow, type of nucleotide bases in each flow cycle, and/or the order of flows in a flow cycle and/or flow order, other than enumerated in Examples A-E, can be varied for different SBS methods.

Subsequent to sequencing, the sequencing signals collected and/or generated may be subjected to data analysis (108). The sequencing signals may be processed to generate base calls and/or sequencing reads. In some cases, the sequencing reads may be processed to generate diagnostics data to the biological sample, or the subject from which the biological sample was derived from. In some cases, the sequencing reads may be processed to determine whether an amplification cluster has derived from only one or both strands of a double-stranded template nucleic acid molecule and/or determine a ratio or % of amplified strands derived from each strand of the double-stranded template nucleic acid molecule, as described elsewhere herein.

While the sequencing workflow 100 with respect to FIG. 1 has been described with respect to the use of supports to bind template molecules, it will be appreciated that the different supports may be effectively replaced by using spatially distinct locations on one or more surfaces, which do not necessarily have to be the surfaces of individual supports (e.g., beads). For example, a first spatially distinct location on a surface may be capable of directly immobilizing a first colony of a first template nucleic acid and a second spatially distinct location on the same surface (or a different surface) may be capable of directly immobilizing a second colony of a second template nucleic acid to distinguish from the first colony. In some cases, the surface comprising the spatially distinct locations may be a surface of the substrate on which the sample is sequenced, thus streamlining the amplification-sequencing workflow.

It will be appreciated that in some instances, the different operations described in the sequencing workflow 100 may be performed in a different order. It will be appreciated that in some instances, one or more operations described in the sequencing workflow 100 may be omitted or replaced with other comparable operation(s). It will be appreciated that in some instances, one or more additional operations described in the sequencing workflow 100 may be performed.

The different operations described with respect to sequencing workflow 100 may be performed with the help of open substrate systems described herein.

### Open Substrate Systems

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency, such as from faster reagent delivery and lower volumes of reagents required per surface area. The devices, systems, and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used to process any analyte from any sample, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of reagents onto the substrate. The sample processing may permit highly efficient imaging of one or more analytes, or signals corresponding thereto, on the substrate. The sample processing system may comprise an imaging system comprising a detector. Substrates and detectors that can be used in the sample processing system are described in further detail in U.S. Patent Pub. No. 20200326327A1, U.S. Patent Pub. No. 20210354126A1, and U.S. Patent Pub. No. 20210079464A1, each of which is entirely incorporated by reference herein for all purposes.

### Substrates

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. The substrate may comprise multiple layers of the same or different type of material. The substrate may be fully or partially opaque to visible light. The substrate may be fully or partially transparent to visible light. A surface of the substrate may be modified to comprise active chemical groups, such as amines, esters, hydroxyls, epoxides, and the like, or a combination thereof. A surface of the substrate may be modified to comprise any of the binders or linkers described herein. In some instances, such binders, linkers, active chemical groups, and the like may be added as an additional layer or coating to the substrate.

The substrate may have the general form of a cylinder, a cylindrical shell or disk, a rectangular prism, or any other geometric form. The substrate may have a thickness (e.g., a minimum dimension) of at least 100 micrometers (μm), at least 200 μm, at least 500 μm, at least 1 mm, at least 2 millimeters (mm), at least 5 mm, at least 10 mm, or more. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least 1 mm, at least 2 mm, at least 5 mm, at least 10 mm, at least 20 mm, at least 50 mm, at least 100 mm, at least 200 mm, at least 500 mm, at least 1,000 mm, or more.

One or more surfaces of the substrate may be exposed to a surrounding open environment, and accessible from such surrounding open environment. For example, the array may be exposed and accessible from such surrounding open environment. In some cases, as described elsewhere herein, the surrounding open environment may be controlled and/or confined in a larger controlled environment.

The substrate may comprise a plurality of individually addressable locations. The individually addressable locations may comprise locations that are physically accessible for manipulation. The manipulation may comprise, for example, placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings. The individually addressable locations may comprise locations that are digitally accessible. For example, each individually addressable location may be located, identified, and/or accessed electronically or digitally for indexing, mapping, sensing, associating with a device (e.g., detector, processor, dispenser, etc.), or otherwise processing.

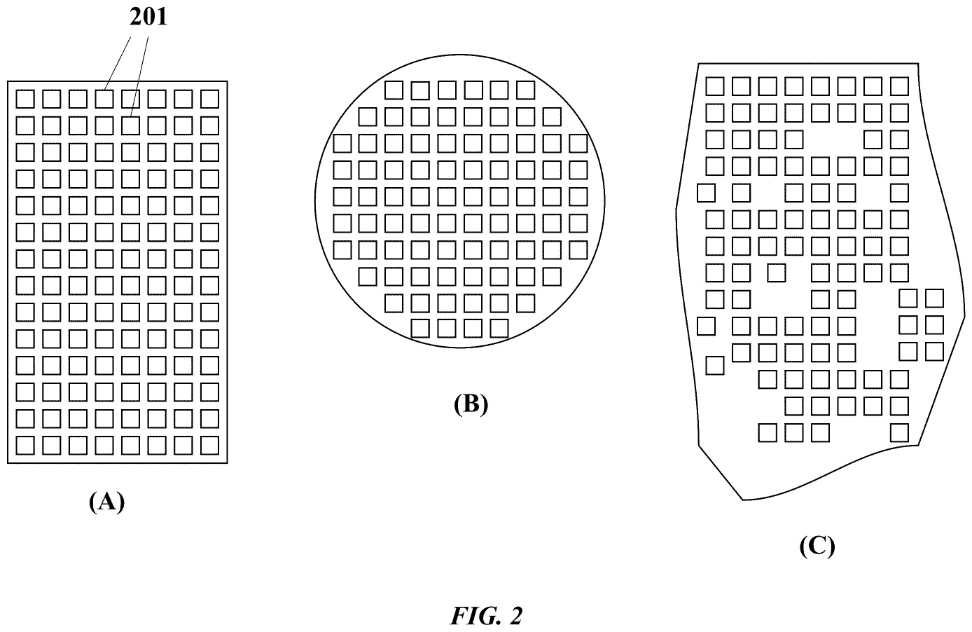

The plurality of individually addressable locations may be arranged as an array, randomly, or according to any pattern, on the substrate. FIG. 2 illustrates different substrates (from a top view) comprising different arrangements of individually addressable locations 201, with panel A showing a substantially rectangular substrate with regular linear arrays, panel B showing a substantially circular substrate with regular linear arrays, and panel C showing an arbitrarily shaped substrate with irregular arrays. The substrate may have any number of individually addressable locations, for example, at least 1, at least 2, at least 5, at least 10, at least 20, at least 50, at least 100, at least 200, at least 500, at least 1,000, at least 2,000, at least 5,000, at least 10,000, at least 20,000, at least 50,000, at least 100,000, at least 200,000, at least 500,000, at least 1,000,000, at least 2,000,000, at least 5,000,000, at least 10,000,000, at least 20,000,000, at least 50,000,000, at least 100,000,000, at least 200,000,000, at least 500,000,000, at least 1,000,000,000, at least 2,000,000,000, at least 5,000,000,000, at least 10,000,000,000, at least 20,000,000,000, at least 50,000,000,000, at least 100,000,000,000 or more individually addressable locations. The substrate may have a number of individually addressable locations that is within a range defined by any two of the preceding values.

Each individually addressable location may have the general shape or form of a circle, pit, bump, rectangle, or any other shape or form (e.g., polygonal, non-polygonal). A plurality of individually addressable locations can have uniform shape or form, or different shapes or forms. An individually addressable location may have any size. In some cases, an individually addressable location may have an area of about 0.1 square micron (μm²), about 0.2 μm², about 0.25 μm², about 0.3 μm², about 0.4 μm², about 0.5 μm², about 0.6 μm², about 0.7 μm², about 0.8 μm², about 0.9 μm², about 1 μm², about 1.1 μm², about 1.2 μm², about 1.25 μm², about 1.3 μm², about 1.4 μm², about 1.5 μm², about 1.6 μm², about 1.7 μm², about 1.75 μm², about 1.8 μm², about 1.9 μm², about 2 μm², about 2.25 μm², about 2.5 μm², about 2.75 μm², about 3 μm², about 3.25 μm², about 3.5 μm², about 3.75 μm², about 4 μm², about 4.25 μm², about 4.5 μm², about 4.75 μm², about 5 μm², about 5.5 μm², about 6 μm², or more. An individually addressable location may have an area that is within a range defined by any two of the preceding values. An individually addressable location may have an area that is less than about 0.1 μm² or greater than about 6 μm².

The individually addressable locations may be distributed on a substrate with a pitch determined by the distance between the center of a first location and the center of the closest or neighboring individually addressable location. Locations may be spaced with a pitch of about 0.1 micron (μm), about 0.2 μm, about 0.25 μm, about 0.3 μm, about 0.4 μm, about 0.5 μm, about 0.6 μm, about 0.7 μm, about 0.8 μm, about 0.9 μm, about 1 μm, about 1.1 μm, about 1.2 μm, about 1.25 μm, about 1.3 μm, about 1.4 μm, about 1.5 μm, about 1.6 μm, about 1.7 μm, about 1.75 μm, about 1.8 μm, about 1.9 μm, about 2 μm, about 2.25 μm, about 2.5 μm, about 2.75 μm, about 3 μm, about 3.25 μm, about 3.5 μm, about 3.75 μm, about 4 μm, about 4.25 μm, about 4.5 μm, about 4.75 μm, about 5 μm, about 5.5 μm, about 6 μm, about 6.5 μm, about 7 μm, about 7.5 μm, about 8 μm, about 8.5 μm, about 9 μm, about 9.5 μm, or about 10 μm. In some cases, the locations may be positioned with a pitch that is within a range defined by any two of the preceding values. The locations may be positioned with a pitch of less than about 0.1 μm or greater than about 10 μm. In some cases, the pitch between two individually addressable locations may be determined as a function of a size of a loading object (e.g., bead). For example, where the loading object is a bead having a maximum diameter, the pitch may be at least about the maximum diameter of the loading object.

Each of the plurality of individually addressable locations, or each of a subset of such locations, may be capable of immobilizing thereto an analyte (e.g., a nucleic acid molecule, a protein molecule, a carbohydrate molecule, etc.) or a reagent (e.g., a nucleic acid molecule, a probe molecule, a barcode molecule, an antibody molecule, a primer molecule, a bead, etc.). In some cases, an analyte or reagent may be immobilized to an individually addressable location via a support, such as a bead. In an example, a bead is immobilized to the individually addressable location, and the analyte or reagent is immobilized to the bead. In some cases, an individually addressable location may immobilize thereto a plurality of analytes or a plurality of reagents, such as via the support. The substrate may immobilize a plurality of analytes or reagents across multiple individually addressable locations. The plurality of analytes or reagents may be of the same type of analyte or reagent (e.g., a nucleic acid molecule) or may be a combination of different types of analytes or reagents (e.g., nucleic acid molecules, protein molecules, etc.). In an example, a first bead comprising a first colony of nucleic acid molecules each comprising a first template sequence is immobilized to a first individually addressable location, and a second bead comprising a second colony of nucleic acid molecules each comprising a second template sequence is immobilized to a second individually addressable location.

A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.). For example, a first type of individually addressable location may bind a first type of biological analyte but not a second type of biological analyte, and a second type of individually addressable location may bind the second type of biological analyte but not the first type of biological analyte.

In some cases, an individually addressable location may comprise a distinct surface chemistry. The distinct surface chemistry may distinguish between different addressable locations. The distinct surface chemistry may distinguish an individually addressable location from a surrounding location on the substrate. For example, a first location type may comprise a first surface chemistry, and a second location type may lack the first surface chemistry. In another example, the first location type may comprise the first surface chemistry and the second location type may comprise a second, different surface chemistry. A first location type may have a first affinity towards an object (e.g., a bead comprising nucleic acid molecules, e.g., amplicons, immobilized thereto) and a second location type may have a second, different affinity towards the same object due to different surface chemistries. In other examples, a first location type comprising a first surface chemistry may have an affinity towards a first sample type (e.g., a bead comprising nucleic acid molecules, e.g., amplicons, immobilized thereto) and exclude a second sample type (e.g., a bead lacking nucleic acid molecules, e.g., amplicons, immobilized thereto). The first location type and the second location type may or may not be disposed on the surface in alternating fashion. For example, a first location type or region type may comprise a positively charged surface chemistry and a second location type or region type may comprise a negatively charged surface chemistry. In another example, a first location type or region type may comprise a hydrophobic surface chemistry and a second location type or region type may comprise a hydrophilic surface chemistry. In another example, a first location type comprises a binder, as described elsewhere herein, and a second location type does not comprise the binder or comprises a different binder. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the surface, such as by etching or notching the surface, using a dye or ink, depositing a topographical mark, depositing a sample (e.g., a control nucleic acid sample), depositing a reference object (e.g., e.g., a reference bead that always emits a detectable signal during detection), and the like, and the individually addressable locations may be indexed with reference to such demarcations. As will be appreciated, a combination of positive demarcations and negative demarcations (lack thereof) may be used to index the individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

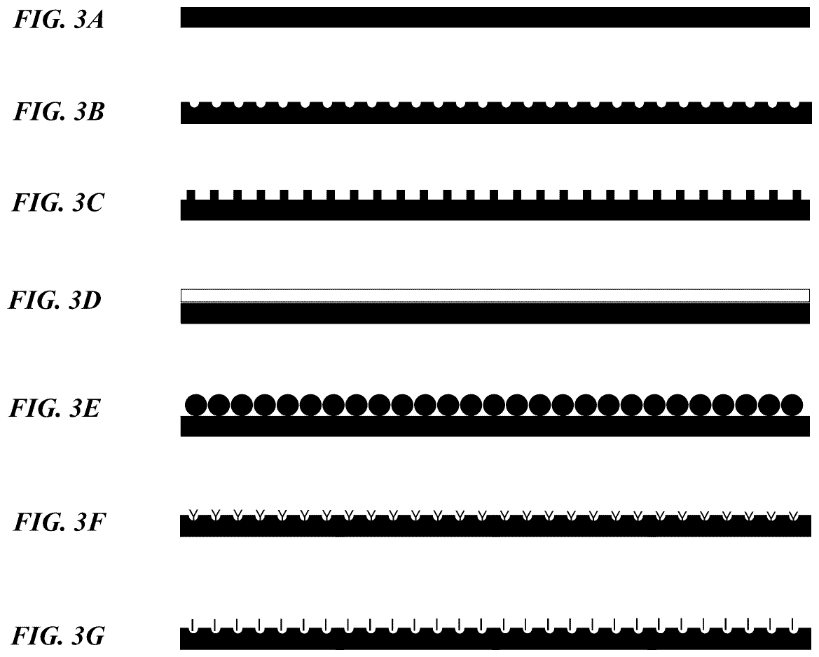

The substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). Alternatively or in addition, a surface of the substrate may be textured or patterned. For example, the substrate may comprise grooves, troughs, hills, and/or pillars. The substrate may define one or more cavities (e.g., micro-scale cavities or nano-scale cavities). The substrate may define one or more channels. The substrate may have regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. In some instances, a texture of the substrate may comprise structures having a maximum dimension of at most about 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. FIGS. 3A-3G illustrate different examples of cross-sectional surface profiles of a substrate. FIG. 3A illustrates a cross-sectional surface profile of a substrate having a completely planar surface. FIG. 3B illustrates a cross-sectional surface profile of a substrate having semi-spherical troughs or grooves. FIG. 3C illustrates a cross-sectional surface profile of a substrate having pillars, or alternatively or in conjunction, wells. FIG. 3D illustrates a cross-sectional surface profile of a substrate having a coating. FIG. 3E illustrates a cross-sectional surface profile of a substrate having spherical particles. FIG. 3F illustrates a cross-sectional surface profile of FIG. 3B, with a first type of binders seeded or associated with the respective grooves. FIG. 3G illustrates a cross-sectional surface profile of FIG. 3B, with a second type of binders seeded or associated with the respective grooves.

A binder may be configured to immobilize an analyte or reagent to an individually addressable location. In some cases, a surface chemistry of an individually addressable location may comprise one or more binders. In some cases, a plurality of individually addressable locations may be coated with binders. In some cases, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the total number of individually addressable locations, or of the surface area of the substrate, are coated with binders. The binders may be integral to the array. The binders may be added to the array. For instance, the binders may be added to the array as one or more coating layers on the array. The substrate may comprise an order of magnitude of at least about 10, 100, 10³, 10⁴, 10⁵, 10⁶, 10⁷, 10⁸, 10⁹, 10¹⁰, 10¹¹, or more binders. Alternatively or in addition, the substrate may comprise an order of magnitude of at most about 10¹¹, 10¹⁰, 10⁹, 10⁸, 10⁷, 10⁶, 10⁵, 10⁴, 10³, 100, 10 or fewer binders.

The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions. For instance, where the analyte or reagent is a nucleic acid molecule, the binders may comprise oligonucleotide adaptors configured to bind to the nucleic acid molecule. In other examples, the binders may comprise one or more of antibodies, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. In some instances, a single binder may bind a single analyte (e.g., nucleic acid molecule) or single reagent. In some instances, a single binder may bind a plurality of analytes (e.g., plurality of nucleic acid molecules) or a plurality of reagents. In some instances, a plurality of binders may bind a single analyte or a single reagent. Though examples herein describe interactions of binders with nucleic acid molecules, the binders may immobilize other molecules (such as proteins), other particles, cells, viruses, other organisms, or the like. Though examples herein describe interactions of binders with samples or analytes, the binders may similarly immobilize reagents. In some instances, the substrate may comprise a plurality of types of binders, for example to bind different types of analytes or reagents. For example, a first type of binders (e.g., oligonucleotides) are configured to bind a first type of analyte (e.g., nucleic acid molecules) or reagent, and a second type of binders (e.g., antibodies) are configured to bind a second type of analyte (e.g., proteins) or reagent. In another example, a first type of binders (e.g., first type of oligonucleotide molecules) are configured to bind a first type of nucleic acid molecules and a second type of binders (e.g., second type of oligonucleotide molecules) are configured to bind a second type of nucleic acid molecules. For example, the substrate may be configured to bind different types of analytes or reagents in certain fractions or specific locations on the substrate by having the different types of binders in the certain fractions or specific locations on the substrate.

The substrate may be rotatable about an axis. The axis of rotation may or may not be an axis through the center of the substrate. In some instances, the systems, devices, and apparatus described herein may further comprise an automated or manual rotational unit configured to rotate the substrate. The rotational unit may comprise a motor and/or a rotor to rotate the substrate. For instance, the substrate may be affixed to a chuck (such as a vacuum chuck). The substrate may be rotated at a rotational speed of at least 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with a rotational velocity that is within a range defined by any two of the preceding values. The substrate may be configured to rotate with different rotational velocities during different operations described herein. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For controlled dispensing, the substrate may be rotating with a rotational frequency of no more than 60 rpm, no more than 50 rpm, no more than 40 rpm, no more than 30 rpm, no more than 25 rpm, no more than 20 rpm, no more than 15 rpm, no more than 14 rpm, no more than 13 rpm, no more than 12 rpm, no more than 11 rpm, no more than 10 rpm, no more than 9 rpm, no more than 8 rpm, no more than 7 rpm, no more than 6 rpm, no more than 5 rpm, no more than 4 rpm, no more than 3 rpm, no more than 2 rpm, or no more than 1 rpm. In some cases the rotational frequency may be within a range defined by any two of the preceding values. In some cases the substrate may be rotating with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, etc.).

In some cases, the substrate may be movable in any vector or direction. For example, such motion may be non-linear (e.g., in rotation about an axis), linear, or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

Loading Reagents onto an Open Substrate

The surface of the substrate may be in fluid communication with at least one fluid nozzle (of a fluid channel). The surface may be in fluid communication with the fluid nozzle via a non-solid gap, e.g., an air gap. In some cases, the surface may additionally be in fluid communication with at least one fluid outlet. The surface may be in fluid communication with the fluid outlet via an air gap. The nozzle may be configured to direct a solution to the array. The outlet may be configured to receive a solution from the substrate surface. The solution may be directed to the surface using one or more dispensing nozzles. For example, the solution may be directed to the array using at least 1, 2, 3, 4, 5, 6, 7, 8, 9, 10, 11, 12, 13, 14, 15, 16, 17, 18, 19, 20 or more dispensing nozzles. The solution may be directed to the array using a number of nozzles that is within a range defined by any two of the preceding values. In some cases, different reagents (e.g., nucleotide solutions of different types, different probes, washing solutions, etc.) may be dispensed via different nozzles, such as to prevent contamination. Each nozzle may be connected to a dedicated fluidic line or fluidic valve, which may further prevent contamination. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Alternatively or in combination, one or more nozzles may be directed closer to the center of the substrate than one or more of the other nozzles. For instance, one or more nozzles used for dispensing washing reagents may be directed closer to the center of the substrate than one or more nozzles used for dispensing active reagents. The one or more nozzles may be arranged at different radii from the center of the substrate. Two or more nozzles may be operated in combination to deliver fluids to the substrate more efficiently. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles.

In some cases, the solution may be dispensed on the substrate while the substrate is stationary; the substrate may then be subjected to rotation (or other motion) following the dispensing of the solution. Alternatively, the substrate may be subjected to rotation (or other motion) prior to the dispensing of the solution; the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film of a nearly constant thickness across the array.

One or more conditions such as the rotational velocity of the substrate, the acceleration of the substrate (e.g., the rate of change of velocity), viscosity of the solution, angle of dispensing (e.g., contact angle of a stream of reagents) of the solution, radial coordinates of dispensing of the solution (e.g., on center, off center, etc.), temperature of the substrate, temperature of the solution, and other factors may be adjusted and/or otherwise optimized to attain a desired wetting on the substrate and/or a film thickness on the substrate, such as to facilitate uniform coating of the substrate. For instance, one or more conditions may be applied to attain a film thickness of at least 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm), or more. Alternatively or in addition, one or more conditions may be applied to attain a film thickness of at most 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm) or less. One or more conditions may be applied to attain a film thickness that is within a range defined by any two of the preceding values. The thickness of the film may be measured or monitored by a variety of techniques, such as thin film spectroscopy with a thin film spectrometer, such as a fiber spectrometer. In some cases, a surfactant may be added to the solution, or a surfactant may be added to the surface to facilitate uniform coating or to facilitate sample loading efficiency. Alternatively or in conjunction, the thickness of the solution may be adjusted using mechanical, electric, physical, or other mechanisms. For example, the solution may be dispensed onto a substrate and subsequently leveled using, e.g., a physical scraper such as a squeegee, to obtain a desired thickness of uniformity across the substrate.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used in reference to a reagent or a sample herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate (or a surface of the substrate). The substrate (or a surface thereof) may then be subjected to vibration, which may spread the reagent to different locations across the substrate (or the surface). Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate. In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

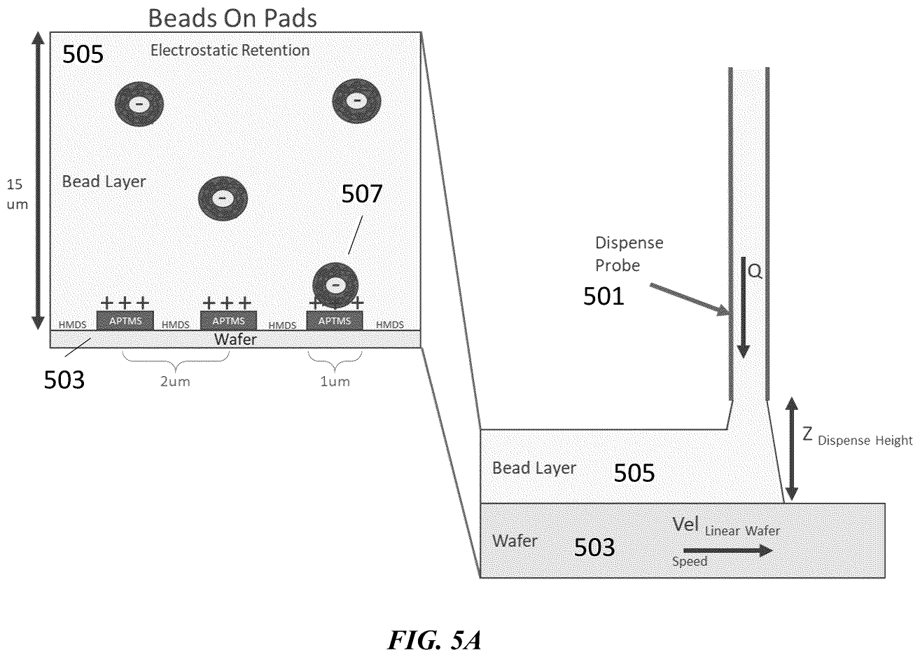

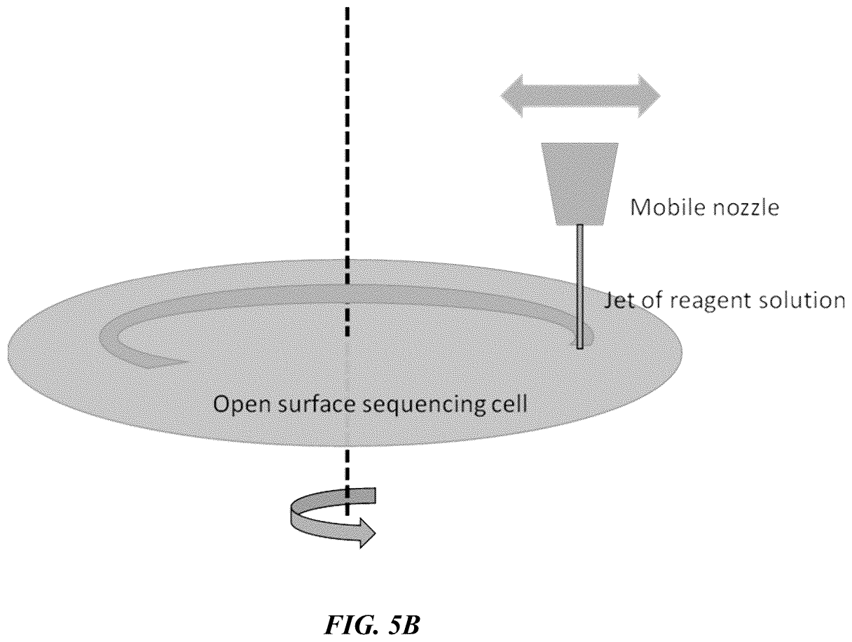

Aerosol delivery may comprise delivering a solution to the substrate in aerosol form by directing the solution to the substrate using a pressure nozzle or an ultrasonic nozzle. Applying the solution using an applicator may comprise contacting the substrate with an applicator comprising the solution and translating the applicator relative to the substrate. For example, applying the solution using an applicator may comprise painting the substrate. The solution may be applied in a pattern by translating the applicator, rotating the substrate, translating the substrate, or a combination thereof. Curtain-coating may comprise dispensing the solution from a dispense probe to the substrate in a continuous stream (e.g., a curtain or a flat sheet) and translating the dispense probe relative to the substrate. A solution may be curtain-coated in a pattern by translating the dispense probe, rotating the substrate, translating the substrate, or a combination thereof. Slot-die coating may comprise dispensing the solution from a dispense probe positioned near the substrate such that the solution forms a meniscus between the substrate and the dispense probe and translating the dispense probe relative to the substrate. A solution may be slot-die coated in a pattern by translating the dispense probe, rotating the substrate, translating the substrate, or a combination thereof. Dispensing the solution from a translating dispense probe may comprise translating the dispense probe relative to the substrate in a pattern (e.g., a spiral pattern, a circular pattern, a linear pattern, a striped pattern, a cross-hatched pattern, or a diagonal pattern). Dispensing the solution from an array of dispense probes may comprise dispensing the solution from an array of nozzles (e.g., a shower head) positioned above the substrate such that the solution is dispensed across an area of the substrate substantially simultaneously. Dipping the substrate into the solution may comprise dipping the substrate into a reservoir comprising the solution. In some embodiments, the reservoir may be a shallow reservoir to reduce the volume of the solution required to coat the substrate. Contacting the substrate to a sheet comprising the solution may comprise bringing the substrate in contact with a sheet of material (e.g., a porous sheet or a fibrous sheet) permeated with the solution. The solution may be transferred to the substrate. In some embodiments, the sheet of material may be a single-use sheet. In some embodiments, the sheet of material may be a reusable sheet. In some embodiments, a solution may be dispensed onto a substrate using the method illustrated in FIG. 5B, where a jet of a solution may be dispensed from a nozzle to a rotating substrate. The nozzle may translate radially relative to the rotating substrate, thereby dispensing the solution in a spiral pattern onto the substrate.

One or more solutions or reagents may be delivered to a substrate by any of the delivery methods disclosed herein. In some embodiments, two or more solutions or reagents are delivered to the substrate using the same or different delivery methods. In some embodiments, two or more solutions are delivered to the substrate such that the time between contacting a solution or reagent and a subsequent solution or reagent is substantially similar for each region of the substrate contacted to the one or more solutions or reagents. In some embodiments, a solution or reagent may be delivered as a single mixture. In some embodiments, the solution or reagent may be dispensed in two or more component solutions. For example, each component of the two or more component solutions may be dispensed from a distinct nozzle. The distinct nozzles may dispense the two or more component solutions substantially simultaneously to substantially the same region of the substrate such that a homogenous solution forms on the substrate. In some embodiments, dispensing of each component of the two or more components may be temporally separated. Dispensing of each component may be performed using the same or different delivery methods. In some embodiments, direct delivery of a solution or reagent may be combined with spin-coating.

A solution may be incubated on the substrate for any desired duration (e.g., minutes, hours, etc.). In some embodiments, the solution may be incubated on the substrate under conditions that maintain a layer of fluid on the surface. One or more of the temperature of the chamber, the humidity of the chamber, the rotation of the substrate, or the composition of the fluid may be adjusted such that the layer of fluid is maintained during incubation. In some instances, during incubation, the substrate may be rotated at an rotational frequency of no more than 60 rpm, 50 rpm, 40 rpm, 30 rpm, 25 rpm, 20 rpm, 15 rpm, 14 rpm, 13 rpm, 12 rpm, 11 rpm, 10 rpm, 9 rpm, 8 rpm, 7 rpm, 6 rpm, 5 rpm, 4 rpm, 3 rpm, 2 rpm, 1 rpm or less. In some cases, the substrate may be rotating with a rotational frequency of about 5 rpm during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

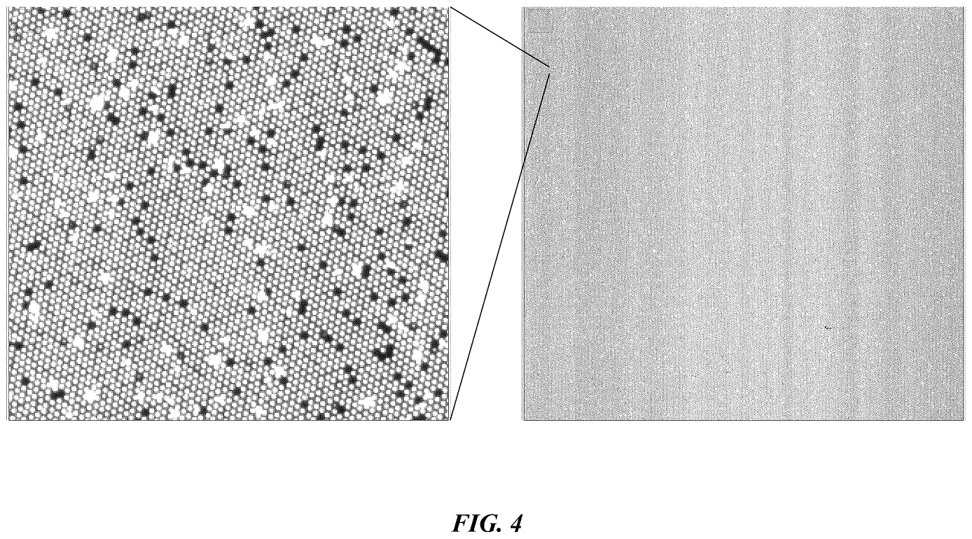

In some cases, a sample may be diluted such that the approximate occupancy of the individually addressable locations is controlled. In some cases, a sample may comprise beads, as described elsewhere herein, for example beads comprising nucleic acid colonies bound thereto. In some cases, an order of magnitude of at least about 10, 100, 1000, 10,000, 100,000, 1,000,000, 10,000,000, 100,000,000, 1,000,000,000, 10,000,000,000, 100,000,000,000 or more beads may be loaded on the substrate, such as to immobilize to as many individually addressable locations. Alternatively or in addition, an order of magnitude of at most about 100,000,000,000, 10,000,000,000, 1,000,000,000, 100,000,000, 10,000,000, 1,000,000, 100,000, 10,000, 1000, 100, or 10 beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag that identifies a bead amongst a plurality of beads. FIG. 4 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface. In some cases, after sample loading, a “bead occupancy” may generally refer to the number of individually addressable locations of a type comprising at least one bead out of the total number of individually addressable locations of the same type. A bead “landing efficiency” may generally refer to the number of beads that bind to the surface out of the total number of beads dispensed on the surface.

In some cases, beads may be dispensed to the substrate according to one or more systems and methods shown in FIGS. 5A-5B. As shown in FIG. 5A, a solution comprising beads may be dispensed from a dispense probe 501 (e.g., a nozzle) to a substrate 503 (e.g., a wafer) to form a layer 505. The dispense probe may be positioned at a height (“Z”) above the substrate. In the illustrated example, the beads are retained in the layer 505 by electrostatic retention and may immobilize to the substrate at respective individually addressable locations. A set of beads in the solution may each comprise a population of amplified products (e.g., nucleic acid molecules) immobilized thereto, which amplified products accumulate to a negative charge on the bead with affinity to a positive charge. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 505 a bead may successfully land on a first location of the first location type (as in 507). In the illustrated example, the location size is 1 micron, the pitch between the different locations of the same location type (e.g., first location type) is 2 microns, and the layer has a depth of 15 micron. FIG. 5B illustrates a reagent (e.g., beads) being dispensed along a path on an open surface of the substrate. As shown in FIG. 5B, a reagent solution may be dispensed from a dispense probe (e.g., a nozzle). The reagent may be dispensed on the surface in any desired pattern or path. This may be achieved by moving one or both of the substrate and the dispense nozzle. The substrate and the dispense probe may move in any configuration with respect to each other to achieve any pattern (e.g., linear pattern, substantially spiral pattern, etc.).

In some instances, a subset or an entirety of the solution(s) may be recycled after the solution(s) have contacted the substrate. Recycling may comprise collecting, filtering, and reusing the subset or entirety of the solution. The filtering may be molecule filtering.

### Detection

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

A detectable signal, such as an optical signal (e.g., fluorescent signal), may be generated upon a reaction between a probe in the solution and the analyte. For example, the signal may originate from the probe and/or the analyte. The detectable signal may be indicative of a reaction or interaction between the probe and the analyte. The detectable signal may be a non-optical signal. For example, the detectable signal may be an electronic signal. The detectable signal may be detected by a detector (e.g., one or more sensors). For example, an optical signal may be detected via one or more optical detectors in an optical detection scheme described elsewhere herein. The signal may be detected during rotation of the substrate. The signal may be detected following termination of the rotation. The signal may be detected while the analyte is in fluid contact with a solution. The signal may be detected following washing of the solution. In some instances, after the detection, the signal may be muted, such as by cleaving a label from the probe and/or the analyte, and/or modifying the probe and/or the analyte. Such cleaving and/or modification may be affected by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of (i) directing a solution to the substrate and (ii) detection of one or more signals indicative of a reaction between a probe in the solution and an analyte immobilized to the substrate, may be repeated any number of times. Such operations may be repeated in an iterative manner. For example, the same analyte immobilized to a given location in the array may interact with multiple solutions in the multiple repetition cycles. For each iteration, the additional signals detected may provide incremental, or final, data about the analyte during the processing. For example, where the analyte is a nucleic acid molecule and the processing is sequencing, additional signals detected for each iteration may be indicative of a base in the nucleic acid sequence of the nucleic acid molecule. In some cases, multiple solutions can be provided to the substrate without intervening detection events. In some cases, multiple detection events can be performed after a single flow of solution. In some instances, a washing solution, cleaving solution (e.g., comprising cleavage agent), and/or other solutions may be directed to the substrate between each operation, between each cycle, or a certain number of times for each cycle.

The optical system may be configured for continuous area scanning of a substrate during rotational motion of the substrate. The term “continuous area scanning (CAS),” as used herein, generally refers to a method in which an object in relative motion is imaged by repeatedly, electronically or computationally, advancing (clocking or triggering) an array sensor at a velocity that compensates for object motion in the detection plane (focal plane). CAS can produce images having a scan dimension larger than the field of the optical system. TDI scanning may be an example of CAS in which the clocking entails shifting photoelectric charge on an area sensor during signal integration. For a TDI sensor, at each clocking step, charge may be shifted by one row, with the last row being read out and digitized. Other modalities may accomplish similar function by high speed area imaging and co-addition of digital data to synthesize a continuous or stepwise continuous scan.

The optical system may comprise one or more sensors. The sensors may detect an image optically projected from the sample. The optical system may comprise one or more optical elements. An optical element may be, for example, a lens, prism, mirror, wave plate, filter, attenuator, grating, diaphragm, beam splitter, diffuser, polarizer, depolarizer, retroreflector, spatial light modulator, or any other optical element. The system may comprise any number of sensors. In some cases, a sensor is any detector as described herein. In some examples, the sensor may comprise image sensors, CCD cameras, CMOS cameras, TDI cameras (e.g., TDI line-scan cameras), pseudo-TDI rapid frame rate sensors, or CMOS TDI or hybrid cameras. The optical system may further comprise any optical source. In some cases, where there are multiple sensors, the different sensors may image the same or different regions of the rotating substrate, in some cases simultaneously. Each sensor of the plurality of sensors may be clocked at a rate appropriate for the region of the rotating substrate imaged by the sensor, which may be based on the distance of the region from the center of the rotating substrate or the tangential velocity of the region. In some cases, multiple scan heads can be operated in parallel along different imaging paths (e.g., interleaved spiral scans, nested spiral scans, interleaved ring scans, nested ring scans). A scan head may comprise one or more of a detector element such as a camera (e.g., a TDI line-scan camera), an illumination source (e.g., as described herein), and one or more optical elements (e.g., as described herein).

The system may further comprise a controller. The controller may be operatively coupled to the one or more sensors. The controller may be programmed to process optical signals from each region of the rotating substrate. For instance, the controller may be programmed to process optical signals from each region with independent clocking during the rotational motion. The independent clocking may be based at least in part on a distance of each region from a projection of the axis and/or a tangential velocity of the rotational motion. The independent clocking may be based at least in part on the angular velocity of the rotational motion. While a single controller has been described, a plurality of controllers may be configured to, individually or collectively, perform the operations described herein.

In some cases, the optical system may comprise an immersion objective lens. The immersion objective lens may be in contact with an immersion fluid that is in contact with the open substrate. The immersion fluid may comprise any suitable immersion medium for imaging (e.g., water, aqueous, organic solution). In some cases, an enclosure may partially or completely surround a sample-facing end of the optical imaging objective. The enclosure may be configured to contain the fluid. The enclosure may not be in contact with the substrate; for example, a gap between the enclosure and the substrate may be filled by the fluid contained by the enclosure (e.g., the enclosure can retain the fluid via surface tension). In some cases, an electric field may be used to regulate a hydrophobicity of one or more surfaces of the container to retain at least a portion of the fluid contacting the immersion objective lens and the open substrate

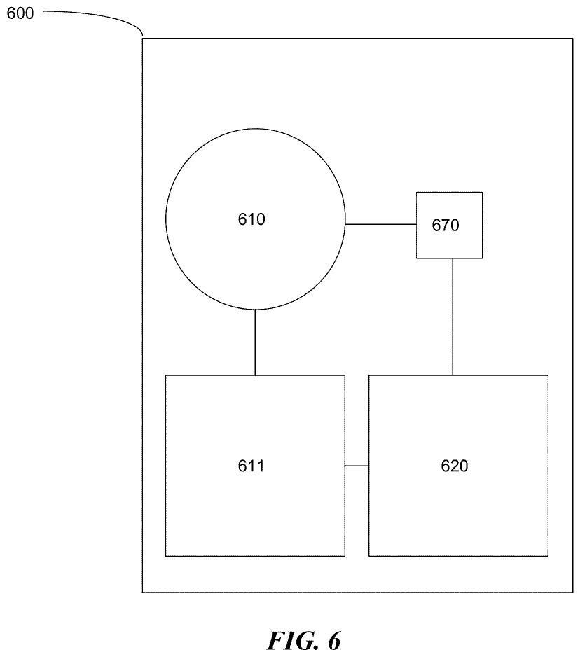

FIG. 6 shows a computerized system 600 for sequencing a nucleic acid molecule. The system may comprise a substrate 610, such as any substrate described herein. The system may further comprise a fluid flow unit 611. The fluid flow unit may comprise any element associated with fluid flow described herein. The fluid flow unit may be configured to direct a solution comprising a plurality of nucleotides described herein to an array of the substrate prior to or during rotation of the substrate. The fluid flow unit may be configured to direct a washing solution described herein to an array of the substrate prior to or during rotation of the substrate. In some instances, the fluid flow unit may comprise pumps, compressors, and/or actuators to direct fluid flow from a first location to a second location. The fluid flow unit may be configured to direct any solution to the substrate 610. The fluid flow system may be configured to collect any solution from the substrate 610. The system may further comprise a detector 670, such as any detector described herein. The detector may be in sensing communication with the substrate surface.

The system may further comprise one or more processors 620. The one or more processors may be individually or collectively programmed to implement any of the methods described herein. For instance, the one or more processors may be individually or collectively programmed to implement any or all operations of the methods of the present disclosure. In particular, the one or more processors may be individually or collectively programmed to: (i) direct the fluid flow unit to direct the solution comprising the plurality of nucleotides across the array during or prior to rotation of the substrate; (ii) subject the nucleic acid molecule to a primer extension reaction under conditions sufficient to incorporate at least one nucleotide from the plurality of nucleotides into a growing strand that is complementary to the nucleic acid molecule; and (iii) use the detector to detect a signal indicative of incorporation of the at least one nucleotide, thereby sequencing the nucleic acid molecule.

### High Throughput

An open substrate system of the present disclosure may comprise a barrier system configured to maintain a fluid barrier between a sample processing environment and an exterior environment. The barrier system is described in further detail in U.S. Patent Pub. No. 20210354126A1, which is entirely incorporated by reference herein. A sample environment system may comprise a sample processing environment defined by a chamber and a lid plate, where the lid plate is not in contact with the chamber. The gap between the lid plate and the chamber may comprise the fluid barrier. The fluid barrier may comprise fluid (e.g., air) from the sample processing environment and/or the exterior environment and may have lower pressure than the sample environment, the external environment, or both. The fluid in the fluid barrier may be in coherent motion or bulk motion.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit(ies), or ranges thereof, within the sample processing environment during various processing operations.

The systems described herein, or any element thereof, may be environmentally controlled. For instance, the systems may be maintained at a specified temperature or humidity. For an operation, the systems (or any element thereof) may be maintained at a temperature of at least 20 degrees Celsius (° C.), 25° C., 30° C., 35° C., 40° C., 45° C., 50° C., 55° C., 60° C., 65° C., 70° C., 75° C., 80° C., 85° C., 90° C., 95° C., 100° C., or more. Alternatively or in addition, for an operation, the systems (or any element thereof) may be maintained at a temperature of at most 100° C., 95° C., 90° C., 85° C., 80° C., 75° C., 70° C., 65° C., 60° C., 55° C., 50° C., 45° C., 40° C., 35° C., 30° C., 25° C., 20° C., or less. Different elements of the system may be maintained at different temperatures or within different temperature ranges, such as the temperatures or temperature ranges described herein. Elements of the system may be set at temperatures above the dew point to prevent condensation. Elements of the system may be set at temperatures below the dew point to collect condensation. In one example, a sample processing environment comprising a substrate as described elsewhere herein may be environmentally controlled from an exterior environment. The sample processing environment may be further divided into separate regions which are maintained at different local temperatures and/or relative humidities, such as a first region contacting or in proximity to a surface of the substrate, and a second region contacting or in proximity to a top portion of the sample processing environment (e.g., a lid). For example, the local environment of the first region may be maintained at a first set of temperatures and first set of humidities configured to prevent or minimize evaporation of one or more reagents on the surface of the substrate, and the local environment of the second region may be maintained at a second set of temperatures and second set of humidities configured to enhance or restrict condensation. The first set of temperatures may be the lowest temperatures within the sample processing environment and the second set temperatures may be the highest temperatures within the sample processing environment.

In some instances, the environmental conditions of the different regions may be achieved by controlling the temperature of the enclosure. In some instances, the environmental conditions of the different regions may be achieved by controlling the temperature of selected parts or whole of the container. In some instances, the environmental conditions of the different regions may be achieved by controlling the temperature of selected parts or whole of the substrate. In some instances, the environmental conditions of the different regions may be achieved by controlling the temperature of reagents dispensed to the substrate. Any combination thereof may be used to control the environmental conditions of the different regions. Heat transfer may be achieved by any method, including for example, conductive, convective, and radiative methods.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

In some instances, an open substrate is retained in the same or approximately the same physical location during processing of an analyte and subsequent detection of a signal associated with a processed analyte.

In some instances, different operations on or with the open substrate are performed in different stations. Different stations may be disposed in different physical locations. For example, a first station may be disposed above, below, adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. In an example, a processing station may comprise a first atmosphere comprising a first set of conditions and a second atmosphere comprising a second set of conditions. The barrier systems may be used to maintain different physical conditions of one or more different stations of the system, or portions thereof, as described elsewhere herein.

The open substrate may transition between different stations by transporting a sample processing environment containing the open substrate (such as the one described with respect to the barrier system) between the different stations. One or more mechanical components or mechanisms, such as a robotic arm, elevator mechanism, actuators, rails, and the like, or other mechanisms may be used to transport the sample processing environment.

An environmental unit (e.g., humidifiers, heaters, heat exchangers, compressors, etc.) may be configured to regulate one or more operating conditions in each station. In some instances, each station may be regulated by independent environmental units. In some instances, a single environmental unit may regulate a plurality of stations. In some instances, a plurality of environmental units may, individually or collectively, regulate the different stations. An environmental unit may use active methods or passive methods to regulate the operating conditions. For example, the temperature may be controlled using heating or cooling elements. The humidity may be controlled using humidifiers or dehumidifiers. In some instances, a part of a particular station, such as within a sample processing environment, may be further controlled from other parts of the particular station. Different parts may have different local temperatures, pressures, and/or humidity.

In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

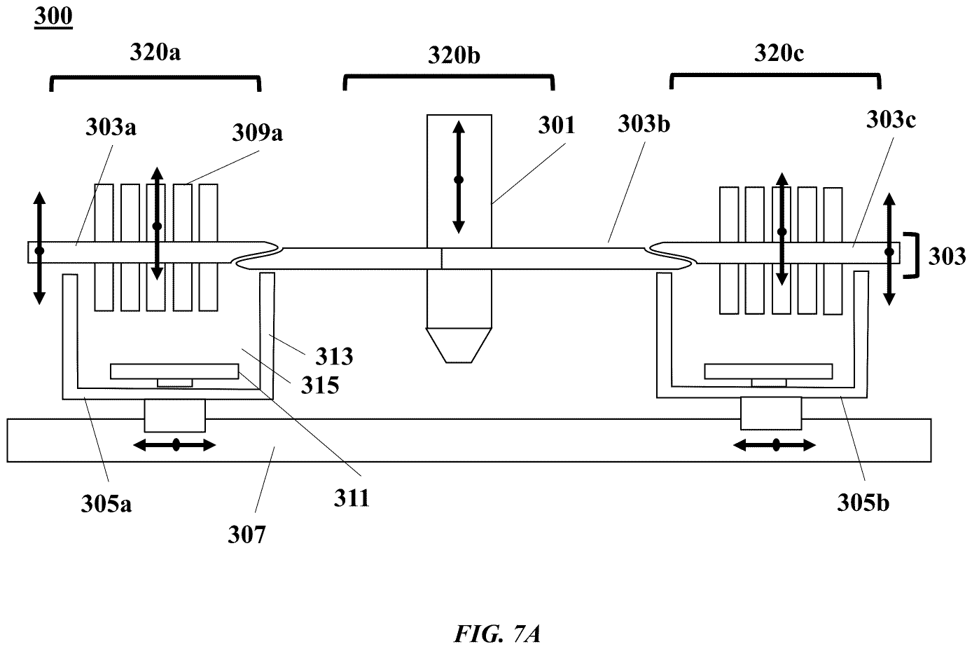

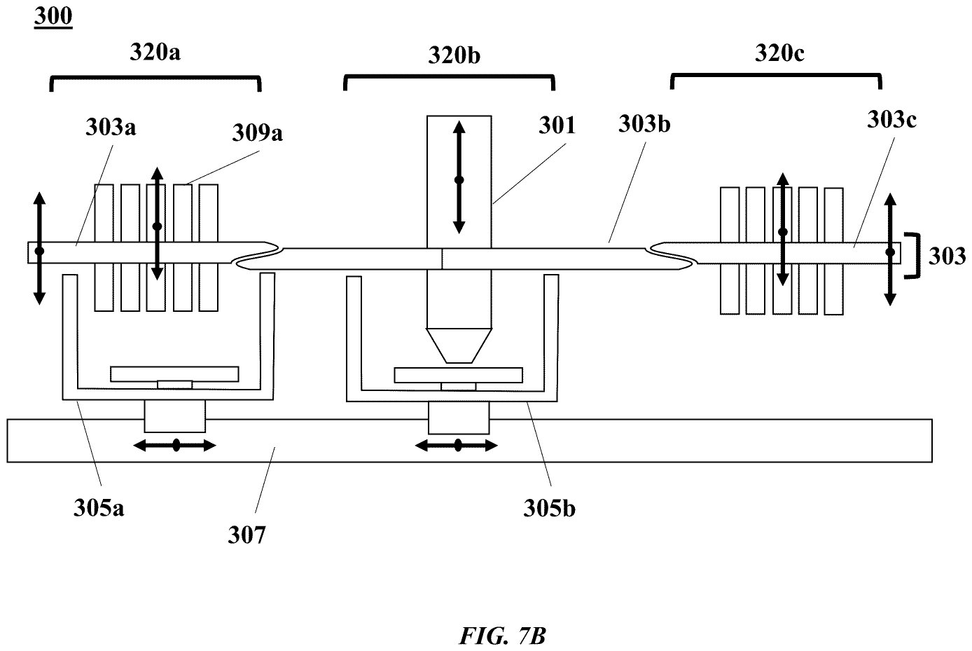

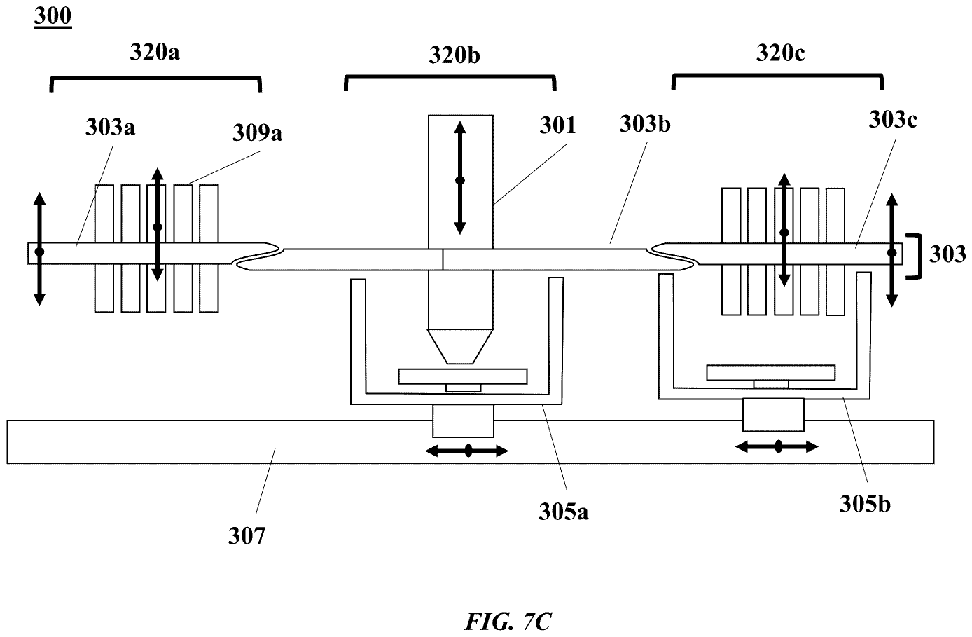

One or more modular sample environment systems (each having its own barrier system) can be used between the different stations. In some instances, the systems described herein may be scaled up to include two or more of a same station type. For example, a sequencing system may include multiple processing and/or detection stations. FIGS. 7A-7C illustrate a system 300 that multiplexes two modular sample environment systems in a three-station system. In FIG. 7B, a first chemistry station (e.g., 320a) can operate (e.g., dispense reagents, e.g., to incorporate nucleotides to perform sequencing by synthesis) via at least a first operating unit (e.g., fluid dispenser 309a) on a first substrate (e.g., 311) in a first sample environment system (e.g., 305a) while substantially simultaneously, a detection station (e.g., 320b) can operate (e.g., scan) on a second substrate in a second sample environment system (e.g., 305b) via at least a second operating unit (e.g., detector 301), while substantially simultaneously, a second chemistry station (e.g., 320c) sits idle. An idle station may not operate on a substrate. An idle station (e.g., 320c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 7C, where the second substrate in the second sample environment system (e.g., 305b) is re-stationed from the detection station (e.g., 320b) to the second chemistry station (e.g., 320c) for operation (e.g., dispensing of reagents, e.g., to incorporate nucleotides to perform sequencing by synthesis) by the second chemistry station, and the first substrate in the first sample environment system (e.g., 305a) is re-stationed from the first chemistry station (e.g., 320a) to the detection station (e.g., 320b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 307) to the different stations and/or the different stations may be physically moved to the different sample environment systems. One or more components of a station, such as modular plates 303a, 303b, 303c of plate 303 defining a particular station(s), may be physically moved to allow a sample environment system to exit the station, enter the station, or cross through the station. During processing of a substrate at station, the environment of a sample environment region (e.g., 315) of a sample environment system (e.g., 305a) may be controlled and/or regulated according to the station's requirements. After the next operating cycle is complete, the sample environment systems can be re-stationed again, such as back to the configuration of FIG. 7B, and this re-stationing can be repeated (e.g., between the configurations of FIGS. 7B and 7C) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations (e.g., 320a, 320c) substantially simultaneously while the detection station (e.g., 320b) is kept idle, such as illustrated in FIG. 7A.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

The nucleic acid sequencing systems and optical systems described herein (or any elements thereof) may be combined in a variety of architectures.

### Retaining and Using Forward and Reverse Strands for Sequencing

Provided herein are devices, systems, methods, compositions, and kits that (i) preserve both strands of a double-stranded template nucleic acid molecule during amplification and/or (ii) enable recognition or quantitative measurement of the fractions of a cluster of amplified molecules which have derived from the respective two strands. Provided herein are devices, systems, methods, compositions, and kits that allow for the simultaneous sequencing of material derived from both strands of a double-stranded template nucleic acid molecule. Such devices, systems, methods, compositions, and kits can be applied alternatively or in addition to one or more of the operations 101-108 described with respect to sequencing workflow 100 of FIG. 1. Such devices, systems, methods, compositions, and kits can be used in conjunction with the sample processing systems and methods, or components thereof (e.g., substrates, detectors, reagent dispensing, continuous scanning, etc.) described herein.

A template nucleic acid molecule that is input to a sequencing library may be double-stranded. In other cases, the template nucleic acid molecule may exist in and/or be converted to double-stranded form during various points in time during the sequencing process, such as during one or more operations of library prep, amplification, and/or enrichment.

Prior to sequencing, a template nucleic acid molecule may be subjected to amplification, such as to generate a colony of multiple copies of the template nucleic acid molecule in a cluster (e.g., on a support such as a bead or a spot on a surface). Amplification may generate multiple distinct molecules that each comprises a copy of the template nucleic acid molecule (or strand thereof) or a single molecule (e.g., a concatemer molecule) that comprises multiple copies of the template nucleic acid molecule (or strand thereof)—either type of amplification product may be referred to herein as a colony or cluster. However, some amplification protocols may amplify only one of the two strands of a double-stranded template nucleic acid molecule, discarding the information (e.g., sequence information) in the other strand. While in some cases the two strands in the double-stranded template nucleic acid molecule may be perfect reverse complements of each other, in which case discarding one of the two strands does not result in loss of information, in some other cases, the two strands may contain site(s) of base mismatch, in which case discarding one of the strands results in loss of valuable information from the template nucleic acid molecule, including a potential alternative base callout at the site(s) in the sequence as well as the fact that there exist site(s) of base mismatch in the first place. For example, PCR-free DNA can contain a high percentage of damaged bases that carry a base mismatch between the two strands.

Sequencing error may be quantified as a rate of error in a total number of sequenced bases. For example, if a human genome (˜3.4 billion bases) is sequenced to 30× depth in whole genome sequencing, at a sequencing error rate of 1e-5, this means that 3.4e9×30×1e-5=approximately 1e6 or 1 million bases are sequenced in error. The methods provided herein may significantly reduce sequencing error rate, such as by an order of magnitude.

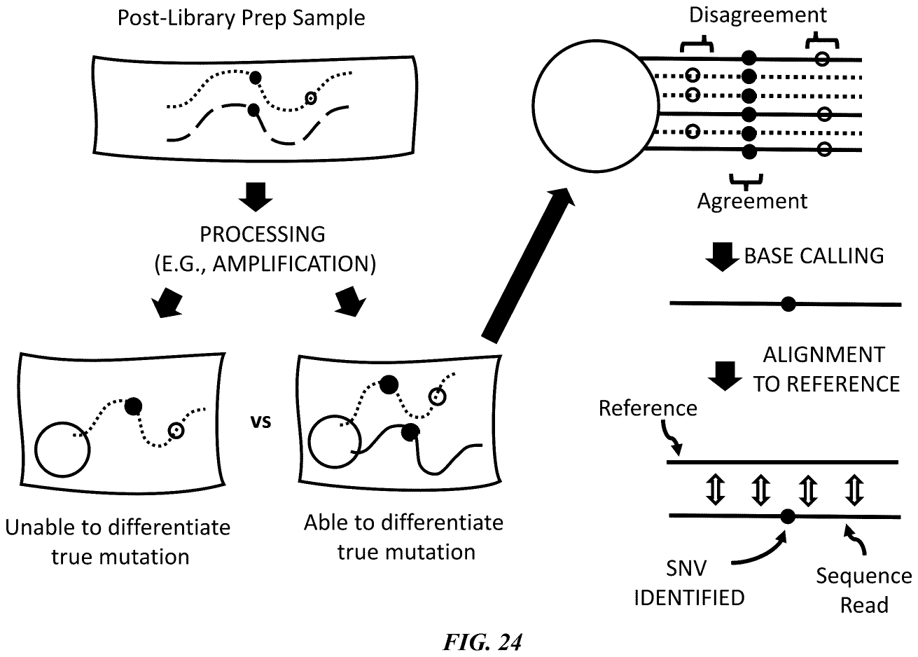

FIG. 24 illustrates one example of how preserving information from both strands of a template nucleic acid molecule for sequencing can permit differentiation of a true mutation from an artificial (false) mutation in a sample. Excluding artificial mutations may reduce sequencing error rate. A single nucleotide variant (SNV) in a sample may be identified by sequencing the sample to generate a sequencing read, aligning the sequencing read to a reference sequence, and comparing the base called at each locus in the sequencing read to that in the reference sequence to identify and call variants from the reference. However, as described elsewhere herein, processing the sample may introduce artificial errors (e.g., artificial base mismatch) that are not native to the sample, which errors may be falsely identified as a true SNV post-sequencing, increasing sequencing error rate and decreasing overall accuracy. Such artificial errors are typically introduced to only one of the two strands in the sample. One example is the spontaneous (or stimulated) deamination of a C residue to a U residue-such deamination may be ‘repaired’ in a living cell by removing the uracil and filing the gap with the correct base (C) by various enzymes (e.g., by uracil-DNA glycosylase, AP endonuclease, polymerase, etc.), but outside of the cell, e.g., in the lab, the deamination cites will carry through to downstream sequencing if not otherwise accounted for or corrected.

In FIG. 24, a filled circle on a line represents a base site in a strand where a true mutation (e.g., C->T mutation) has occurred (compared to a ‘Reference’) and an unfilled circle on a line represents a base site in a strand where an artificial mutation (e.g., C->T mutation) has occurred. In the illustration, the post-library prep sample molecule contains both sites for a true mutation and an artificial mutation, the true mutation occurring in both strands (at corresponding base sites) and the artificial mutation occurring in only one of the strands. For clarity, the solid and dotted lines each represent nucleic acid strands that are otherwise reverse complements of each other (other than at site of artificial base mismatch). The post-library sample may be processed for sequencing, such as to undergo amplification. As shown in the left panel after processing, when information from only one of the strands (e.g., dotted line) is retained for sequencing, such as on a bead, both the base sites for a true mutation and an artificial base mismatch may be identified as variants from the Reference with no way to differentiate what is a true mutation and what is an artificial base mismatch. In contrast, as shown in the right panel after processing, when information from both strands (e.g., dotted line, solid line) is retained for sequencing, such as on a bead, it is possible to differentiate the true mutation from an artificial base mismatch by recognizing that (1) both strand derivatives are being sequenced, (2) where there is a relatively high agreement for a base call for a variant at a locus (e.g., represented by a high confidence level or sequencing quality score, etc.), confirming the base call in the sequencing read, and identifying the locus as a site of true mutation when compared to the Reference, and (3) where there is a relatively high disagreement for a base call for a variant at a locus (e.g., represented by a decreased or diluted sequencing signal, low confidence level or sequencing quality score, etc.), rejecting the base call for the variant in the sequencing read, and thus not identifying the locus as a site of true mutation when compared to the Reference. In some instances, recognizing that (1) both strand derivatives are being sequenced may be achieved by using a processing (e.g., amplification) protocol that retains, or guarantees to retain, both strand information in the sequence-able products, and/or tagging each strand type (e.g., plus strand and minus strand) with a strand recognition element (e.g., sequence) and identifying the strand recognition element prior to, during, or post sequencing to confirm that information from both strand derivatives are present.

Thus, it may be beneficial to preserve information from both strands of a template nucleic acid molecule during amplification. Such preservation may be particularly advantageous in detecting or ruling out single nucleotide variants (SNVs) and/or single nucleotide polymorphisms (SNPs) and improving SNV and/or SNP error rates. A SNP is a genetic variant in a subject's DNA that is especially vulnerable to erroneous detection. When information from both strands in the double-stranded template nucleic acid molecule are preserved for amplification, it may be important that a significant amount from both strands have in fact amplified and are represented in the amplified cluster, as if only 0.01% of material derive from one strand and 99.99% of material derive from the other strand any signal detected from the cluster may effectively be attributed to only the other strand.

### Strand Recognition Mismatch Adaptors for Forward-Reverse Amplified Strand Detection

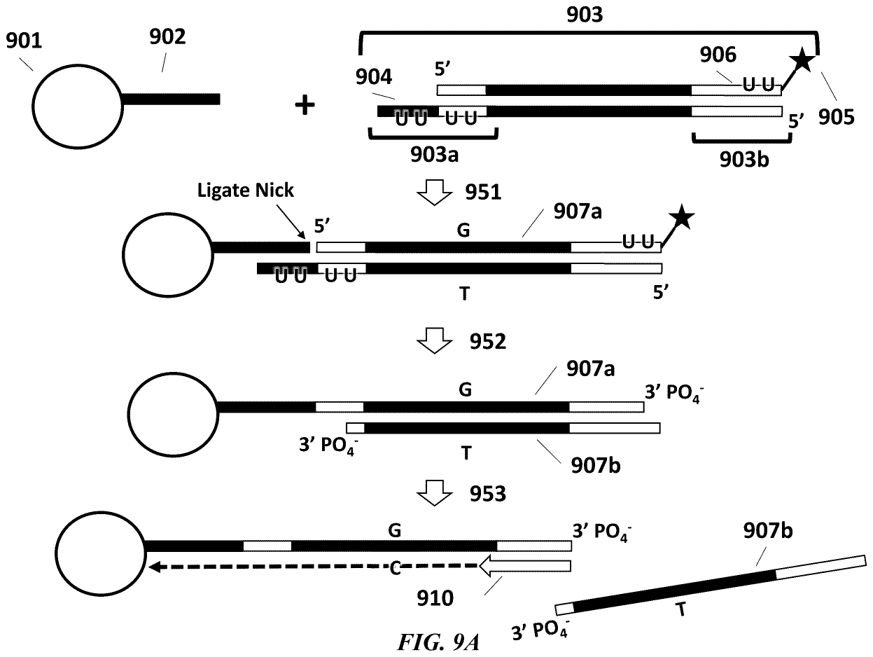

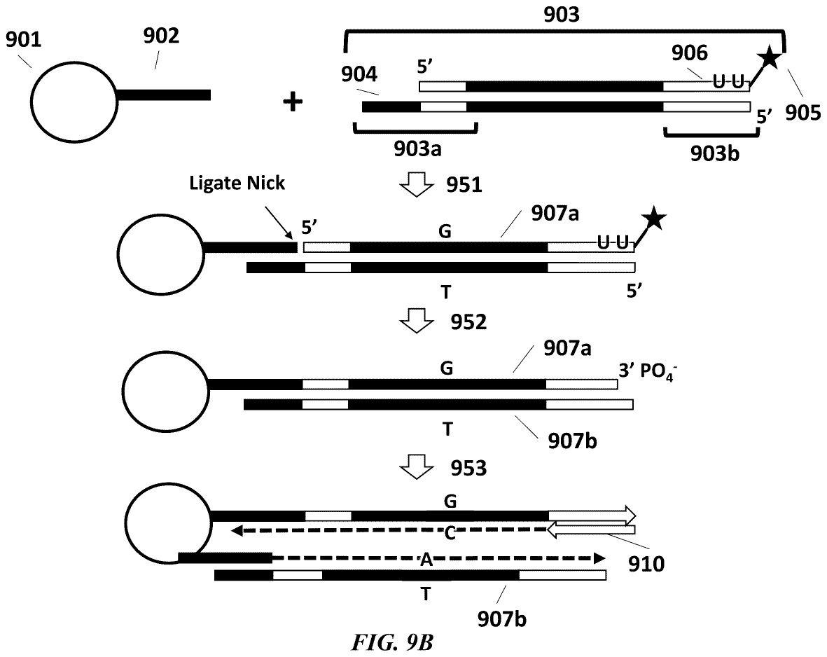

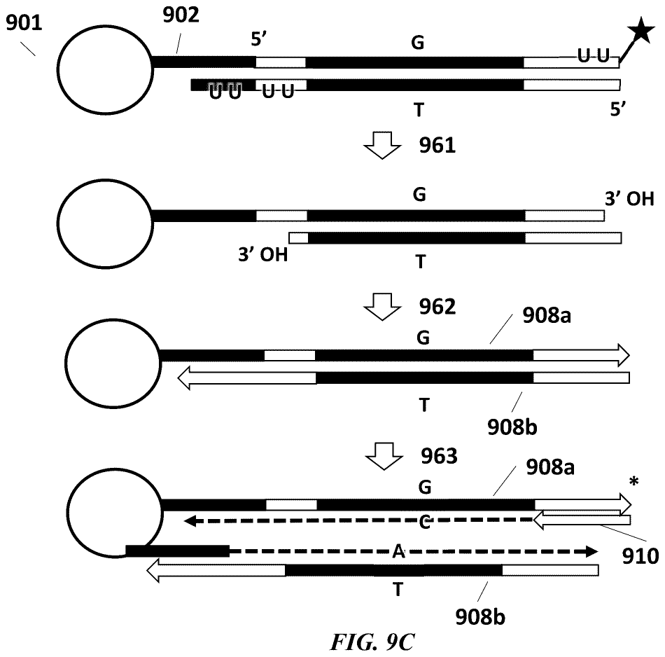

FIG. 9A illustrates a first workflow for extension and amplification on a support, in which one of the template strands is not amplified and thus information not retained in the downstream product. A support 901 and template nucleic acid molecule 903 may be provided. The support 901 may comprise a plurality of surface primers, e.g., 902 (only one illustrated in FIG. 9A for clarity), which may be identical and/or comprise a common primer sequence. The template nucleic acid molecule 903 may comprise a first adaptor 903a attached at one end and a second adaptor 903b attached at another end. The first adaptor may be a partially double-stranded adaptor comprising an overhang 904 configured to bind to surface primer 902. One or more strands of the first adaptor may comprise one or more cleavable moieties (denoted as “U”), such as uracil residues. In the example illustrated in FIG. 9A, the overhang-containing strand comprises the cleavable moieties. The second adaptor 903b may be a double-stranded adaptor. The second adaptor may comprise a capture moiety 905, such as biotin, and one or more cleavable moieties 906 (denoted as “U”), such as uracil residues, which capture moiety is disposed 5′ of the cleavable moieties. In the example illustrated in FIG. 9A, the overhang of the first adaptor and the capture moiety of the second adaptor are attached to different strands of the template insert of the template nucleic acid molecule 903. The template nucleic acid molecule 903 may be attached to the support 901 by annealing the overhang 904 with the surface primer 902 and ligating the nick (951) in the top strand 907a. Prior to or subsequent to nicking, pre-enrichment may be performed where the capture moiety is used to isolate support-template assemblies from negative supports (unbound to a template). In an example, the capture moiety is biotin, and streptavidin-magnetic beads are used to capture the biotin. Then, the U-based cleavage sites may be cleaved using a mix of Uracil-DNA Glycosylase (UDG) and Endonuclease VIII enzymes or USER enzyme mix (952). The UDG may catalyze the hydrolysis of the N-glycosidic bond from deoxyuridine to release uracil, and Endonuclease VIII may cleave the DNA phosphodiester backbone at AP, creating a 1-nucleotide DNA gap with 5′ and 3′ phosphate termini. That is, use of the UDG/Endo VIII enzyme combination results in a blocked 3′ end of the bottom strand 907b of the template. Prior to, during, or subsequent to commencement of extension and/or amplification reactions (953), the blocked bottom strand 907b may be detached from the top strand 907a which is covalently bound to support 901 (e.g., a bead or the surface of a substrate). A primer 910 may anneal to the top strand 907a and a primer extension reaction may commence using the top strand 907a as a template. The template and/or copied strand may then be extended and/or amplified such that a plurality of surface primers on the support 901 is extended to copy the top strand 907a. The extension and/or amplification reactions may be performed in partitions (e.g., wells, emulsions) or in bulk. In the workflow of FIG. 9A, one of the template strands does not get extended because of its blocked end, and thus the resulting amplified support includes only derivatives (e.g., copies) of only one of the strands (top strand 907a). This means that certain errors, such as an error SNP, (e.g., FIGS. 9A-9B illustrate an artificial SNP of a G/T locus that was introduced during a deamination reaction), are carried forward (or their sources lost) during the extension and/or amplification phase as the support retains information only from a first strand (e.g., only the ‘G’ of the G/T locus). The errors may be sequencing or processing errors (e.g., chimeric byproducts, etc.) that contaminate the original template sequence (as sourced from the sample). The error may be a base mismatch error, such as a SNP, an indel, or an artificial base mismatch error. It will be appreciated that alternative enzymes that perform the same or similar functions as those described herein (e.g., any enzyme which cleaves the DNA phosphodiester backbone at AP, creating a 1-nucleotide DNA gap with 5′ and 3′ phosphate termini) may be used in the alternative or in addition. In another example, the cleavable moiety may comprise a ribonucleotide and the enzyme may comprise ribonuclease HII (RNase HII), where upon nicking, a polymerase is used to fill in downstream.

FIG. 9B illustrates a second workflow for extension and amplification on a support, in which both template strands are amplified. A support 901 and template nucleic acid molecule 903 may be provided. The support 901 may comprise a plurality of surface primers, e.g., 902 (only one illustrated in FIG. 9B for clarity), which may be identical and/or comprise a common primer sequence. The template nucleic acid molecule 903 may comprise a first adaptor 903a attached at one end and a second adaptor 903b attached at another end. The first adaptor may be a partially double-stranded adaptor comprising an overhang 904 configured to bind to surface primer 902. Different from FIG. 9A, the first adaptor 903a may not comprise any cleavable moieties. The second adaptor 903b may be a double-stranded adaptor. The second adaptor may comprise a capture moiety 905, such as biotin, and one or more cleavable moieties 906 (denoted as “U”), such as uracil residues, which capture moiety is disposed 5′ of the cleavable moieties. In the example illustrated in FIG. 9B, the overhang of the first adaptor and the capture moiety of the second adaptor are attached to different strands of the template insert of the template nucleic acid molecule 903. The template nucleic acid molecule 903 may be attached to the support 901 (e.g., a bead or the surface of a substrate) by annealing the overhang 904 with the surface primer 902 and ligating the nick (951) in the top strand 907a. Prior to or subsequent to nicking, pre-enrichment may be performed (952) where the capture moiety is used to isolate support-template assemblies from negative supports (unbound to a template). In an example, the capture moiety is biotin, and streptavidin-magnetic beads are used to capture the biotin. Then, the U-based cleavage sites may be cleaved using a mix of Uracil-DNA Glycosylase (UDG) and Endonuclease VIII enzymes or USER enzyme mix. Prior to, during, or subsequent to commencement of extension and/or amplification reactions (953), the bottom strand 907b may be detached from the top strand 907a which is covalently bound to the support 901, and anneal to another surface primer on support 901 (shown in last panel in FIG. 9B). A primer 910 may anneal to the top strand 907a and a primer extension reaction may commence using the top strand 907a as a template. Separately, the other surface primer annealed to the bottom strand 907b may be extended using the bottom strand as a template. The template strands, and/or their copies, may then be extended and/or amplified such that a plurality of surface primers on the support 901 is extended to copy the top strand 907a and the bottom strand 907b. The extension and/or amplification reactions may be performed in partitions (e.g., wells, emulsions) or in bulk. In the workflow of FIG. 9B, unlike in the workflow of FIG. 9A, both template strands are extended, and thus the resulting amplified support includes derivatives (e.g., copies) from both strands (top strand 907a, bottom strand 907b). This means that certain sequencing information, such as an artificial base mismatch error, (e.g., FIGS. 9A-9B illustrate an artificial SNP of a G/T locus that was introduced during a deamination reaction), is retained on the support via the first strand copies (e.g., ‘G’ of the G/T locus) and the second strand copies (e.g., ‘T’ of the G/T locus). It will be appreciated that alternative enzymes that perform the same or similar functions as those described herein (e.g., any enzyme which cleaves the DNA phosphodiester backbone at AP sites via hydrolysis leaving a 1-nucleotide gap with 3-hydroxyl and 5′ deoxyribose phosphate (dRP) termini) may be used in the alternative or in addition. In another example, the cleavable moiety may comprise a ribonucleotide and the enzyme may comprise RNase HII, where upon nicking, a polymerase is used to fill in downstream.

FIG. 9C illustrates a third workflow for extension and amplification on a support, in which the adaptors for the template nucleic acid molecule are the same as in the workflow of FIG. 9A, but in which both template strands are amplified. The support 901 and template nucleic acid molecule 903 may be attached together, and pre-enrichment performed as described with respect to FIG. 9A. The U-based cleavage sites may be cleaved using a mix of Uracil-DNA Glycosylase (UDG) and Apurinic/apyrimidinic Endonuclease 1 (APE1) (961). The UDG may catalyze the hydrolysis of the N-glycosidic bond from deoxyuridine to release uracil, and the APE1 may cleave the DNA phosphodiester backbone at AP sites via hydrolysis leaving a 1-nucleotide gap with 3′-hydroxyl and 5′ deoxyribose phosphate (dRP) termini. That is, use of the UDG/APE1 enzyme combination results in an extendable 3′ end of the bottom strand 908b of the template. Taq or similar polymerizing enzymes and dNTPs may be added to extend the 3′-hydroxyl site (962). The bottom strand 908b may be extended using the top strand 908a bound to the support 901 (e.g., a bead or the surface of a substrate) as a template to generate an extended bottom strand which includes a sequence complementary to the surface primer 902. Then, the extended bottom strand 908b may be detached from the top strand 908a and anneal to another surface primer on support 901. A primer 910 may anneal to the top strand 908a and a primer extension reaction may commence using the top strand 908a as a template (963). Separately, the other surface primer annealed to the extended bottom strand may be extended using the extended bottom strand as a template (963). The template strands, and/or their copies, may be extended and/or amplified such that a plurality of surface primers on the support 901 is extended to copy the top strand 908a and the bottom strand 908b. The extension and/or amplification reactions may be performed in partitions (e.g., wells, emulsions) or in bulk. In some cases, only the first stage extension reaction (e.g., extending the bottom strand 908b to generate the extended bottom strand) may be performed out of partition and the next stage extension reaction(s) may be performed in partition, such that within the partition a support comprising one copy of the top strand and one copy of the bottom strand is subjected to amplification. In the workflow of FIG. 9C, unlike in the workflow of FIG. 9A, both template strands are extended, and thus the resulting amplified support includes derivatives (e.g., copies) from both strands (top strand 908a, bottom strand 908b). This means that certain sequencing information, such as an artificial base mismatch error, (e.g., FIGS. 9A-9C illustrate an artificial SNP of a G/T locus that was introduced during a deamination reaction), is retained on the support via the first strand copies (e.g., ‘G’ of the G/T locus) and the second strand copies (e.g., ‘T’ of the G/T locus). It will be appreciated that alternative enzymes that perform the same or similar functions as those described herein (e.g., any enzyme which cleaves the DNA phosphodiester backbone at AP sites via hydrolysis leaving a 1-nucleotide gap with 3′-hydroxyl and 5′ deoxyribose phosphate (dRP) termini) may be used in the alternative or in addition. In another example, the cleavable moiety may comprise a ribonucleotide and the enzyme may comprise RNase HII, where upon nicking, a polymerase is used to fill in downstream.

The resulting amplified support from workflows where amplified strands attached to the support can be derived from both strands may be a pseudo-polyclonal bead wherein a first set of strands covalently attached to the support are copies of the top strand 908a and a second set of strands covalently attached to the support are copies of the bottom strand 908b. The ratio of top strand and/or top strand copies in all extended strands on the support may be at least about 0.001, 0.01, 0.1, 0.2, 0.3, 0.4, 0.5, 0.6, 0.7, 0.8, 0.9, 0.99, 0.999. The ratio of top strand and/or top strand copies in all extended strands on the support may be at most about 0.001, 0.01, 0.1, 0.2, 0.3, 0.4, 0.5, 0.6, 0.7, 0.8, 0.9, 0.99, 0.999. The ratio of bottom strand and/or bottom strand copies in all extended strands on the support may be at least about 0.001, 0.01, 0.1, 0.2, 0.3, 0.4, 0.5, 0.6, 0.7, 0.8, 0.9, 0.99, 0.999. The ratio of bottom strand and/or bottom strand copies in all extended strands on the support may be at most about 0.001, 0.01, 0.1, 0.2, 0.3, 0.4, 0.5, 0.6, 0.7, 0.8, 0.9, 0.99, 0.999. In some cases, the ratio of top strand copies to bottom strand copies may be approximately 5:5.

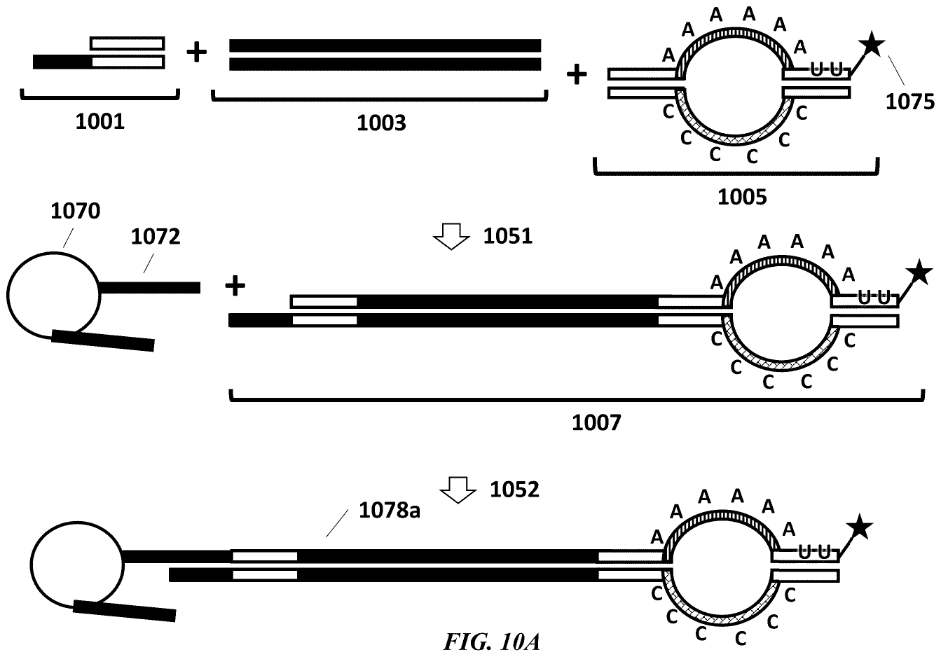

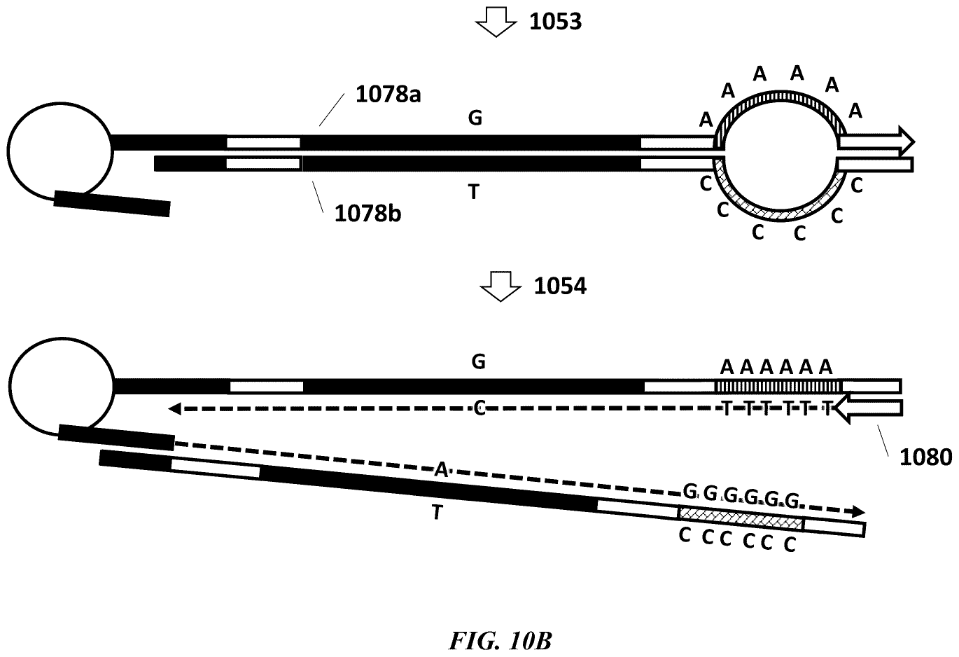

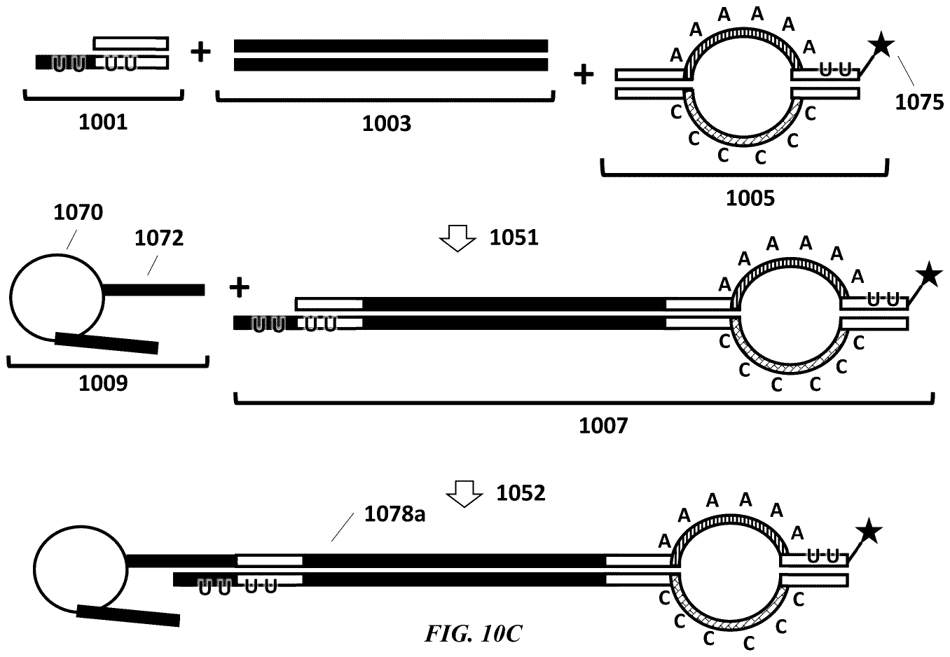

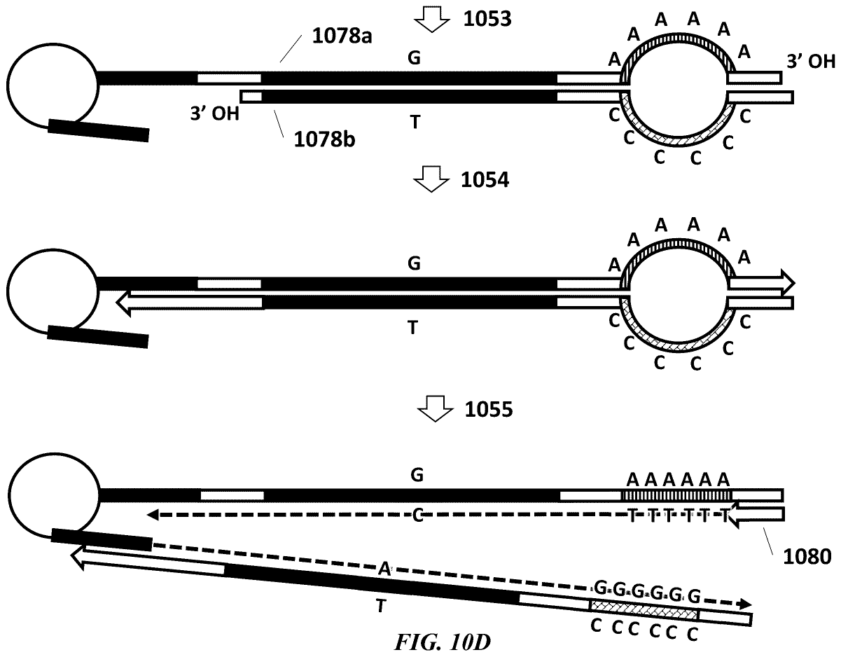

FIGS. 10A-10B illustrate a fourth workflow for extension and amplification on a support, in which both template strands are amplified, and in which an adaptor comprising a mismatch portion is used. A support 1070 and template nucleic acid molecule 1007 may be provided. The support 1070 may comprise a plurality of surface primers, e.g., 1072 (only two illustrated in FIG. 10A for clarity), which may be identical and/or comprise a common primer sequence. The template nucleic acid molecule 1007 may comprise an insert molecule 1003, a first adaptor 1001 attached (1051) at one end of the insert molecule, and a second adaptor 1005 attached (1051) at another end of the insert molecule.

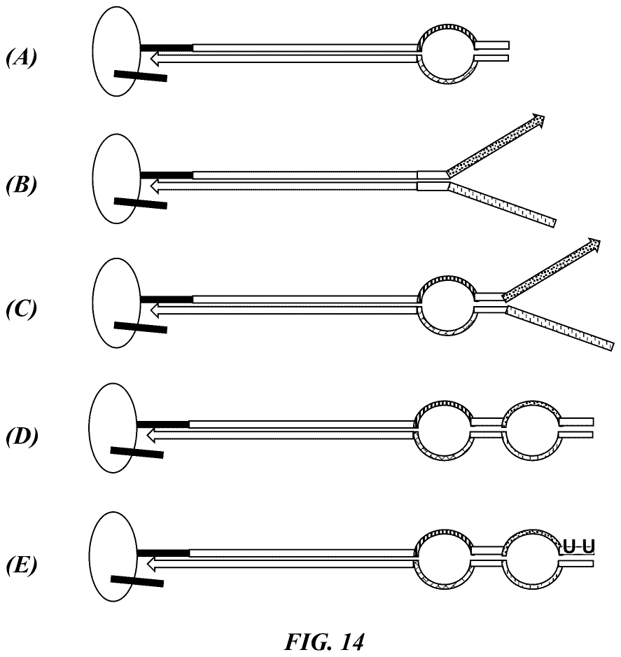

The first adaptor 1001 may be a partially double-stranded adaptor comprising an overhang configured to bind to surface primers 1072. The first adaptor 1001 may not comprise any cleavable moieties. In some cases, the first adaptor 1001 may correspond to the first adaptor 903a of FIG. 9B. The second adaptor 1005 may be a double-stranded adaptor. The second adaptor 1005 may comprise a mismatched portion, where the two strands have a sequence mismatch (have non-complementary sequences) such that they are not annealed in the mismatched portion. In some cases, the second adaptor may comprise the mismatched portion in between two double-stranded portions to form the structure of: (double-stranded portion)-(mismatched portion)-(double-stranded portion). Where the mismatched portion is between two double-stranded portions, it may be referred to as a ‘looped’ structure. In some cases, the second adaptor may comprise the mismatched portion at one end of the adaptor to form the structure of: (double-stranded portion)-(mismatched portion). In some cases, the second adaptor may comprise more than one mismatched portion. In some cases, the second adaptor may comprise an overhang (single-stranded portion) at one end. See FIG. 14 for non-limiting example configurations of template nucleic acid molecules comprising a second adaptor. An at least partially double-stranded adaptor comprising the mismatched portion as described herein may be referred to herein as a strand recognition adaptor.

In some cases, the two strands in the mismatched portion may each comprise a homopolymer sequence that are non-complementary to each other. For example, the top strand/bottom strand pair in the mismatched portion may be selected from the following pairs of homopolymer sequences: (poly-T/poly-G); (poly-T/poly-C); (poly-T/poly-T); (poly-G/poly-G); (poly-G/poly-T); (poly-G/poly-A); (poly-C/poly-C); (poly-C/poly-T); (poly-C/poly-A); (poly-A/poly-G); (poly-A/poly-C); and (poly-A/poly-A). In some cases, the two strands in the mismatched portion may each comprise any sequence that are non-complementary to each other. In some cases, the two strands in the mismatched portion may have the same length or different lengths (e.g., a 5-mer/5-mer pair or a 5-mer/8-mer pair). In some cases, the mismatched portion may comprise a single base mismatch or a bulge loop (e.g., insertion in one strand). The mismatched sequences in the mismatched portion may function as a strand recognition element(s). The second adaptor may comprise a capture moiety 1075, such as biotin, and one or more cleavable moieties (denoted as “U”), such as uracil residues, which capture moiety is disposed 5′ of the cleavable moieties. The first adaptor and/or the second adaptor (e.g., strand recognition adaptor) may comprise any other functional sequence, such as a capture sequence, a primer sequence, an amplification primer sequence, a sequencing primer sequence, a barcode sequence, a sample index sequence, a unique molecular identifier (UMI), a flow cell adaptor sequence, an adaptor sequence, a binding sequence for any molecule (e.g., splint, primer, template nucleic acid, capture sequence, etc.), or any other functional sequence useful for a downstream operation, or any combination thereof. In the example illustrated in FIGS. 10A-10B, the overhang of the first adaptor and the capture moiety of the second adaptor are attached to different strands of the template nucleic acid molecule 1007.

The template nucleic acid molecule 1007 may be attached (1052) to the support 1070 by annealing the overhang of the first adaptor with a surface primer (e.g., 1072) and ligating the nick in the top strand 1078a. Prior to or subsequent to nicking, pre-enrichment may be performed (1053) where the capture moiety 1075 is used to isolate support-template assemblies from negative supports (unbound to a template). In an example, the capture moiety is biotin, and streptavidin-magnetic beads are used to capture the biotin. Then, the U-based cleavage sites may be cleaved using a mix of Uracil-DNA Glycosylase (UDG) and Endonuclease VIII enzymes or USER enzyme mix. Prior to, during, or subsequent to commencement of extension and/or amplification reactions (1054), the bottom strand 1078b may be detached from the top strand 1078a which is covalently bound to the support 1070, and anneal to another surface primer on support 1070. A primer 1080 may anneal to the top strand 1078a and a primer extension reaction may commence using the top strand 1078a as a template. Separately, the other surface primer annealed to the bottom strand 1078b may be extended using the bottom strand as a template. The template strands, and/or their copies, may then be extended and/or amplified such that a plurality of surface primers on the support 1070 is extended to copy the top strand 1078a and the bottom strand 1078b. The extension and/or amplification reactions may be performed in partitions (e.g., wells, emulsions) or in bulk.

FIGS. 10C-10D illustrate a fifth workflow for extension and amplification on a support, in which both template strands are amplified, and in which an adaptor comprising a mismatch portion is used. A support 1070 and template nucleic acid molecule 1007 may be provided. The support 1070 may comprise a plurality of surface primers, e.g., 1072 (only two illustrated in FIG. 10A for clarity), which may be identical and/or comprise a common primer sequence. The template nucleic acid molecule 1007 may comprise an insert molecule 1003, a first adaptor 1001 attached (1051) at one end of the insert molecule, and a second adaptor 1005 attached (1051) at another end of the insert molecule.

The first adaptor may be a partially double-stranded adaptor comprising an overhang configured to bind to surface primers 1072. The first adaptor 1001 may comprise cleavable moieties, denoted as “U”, such as uracil residues. In some cases, the first adaptor 1001 may correspond to the first adaptor 903a of FIG. 9A. The second adaptor 1005 may be the double-stranded adaptor comprising a mismatched portion, as described with respect to FIGS. 10A-10B. The second adaptor may comprise a capture moiety 1075, such as biotin, and one or more cleavable moieties (denoted as “U”), such as uracil residues, which capture moiety is disposed 5′ of the cleavable moieties. In the example illustrated in FIGS. 10C-10D, the overhang of the first adaptor and the capture moiety of the second adaptor are attached to different strands of the template nucleic acid molecule 1007.

The template nucleic acid molecule 1007 may be attached (1052) to the support 1070 by annealing the overhang of the first adaptor with a surface primer (e.g., 1072) and ligating the nick in the top strand 1078a. Prior to or subsequent to nicking, pre-enrichment may be performed (1053) where the capture moiety 1075 is used to isolate support-template assemblies from negative supports (unbound to a template). In an example, the capture moiety is biotin, and streptavidin-magnetic beads are used to capture the biotin. The U-based cleavage sites may be cleaved using a mix of Uracil-DNA Glycosylase (UDG) and Apurinic/apyrimidinic Endonuclease 1 (APE1). Use of the UDG/APE1 enzyme combination may result in an extendable 3′ end of the bottom strand 1078b. Taq or similar polymerizing enzymes and dNTPs may be added to extend the 3′-hydroxyl site (1054). The bottom strand 1078b may be extended using the top strand 1078a bound to the support 1070 as a template to generate an extended bottom strand which includes a sequence complementary to the surface primers 1072. Prior to, during, or subsequent to commencement of extension and/or amplification reactions (1055), the extended bottom strand 1078b may be detached from the top strand 1078a, which is covalently bound to the support 1070 (e.g., a bead or the surface of a substrate), and anneal to another surface primer on support 1070. A primer 1080 may anneal to the top strand 1078a and a primer extension reaction may commence using the top strand 1078a as a template. Separately, the other surface primer annealed to the extended bottom strand 1078b may be extended using the extended bottom strand as a template. The template strands, and/or their copies, may then be extended and/or amplified such that a plurality of surface primers on the support 1070 is extended to copy the top strand 1078a and the bottom strand 1078b. The extension and/or amplification reactions may be performed in partitions (e.g., wells, emulsions) or in bulk.

After amplification, such as using a template nucleic acid molecule that comprises an adaptor comprising a mismatched portion, such as per the workflows of FIGS. 10A-10B and FIGS. 10C-10D, the support may comprise at least two types of amplified strands. Amplified strands on the support which derive from the top strand 1078a may comprise the mismatch sequence (e.g., homopolymer sequence) in the top strand of the mismatched portion of the second adaptor 1005, and amplified strands on the support which derive from the bottom strand 1078b may comprise a complement of the mismatch sequence (e.g., homopolymer sequence) in the bottom strand of the mismatched portion of the second adaptor 1005. That is, the two sets of amplified strands on the support comprise different mismatch sequences (e.g., homopolymer sequences) corresponding to the mismatched portion of the second adaptor. In the example illustrated in FIGS. 10A-10B and FIGS. 10C-10D, the first adaptor comprises a poly-A homopolymer sequence in the top strand of the mismatched portion and a poly-C homopolymer sequence in the bottom strand of the mismatched portion. The resulting amplified bead comprises (i) a first type of amplified strand (derived from the top strand) which comprises the poly-A homopolymer sequence from the top strand of the mismatched portion, and (ii) a second type of amplified strand (derived from the bottom strand) which comprises a poly-G homopolymer sequence which is the complement of the poly-C homopolymer sequence from the bottom strand of the mismatched portion.

It will be appreciated that any of these workflows may omit pre-enrichment and/or enrichment operations, and thus may be implemented where the adaptors for the template do not include a capture moiety and/or cleavable moieties adjacent to the capture moiety.

A sequencing read for an amplified cluster (e.g., on an amplified bead, on a substrate, etc.) may be used to determine whether the amplified cluster comprises amplified strands derived from only one or both of the double-stranded template molecule, and/or determine a % or ratio of amplified strands that derive from a first (e.g., top or bottom) strand over a second (e.g., top or bottom) strand of the double-stranded template molecule. As used herein, the term “forward” with respect to an amplified strand may correspond to an amplified strand that is derived from a first strand of the double-stranded template (expected to contain a first homopolymer sequence of the first strand of the mismatch portion) and the term “reverse” with respect to an amplified strand may correspond to an amplified strand that is derived from a second strand (not the first strand) of the double-stranded template (expected to contain a homopolymer sequence which is complementary to a second homopolymer sequence of the second strand of the mismatch portion). It will be appreciated that the terms “forward” and “reverse” may be flipped with respect to the two strands (first strand, second strand) of the double-stranded template molecule but will generally refer to different strands of the two strands. The terms “forward” and “reverse” strands with reference to a template molecule, as used herein, are generally interchangeable with the terms “plus” and “minus” strands with reference to the template molecule.

An amplified cluster may be sequenced with any sequencing workflow(s) or sequencing method(s) described herein to generate a sequencing read. The sequencing may comprise flow-based sequencing. Flow-based sequencing methods and errors associated with such methods, such as in the flow space (vs base space), are described in U.S. Patent Pub. No. 2020/0372971, which is entirely incorporated herein for all purposes. The sequencing may comprise non-terminated sequencing. The sequencing may comprise reversibly terminated sequencing. The strand recognition element in the mismatched portion may be identified in the flow space or the base space. The mismatch portion of a population of amplified copies (e.g., in a colony or cluster and/or a concatemer) may be identified by any sequencing method, such as flow-based sequencing methods, non-terminated sequencing methods, or reversibly terminated sequencing methods. For example, in non-terminated sequencing methods, e.g., where single base flows are used (e.g., A flow->T flow->G flow->C flow->repeat), sequencing signals read in the flow space may be used to identify the mismatch portion. In another example, in terminated sequencing methods, e.g., where 4-base flows are used (e.g., A/T/G/C flow->A/T/G/C flow->repeat), sequencing signals read in the base space may be used to identify the mismatch portion. One or more photometry and/or base caller algorithms may be used to process sequencing signals and/or sequencing reads.

For illustration purposes, when sequencing in the flow space (e.g., with non-terminated nucleotide flows), where the mismatch portion in the adaptor comprises the following pair of 6-mer homopolymer sequences: TTTTTT/CCCCCC,
for an amplified bead which includes 100% forward strands and 0% reverse strands, at the mismatch portion, the sequencing signals collected from the bead may correspond to 6 T's at 100% intensity and the base caller or photometry algorithm(s) may call the sequence of: TTTTTT;
for an amplified bead which includes 0% forward strands and 100% reverse strands, at the mismatch portion, the sequencing signals collected from the bead may correspond to 6 G's at 100% intensity and the base caller or photometry algorithm(s) may call the sequence of: GGGGGG;
for an amplified bead which includes 50% forward strands and 50% reverse strands, at the mismatch portion, the sequencing signals collected from the bead may correspond to 6 T's at 50% intensity and 6 G's at 50% intensity (or 3 T's at 100% intensity and 3 G's at 100% intensity) and the base caller or photometry algorithm(s) may call the sequence of TTTGGG or GGGTTT (depending on the flow order);
for an amplified bead which includes 66% forward strands and 33% reverse strands, at the mismatch portion, the sequencing signals collected from the bead may correspond to 6 T's at 66% intensity and 6 G's at 33% intensity (or 4 T's at 100% intensity and 2 G's at 100% intensity) and the base caller or photometry algorithm(s) may call the sequence of TTTTGG or GGTTTT (depending on the flow order); and for an amplified bead which includes 17% forward strands and 83% reverse strands, at the mismatch portion, the sequencing signals collected from the bead may correspond to 6 T's at 17% intensity and 6 G's at 83% intensity (or 1 T at 100% intensity and 5 G's at 100% intensity) and the base caller or photometry algorithm(s) may call the sequence of TGGGGG or GGGGGT (depending on the flow order).

In another example, when sequencing in the base space (e.g., with reversibly terminated nucleotide flows), where the mismatch portion in the adaptor comprises the following pair of mismatch sequences: AT/GC,
for an amplified cluster which includes 100% forward strands and 0% reverse strands, at the mismatch portion, the sequencing signals collected from the bead may correspond to: [A at 100% intensity; T at 100% intensity] values. In some cases, the base caller or photometry algorithm(s) may call the sequence of: AT from this data. In other cases, the base caller or photometry algorithm(s) may not output any call for the corresponding portion. In some cases, a sequencing algorithm may output a confirmation of lack of both strand representation and/or % strand type representation data corresponding to: 100% forward and 0% reverse strand representation;
for an amplified cluster which includes 0% forward strands and 100% reverse strands, at the mismatch portion, the sequencing signals collected from the bead may correspond to: [C at 100% intensity; G at 100% intensity] values. In some cases, the base caller or photometry algorithm(s) may call the sequence of: CG. In other cases, the base caller or photometry algorithm(s) may not output any call for the corresponding portion. In some cases, a sequencing algorithm may output a confirmation of lack of both strand representation and/or % strand type representation data corresponding to: 0% forward and 100% reverse strand representation;
for an amplified cluster which includes 50% forward strands and 50% reverse strands, at the mismatch portion, the sequencing signals collected from the bead may correspond to: [A at 50% intensity and C at 50% intensity; T at 50% intensity and G at 50% intensity]. Based on these signals, in some cases, the base caller or photometry algorithm(s) may call the sequence of MK (IUPAC nomenclature, where M represents (A or C) and K represents (G or T)) or NN (IUPAC nomenclature, where N represents (any base)). In other cases, the base caller or photometry algorithm(s) may not output any call for the corresponding portion. In some cases, a sequencing algorithm may output a confirmation of both strand representation and/or % strand type representation data corresponding to: 50% forward and 50% reverse strand representation;
for an amplified cluster which includes 66% forward strands and 33% reverse strands, at the mismatch portion, the sequencing signals collected from the bead may correspond to: [A at 66% intensity and C at 33% intensity; T at 66% intensity and G at 33% intensity]. Based on these signals, in some cases, the base caller or photometry algorithm(s) may call the sequence of AT, MK (IUPAC nomenclature, where M represents (A or C) and K represents (G or T)), or NN (IUPAC nomenclature, where N represents (any base)). In other cases, the base caller or photometry algorithm(s) may not output any call for the corresponding portion. In some cases, a sequencing algorithm may output a confirmation of both strand representation and/or % strand type representation data corresponding to: 66% forward and 33% reverse strand representation; and
for an amplified cluster which includes 17% forward strands and 83% reverse strands, at the mismatch portion, the sequencing signals collected from the bead may correspond to [A at 17% intensity and C at 83% intensity; T at 17% intensity and G at 83% intensity]. Based on these signals, in some cases, the base caller or photometry algorithm(s) may call the sequence of CG, MK (IUPAC nomenclature, where M represents (A or C) and K represents (G or T)), or NN (IUPAC nomenclature, where N represents (any base)). In other cases, the base caller or photometry algorithm(s) may not output any call for the corresponding portion. In some cases, a sequencing algorithm may output a confirmation of both strand representation and/or % strand type representation data corresponding to: 17% forward and 83% reverse strand representation.

As such, the sequencing reads at the mismatch portion of the adaptor may be processed against the pair of sequences (e.g., non-complementary sequences, non-complementary homopolymer sequences, etc.) of the mismatch portion to determine % forward and % reverse strands on the amplified supports. A sequencing algorithm may output a confirmation (or lack thereof) of both strand type representation and/or % strand type representation data (e.g., 17% forward strands and 83% reverse strands in the cluster). In some cases, a confirmation (or lack thereof) of both strand type representation and/or % strand type representation data (e.g., 17% forward strands and 83% reverse strands in the cluster) may be determined solely by photometry algorithms (instead of, or in addition to, base calling algorithms). For example, the mismatch portion may be classified as a preamble (e.g., which can additionally contain other functional sequences, such as calibrating sequences, barcode sequences, adaptor sequences, etc.), whose sequencing signals are deciphered by photometry algorithms but not base-called. In some cases, the % strand type representation data may be used downstream by a base caller or photometry algorithm to call the sequence of a template insert portion. In some cases, a locus where a disagreement in sequencing signal, base call, and/or sequencing read is identified between the plus and minus strand derivatives may be excluded from downstream variant calling, such as SNV or SNP calling.

High accuracy sequencing, high accuracy SNV or SNP calling, or both as achieved by the amplification and/or sequencing methods described herein can have particularly advantageous benefits for applications such as detecting residual disease or minimal residual disease (MRD). Detection of circulating tumor DNA (ctDNA) in blood (e.g., from cell free DNA (cfDNA) samples) can help identify patients whose cancers are more likely to recur and monitor treatment efficacy based on detection and quantification of residual ctDNA before, during, and after treatment. Compared to currently available assays for MRD that rely on targeted sequencing based on enriching a limited number of targeted mutations or methylation markers, where the redundancy in data points (from the targeting) is used to forgive sequencing errors (e.g., SNV call errors), the ability to perform high accuracy SNV calling enables the detection of residual diseases or MRDs using relatively low-depth whole genome sequencing (WGS). SNVs or SNPs called using any of the strand recognition adaptor compositions, kits, methods or systems described herein may be used to determine a level of residual disease, MRD, tumor fraction, or circulating tumor fraction in a subject whose sample was sequenced.

While one example illustrates that the mismatch portion comprises a 6-mer homopolymer sequence pair, the mismatch portion of the adaptor may comprise any length of homopolymer sequences, such as a 2-mer, 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 25-mer, 30-mer or more. In some cases, the mismatch portion may comprise at least a 2-mer, 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 25-mer, 30-mer or longer homopolymer sequence length. Alternatively or in addition, the mismatch portion may comprise at most a 2-mer, 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 25-mer, or 30-mer homopolymer sequence length.

As described elsewhere herein, the mismatch portion of the adaptor may comprise any pair of non-complementary sequences. A sequence of the pair of non-complementary sequences may comprise a homopolymer sequence. A sequence of the pair of non-complementary sequences may not comprise a homopolymer sequence. A sequence of the pair of non-complementary sequences may comprise multiple homopolymer sequences (e.g., AAAGATTT/GGGTCCCC). The pair of non-complementary sequences may comprise a same length (e.g., a 5-mer/5-mer pair). The pair of non-complementary sequences may comprise different lengths (e.g., a 5-mer/8-mer pair). A sequence of the pair of non-complementary sequences may comprise any length of sequences, such as about, at least about, and/or at most about a 1-mer, 2-mer, 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 25-mer, 30-mer, 35-mer, 40-mer, 45-mer, 50-mer or longer lengths. In some cases, portions of the pair of non-complementary sequences may be reverse complements, but nonetheless the entire stretch of the pair of sequences may be non-complementary (e.g., in the pair of non-complementary sequences: AATTGCA/CCAACTG, the TTG/AAC portions are complementary but the other portions are not complementary). In some cases, the mismatched portion may comprise a single base mismatch or a bulge loop (e.g., insertion of any number of bases in one strand).

In some cases, the template nucleic acid molecules may be subjected to one or more resynchronization flows after sequencing through the mismatch portion and prior to sequencing through the insert sequence portions of the template nucleic acid molecules.

Provided herein are methods comprising (1) providing processed product(s) (e.g., amplified product(s)) that retain paired association of two strands of a template nucleic acid, and (2) generating an error-corrected sequencing read of the template nucleic acid by sequencing two sequence portions of the processed product(s) that derive from the two strands of the template nucleic acid, respectively, simultaneously, in a temporally distinct manner, and/or in a spatially distinct manner. For example, two sequencing primers hybridized to the processed product(s) may simultaneously and synchronously extend through the two sequence portions (which may or may not be in the same cluster) to generate sequencing data. In another example, two sequencing primers may extend through the two sequence portions (which may or may not be in the same cluster) at different points in time and/or asynchronously to generate sequencing data. In another example, a sequencing primer may extend through both of the two sequence portions (e.g., in a same nucleic acid strand of the processed product(s)) to generate sequencing data. In some cases, paired association information of two strands of a template nucleic acid may be retained via at least two molecules (e.g., one for each strand of the template nucleic acid), which may be associated via immobilization to a same spatial location and/or support. In some cases, paired association information of two strands of a template nucleic acid may be retained via a single molecule (e.g., comprising information from both strands), such as via a concatemeric product or other product that joins, or derived from a joined version of, the two strands of the template nucleic acid.

Advantageously, the methods described herein may simultaneously sequence material derived from both strands of a library molecule, and/or generate a single sequencing read that represents both strand derivatives of a template nucleic acid. For example, the same nucleotide flow or sets of nucleotide flows may simultaneously interrogate material derived from both strands. Such simultaneous sequencing of both strand derivatives is distinguished from, and more efficient compared to, generating two distinct sequencing reads each pertaining to a different strand of the library molecule—for example, some paired end sequencing methods comprise generating a first read by sequencing a first strand of the template nucleic acid and generating a second read (distinct from the first read) by sequencing a second strand of the template nucleic acid (and/or a reverse complement of the first strand), and then pairing the first and second reads to generate a consensus paired read. Advantageously, simultaneous sequencing of both strand derivatives also permit association and recognition of the presence of both strand derivatives without needing barcodes, or having to sequence additional bases in the barcode region—for example, some barcoded duplex methods barcode each molecule with a unique barcode, and then later associate different strand derivatives via the same barcode; however such barcoded duplex methods require large overhead, including significant barcode reagents and extraneous sequencing and data processing of barcode regions. In some of the methods provided herein, both strand derivatives are already provided in a single amplified cluster, such that a signal read from the amplified cluster (spatially indexed to the amplified cluster) automatically associates both strand derivatives together.

Provided herein are methods for generating amplified products that retain or preserve information from both strands of a library nucleic acid molecule. The amplified products may be generated via any amplification method, such as PCR, ePCR, RPA, eRPA, RCA, MDA, or bridge amplification. Examples of preserving both strand information through various amplification methods are detailed herein. In some cases, the two strands of a library nucleic acid molecule may be joined into a single strand prior to amplification. The amplified products may comprise concatemer molecules (each comprising multiple copies of a template strand or multiple copies of both template strands) or distinct molecules (each comprising a copy of a template strand or each comprising a copy of each of both template strands). Amplified products may be generated as clusters or colonies, each cluster or colony having derived from a distinct library molecule. Amplified products may be generated on, or while attached on, a support. Amplified products may be generated in solution. Amplified products may be sequenced while immobilized on a substrate, as described elsewhere herein.

Provided herein are methods for generating detectable amplified supports which comprises amplified strands derived from both strands of a double-stranded template nucleic acid molecule. A method may comprise (a) attaching a double-stranded template molecule to a support to generate a template-attached support, wherein the double-stranded template molecule comprises a first strand and a second strand, wherein the double-stranded template molecule comprises an adaptor comprising a mismatch portion, wherein the mismatch portion comprises a first sequence in the first strand and a second sequence in the second strand that is not complementary to the first sequence; and (b) subjecting the double-stranded template molecule of the template-attached support to amplification to generate an amplified support comprising a plurality of amplified strands attached thereto. The plurality of amplified strands may comprise derivatives from both the first strand and the second strand. The method may further comprise hybridizing a plurality of sequencing primers to the plurality of amplified strands and extending the plurality of sequencing primers to sequence the amplified strands. A sequencing signal collected from an individually addressable location after a sequencing flow may represent both strand derivatives.

Provided herein are methods for detecting amplified strands on a support, such as a % or ratio of amplified strands that derive from one or both of the strands in a double-stranded template nucleic acid molecule. A method may comprise: (a) attaching a double-stranded template molecule to a support to generate a template-attached support, wherein the double-stranded template molecule comprises a first strand and a second strand, wherein the double-stranded template molecule comprises an adaptor comprising a mismatch portion, wherein the mismatch portion comprises in the first strand a first sequence and in the second strand a second sequence that is not complementary to the first sequence; (b) subjecting the double-stranded template molecule of the template-attached support to amplification to generate an amplified support comprising a plurality of amplified strands attached thereto; (c) sequencing the amplified support to generate a sequencing read; and (d) based at least in part on a portion of the sequencing read that corresponds to the mismatch portion, determining a percentage of amplified strands in the plurality of amplified strands in the amplified support that derives from the first strand. The sequencing in (c) may comprise hybridizing a plurality of sequencing primers to the plurality of amplified strands, which derive from both the first strand the second strand, and extending the plurality of sequencing primers to sequence the plurality of amplified strands. A sequencing signal collected from an individually addressable location after a sequencing flow may represent both strand derivatives.

Provided herein are methods for generating detectable amplified supports which comprises amplified strands derived from both strands of a double-stranded template nucleic acid molecule. A method may comprise (a) attaching a double-stranded template molecule to a support to generate a template-attached support, wherein the double-stranded template molecule comprises a first strand and a second strand, wherein the double-stranded template molecule comprises an adaptor comprising a mismatch portion, wherein the mismatch portion comprises in the first strand a first homopolymer sequence and in the second strand a second homopolymer sequence that is not complementary to the first homopolymer sequence; and (b) subjecting the double-stranded template molecule of the template-attached support to amplification to generate an amplified support comprising a plurality of amplified strands attached thereto.

Provided herein are methods for detecting amplified strands on a support, such as a % or ratio of amplified strands that derive from one or both of the strands in a double-stranded template nucleic acid molecule. A method may comprise: (a) attaching a double-stranded template molecule to a support to generate a template-attached support, wherein the double-stranded template molecule comprises a first strand and a second strand, wherein the double-stranded template molecule comprises an adaptor comprising a mismatch portion, wherein the mismatch portion comprises in the first strand a first homopolymer sequence and in the second strand a second homopolymer sequence that is not complementary to the first homopolymer sequence; (b) subjecting the double-stranded template molecule of the template-attached support to amplification to generate an amplified support comprising a plurality of amplified strands attached thereto; (c) sequencing the amplified support to generate a sequencing read; and (d) based at least in part on a portion of the sequencing read that corresponds to the mismatch portion, determining a percentage of amplified strands in the plurality of amplified strands in the amplified support that derives from the first strand.

Provided herein are kits for generating detectable amplified supports and detecting amplified strands on a support. A kit may comprise a double-stranded adaptor comprising a mismatch portion, wherein the double-stranded adaptor comprises a first strand and a second strand, wherein the mismatch portion comprises in the first strand a first sequence and in the second strand a second sequence that is not complementary to the first sequence. The kit may comprise a plurality of double-stranded adaptors comprising the mismatch portion, wherein the mismatch portion of the plurality of double-stranded adaptors is identical.

Provided herein are kits for generating detectable amplified supports and detecting amplified strands on a support. A kit may comprise a double-stranded adaptor comprising a mismatch portion, wherein the double-stranded adaptor comprises a first strand and a second strand, wherein the mismatch portion comprises in the first strand a first homopolymer sequence and in the second strand a second homopolymer sequence that is not complementary to the first homopolymer sequence. The kit may comprise a plurality of double-stranded adaptor comprising the mismatch portion, wherein the mismatch portion of the plurality of double-stranded adaptor is identical.

Provided herein are compositions for generating detectable amplified supports and detecting amplified strands on a support. A composition can comprise a double-stranded adaptor comprising a mismatch portion, wherein the double-stranded adaptor comprises a first strand and a second strand, wherein the mismatch portion comprises in the first strand a first sequence and in the second strand a second sequence that is not complementary to the first sequence. The composition may comprise a plurality of double-stranded adaptors each comprising the mismatch portion, wherein the mismatch portion of the plurality of double-stranded adaptor is identical. The composition may comprise a plurality of template molecules, wherein the plurality of template molecules comprises a plurality of double-stranded template insert molecules ligated to the plurality of double-stranded adaptors. The composition may comprise a template molecule, wherein the template molecule comprises a double-stranded template insert molecule ligated to the double-stranded adaptor. The composition may comprise a support. The composition may comprise a plurality of supports. The support may be attached to the template molecule.

Provided herein are compositions for generating detectable amplified supports and detecting amplified strands on a support. A composition can comprise a double-stranded adaptor comprising a mismatch portion, wherein the double-stranded adaptor comprises a first strand and a second strand, wherein the mismatch portion comprises in the first strand a first homopolymer sequence and in the second strand a second homopolymer sequence that is not complementary to the first homopolymer sequence. The composition may comprise a plurality of double-stranded adaptors each comprising the mismatch portion, wherein the mismatch portion of the plurality of double-stranded adaptor is identical. The composition may comprise a plurality of template molecules, wherein the plurality of template molecules comprises a plurality of double-stranded template insert molecules ligated to the plurality of double-stranded adaptors. The composition may comprise a template molecule, wherein the template molecule comprises a double-stranded template insert molecule ligated to the double-stranded adaptor. The composition may comprise a support. The composition may comprise a plurality of supports. The support may be attached to the template molecule.

### Enrichment of Balanced Supports

A plurality of balanced supports may be generated using any of the methods described herein. A balanced support may generally refer to a support comprising amplified strands, wherein the amplified strands can be derived from both strands of a double-stranded template nucleic acid molecule, wherein the template nucleic acid molecule comprises a mismatch portion. A balanced support may comprise any ratio of a first set of amplified strands and a second set of amplified strands derived from a first strand and a second strand of a template nucleic acid molecule, respectively. In some cases, the balanced support may comprise amplified strands derived from only one of the two strands of the template nucleic acid molecule (forward only or reverse only).

A plurality of balanced supports may be enriched prior to loading onto a substrate for sequencing, for example to enrich for balanced supports that include amplified strands derived from both strands of a template nucleic acid molecule (as opposed to balanced supports that include only forward or reverse strands). A portion of an amplified strand which corresponds to the mismatch portion of the template nucleic acid molecule may be used for enrichment. In some cases, the mismatch portion may be designed to comprise a relatively longer sequence, for example at least a 5, 6, 7, 8, 9, 10, 15, 20, 25, 30, 35, 40-mer or longer homopolymer sequence, or designed to comprise an additional capture sequence, to aid in the enrichment methods.

In some cases, a high affinity peptide nucleic acid (PNA) or locked nucleic acid (LNA) may be used as enrichment molecules to capture (e.g., hybridize with) the mismatch portion. Any nucleic acid molecule may be used as enrichment molecules.

Enrichment may be performed by, in one or more enrichment steps, capturing balanced supports comprising amplified strands comprising a particular sequence using enrichment molecules, retaining the captured balanced supports and removing the non-captured balanced supports from the mixture.

Accordingly, a method for enrichment may comprise (a) providing a plurality of balanced supports which comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first enrichment molecules to the plurality of amplified strands, wherein the plurality of first enrichment molecules comprises a first capture sequence complementary to the first mismatch sequence, to generate a first set of enriched balanced supports; and (c) contacting a plurality of second enrichment molecules to the first set of enriched balanced supports, wherein the plurality of second enrichment molecules comprises a second capture sequence comprising the second mismatch sequence, to generate a second set of enriched balanced supports. The two types of enrichment molecules may be introduced in any order. For example, a method for enrichment may comprise (a) providing a plurality of balanced supports which comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first enrichment molecules to the plurality of amplified strands, wherein the plurality of first enrichment molecules comprises a first capture sequence comprising the second mismatch sequence, to generate a first set of enriched balanced supports comprising the second mismatch sequence; and (c) contacting a plurality of second enrichment molecules to the first set of enriched balanced supports, wherein the plurality of second enrichment molecules comprises a second capture sequence complementary to the first mismatch sequence, to generate a second set of enriched balanced supports.

Upon contact of a set of enrichment molecules with a set of balanced supports, an enriched set of balanced supports may be pulled down and/or otherwise isolated. In some cases, the enrichment molecules may comprise a capture moiety which is subsequently captured by a capturing moiety, according to any capture/capturing moiety pair described elsewhere herein. In some cases, the enrichment molecules may be immobilized, and/or the capturing moiety may be immobilized. Following these methods, in which derivatives of both strands of the mismatch portion are targeted and enriched, the second set of enriched balanced supports may represent enriched balanced supports where each support comprises amplified strands that are derived from both strands of the template nucleic acid molecule.

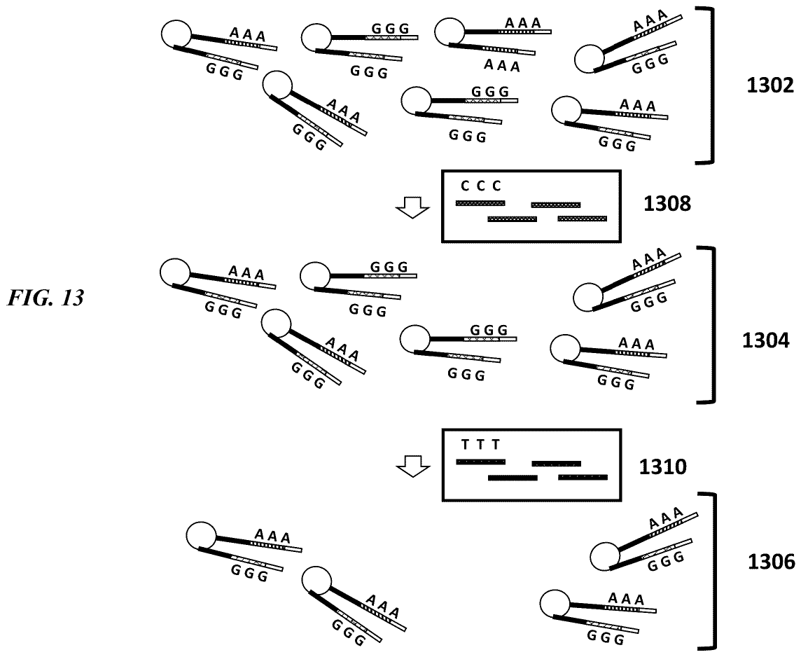

FIG. 13 illustrates a two-step enrichment protocol, according to embodiments of the present disclosure. A plurality of balanced supports 1302 may be contacted with a plurality of first enrichment molecules 1308 to isolate and/or generate a first set of enriched, balanced supports 1304. The plurality of balanced supports 1302 may comprise a plurality of amplified strands, each amplified strand comprising a portion corresponding to a mismatch portion of a template nucleic acid molecule, the mismatch portion comprising a first mismatch sequence (e.g., AAA) and a second mismatch sequence (e.g., CCC) that is not complementary to the first mismatch sequence in the first strand and second strand respectively. Amplified strands which derive from the first strand may comprise the first mismatch sequence (e.g., AAA) and amplified strands which derive from the second strand may comprise a complement of the second mismatch sequence (e.g., GGG) in the respective portions corresponding to the mismatch portion. The first enrichment molecules 1308 may comprise a first capture sequence that is complementary to one of the two sequences in the portions corresponding to the mismatch portion in the amplified strands. In this figure, the first enrichment molecules comprise a first capture sequence that comprises the second mismatch sequence (e.g. CCC), which is complementary to the complement of the second mismatch sequence (e.g., GGG) in the amplified strands. Thus, the first set of enriched, balanced supports 1304 comprise only balanced supports that comprise amplified strands that derived from the second strand. That is, balanced supports comprising only amplified strands derived from the first strand (e.g., forward only supports) are removed. In the second step, the first set of enriched, balanced supports 1304 may be contacted with a plurality of second enrichment molecules 1310 to isolate and/or generate a second set of enriched, balanced supports 1306. The second enrichment molecules 1310 may comprise a second capture sequence that is complementary to the other of the two sequences in the portions corresponding to the mismatch portion in the amplified strands. In this figure, the second enrichment molecules comprise a second capture sequence (e.g., TTT) that is complementary to the first mismatch sequence (e.g., AAA) in the amplified strands. Thus, the second set of enriched, balanced supports 1306 comprise only balanced supports that comprise amplified strands that derived from the first strand. That is, balanced supports comprising only amplified strands derived from the second strand (e.g., reverse only supports) are removed. After performing the two steps, the second set of enriched, balanced supports comprise only balanced supports that comprise amplified strands that derived from both strands of the template nucleic acid molecule.

Enrichment may alternatively or additionally be performed by, in one or more enrichment steps, capturing balanced supports comprising amplified strands comprising a particular sequence using enrichment molecules, and depleting or partially depleting the captured balanced supports from the mixture.

Accordingly, a method for enrichment may comprise (a) providing a plurality of balanced supports which comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first enrichment molecules to the plurality of amplified strands, wherein the plurality of first enrichment molecules comprises a first capture sequence complementary to the first mismatch sequence, to capture a subset of balanced supports; and (c) removing at least a subset of the subset of balanced supports from the plurality of plurality of balanced supports to generate a first set of enriched, balanced supports. In some cases, a method for enrichment may comprise (a) providing a plurality of balanced supports which comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first enrichment molecules to the plurality of amplified strands, wherein the plurality of first enrichment molecules comprises a first capture sequence comprising the second mismatch sequence, to capture a subset of balanced supports; and (c) removing at least a subset of the subset of balanced supports from the plurality of plurality of balanced supports to generate a first set of enriched, balanced supports.

In these partial depletion methods, the first enrichment molecules may be designed to capture only a fraction of (less than 100%) of balanced supports comprising amplified strands derived from one strand. Such fraction of balanced supports may include a subset of balanced supports that only contain amplified strands derived from the one strand (e.g., forward only or reverse only balanced support). Then, all or a subset of the captured balanced supports may be removed from the mixture of balanced supports to generate the first set of enriched, balanced supports—the first set of enriched, balanced supports may comprise supports comprising amplified strands derived from both strands of the template nucleic acid molecule. Alternatively, the first enrichment molecules may be designed to capture substantially all of the balanced supports comprising amplified strands derived from the first strand, and only a subset of the captured balanced supports may be removed from the mixture of balanced supports to generate the first set of enriched, balanced supports—the first set of enriched, balanced supports may comprise supports comprising amplified strands derived from both strands of the template nucleic acid molecule.

Any of the methods described herein may further comprise additional enrichment steps in which a subset of a set of enriched, balanced supports are captured using enrichment molecules comprising a capture sequence that enrich using respective portions of the amplified strands that correspond to the mismatch portion, and (i) such subset is retained and non-captured subset removed; or (ii) at least a portion of such subset is removed, to generate a second set of enriched, balanced supports. Enrichment methods described herein may comprise any number of steps of isolation and/or removal of a subset of captured supports using distinct sets of enrichment molecules, such as 1, 2, 3, 4, 5, 6, 7, 8, or more steps.

While the example in FIG. 13 illustrates that the mismatch portion comprises a pair of non-complementary homopolymer sequences (e.g., poly-A/poly-C), it will be appreciated that the mismatch portion can comprise any non-complementary sequence pair (e.g., non-homopolymer sequences), and in any configuration. The template nucleic acid molecule may comprise an alternative or additional strand recognition element (e.g., mismatch portion). FIG. 14 illustrates a support comprising a template nucleic acid molecule, pre-amplification, with different example configurations of strand recognition elements, where panel: (A) comprises a looped mismatch portion, (B) comprises a diverging mismatch portion (Y-shaped), (C) comprises both a looped mismatch portion and a diverging mismatch portion (Y-shaped) at a distal end, (D) comprises two looped mismatch portions, (E) comprises two looped mismatch portions, and an annealed double-stranded portion at a distal end comprising a cleavable moiety which upon cleavage (e.g., USER treatment during support preparation) converts the distal looped mismatch portion to a diverging mismatch portion (Y-shaped) as in the configuration shown in panel (C). The template nucleic acid molecule may comprise any number of mismatch portions, for example, 1, 2, 3, 4, 5, or more mismatch portions. A mismatch portion may be disposed more proximal to the support relative to a sequencing primer binding site such that a portion corresponding to the mismatch portion is sequenced along with a portion corresponding to the insert molecule portion. A sequencing read corresponding to the mismatch portion may be used to determine the presence of amplified strands derived from one or both strands of a template nucleic acid molecule, and/or to determine a ratio of the forward and/or reverse strand in the total amplified strands of a balanced support. A mismatch portion may be disposed more distal to the support relative to a sequencing primer binding site such that a portion corresponding to the mismatch portion is not sequenced. In this case, the mismatch portion may be used for enrichment purposes, as described elsewhere herein. In some cases, where a template nucleic acid molecule comprises multiple mismatch portions, including a first mismatch portion that is proximal to the support and a second mismatch portion that is distal to the support, a sequencing primer binding site may be disposed between the first and second mismatch portions such that a portion corresponding to the first mismatch portion that is more proximal to the support may be sequenced and used for strand ratio detection purposes and/or a portion corresponding to the second mismatch portion that is more distal to the support may not be sequenced and used for enrichment purposes, as described herein. In some cases, a portion corresponding to a mismatch portion may be used for both strand detection and enrichment purposes.

Systems, kits, and compositions of the present disclosure may comprise any component of any of the methods described herein, such as supports, amplified supports, template nucleic acids, template-attached supports, adaptors, strand displacement adaptors (e.g., adaptors comprising a mismatch portion), template nucleic acids comprising mismatch portions, first enrichment molecules, second enrichment molecules, capture moieties, capturing moieties, different enzymes, different primers, amplification reagents, sequencing reagents, etc.

### Tuning Strand Ratios in Balanced Supports

Provided herein are methods for tuning the forward-reverse strand ratio in balanced supports. The mismatch portion in a template nucleic acid molecule, and/or portions corresponding thereto in derivative strands, may be used as amplification primer binding sites during amplification. Where the template nucleic acid molecule comprises a mismatch portion comprising a first mismatch sequence and a second mismatch sequence in the first strand and the second strand, respectively, (a) a forward amplification primer may comprise a reverse complement of the first mismatch sequence to hybridize to first amplified strands derived from the first strand and prime extension reactions from the first amplified strands, and (b) a reverse amplification primer may comprise a the second mismatch sequence to hybridize to second amplified strands derived from the second strand and prime extension reactions from the second amplified strands. The respective concentrations of forward and reverse amplification primers provided during amplification may be adjusted to tune the resulting forward-reverse strand ratio in the generated balanced supports. For example, where more forward amplified strands are desired in the balanced supports, the concentration of the forward amplification primers may be increased relative to the reverse amplification primers, and vice versa.

Accordingly, a method for generating an amplified support with a predetermined forward-reverse strand ratio comprises: contacting (i) a template-attached support, wherein the template-attached support comprise a support attached to a double-stranded template molecule, wherein the double-stranded template molecule comprises a first strand and a second strand, wherein the double-stranded template molecule comprises an adaptor comprising a mismatch portion, wherein the mismatch portion comprises in the first strand a first mismatch sequence and in the second strand a second mismatch sequence that is not complementary to the first mismatch sequence and (ii) a plurality of forward amplification primers at a first predetermined concentration and a plurality of reverse amplification primers at a second predetermined concentration, to generate the amplified support comprising a plurality of amplified strands with a predetermined forward-reverse strand ratio, wherein each forward amplification primer of the plurality of forward amplification primers comprises a reverse complement of the first mismatch sequence to hybridize to first amplified strands derived from the first strand, and wherein each reverse amplification primer of the plurality of reverse amplification primers comprises the second mismatch sequence to hybridize to second amplified strands derived from the second strand.

Alternatively or in addition to adjusting the respective concentrations of the forward and reverse amplification primers, various other parameters, such as affinity of the respective primers to the amplified strands at the portions corresponding to the mismatch portion, can be adjusted to tune the forward-reverse strand ratio in the generated balanced support. Alternatively or in addition to adjusting the respective parameters of the forward and reverse amplification primers, various parameters of the amplification procedure may be adjusted to tune the forward-reverse strand ratio. For example, during one or more PCR cycles of the amplification procedure, the temperature may be adjusted to favor the annealing temperature of one type of primer over the other type of primer. In these cases, forward and reverse amplification primers may be designed to have different annealing temperatures with the respective portions corresponding to the mismatch portion.

Systems, kits, and compositions of the present disclosure may comprise any component of the methods described herein, such as supports, amplified supports, template nucleic acids, template-attached supports, adaptors, adaptors comprising a mismatch portion, template nucleic acids comprising mismatch portions, forward amplification primers, reverse amplification primers, amplification reagents, sequencing reagents, etc.

### Detecting Strand Ratios Independent of Sequencing

Provided are methods for detecting forward-reverse strand ratios of a balanced support independent of and/or in addition to sequencing. These methods may detect the forward-reverse strand ratio of a balanced support without the need for sequencing or generating a sequencing read. A probe comprising a capture sequence and a detectable moiety may be configured to bind its capture sequence to an amplified strand at a portion corresponding to the mismatch portion of the template nucleic acid molecule. The probe may be a labeled oligonucleotide probe. Where a template nucleic acid molecule comprises a mismatch portion comprising a first mismatch sequence and a second mismatch sequence in a first strand and a second strand, respectively, a first type of probe may be configured to detect amplified strands derived from the first strand, which each comprises the first mismatch sequence. The first type of probe may comprise a first capture sequence that comprises a reverse complement of the first mismatch sequence and a first detectable moiety. A second type of probe may be configured to detect amplified strands derived from the second strand, which each comprises a reverse complement of the second mismatch sequence. The second type of probe may comprise a second capture sequence that comprises the second mismatch sequence and a second detectable moiety.

Only one type of probe may be used. Both types of probes may be used, sequentially or simultaneously. The first and second type of detectable moiety may be the same type or different types of detectable moiety. The detectable moiety may be any label as described herein, such as a fluorescent dye. In some cases, the first and second types of detectable moiety may be detectable at the same frequency or frequency range. In some cases, the first and second types of detectable moieties may be detectable at different frequencies or frequency ranges.

The probes may be contacted with balanced supports which are immobilized to a substrate, unbound probes washed away, and signals detected form the balanced supports. After detection, the bound probes may be removed from (e.g., denatured from) the balanced supports and/or detectable moiety deactivated (e.g., dye cleaved) prior to collecting sequencing signals from the balanced supports. The signals detected from the probes may be used to determine the relative amounts of strands of the forward and/or reverse strands on a balanced support. In some cases, a first type of probes and a second type of probes may be contacted with the balanced supports, simultaneously or in a temporally distinct manner. Signals from each probe type may be detected simultaneously, cumulatively, or in a temporally distinct manner. Signals from each probe type collected at each location may be processed to determine a forward-reverse strand ratio for a balanced support immobilized at such location. In some cases, one type of probes may contact the balanced supports and first signals detected, before a second type of probes contact the balanced supports. In some cases, the first type of probes may be removed from and/or detectable moiety deactivated before contacting the balanced supports with the second type of probes and collecting second signals. In other cases, if the first detectable moieties from the first type of probes are still present and active when the second type of probes are contacted, the second signals may be detected at a different frequency or channel to differentiate from the first detectable moieties, or the second signals may be detected at a same frequency or channel as the first detectable moieties and represent a cumulative signal from the first and second type of probes, in which case the second and first signals may be differentially processed to determine the signals collected from only the second type of probes. In some cases, signals from the first and second types of probes may be collected after both probes are contacted with the balanced supports and detected at different frequencies or channels.

Accordingly, a method for detecting strands on a support may comprise (a) providing a plurality of balanced supports immobilized to a substrate, wherein the plurality of balanced supports comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first type of probes to the plurality of amplified strands, wherein the plurality of first type of probes each comprises a first capture sequence complementary to the first mismatch sequence and a first detectable moiety; and (c) detecting first signals from the first detectable moiety of a subset of the plurality of first type of probes bound to a subset of the plurality of amplified strands. The method may further comprise, (d) contacting a plurality of second type of probes to the plurality of amplified strands, wherein the plurality of second type of probes each comprises a second capture sequence comprising the second mismatch sequence and a second detectable moiety; and (e) detecting second signals from the second detectable moiety of a subset of the plurality of second type of probes bound to a second subset of the plurality of amplified strands. Systems, kits, and compositions of the present disclosure may comprise any component of the methods described herein, such as supports, amplified supports, template nucleic acids, template-attached supports, adaptors, adaptors comprising a mismatch portion, template nucleic acids comprising mismatch portions, substrates, first probes, second probes, detectable moieties, amplification reagents, sequencing reagents, detectors, etc.

In any of the methods of the present disclosure, a template insert molecule, or derivative, may be ligated on one end to a strand recognition adaptor such that the sequenced product comprises strand recognition elements on only one end. Alternatively, in any of the methods of the present disclosure, a template insert molecule, or derivative, may be ligated on both ends to a strand recognition adaptor (which may or may not comprise the same pair of mismatch sequences) such that the sequenced product comprises strand recognition elements on both ends. For example, in the workflows illustrated in FIGS. 10A-10D, adaptor 1001 may also comprise a mismatch portion (making adaptor 1001 also a strand recognition adaptor in addition to adaptor 1005). In some cases, attaching a strand recognition adaptor to both ends of the template insert molecule may allow for strand recognition and/or quantification when reading from either end, which may be helpful for paired end sequencing workflows. In some cases, one or both strand recognition sequences may be identified when an entire strand sequence is sequenced through to the end with sufficient sequencing quality for additional verification.

### Additional Amplification Methods for Maintaining Double Strand Association

In some cases, it is desirable to amplify sample nucleic acids prior to sequencing (e.g., in cases where a sample contains only a small number of template nucleic acid molecules, for the purpose of targeted sequencing, etc.). Beneficially, amplification methods described herein enable the preservation of both strands of a double-stranded template molecule (e.g., prior to or concurrent with library preparation). In some cases, this amplification can be performed prior to further processing for sequencing (e.g., PCR, ePCR, RPA, eRPA, or bridge amplification for the purpose of colony formation on a support). In some cases, amplification methods can provide amplification products which maintain double strand association and have strand recognition elements. In some cases, amplification methods can provide amplification products which maintain double strand association but without strand recognition elements-such amplification products may subsequently be tagged with strand recognition elements (e.g., ligated to mismatch adaptors as described herein) and then further amplified to generate amplified clusters comprising strands with strand recognition elements. Such further amplification of amplification products may yield duplicate colonies in different clusters (optionally immobilized to separate supports), which duplication may further reduce sequencing errors by providing comparative data points within the sample (e.g., one duplicate may be compared to another duplicate for internal error correction, such as by discarding base calls or reads with discrepancies between duplicates). Amplified clusters may be sequenced with error correction based on the strand recognition elements, as described elsewhere herein. The following methods can be used to maintain double strand association and preserve information from both strands of a library molecule alternatively or in addition to the workflows described with respect to FIGS. 9B-10D.

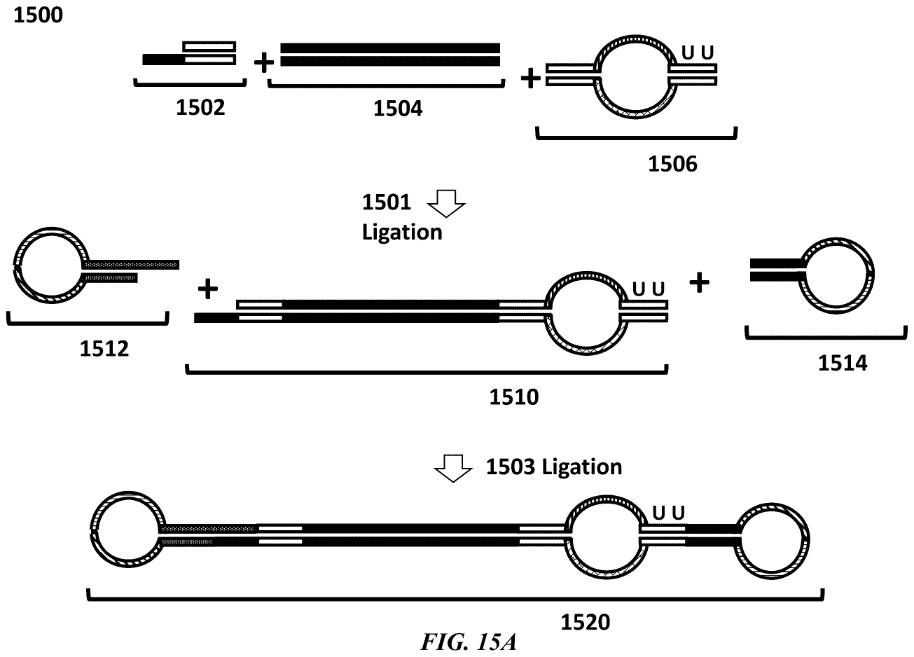

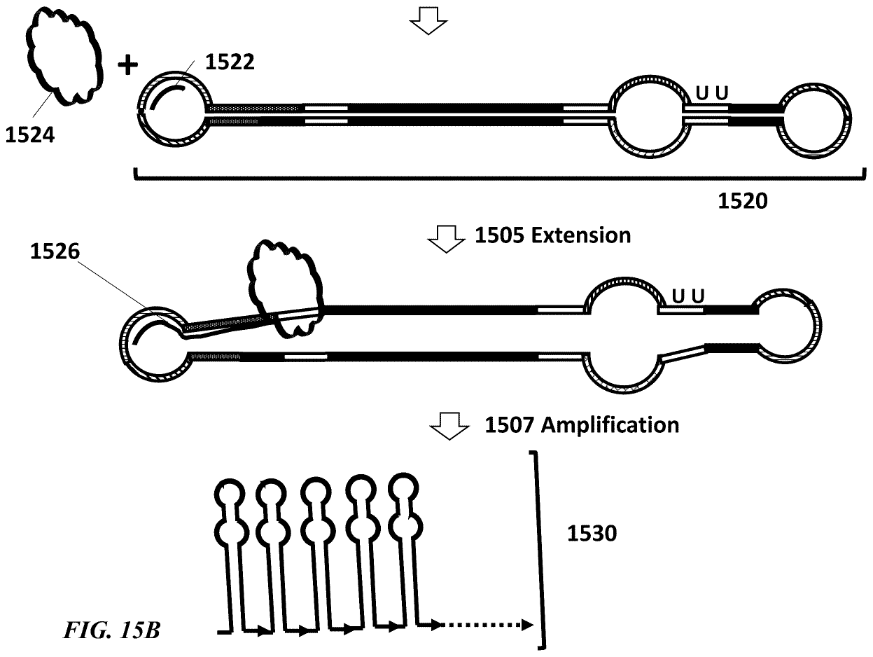

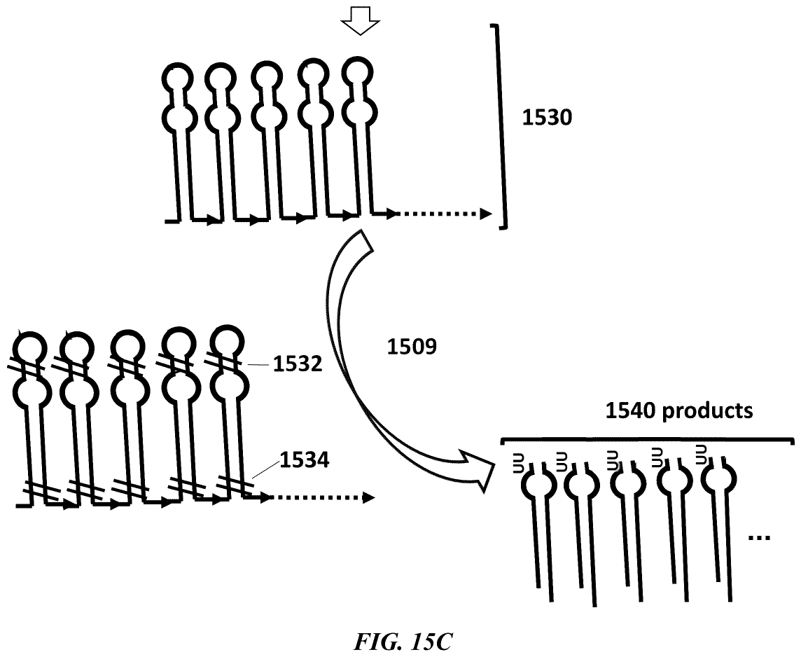

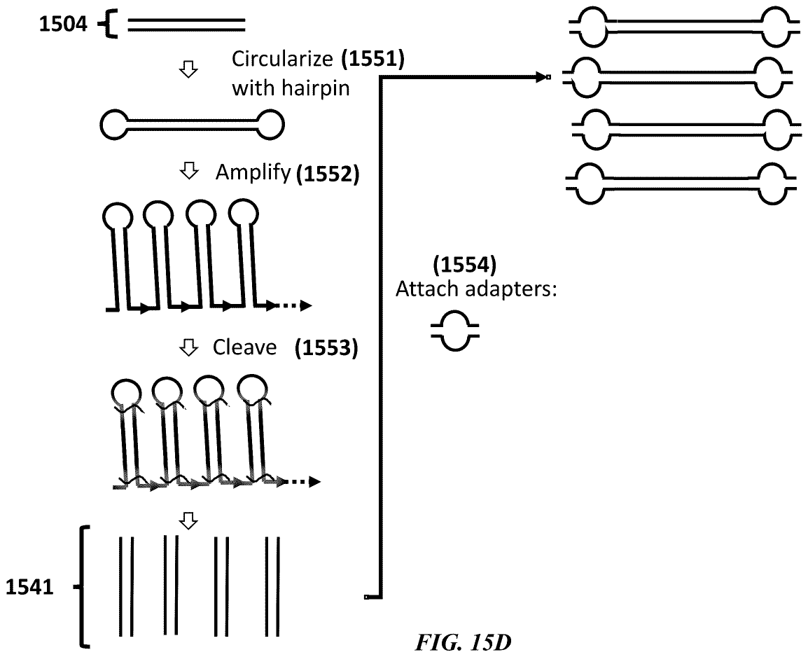

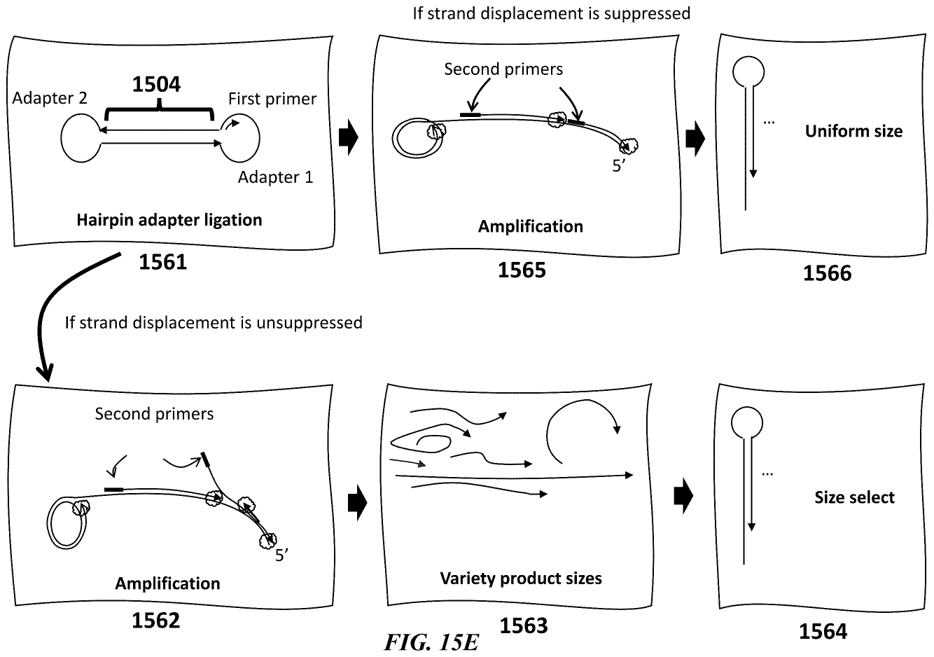

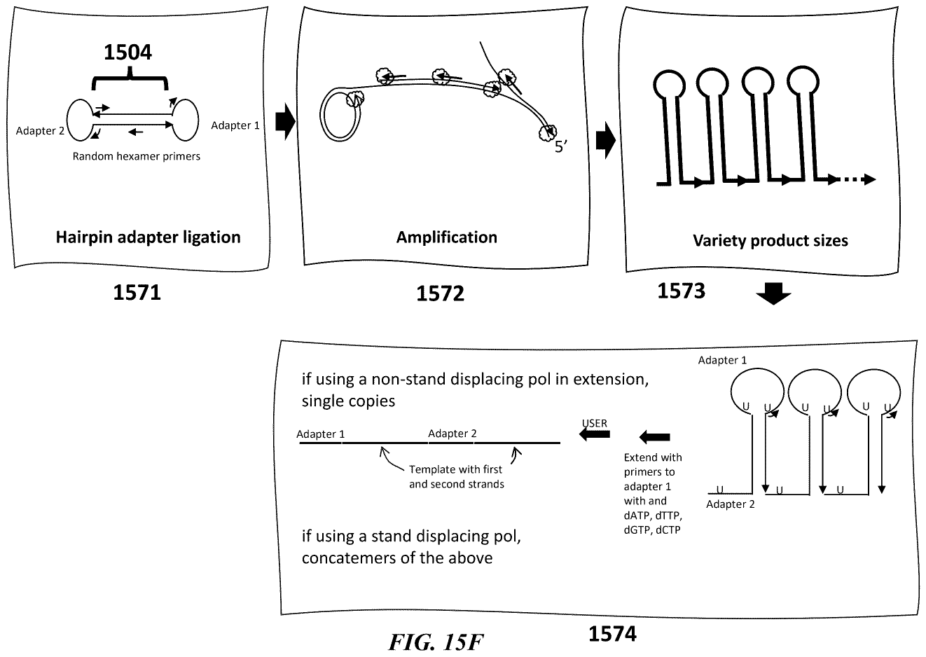

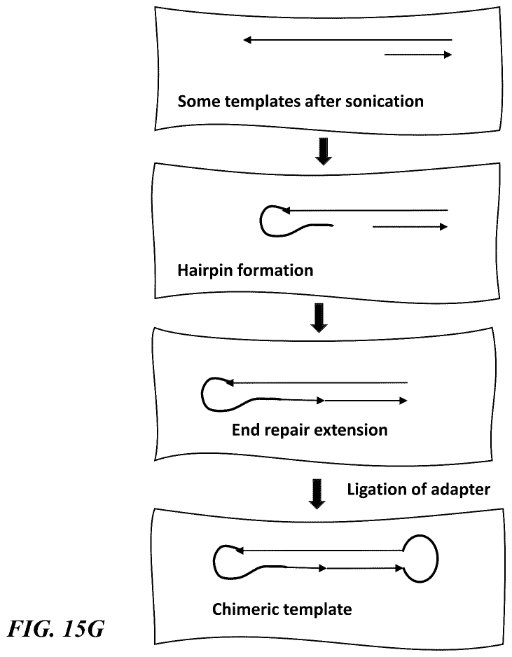

FIGS. 15A-15C illustrate a workflow for amplifying double-stranded template molecules while retaining forward-reverse strand associations (e.g., amplification without losing double-stranded context). An insert molecule 1504 (e.g., a sample nucleic acid) is provided with a plurality of adaptors, where at least one type of adaptor in the plurality of adaptors comprises a mismatch region and functions as a strand recognition adaptor. As illustrated in FIG. 14 and described elsewhere herein, a mismatch region may comprise a single base mismatch, a multiple base mismatch, a hairpin region, etc. FIGS. 15A-15B illustrate the mismatch region as an internal loop (e.g., internal hairpin) in adaptor 1506, but this is for illustrative purposes only and other configurations are contemplated.

In FIG. 15A, a double-stranded target molecule 1504 and adaptors 1502 and 1506 are provided. These are ligated 1501 to produce double-stranded template molecule 1510. Double-stranded template molecule 1510 is further ligated 1503 to additional adaptors 1512 and 1514 to produce double-stranded double-adaptor template molecule 1520. Each ligation 1501 and 1503 could alternatively be tagmentation reactions.

In FIG. 15B, double-stranded double-adaptor template molecule 1520 anneals to primer 1522, where primer 1522 anneals to a single-stranded region in one of the adaptors in molecule 1520. Polymerase 1524 extends 1505 primer 1522 to produce molecule 1526 which is a copy of molecule 1520. Polymerase 1524 may be a strand-displacing polymerase. The extension may be repeated any number of times to amplify 1507 the double-stranded double-adaptor template molecule 1520. Amplification 1507 may be rolling circle amplification, thus the amplification product 1530 is a large nucleic acid molecule comprising a plurality of copies of the double-stranded double-adaptor template molecule 1520.

In FIG. 15C, the plurality of copies of double-stranded double-adaptor template molecule 1530 is subjected to conditions sufficient to cleave 1509 one or more cleavage sites (sites 1532 and 1534). In some cases, cleavage 1509 comprises enzymatic digestion and cleavage sites 1532 and 1534 comprise restriction sites. After cleavage/digestion 1509, the resulting copy molecules 1540 comprise copies of the double-stranded template molecule 1510. These copies preserve the double-strand context of the original target molecules 1504 for the purposes of additional processing, as described herein. Additional methods described herein, for instance detecting strand ratios independent of sequencing, may be performed using copy molecules 1540. Copy molecules 1540 may be sequenced to provide sequencing information on original double-stranded target molecule 1504. In some cases, prior to sequencing, the copy molecules 1540 may be further amplified via any of the amplification methods described herein. Amplification of the copy molecules may yield duplicate colonies in different clusters (optionally immobilized to separate supports), which duplication may further reduce sequencing errors by providing comparative data points within the sample (e.g., one duplicate may be compared to another duplicate for internal error correction, such as by discarding base calls or reads with discrepancies between duplicates). For example, the copy molecules may be amplified via ePCR or eRPA, yielding amplified supports.

FIG. 15D illustrates an additional workflow for amplifying double-stranded template molecules while retaining forward-reverse strand associations (e.g., amplification without losing double-stranded context). An insert molecule 1504 (e.g., a sample nucleic acid) is circularized (1551) by attaching (e.g., ligating) to each end an adaptor comprising a hairpin moiety. A primer may anneal to one of or both single-stranded hairpin regions of the circularized molecule and be extended by a polymerase. The polymerase may be a strand-displacing polymerase. The extension may be repeated any number of times (or for any length of time) to amplify (1552) the circularized molecule, such as via RCA. The amplification product may be a concatemeric molecule comprising a plurality of copies of the double-stranded, circularized molecule. The plurality of copies may be subjected to conditions sufficient to cleave (1553) a copy (or portion thereof) from other copies. For example, the cleavage may comprise enzymatic digestion at restriction sites at or near the hairpin moieties. In some cases, the hairpin moieties may be cleaved and/or digested. After cleavage/digestion, the resulting copy molecules 1541 comprise copies which preserve the double-strand context of the original insert molecule 1504. Each of the resulting copy molecules 1541 may be attached to strand recognition adaptors comprising mismatch portions, as described elsewhere herein, such as to each end of the molecule. Additional methods described herein, for instance detecting strand ratios independent of sequencing, may be performed using the copy molecules. The copy molecules may be sequenced to provide sequencing information on original insert molecule 1504. In some cases, prior to sequencing, the copy molecules may be further amplified via any of the amplification methods described herein. Amplification of the copy molecules may yield duplicate colonies in different clusters (optionally immobilized to separate supports), which duplication may further reduce sequencing errors by providing comparative data points within the sample (e.g., one duplicate may be compared to another duplicate for internal error correction, such as by discarding base calls or reads with discrepancies between duplicates). For example, the copy molecules may be amplified via ePCR or eRPA, yielding amplified supports.

FIG. 15E illustrates an additional workflow for dumbbell-based amplification of double-stranded template molecules while retaining forward-reverse strand associations (e.g., amplification without losing double-stranded context) using suppressed strand displacement. An insert molecule 1504 (e.g., a sample nucleic acid) is circularized (1561) by attaching (e.g., ligating) to each end an adaptor comprising a hairpin moiety (e.g., Adaptor 1, Adaptor 2).

A first workflow, where blocked primers are not used, is illustrated through processes 1562-1564. In the first workflow, a first primer may hybridize to a primer binding site in one or both of Adaptor 1 and Adaptor 2 and be extended using strand displacing polymerases to produce a first concatemer product. Second primers may each hybridize to a respective primer binding site in the first concatemer product and extended using strand displacing polymerases to produce secondary concatemer products. One secondary concatemer product may displace another second primer hybridized to the first concatemer product as it is extended. The composition may comprise additional first primers which may bind to secondary concatemer products and are extended to generate tertiary concatemer products, which tertiary concatemer products may hybridize to second primer molecules which are extended to generate fourth order concatemer products, and etc. After amplification (e.g., RCA, MDA) is performed (1562), amplification products of various sizes may be generated (1563). In some cases, the amplification products may be subjected to size selection (1564) to isolate products having at least and/or at most a desired number of copies. In some cases, the amplification products may be cleaved to generate single copy products, such as with described with respect to 1553 in FIG. 15D.

A second workflow, where amplification is subjected to suppressed strand displacement, is illustrated through processes 1565-1566. In the second workflow, a first primer may hybridize to a primer binding site in one or both of Adaptor 1 and Adaptor 2 and be extended using strand displacing polymerases to produce a first concatemer product. In some cases, Adaptor 1 and Adaptor 2 hairpin moieties may comprise different sequences. Second primers may each hybridize to a respective primer binding site in the first concatemer product and be extended under suppressed strand displacement conditions to produce secondary amplification products. One secondary amplification product may stop extension when it reaches a primer binding site that another second primer is hybridized to at the first concatemer product because strand displacement is suppressed. In some cases, the second primer extension is performed with non-strand displacing polymerases to prevent strand displacement. In some cases, the second primer comprises a reversible cross-linking reagent at or adjacent to the 5′ end for reversible cross-linking at one or more bases (e.g., 3-cyanovinylcarbazole (CNVK), 3-cyanovinylcarbozole modified by D-theoninol (CNVD), pyranocarbazole (PCX), pyranocarbazole with D-threoninol (PCXD), etc.) to the first concatemer product, which cross-linking prevents strand displacement but can be reversed, such as via stimulus (e.g., photostimulus), to release the second amplification products; the second primer comprises synthetic nucleic acids and/or mimic nucleic acids, such as peptide nucleic acids (PNAs), bridged nucleic acids (BNAs), or locked nucleic acids (LNAs), at or adjacent to the 5′ end to prevent strand displacement; the second primer comprises another blocker (e.g., methylated RNA bases, etc.) at or adjacent to the 5′ end to prevent strand displacement; or the like and/or a combination thereof. In some cases, a modification may be included in the first concatemer product at portions corresponding to one of the adaptors such that a polymerase falls off the template after reaching such modification (e.g., after generating only one copy) or a modification may be included in one of the adaptors such that a polymerase falls off the template after reaching such modification (e.g., after generating only one copy). The template may be re-primed, such as via a nickase sequence in the loop. After amplification is performed (1565), second amplification products of substantially uniform size (e.g., corresponding to one copy of the template) may be generated (1566). Beneficially, in the second workflow, the first concatemer product (e.g., a long amplicon) is maintained in substantially double-stranded form instead of single-stranded form during amplification, which prevents the first concatemer product from folding on itself—this may reduce biases that are created due to interference from folded molecules. Further, the second workflow obviates the need for cleaving a long concatemeric product, as with the first workflow, which may improve yield. The second amplification products may be detached from the first concatemer product, each of the second amplification products comprising a single copy of the insert molecule.

The resulting copy molecules, in either first or second workflow, comprise copies of the insert 1504 which preserve the double-strand context. In some cases, the hairpin may be cleaved and/or the hairpin moiety portions may be cleaved or digested in the copy molecules. Each of the resulting copy molecules may be attached to strand recognition adaptors comprising mismatch portions, as described elsewhere herein, such as to each end of the molecule (e.g., as in the workflow of FIG. 15D). Additional methods described herein, for instance detecting strand ratios independent of sequencing, may be performed using the copy molecules. The copy molecules may be sequenced to provide sequencing information on original insert molecule 1504. In some cases, prior to sequencing, the copy molecules may be further amplified via any of the amplification methods described herein. Amplification of the copy molecules may yield duplicate colonies in different clusters (optionally immobilized to separate supports), which duplication may further reduce sequencing errors by providing comparative data points within the sample (e.g., one duplicate may be compared to another duplicate for internal error correction, such as by discarding base calls or reads with discrepancies between duplicates). For example, the copy molecules may be amplified via ePCR or eRPA, yielding amplified supports.

FIG. 15F illustrates an additional workflow for dumbbell-based amplification of double-stranded template molecules while retaining forward-reverse strand associations (e.g., amplification without losing double-stranded context) using random primers. An insert molecule 1504 (e.g., a sample nucleic acid) is circularized (1571) by attaching (e.g., ligating) to each end an adaptor comprising a hairpin moiety (e.g., Adaptor 1, Adaptor 2). In some cases, random primers, such as random hexamer primers, may be contacted with the circular template and one or more primers may hybridize to one or more sites in the circular template and be extended using strand displacing polymerases to produce at least a first concatemer product. In some cases, the first concatemer product may be generated by hybridizing a primer comprising a sequence specific to the adaptor sequence in one or both of Adaptor 1 and Adaptor 2 and extending to generate the first concatemer product, and random primers may be added and/or be present. A random primer, though exemplified here as random hexamers, may have any length (e.g., 2-mer, 3-mer, 4-mer, 5-mer, 6-mer, 7-mer, 8-mer, 9-mer, etc.). The composition may be provided with dNTPs comprising dUTPs instead of dTTPs. Random primers may bind to various sites of the first concatemer product(s) and be extended using strand displacing polymerases to generate secondary amplification products comprising uracil residues. During amplification (1572), random primers may bind to the secondary or higher order amplification products to generate various amplification products with uracil residues. After washing, a second set of primers may be introduced to the amplification products, a second primer of the second set of primer comprising a sequence that binds to portions of the concatemer products corresponding to one or both adaptors. The composition may be provided with dNTPs comprising dTTPs instead of dUTPs. The second set of primers may be extended to generate additional amplification products comprising thymine residues (as opposed to uracil residues). The composition may be heated to enable primer binding. The composition may then be subjected to enzymatic degradation (1574), such as using uracil-specific excision reagent (USER) enzymes, to remove amplified material that contain uracil residues (e.g., only one type of amplified strand). In some cases, the second set of primers may be extended using non-strand displacing polymerases, in which case the resulting products will be copy molecules each comprising a single copy of the insert molecule 1504. In some cases, the second set of primers may be extended using strand displacing polymerases, in which case the resulting products may be concatemers each comprising multiple copies of the insert molecule 1504. For example, a concatemer may comprise chains of hairpin-containing copies, which may be cleaved and/or hairpins digested to generate double-stranded copy molecules. It will be appreciated that alternatives to dUTPs, such as other degradable or excisable bases, e.g., ribonucleotides) may be used for the secondary amplification product generation which can otherwise mediate degradation (e.g., alternative to USER) downstream after the additional amplification products.

The copy molecules may comprise one or more copies of the insert 1504 which preserve the double-strand context. In some cases, the hairpin may be cleaved and/or the hairpin moiety portions may be cleaved or digested. Each of the resulting copy molecules may be attached to strand recognition adaptors comprising mismatch portions, as described elsewhere herein, such as to each end of the molecule (e.g., as in the workflow of FIG. 15D). Additional methods described herein, for instance detecting strand ratios independent of sequencing, may be performed using the copy molecules. The copy molecules may be sequenced to provide sequencing information on original insert molecule 1504. In some cases, prior to sequencing, the copy molecules may be further amplified via any of the amplification methods described herein. Amplification of the copy molecules may yield duplicate colonies in different clusters (optionally immobilized to separate supports), which duplication may further reduce sequencing errors by providing comparative data points within the sample (e.g., one duplicate may be compared to another duplicate for internal error correction, such as by discarding base calls or reads with discrepancies between duplicates). For example, the copy molecules may be amplified via ePCR or eRPA, yielding amplified supports.

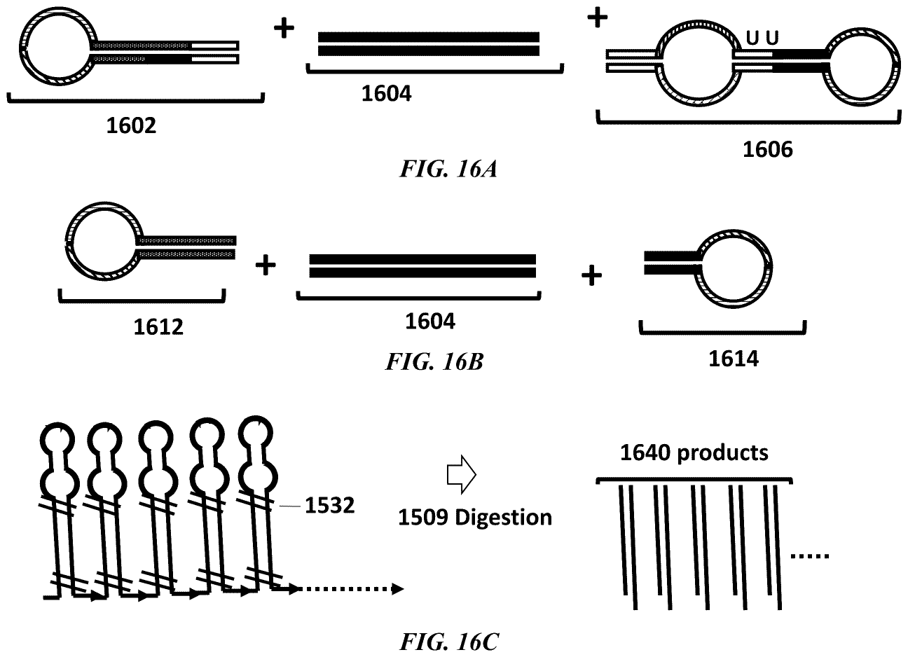

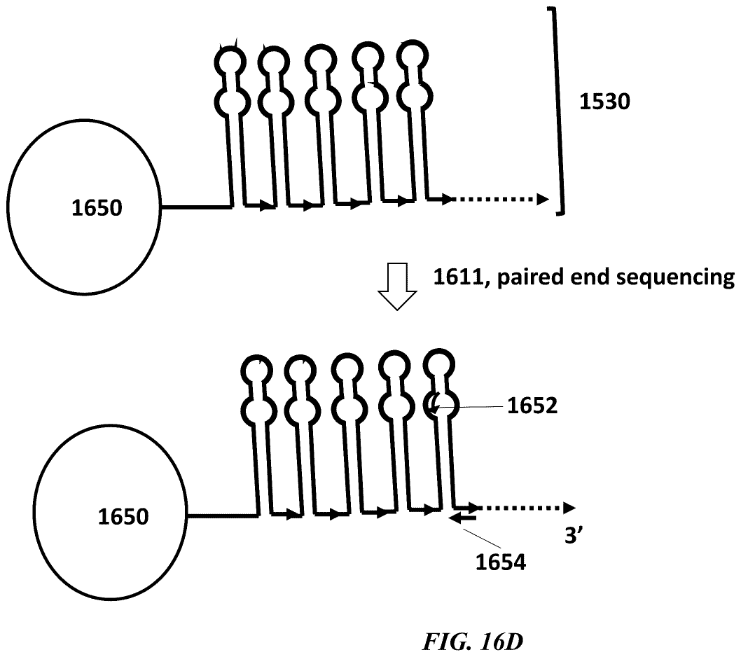

In any of the FIG. 15E-15F workflows, the initial hairpin adapters ligated to the insert 1504 to generate the dumbbell circular templates may comprise strand recognition elements. For example, the hairpin adapter may comprise strand recognition adaptors, as shown in FIG. 16A. In some cases, the insert may be pre-ligated to a strand recognition adaptor, on one or both ends, and then ligated to hairpin adapters, such as according to the workflow depicted in FIG. 15A. Beneficially, each strand copy in the resulting copy molecules may be pre-associated with a strand recognition element, prior to cleaving and/or digesting the hairpin moieties in the concatemers. This may obviate the need for re-attaching strand recognition adaptors.

FIG. 15G illustrates a possible source of error during library preparation when performing blunt end shearing and ligating hairpin adaptors, such as to proceed with amplification workflows provided in FIGS. 15A-15F. During shearing, such as after sonification, some templates may comprise a single-stranded overhang instead of a blunt end at one or both ends, as shown in the first panel in FIG. 15G. If there is incomplete or inefficient end repair, the single-stranded overhang may fold and self-hybridize to create a hairpin on the template molecule, as shown in the second panel in FIG. 15G. After end repair, as shown in the third panel, and upon ligation of hairpin adaptors, the self-hybridized template may only ligate to one hairpin adaptor on end(s) that do not have the self-hybridized hairpin, as shown in the last panel in FIG. 15G. When these templates are processed, such as according to various amplification workflows described herein, and sequenced, the self-hybridization causes the formation of many chimeric reads, that is pairs of complementary strands in the same genomic location. Provided herein are methods that prevent or reduces this phenomenon.

In some cases, after sonication, end repair may be performed at higher temperatures. The higher temperatures will require more bases to stabilize possible hairpin structures, reducing the likelihood that hairpin structures will form. In some cases, the blunting may be performed in the presence of, or after pre-treatment with, exo- or endo-nucleases, such as Si and mung bean endonucleases, to prevent the possibility of hairpin structure formation. In some cases, hairpin adapters and/or strand recognition adapters may be added to sample DNA without shearing using transposases, such as Tn5 transposases, in a tagmentation reaction. Such transposase-based hairpin adapter addition may not require end repair.

There are many possible variations of the method illustrated in FIGS. 15A-15F. For instance, as shown in FIG. 16A, in some cases, a target molecule may be attached to two adaptors, adaptor 1602 and 1606. Adaptor 1606 may comprise both a mismatch region (shown here as loop, but which could also be a single base alteration) and a hairpin region (e.g., a sequence that forms a stem-loop structure) (e.g., combining adaptors 1506 and 1514). Likewise, another adaptor 1602 may comprise both a hairpin region and a region that enables additional processing such as annealing to a support (e.g., combining adaptors 1502 and 1512). In FIG. 16B, adaptors 1612 and 1614 attached to target molecule 1604 do not comprise a mismatch region. In such instances, adaptors comprising a mismatch region may subsequently be attached to copy molecules 1540 resulting from amplification of an original target molecule 1604, similar to the workflow in FIG. 15D. FIG. 16C illustrates a possible modification of cleavage step 1509, where cleavage sites 1532 are located so as to result in the removal of all adaptor regions in the amplified copy molecules 1640.

FIG. 16D illustrates an exemplary sequencing method for use with the amplified molecule 1530 (e.g., generated in FIG. 15B). Amplification product 1530 is a large nucleic acid molecule comprising a plurality of copies of the double-stranded double-adaptor template molecule 1520. Amplification product 1530 can be attached to a support 1650 (e.g., bead, planar substrate) for sequencing with primers 1652 and 1654. In some cases, primers 1642 and 1654 can be extended simultaneously. In some cases, primers 1652 and 1654 can be extended sequentially. That is, these primers comprise distinct sequences and anneal to separate portions of molecule 1530. This can provide paired-end sequencing information. Either sequencing method provides information from each original strand (e.g., copies of the original target molecule forward and reverse strands).

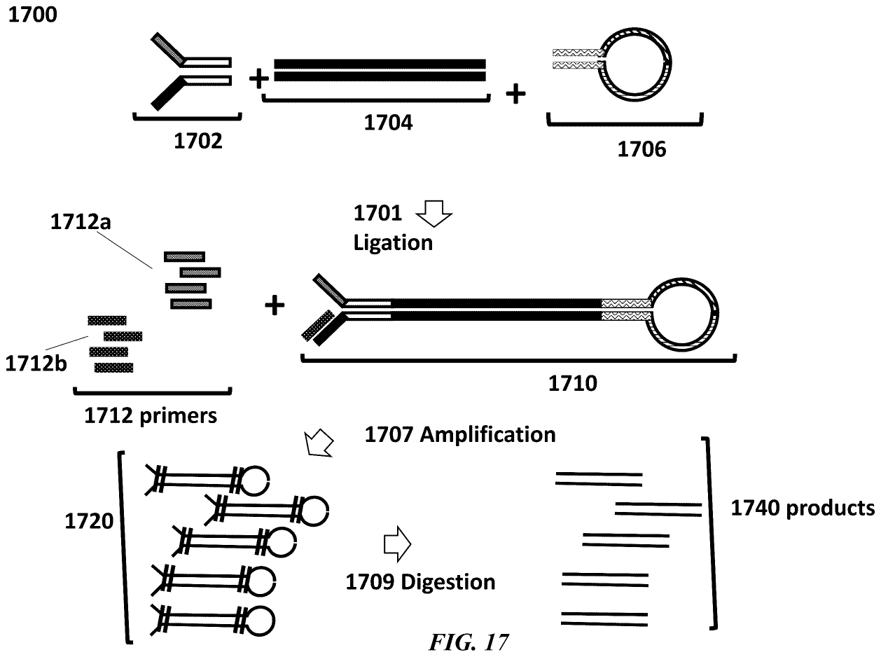

FIG. 17 illustrates another workflow for amplifying double-stranded template molecules while retaining forward-reverse strand associations (e.g., amplification without losing double-stranded context). This workflow differs from that described with respect to FIGS. 15A-15F, in that only one type of hairpin adaptor is used, a y-shaped adaptor is used instead of a second hairpin adaptor, and the amplification method is not RCA (e.g., may be PCR, LAMP, etc.). As illustrated in FIGS. 14 and 16A-16B, many different adaptor configurations are possible. Double-stranded target molecule 1704 is ligated 1701 (or tagmented) to y-shaped adaptor 1702 comprising a mismatch region and adaptor 1706 with a hairpin region. Template molecule 1710 is provided with a plurality of primers 1712. The plurality of primers 1712 comprises at least a first subset 1712b and a second subset 1712a, where the first subset 1712b can anneal to a first region of adaptor 1702 and the second subset 1712a has sequence complementarity to a different region of adaptor 1702. The plurality of primers is used to amplify 1707 double-stranded template molecule 1710 to produce a plurality of copies 1720. The plurality of copies 1720 are exposed to conditions sufficient for cleavage of one or more cleavage sites in copies 1720 (e.g., cleavage sites in adaptors 1702 and/or 1706) to provide double-stranded template molecules 1740, where these double-stranded template molecules 1740 are copies of the target molecule 1704 and retain the forward/reverse strand context of the target molecule. Additional methods described herein, for instance detecting strand ratios independent of sequencing, may be performed using copy molecules 1740. Copy molecules 1740 may be sequenced to provide sequencing information on original double-stranded target molecule 1704. In some cases, adaptors comprising a mismatch region may subsequently be attached to copy molecules 1740, similar to the workflow in FIG. 15D. In some cases, the copy molecules may be further amplified.

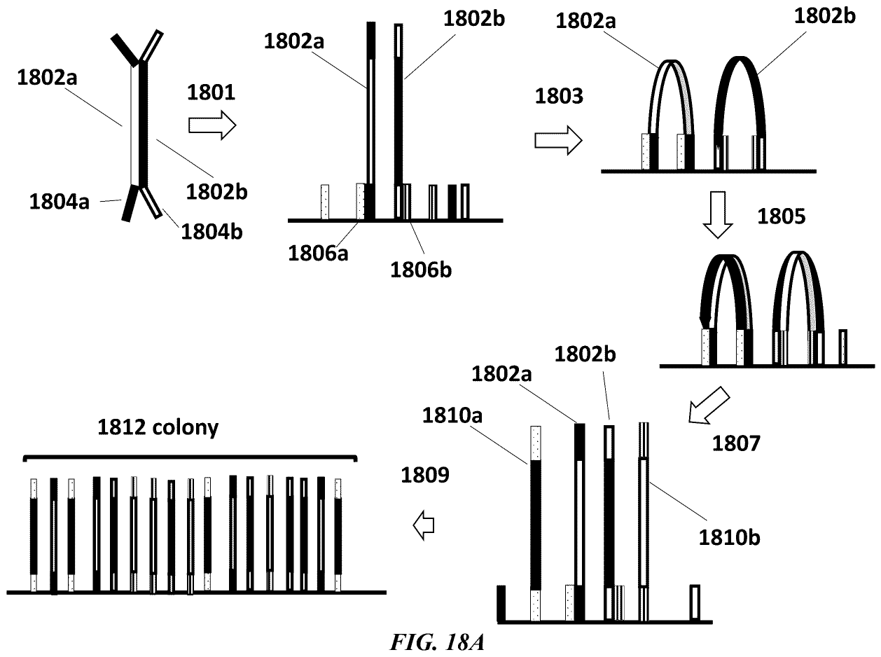

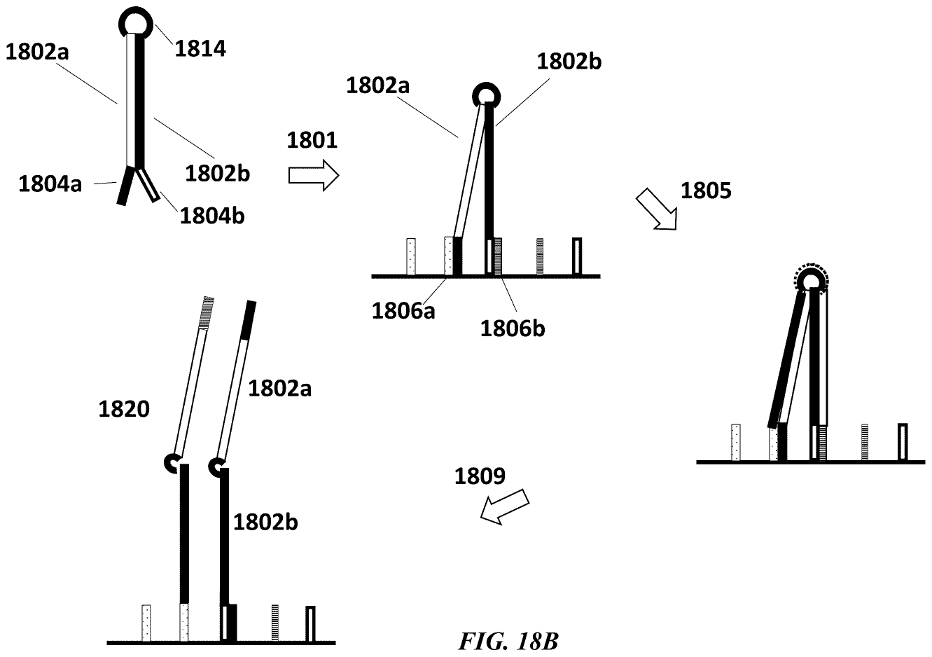

FIGS. 18A and 18B illustrate additional workflows for amplifying double-stranded template molecules while retaining forward-reverse strand associations (e.g., amplification without losing double-stranded context). The workflows utilize bridge amplification to provide a colony comprising forward and reverse strand copies on a support (e.g., a wafer, a bead, etc.). In either case, the double-stranded template molecules may further comprise a mismatch adaptor (e.g., strand recognition adaptor) and/or an identification sequence (e.g., a UMI), as described elsewhere herein.

In FIG. 18A, a double-stranded template molecule comprising a first strand 1802a and a second strand 1802b is provided. The double-stranded template molecule further comprises a double-stranded adaptor at each end, where the double-stranded adaptors each comprise a first region 1804a and a second region 1804b that do not have strand complementarity to each other. Here, the adaptors on each end of the double-stranded template molecule are shown as both of a same type. It will be appreciated that different types of adaptors may be used (e.g., where a first adaptor of a first type is attached to a first end of the template molecule and a second adaptor of a second type is attached to the other end of the template molecule). Both strands of the double-stranded template molecule anneal 1801 to a support, where the support comprises primers 1806a and 1806b with sequence complementarity to each of adaptor regions 1804a and 1804b, respectively. Strands 1802a and 1802b each anneal again 1803 to the support (e.g., each forming a bridge). Using the annealed surface primers, each strand is replicated 1805 (e.g., surface primers annealed to a strand 1802 are extended), resulting in the original first and second strands 1802 which are annealed to the support via surface primers and strands 1810 which are covalently attached to surface primers, where 1810a and 1810b are the reverse complements of strands 1802a and 1802b, respectively. These strands may be dissociated 1807 (e.g., separated into single-stranded molecules). Amplification may be repeated 1809, resulting in a colony 1812 that comprises copies of each strand 1802 attached to the support.

Additional methods described herein, for instance detecting strand ratios independent of sequencing, may be performed using colony 1812. Colony 1812 may be sequenced to provide sequencing information on original strands 1802.

In FIG. 18B, a double-stranded template molecule comprising first strand 1802a and second strand 1802b is provided. The double-stranded template molecule further comprises an adaptor of a first type, comprising regions 1804a and 1804b which lack sequence complementarity to each other, and an adaptor of a second type, comprising a single-stranded hairpin region 1814. Thus, strands 1802a and 180b will be covalently attached to each other.

The double-stranded template molecule anneals 1801 to a support, where the support comprises primers with sequence complementarity to each of adaptor regions 1804a and 1804b. the double-stranded template molecule is replicated 1805 (e.g., a surface primer annealed to a strand 1802 is extended). These strands may be dissociated 1807 (e.g., separated into single-stranded molecules), resulting in molecule 1820 that is a reverse complement of strands 1802 and is covalently bound to the support and the original target molecule comprising strands 1802 which is annealed to the support. As in FIG. 18B, amplification may be repeated, resulting in a colony of individual molecules 1820 covalently attached to the support. The resulting colony may be sequenced to provide sequencing information on original strands 1802.

### Duplex Molecules for Retaining Double Strand Information on a Single Strand

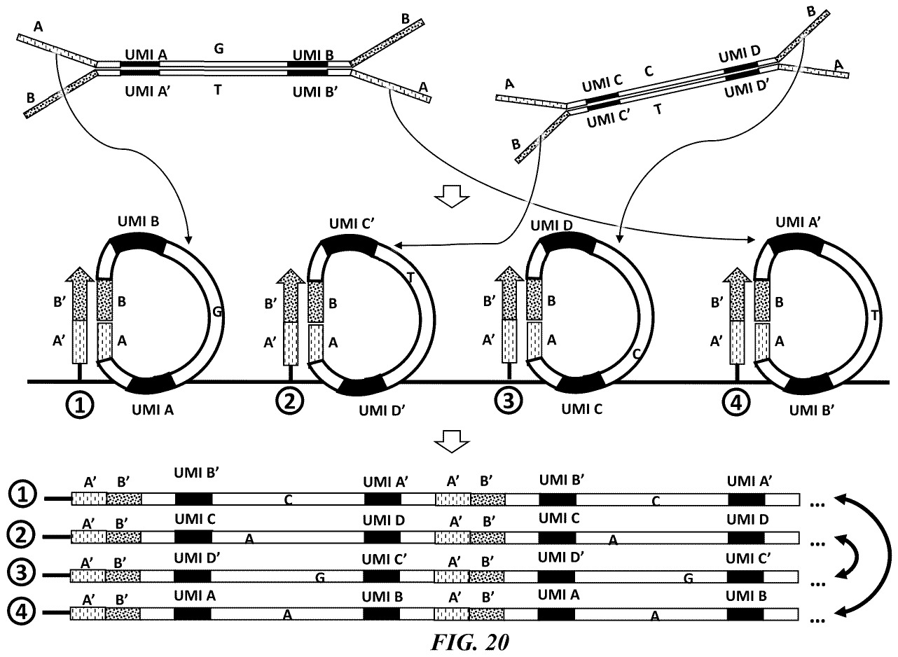

In some embodiments, rolling circle amplification, as depicted in FIG. 20 and elsewhere herein can be used to amplify molecules described in International Patent Pub. Nos. WO2023081883A2 and WO2023164505A2, each of which is hereby incorporated by reference herein in its entirety for all purposes.

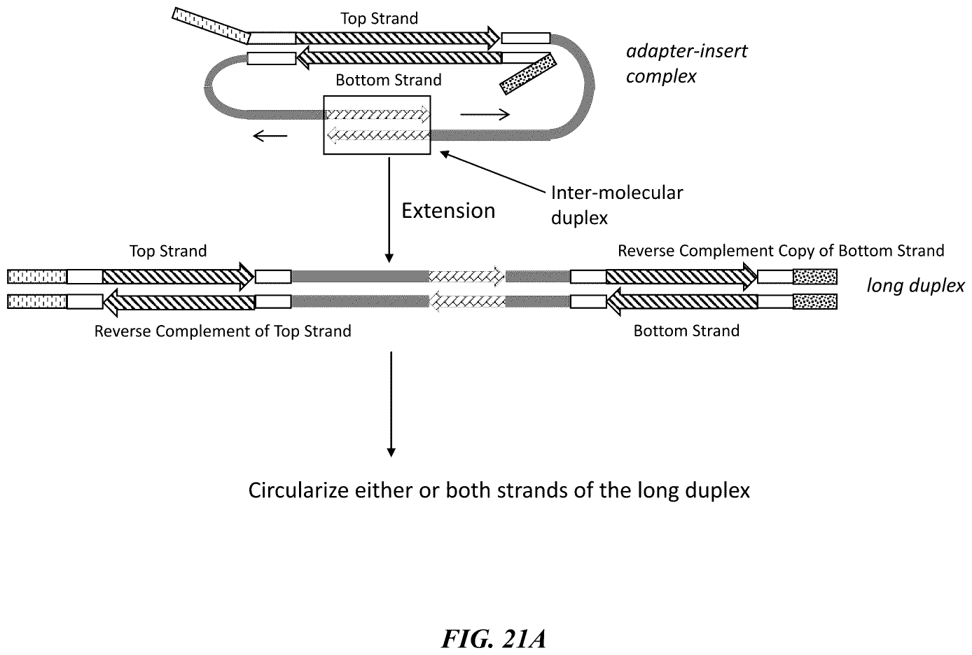

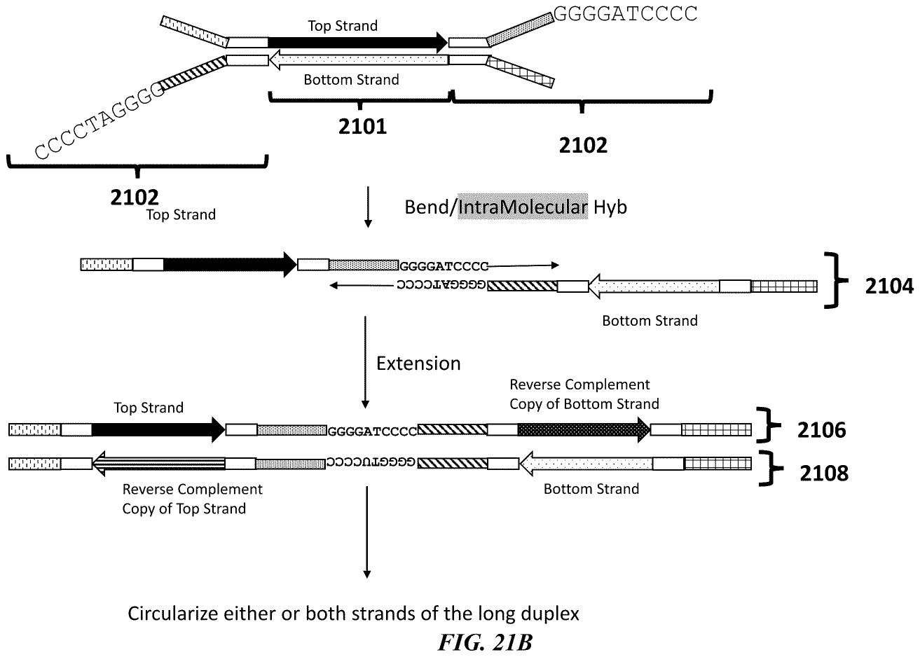

FIG. 21A illustrates an example workflow for generating a duplex molecule, comprising sequences corresponding to both strands of a double-stranded insert molecule. A single strand may retain information from both original strands of a double-stranded insert molecule. A double-stranded insert molecule is ligated to an adaptor molecule at each end. In addition to a duplex region to which the insert molecule is ligated, the adaptor molecules each have a long single-stranded region that can hybridize to form an intra-molecular duplex. Following extension reactions, the adaptor-insert complex illustrated at the top yields a long duplex molecule illustrated at the bottom. In the long duplex molecule, the original top strand from the insert is now connected with a copy of itself (the reverse complement copy of the bottom strand) obtained through the extension reaction. Similarly, the original bottom strand from the insert is also connected with a copy of itself (the reverse complement copy of the top strand) obtained through the extension reaction. Using the method illustrated in FIG. 20 or other methods described elsewhere herein, either or both strands of the long duplex molecule can be circularized and undergo rolling circle amplification.

FIG. 21B illustrates an additional workflow for generating a duplex molecule comprising sequences corresponding to both strands of a double-stranded insert molecule. A single strand may retain information from both original strands of a double-stranded insert molecule. A double-stranded insert molecule 2101 is ligated to an adaptor molecule 2102 at each end. The adaptor molecule comprises a double-stranded region, a bi-partite (or branched or Y-shaped) mismatch region, and a single-stranded binding region that extends from one of the branches of the bi-partite mismatch region. One end of the double-stranded region in the adaptor molecule 2102 is ligated to the insert molecule. The single-stranded binding region of a first adaptor ligated to a first end of the insert may self-hybridize to a single-stranded binding region of a second adaptor ligated to a second end of the insert, for intra-molecule hybridization to generate a complex 2104. Each of the single-stranded binding region may be extended to generate the long duplex molecule illustrated at the bottom. In the long duplex molecule, in the first strand 2106, the original top strand from the insert is now connected with a copy of itself (the reverse complement copy of the bottom strand) obtained through the extension reaction. Similarly, in the second strand 2108, the original bottom strand from the insert is also connected with a copy of itself (the reverse complement copy of the top strand) obtained through the extension reaction. Using the method illustrated in FIG. 20 or other methods described elsewhere herein, either or both strands of the long duplex molecule can be circularized and undergo rolling circle amplification. During intra-molecular hybridization, a bending protein may be used. The bending protein may comprise HU, IHF (Integration Host Factor), HMG1, HMG2, HMG3, or transcription factors in general, for example. The single-stranded binding region may comprise any length. The single-stranded binding region may comprise any palindrome sequence. In some cases, the single-stranded binding region may comprise a non-palindrome sequence. FIG. 21B illustrates an example palindrome sequence of CCCCTAGGGG (SEQ ID NO:392).

In any of the FIG. 21A-21B workflows, the adapters (e.g., 2102) ligated to the insert (e.g., 2101) may comprise strand recognition elements or unique molecular identifiers (UMIs). For example, in adaptor 2102, a double-stranded region, bi-partite mismatch region, and/or single-stranded binding region may comprise a UMI. For example, in adaptor 2102, the double-stranded region may comprise a strand recognition adaptor, as shown in FIG. 14(C) (without the support). In some cases, the insert may be pre-ligated to a strand recognition adaptor, on one or both ends, and then ligated to the intra-molecular hybridizing adaptors. Beneficially, each strand copy in the resulting copy molecules may be pre-associated with a strand recognition element or UMI. In other cases, amplification products from FIG. 21A-21B may not contain strand recognition elements.

One or both strands of the extended molecule may be sequenced. One or both strands of the extended molecule may be amplified, such as via RCA or any of the other amplification methods described herein and then sequenced. Each resulting copy strand, prior to or after amplification, may comprise two sequence portions derived from each of both strands of the double-stranded insert molecule, each sequence portion preceded by a sequencing primer binding site. This allows for simultaneous sequencing of material derived from both strands, such as using the same set of interrogating nucleotide flows. Similar to the methods described herein, where an interrogation(s) yields discrepancies or low quality in signals or base calls at one or more loci, error correction may be performed at such one or more loci.

### Unique Molecular Identifiers (UMI) for Maintaining Double Strand Association

A double-stranded template nucleic acid molecule may be generated using adaptors comprising a mismatch portion and a unique molecular identifier (UMI). For example, an adaptor may be a double-stranded molecule comprising an annealed double-stranded portion (e.g., stem) and a diverging mismatch portion (e.g., branches) to form a general Y-shaped structure, as seen in adaptor 2010 in FIG. 20 or the adaptors shown in FIG. 14, panels (B) or (C). The diverging mismatch portion may comprise a first strand comprising a first adaptor sequence and a second strand comprising a second adaptor sequence, which first and second adaptor sequences are non-complementary and non-annealed to each other. The annealed double-stranded portion may comprise a UMI and corresponding reverse complement thereof in the first and second strands, which UMI is unique to the adaptor molecule. A UMI sequence may have any length.

A double-stranded template nucleic acid molecule may be generated by attaching to a double-stranded insert molecule (i) at a first end a first adaptor and (ii) at a second end a second adaptor, the first adaptor comprising a first UMI and a mismatch portion comprising a first adaptor sequence and a second adaptor sequence, and the second adaptor comprising a second UMI and a mismatch portion comprising the first adaptor sequence and the second adaptor sequence. A plurality of (e.g., library of) double-stranded template nucleic acid molecules may be generated by attaching each of a plurality of double-stranded insert molecules to two adaptors at each end, the adaptors each comprising a UMI and a mismatch portion comprising the first adaptor sequence and the second adaptor sequence. Each double-stranded template nucleic acid molecule generated in this manner may comprise, in a first strand, two unique sequences corresponding to the two UMI's of the two adaptors (a first UMI sequence from the first adaptor and a reverse complement of the second UMI sequence from the second adaptor), and in a second strand, the reverse complements to the two unique sequences (the second UMI sequence from the second adaptor and a reverse complement of the first UMI sequence from the first adaptor). Further, each of the first strand and the second strand of the double-stranded template nucleic acid molecule may comprise the first adaptor sequence at a first end and the second adaptor sequence at the second end. The first strand and the second strand of the double-stranded template nucleic acid molecule may be separated from each other in downstream operations, and distinct sequencing reads generated from the two strands or derivatives thereof (e.g., amplified molecules). A distinct sequencing read may comprise a unique pair of sequences corresponding to the two UMI's of the two adaptors in the double-stranded template nucleic acid molecule, where the unique pair of sequences in a sequencing read derived from the first strand and the unique pair of sequences in a sequencing read derived from the second strand can be linked together via reverse complementarity.

FIG. 20 illustrates an example workflow for using unique molecular identifiers in a double-stranded template molecule. For illustrative purposes, two double-stranded template nucleic acid molecules (e.g., 2015) are provided, each double-stranded template nucleic acid molecule comprising an adaptor (e.g., 2010) at each end, each adaptor comprising a UMI that is unique to the adaptor (UMI A, UMI B, UMI C, UMI D) and a common mismatch portion comprising a first adaptor sequence (A) and a second adaptor sequence (B). A first template nucleic acid molecule shown on the left comprises an artificial SNP of a G/T locus, and a second template nucleic acid molecule shown on the right comprises an artificial SNP of a C/T locus.

The two strands of the double-stranded template nucleic acid molecules may be separated and amplified separately. For example, the two strands may be enzymatically, chemically, thermally, and/or via one or more stimuli, separated. Any one or more amplification methods described herein may be used, e.g., PCR, ePCR, eRPA, bridge amplification, RPA, RCA, MDA, etc. The amplification may occur in solution or on a substrate. The amplification may comprise use of a support, such as a bead to associate amplified strands to a single support. FIG. 20 illustrates an example RCA amplification occurring on a substrate 2025, in which a plurality of amplification primers (1, 2, 3, 4) are immobilized on distinct locations of the substrate. An amplification primer may comprise a first adaptor binding sequence (A′) and a second adaptor binding sequence (B′) such that each strand of the double-stranded template nucleic acid molecules which comprises the first adaptor sequence (A) and the second adaptor sequence (B) at each end can bind to the amplification primer in a substantially circularized form. The two ends may be ligated to form a circular template. In this figure, each of the four strands binds to different amplification primers in substantially circularized form. The amplification primer may be extended using the circularized strand as a template to generate a concatemer, or a repeating amplification product, which is shown in the bottom of the figure. In an example, the amplification primer at location 1 is bound to the top strand of the left template nucleic acid molecule and extended to generate a concatemer with the repeating unit of: [A′]-[B′]-[UMI B]-[insert]-[UMI A′]; the amplification primer at location 2 is bound to the bottom strand of the right template nucleic acid molecule and extended to generate a concatemer with the repeating unit of [A′]-[B′]-[UMI C]-[insert]-[UMI D′]; the amplification primer at location 3 is bound to the top strand of the right template nucleic acid molecule and extended to generate a concatemer with the repeating unit of: [A′]-[B′]-[UMI D]-[insert]-[UMI C′]; the amplification primer at location 4 is bound to the bottom strand of the left template nucleic acid molecule and extended to generate a concatemer with the repeating unit of [A′]-[B′]-[UMI A]-[insert]-[UMI B′].

The amplified products of the two strands, if not amplified on the substrate, may be immobilized onto a substrate for sequencing. Sequencing may be performed on the amplified products of the two strands by hybridizing a sequencing primer, such as to the [A′], [B′], or other functional binding site on the amplified products, and extending stepwise with nucleotide(s) and detecting to generate sequencing reads. For RCA, as shown in FIG. 20, multiple sequencing primers may bind to each single concatemer. Alternatively or in addition, such as for other amplification methods not shown in FIG. 20, a single sequencing primer may bind to a single amplified strand. In an example, the sequencing read generated at location 1, corresponding to the top strand of the left template nucleic acid molecule comprises: [UMI A]-[insert with ‘G’ at G/T locus]-[UMI B′]; the sequencing read generated at location 2, corresponding to the bottom strand of the right template nucleic acid molecule comprises: [UMI D]-[insert with ‘T’ at C/T locus]-[UMI C′]; the sequencing read generated at location 3, corresponding to the top strand of the right template nucleic acid molecule comprises: [UMI C]-[insert with ‘C’ at C/T locus]-[UMI D′]; the sequencing read generated at location 4, corresponding to the bottom strand of the left template nucleic acid molecule comprises: [UMI B]-[insert with ‘T’ at G/T locus]-[UMI A′]. Sequencing reads at locations 1 and 4 may be linked and determined/recognized as having derived from different strands of the same double-stranded template nucleic acid molecule having a G/T locus based at least in part on linking the pair of [UMI A] and [UMI B′] sequences with the pair of [UMI B] and [UMI A′] sequences in the two sequencing reads and recognizing the G/T locus at corresponding base positions. Similarly, sequencing reads at locations 2 and 3 may be linked and determined/recognized as having derived from different strands of the same double-stranded template nucleic acid molecule having a C/T locus based at least in part on linking the pair of [UMI D] and [UMI C′] sequences with the pair of [UMI C] and [UMI D′] sequences in the two sequencing reads and recognizing the C/T locus at corresponding base positions.

It will be appreciated that the amplification primers and adaptors may be varied, such as to contain various functional sequences described elsewhere herein.

### Target Enrichment and Methylation Sequencing

Some applications require high-depth, targeted sequencing (e.g., for cancer diagnosis and tracking). Either of the workflows described with respect to FIGS. 15A-15C and FIG. 17 may be used to amplify target molecules for enrichment purposes. Workflow 1700 may be paused prior to digestion 1709 (e.g., at a point with multiple individual double-stranded template-adaptor molecules comprise a single-stranded hairpin adaptor region). Workflow 1500 may be modified at digestion 1509, so that regions 1534 are digested and region 1532 are not digested (e.g., restriction sites 1532 are not cleaved), leaving individual double-stranded template-adaptor molecules comprising a single-stranded hairpin adaptor region.

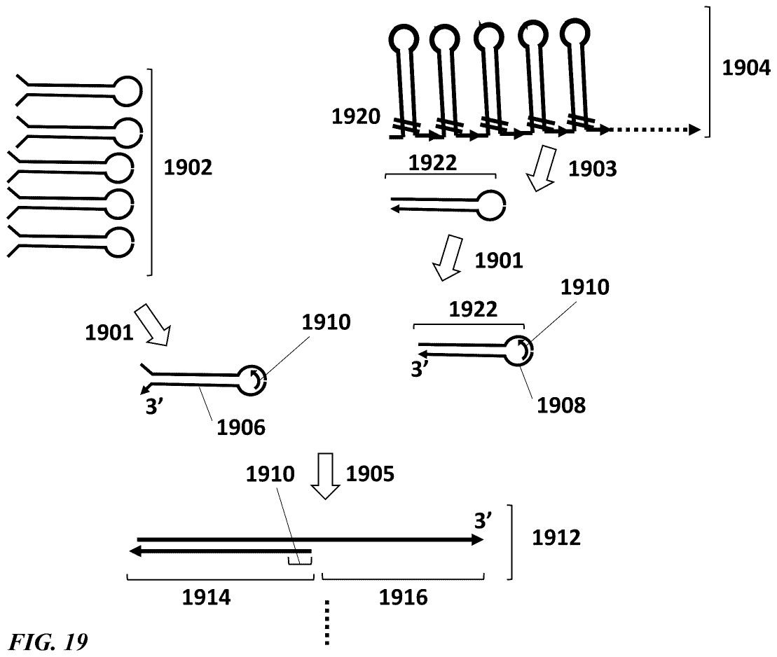

FIG. 19 illustrates workflows for processing different double-stranded target molecules. In FIG. 19, double-stranded target molecules 1902 and 1904 are provided. Molecules 1902 comprise two different types of adaptors, where at least one adaptor type comprises a single-stranded hairpin region comprising a mismatch region. Individual double-stranded regions in molecule 1904 comprise at least one adaptor type comprising a single-stranded hairpin region comprising a mismatch region. Molecule 1904 is cleaved 1903 (e.g., digested) at cleavage sites 1920, resulting in a plurality of double-stranded molecules 1922.

In either workflow, to enable an efficient target capture process, as shown in FIG. 19, a primer 1910, where the primer binds to the single-stranded hairpin region in the double-stranded template-adaptor molecules, is introduced. Primer 1910 is extended 1905 to create a strand complementary to only a single region of the double-stranded template-adaptor molecule (e.g., to either the forward or reverse strand of an initial target molecule). As shown in FIG. 19, the resulting molecule 1912 comprises a first, double-stranded region 1914 and a second, single-stranded region 1916. Resulting molecule 1912 may be further processed, e.g., for targeted sequencing and/or for methylation sequencing. For instance, a plurality of molecules 1912 may be filtered for desired target sequences. Alternatively or in addition, molecules 1912 may be exposed to methylation conversion.

In some instances, a partially single-stranded molecule 1912 is subjected to EM conversion, resulting in a partially converted partially single-stranded molecule: EM-conversion only processes single-stranded sequences, so region 1914 will be protected from methylation conversion, while any unmethylated cytosines in region 1916 will be converted to uracils. Alternatively, in other instances, primer 1910 may be extended under conditions comprising methylated cytosines, such that region 1914 will be protected. Subsequently, molecule 1912 will be exposed to bisulfite conversion. In either case, region 1914 will comprise an unconverted target sequence and region 1916 will comprise a converted target sequence. Advantageously, the use of a molecule 1912 can provide sequencing information on the unconverted region of molecule 1912 and methylation sequencing information on the converted rection of molecule 1912 in a single sequencing read (i.e., because the converted and unconverted regions correspond to both the forward and reverse strands).

Examples of partially converted molecule methylation sequencing is further described in International Patent Appl. No. PCT/US2022/079395, which is hereby incorporated by reference in entirety.

As with other methods described herein, the forward and reverse strand context is maintained for each molecule 1912. It will be appreciated that the methods described with respect to FIG. 19 may also be performed on individual, double-stranded template molecules that have not previously been amplified (e.g., for completely PCR-free workflows and/or for single-molecule sequencing). In some instances, molecules 1902 and 1904 may comprise mismatch regions or other adaptor types, as described herein. However, in each case, molecules 1902 and 1904 must also comprise an adaptor region (e.g., the single-stranded hairpin adaptor region) that connects the forward and reverse strands.

In some cases, after concatemeric molecules comprising a plurality of copies of hairpin-containing molecules (e.g., molecules 1530, 1904, etc.) are generated, the amplified molecules may be enriched, such as for exome enrichment, by contacting the amplified molecules with a plurality of enrichment probes, heated, and selected for enrichment (isolating the molecules that have hybridized to the enrichment probes). In some cases, recombinase can be used to mediate the attachment of the enrichment probes to the hairpin regions while avoiding heating.

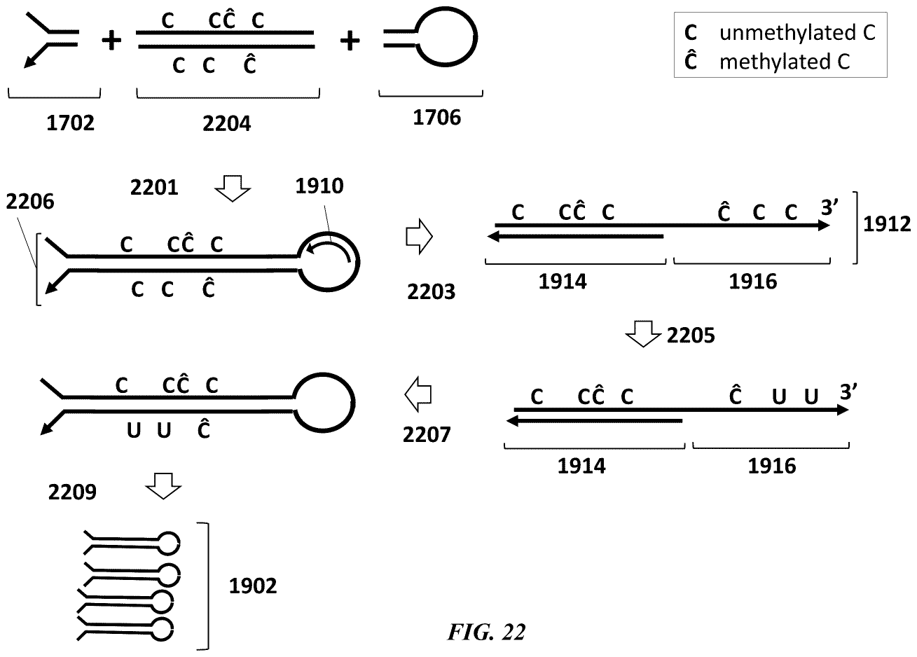

FIG. 22 illustrates a method for generating double-stranded template molecules which comprise a first strand and a second strand, the first strand comprising a sequence of the original DNA sequence and the second strand comprising a reverse complement copy of the original DNA sequence but which was protected from conversion during methylation conversion treatment. The method may be integrated with one or more steps of the workflows described with respect to FIG. 17 and FIG. 19. As described with reference to workflow 1700 of FIG. 17, a double-stranded target molecule 2204 (also referred to as insert molecule) may be ligated 2201 to y-shaped adaptor 1702 and an adaptor 1706 with a hairpin region to generate molecule 2206. As described with reference to workflow 1900 of FIG. 19, a primer 1910 may bind to a sequence in the single-stranded hairpin region of adaptor 1706 and be extended 2203 to generate molecule 1912 which comprises a first, double-stranded region 1914 and a second, single-stranded region 1916. The molecule 1912 may be subjected to methylation conversion treatment 2205, to convert any unmethylated cytosine residues in the single-stranded region 1916 to uracil residues and leave any unmethylated cytosine residues in the double-stranded region 1914 intact. The extended primer molecule may be denatured 2207 from the double-stranded region such that the partially converted strand (top strand in the figure) may resume the hairpin configuration and be amplified 2209 according to various methods described herein to generate amplified molecules 1902. A strand recognition element in the mismatched portions of the adaptors may be included in the amplified molecules 1902 or further added to the amplified molecules in further processing. Both strands of the amplified molecules 1902, or derivatives thereof, may be sequenced. The double-stranded, amplified molecules 1902, or derivatives thereof, for example may be subjected to the workflows described with respect to FIGS. 10A-10D to generate mixed copies of forward and reverse strands on the support.

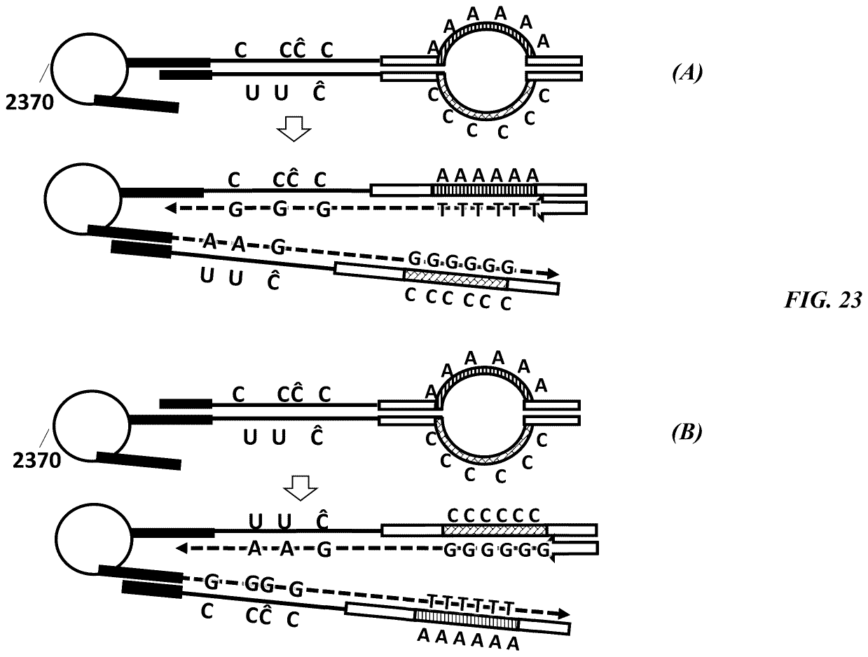

FIG. 23 illustrates two scenarios in panel (A) and panel (B), in which balance constructs are generated from the amplified molecule derivatives of the partially converted molecule. In Panel (A), the methylation-unconverted strand (top strand) is ligated to a primer on support 2370, while the methylation-converted strand (bottom strand) is hybridized. The hybridized strand may denature from the methylation-unconverted strand, and another primer on the support 2370 may hybridize to the methylation-converted strand and be extended using the methylation-converted strand as a template. Thus, in the scenario of Panel (A), the balanced constructs may comprise in the forward strand a methylation-unconverted sequence, and in the reverse strand a reverse complement of the methylation-converted sequence of the partially converted double-stranded molecule. In Panel (B), the methylation-converted strand (bottom strand) is ligated to a primer on support 2370, while the methylation-unconverted strand (top strand) is hybridized. The hybridized strand may denature from the methylation-converted strand, and another primer on the support 2370 may hybridize to the methylation-unconverted strand and be extended using the methylation-unconverted strand as a template. Thus, in the scenario of Panel (B), the balanced constructs may comprise in the forward strand a methylation-converted sequence, and in the reverse strand a reverse complement of the methylation-unconverted sequence of the partially converted double-stranded molecule.

Methods described herein provide a balanced construct comprising a support comprising a mixture of copies of forward strands and reverse strands, each strand comprising a strand recognition element (e.g., mismatched sequence) derived from the mismatched adaptor. In some cases, (i) the forward strand comprises a sequence that corresponds to a methylation-unconverted DNA sequence from the sample, in which during processing of the template (e.g., as in the workflow of FIG. 22), unmethylated and methylated cytosine residues alike from a first strand of the original DNA sequence were not converted to uracil and/or thymine residues subsequent to methylation conversion treatment, and (ii) the reverse strand comprises a sequence that corresponds to a methylation-converted DNA sequence from the sample, in which during processing of the template (e.g., as in the workflow of FIG. 22), unmethylated cytosine residues in a second strand of the original DNA sequence were converted to uracil and/or thymine residues subsequent to methylation conversion treatment. In some cases, the forward strand and the reverse strand may comprise a methylation-unconverted DNA sequence from a first strand of the sample and a reverse complement of the methylation-converted DNA sequence from the second strand of the sample, respectively. In some cases, (i) the forward strand comprises a sequence that corresponds to a methylation-converted DNA sequence from the sample, in which during processing of the template (e.g., as in the workflow of FIG. 22), unmethylated cytosine residues in a second strand of the original DNA sequence were converted to uracil and/or thymine residues subsequent to methylation conversion treatment, and (ii) the reverse strand comprises a sequence that corresponds to a methylation-unconverted DNA sequence from the sample, in which during processing of the template (e.g., as in the workflow of FIG. 22), unmethylated and methylated cytosine residues alike from a first strand of the original DNA sequence were not converted to uracil and/or thymine residues subsequent to methylation conversion treatment. In some cases, the forward strand and the reverse strand may comprise a methylation-converted DNA sequence from a second strand of the sample and a reverse complement of the methylation-unconverted DNA sequence from the first strand of the sample, respectively. The strand recognition element in the mismatched adaptor may be used to measure the mixture rate between the two strands. However, when using flow-based sequencing according to a cyclic four-base flow order (e.g., T-G-C-A), due to the differences in residues at the conversion locations, the extending primers hybridized respectively to the forward and reverse strands go out of sync in the flow space, making it difficult to resolve the correct signal from that support location.

For example, where the original DNA sequence from the sample is TCGTCATCTG (SEQ ID NO:393) (where C is unmethylated and C is methylated), after processing the template molecule with methylation conversion treatment to generate a partially converted molecule and preparing a balanced construct such that the strand with the methylation-unconverted sequence is ligated to the support (e.g., see Panel (A) of FIG. 23), in the balanced construct, the forward strand may comprise the following sequence: TCGTCATCTG (SEQ ID NO:394) and the reverse strand may comprise the following sequence: TTGTCATTTG (SEQ ID NO:395).

When sequenced with the flow order of (T-G-C-A), the forward strand may yield the flow signal of:
[1 0 1 0 0 1 0 1 0 1 1 1 0 1 0 1 1]
   and the reverse strand may yield the flow signal of:
[2 1 0 0 1 0 1 1 3 1],
   which demonstrates a clear diversion of flow signals between the forward and reverse strands at each flow position. Accordingly, composite signals from the forward and reverse strands may be difficult to resolve.

Provided herein are methods for synchronizing flow signals for flow-based methylation sequencing using modified flow orders. A modified flow order comprising a sequence of (T-C-T) in the flow order cycle may be used to synchronize and re-synchronize flow signals in the flow positions subsequent to a conversion location until the next conversion location. In an example, when the balanced construct of the sequences described above is sequenced with the modified flow order of (T-C-T-G-A), the forward strand may yield the flow signal of:
[2 0 0 1 0 1 1 0 0 1 3 0 0 1]
   and the reverse strand may yield the flow signal of:
[1 1 0 1 0 11 0 0 1 1 1 1 1],
   which demonstrates that the flow signals between the forward and reverse strands only diverge at the T-C-T flow segment (flow positions bolded) of the modified flow order and is synchronized for the other segments of the flow orders. Thus, the modified flow order may permit the resolution of flow signals in, and hence sequencing of, methylation-unconverted portions of the template sequence. For example, the flow order can be (T-C-T-G-A) or (G-T-C-T-A), etc.

Alternatively, after processing the template molecule with methylation conversion treatment to generate a partially converted molecule and preparing a balanced construct such that the strand with the methylation-converted sequence is ligated to the support (e.g., see Panel (B) of FIG. 23), in the balanced construct, a modified flow order comprising a sequence of (G-A-G) in the flow order cycle may be used to synchronize and re-synchronize flow signals in the flow positions subsequent to a conversion location until the next conversion location. For example, the flow order can be (T-C-G-A-G) or (T-G-A-G-C), etc.

In scenarios where a support comprises methylation-converted sequences in both forward and reverse strands, a modified flow order comprising a sequence of both (T-C-T) and (G-A-G) in the flow order cycle may be used to synchronize and re-synchronize flow signals in the flow positions subsequent to a conversion location until the next conversion location. For example, the flow order can be (T-C-T-G-A-G), etc.

A method for sequencing may comprise: (a) providing a balanced construct comprising a mixture of a forward strand and a reverse strand, wherein (i) the forward strand comprises a first sequence that is identical to or a reverse complement of a first strand of a double-stranded template molecule of a sample, and (ii) the reverse strand comprises a second sequence that is a reverse complement of or identical to a methylation-converted sequence of a second strand of the double-stranded template molecule of the sample, respectively; and (b) sequencing the forward strand and the reverse strand by (i) hybridizing primers to the forward strand and the reverse strand, respectively, (ii) extending the primers with nucleotides in nucleotide flows provided according to a repeating flow order, wherein a nucleotide flow comprises nucleotides of a single canonical base type, wherein the repeating flow order comprises a consecutive three flow order of a thymine-base flow, cytosine-base flow, and thymine-base flow, and (iii) detecting flow signals indicative of incorporation of nucleotides, or lack thereof, by the primers subsequent to the respective nucleotide flows. The method may further comprise using the flow signals detected in (b)(iii) to determine a methylation status of the double-stranded template molecule. In some cases, the forward strand may comprise a first strand recognition element comprising a first homopolymer sequence and the reverse strand may comprise a second strand recognition element comprising a second homopolymer sequence, wherein the first homopolymer sequence and the second homopolymer sequence comprise different bases. In some cases, the method may further comprise using the flow signals detected in (b)(iii) which corresponds to the first strand recognition element and the second strand recognition element to determine a forward-reverse ratio of a number of copies of the forward strand and a number of copies of the reverse strand on the balance construct. In some cases, the method may further comprise using the forward-reverse ratio to determine a methylation status of the double-stranded template molecule. The methylation status may comprise a location of methylated cytosine residues in the original template sequence of the sample.

As used herein, the terms “balanced construct” may generally refer to a support generated using a double-stranded template comprising a strand recognition adaptor or mismatch adaptor. A balanced construct may comprise a mixture of forward and reverse strands derived from the double-stranded template. As used herein, the terms “strand recognition element” may generally refer to a sequence derived from (e.g., identical to or reverse complement of) one of a pair of mismatched sequences in a mismatch adaptor. As used herein, the terms “conversion location” may generally refer to a location in a nucleic acid sequence in which a cytosine residue was converted to a uracil or thymine residue following methylation conversion treatment.

### Sequencing Methods

During sequencing by synthesis, a sequencing primer may be hybridized to a template (e.g., to a primer binding site on the template) and extended in a stepwise manner by, in each extension step, contacting the complex with nucleotide reagents of known canonical base type(s). The extended or extending sequencing primer may also be referred to herein as a growing strand. An extension step may be a bright step (also referred to herein, in some cases, as labeled step, hot step, or detected step) or a dark step (also referred to herein, in some cases, as an unlabeled step, cold step, or undetected step). A sequencing method may comprise only bright steps. Alternatively, a sequencing method may comprise a mix of bright step(s) and dark step(s). For a bright step, the growing strand may be contacted with nucleotide reagents that include labeled nucleotides (of known canonical base type(s)) and signals indicative of incorporation of the labeled nucleotides, or lack thereof, may be detected to determine a base or sequence of the template. Alternatively or in addition, for a bright step, the growing strand may be contacted with a mixture of labeled and unlabeled nucleotide reagents. For a dark step, the growing strand may be contacted with solely unlabeled nucleotide reagents. Alternatively or in addition, for a dark step, the growing strand may be contacted with labeled nucleotide reagents and detection omitted. Sequencing data can be generated from the signals collected after one or more extension steps. A sequencing by synthesis method may comprise any number of bright steps and any number of dark steps. A sequencing by synthesis method may comprise any number of bright regions (consecutive bright steps) and any number of dark regions (consecutive dark steps). In some cases, the dark steps or dark regions may be used to accelerate or fast forward through certain regions of the template during sequencing. In some cases, the dark steps or dark regions may be advantageous to correct phasing problems.

Sequencing methods of the present disclosure may comprise flow-based sequencing, non-terminated sequencing, and/or terminated sequencing. Sequencing methods of the present disclosure may be applied to colony-based sequencing where template strands are provided in clusters, each cluster comprising copies from a single template molecule, concatemer-based sequencing where template strands are provided as concatemers, each concatemer comprising multiple copies of a single template insert, or single molecule-based sequencing where template strands are provided as single molecules as opposed to colonies, clusters, or concatemers. For non-single molecule-based sequencing methods, multiple sequencing primers may be simultaneously bound to multiple primer binding sites across multiple copies of a template insert (in clusters or in a concatemer), extended in parallel, and provide synchronized and cumulative signals from the multiple copies at bright steps.

### Terminated Sequencing

In terminated sequencing methods, a bright step may comprise terminated nucleotides (e.g., reversibly terminated nucleotides). In some cases, a bright step may comprise a single nucleotide base type (e.g., A, C, G, T, U) or a mixture of nucleotide base types (e.g., 2, 3, 4, or more base types). A dark step may comprise terminated nucleotides, unterminated nucleotides, or a mixture thereof. A dark step may comprise a single nucleotide base type. Alternatively, a dark step may comprise a mixture of nucleotide base types. In an extension step comprising solely reversibly terminated nucleotides (e.g., and not unterminated nucleotides) at most a single nucleotide base may be incorporated into a growing strand. In an extension step comprising a mixture of reversibly terminated and unterminated nucleotides, more than one nucleotide base may be incorporated into a growing strand, the last incorporation being of a terminated nucleotide.

### NON-TERMINATED SEQUENCING, FLOW-BASED SEQUENCING

Sequencing data can be generated using flow-based sequencing methods that include extending a primer bound to a template nucleic acid according to a pre-determined flow cycle and/or flow order where, in one or more flow positions, known canonical base type(s) of nucleotides (e.g., A, C, G, T, U) is accessible to the extending primer. At least some of the nucleotides may include a label, which labeled nucleotides upon incorporation into the extending primer render a detectable signal. The resulting sequence by which nucleotides are incorporated into the extended primer is expected to be the reverse complement of the sequence of the template nucleic acid. A method for sequencing can comprise using a flow sequencing method that includes (1) extending a primer using labeled nucleotides in a flow, and (2) detecting the presence or absence of a labeled nucleotide incorporated into the extending primer to generate sequencing data. Flow sequencing methods may also be referred to as “natural sequencing-by-synthesis,” “mostly natural sequencing-by-synthesis,” or “non-terminated sequencing-by-synthesis” methods. Example methods are described in U.S. Pat. No. 8,772,473 and U.S. Pat. Pub. No. 2022/0170089A1, each of which is incorporated herein by reference in its entirety.

In flow sequencing, iterative nucleotide flows are used to extend the primer hybridized to the template nucleic acid, with detection of incorporated nucleotides between one or more flows. The nucleotides may be, for example, non-terminating nucleotides such that more than one consecutive base can be incorporated into the extending primer strand if more than one consecutive complementary base (or homopolymer region) is present in the template strand. At least a portion of the nucleotides can be labeled so that incorporation can be detected. Generally, only a single nucleotide type is introduced in a flow, although two or three different types of nucleotides may be simultaneously introduced in certain embodiments. This methodology can be contrasted with sequencing methods that use a reversible terminator, where primer extension is stopped after extension of every single base before the terminator is reversed (e.g., by removing a 3′ blocking group) to allow incorporation of the next succeeding base.

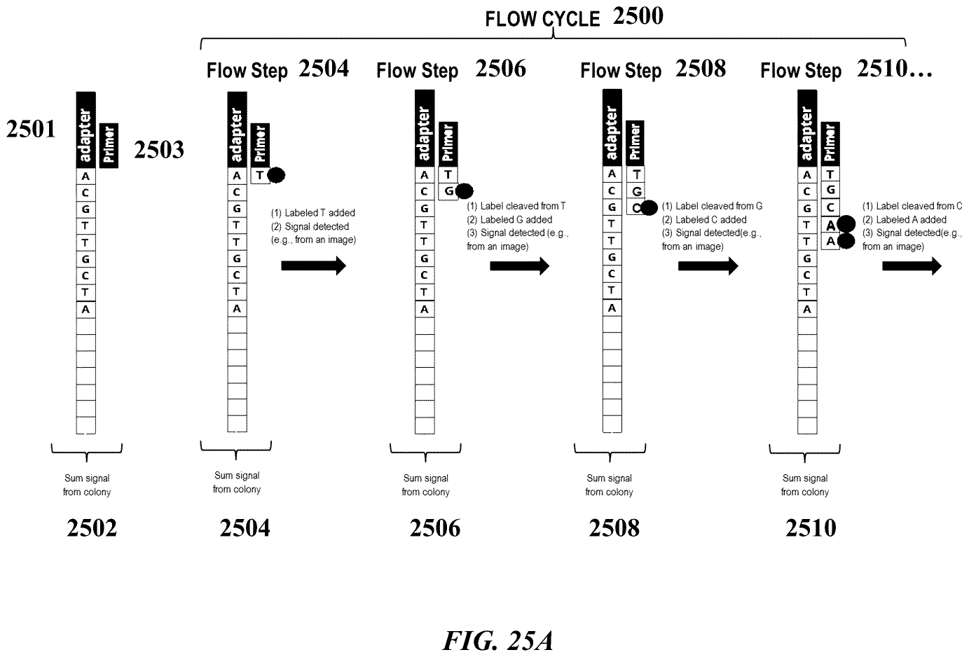

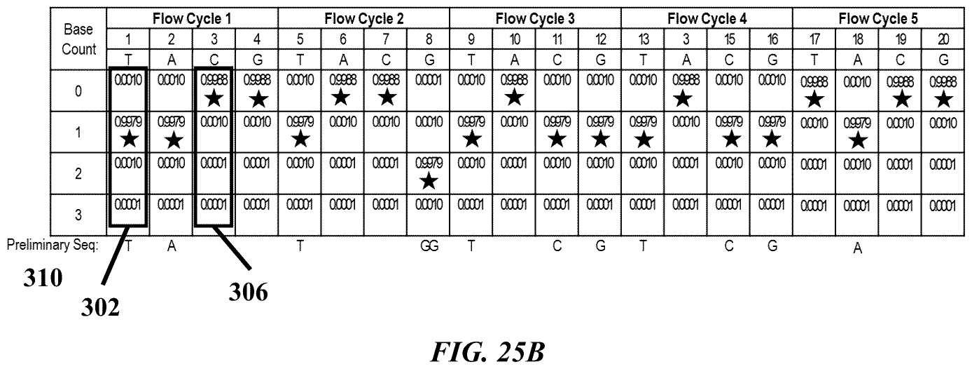

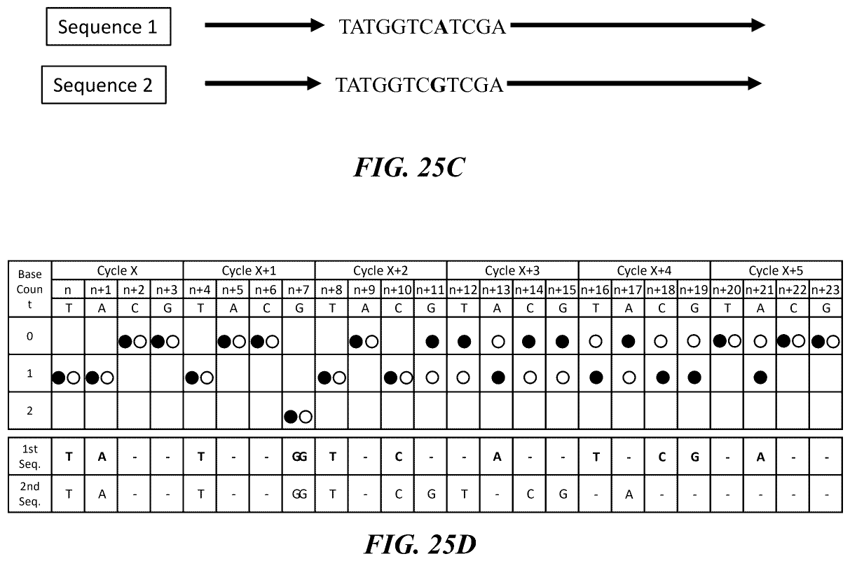

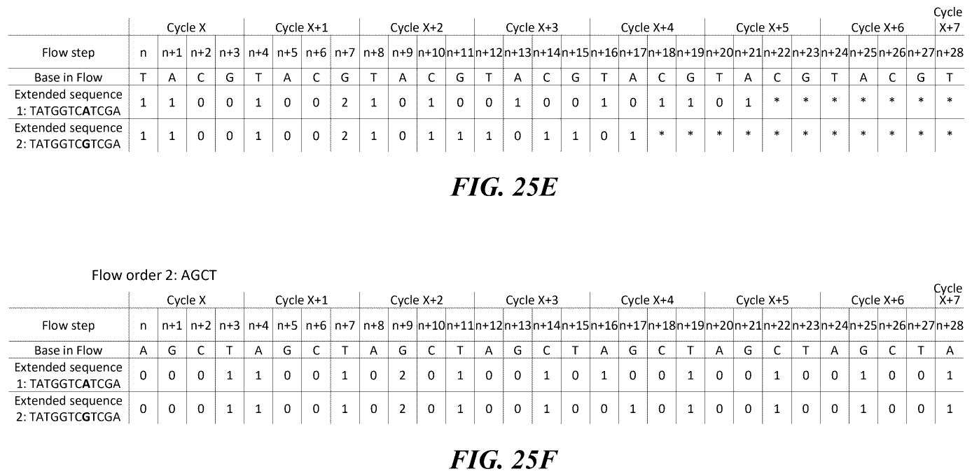

FIG. 25A illustrates an example flow sequencing method that can be used to generate the sequencing data described herein. Template nucleic acids may be immobilized to a surface (e.g., the surface of a bead attached to a substrate or directly to a substrate). In this example, the template nucleic acid includes an adaptor sequence 2501 followed by an insert sequence (“ACGTTGCTA . . . ”). The adaptor sequence 2501 can include a sequencing primer hybridization site. At operation 2502, a sequencing primer 2503 is hybridized to the adaptor sequence 2501 at the sequencing primer hybridization site. The sequencing primer 2503 is then extended in a series of flows according to flow cycle 2500 with flow order: [T G C A]. In this example, the flow cycle 2500 includes four flow steps 2504, 2506, 2508, 2510, and in a given flow step, a single base type is provided to the template-primer hybrid. In flow step 2504, nucleotides comprising labeled T nucleotides are provided; in flow step 2506, nucleotides comprising labeled G nucleotides are provided; in flow step 2508, nucleotides comprising labeled C nucleotides are provided; in flow step 2510, nucleotides comprising labeled A nucleotides are provided. Nucleotides in a single-base flow may comprise a mixture of labeled and unlabeled nucleotides of the single base. At flow step 2504, a labeled T nucleotide is incorporated by the extending sequencing primer 2503 opposite the A base in the template strand. Then, a signal indicative of the incorporation of the labeled T nucleotide can be detected. For example, the signal may be detected by imaging the surface the template nucleic acids are immobilized on and analyzing the resulting image(s). The sequencing platform may be washed with a wash buffer to remove unincorporated nucleotides prior to signal detection. In some cases, prior to the next flow step (e.g., 2506), the label may be removed from the incorporated labeled T nucleotide (e.g., by cleaving the label from the nucleotide), before proceeding. Nucleotide flow, detection, and optionally cleavage, may be repeated according to a flow order that may or may not include repeating the flow cycle 2500 for any number of times. Flow step 2510 illustrates incorporation of two labeled A bases by the extending sequencing primer 2503 opposite the two T bases in the template strand, per the non-terminated nature of the flown nucleotides. The detected signal intensity indicating the incorporation of two A nucleotides may be greater than the signal intensity indicating the incorporation of one nucleotide. In this example, the following flow space values corresponding to flow steps 2504-2510 may be: [1 1 1 2]. For simplicity, this Figure illustrates incorporation of two labeled A nucleotides in the same hybrid. However, flow-based sequencing may be performed on colonies of amplified molecules, e.g., each bead representing one colony, where an optically resolvable location contains multiple copies of the same template nucleic acid molecule (e.g., a location contains one amplified bead), such that the signal detected at an optically resolvable location represents an aggregate signal from the multiple copies of molecules. Thus, when using a nucleotide flow mixture containing labeled and unlabeled nucleotides of a same base type, the incorporation of the labeled nucleotides can be distributed across the multiple copies of the molecules, and the aggregate signal from the multiple copies detected. In some cases, for a majority of hybrids, at most a single labeled nucleotide may be incorporated into a single homopolymer stretch in a hybrid—the longer the homopolymer stretch, the more likely that more hybrids of the plurality of copies of hybrids in an optically resolvable location will incorporate one labeled nucleotide.

While each flow step in the example flow sequencing method in FIG. 25A results in incorporation of one or more nucleotides (and thus a detected signal indicating such incorporation), it should be appreciated that not all flow steps result in incorporation of nucleotides. In some flow steps, no nucleotide base may be incorporated (for example, in the absence of a complementary base in the template).

A nucleotide mixture that is provided during any one flow may comprise only labeled nucleotides, only unlabeled nucleotides, or a mixture of labeled and unlabeled nucleotides. The mixture of labeled and unlabeled nucleotides may be of any fraction of labeled nucleotides, such as at least or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. Labeled nucleotides may comprise a dye, fluorophore, or quantum dot, multiples thereof, and/or combination thereof. In some cases, nucleotides of different canonical base types may be labeled and detectable at a single frequency (e.g., using the same or different dyes). In other cases, nucleotides of different canonical base types may be labeled and detectable at different frequencies (e.g., using the same or different dyes). Labeled nucleotides may comprise an optical moiety (e.g., dye, fluorophore, quantum dot, label, etc.) coupled to a nucleobase via a linker, and the label from the labeled nucleotides may be removed by cleaving the linker to remove the optical moiety. Cleaving may comprise one or more stimuli, such as exposure to a chemical (e.g., reducing agent), an enzyme, light (e.g., UV light), or temperature change (e.g., heat).

Flow-based sequencing may comprise providing non-detected nucleotide flow(s), for example to skip sequencing of a region(s) of the template nucleic acid; to ensure completion of incorporation reactions across all template-primer hybrids in the reaction space; and/or phasing or re-phasing. A non-detected nucleotide flow may be referred to herein as a “dark flow”, “dark tap”, or “dark tap flow.” A detected nucleotide flow may be referred to herein as a “bright flow”, “bright tap”, or “bright tap flow.” Incorporation reactions may be incomplete in the reaction space when not all available incorporation sites in the template-primer hybrids have incorporated a complementary base, such as due to reaction kinetics and/or insufficient incubation time or reagents. In some cases, single-base flows of the same canonical base type may be provided consecutively (without intervening flow of a different nucleotide base type) for any number of consecutive flows, to ensure completion of incorporation reactions. A consecutive same-base flow may be referred to herein as a “double tap” or “double tap flow” if there are two consecutive flows, a “triple tap” or “triple tap flow” if there are three consecutive flows, or a “nth tap” or “nth tap flow” if there are n consecutive flows of the same base type. A double tap, triple tap, or nth tap flow may or may not be detected. Labels in a flow may or may not be removed (e.g., cleaved) prior to the double tap, triple tap, or nth tap flow. Detection of labeled nucleotides from a particular flow may be performed prior to, during, or subsequent to the double tap, triple tap, or nth tap flow. Accordingly, below are non-limiting examples of flow cycles that can be used in a larger flow order of flow-based sequencing methods, which may or may not be repeated and/or mixed and matched with other flow cycles, where * after a base represents a detected flow step and/between bases represents a mixed base flow:
Single-base flow: e.g., [T* A* C * G*]
Single-base flow with double tap: e.g., [T* T A* A C* C G* G]
Mixed base flow, all labeled: e.g., [T* A*/C*/G*]
Mixed base flow, some unlabeled: e.g., [T* A/C*/G]
Mixed base flow, some unlabeled: e.g., [T A*/C*/G*]
Skip region base flow: e.g., [T/A/C G/A/T]
Three base flow cycle: e.g., [T A C]

FIG. 25B illustrates an example flowgram of signals detected after five exemplary flow cycles of [T A C G] are performed to extend a sequencing primer, in accordance with some cases. Each column in the flowgram corresponds to a detected flow step (e.g., 302, 306), and the values in each column collectively represent the detected signal intensity in the flow step. In each detected flow step, the flow signal can be determined from an analog signal that is detected during the sequencing process, such as a fluorescent signal of the one or more bases incorporated. In some cases, for a flow step, the detected signal intensity can be expressed in probabilistic terms. Specifically, the detected signal intensity can be expressed in a series of likelihood values corresponding to different integer homopolymer base lengths (e.g., 0 base, 1 base, 2 bases, 3 bases, etc.) for the flow position. For flow step 302, the detected signal intensity is expressed by a first likelihood value of 0.001 for 0 base, a second likelihood value of 0.9979 for 1 base, a third likelihood value of 0.001 for 3 bases, and a fourth likelihood value of 0.0001 for 4 bases. This can be interpreted to indicate that there is a high statistical likelihood that one nucleotide base has been incorporated. In this flow step, a single T was determined to be incorporated, which means there is an A in the template. Similarly, for flow step 306, the column values can collectively indicate that there is a high statistical likelihood that no base has been incorporated (with 0.9988 likelihood value for 0 bases). With similar analyses performed at each flow position, a preliminary sequence 310 (SEQ ID NO: 390 TATGGTCGTCGA) of the extending primer can be determined, and reverse complement (i.e., the template strand sequence) readily determined from the preliminary sequence. For example, the most likely sequence can be determined by selecting the base count with the highest likelihood at each flow position, as shown by the stars in the flowgram. Further, the likelihood of this sequencing data set can be determined as the product of the selected likelihood at each flow position. Accordingly, the flowgram may be formatted as a sparse matrix, with a flow signal represented by a plurality of likelihood values indicative of a plurality of base homopolymer length counts at each flow position. The homopolymer length likelihood may vary, for example, based on the noise or other artifacts present during detection of the analog signal during sequencing. In some cases, if the homopolymer length likelihood statistical parameter or likelihood is below a predetermined threshold, the parameter may be set to a predetermined non-zero value that is substantially zero (i.e., some very small value or negligible value) to aid the downstream statistical analysis further discussed herein, wherein a true zero value may give rise to a computational error or insufficiently differentiate between levels of unlikelihood, e.g., very unlikely (0.0001) and inconceivable (0). Thus, a method for sequencing may comprise generating a flowgram using analog signals (e.g., fluorescent signals) detected from a template nucleic acid or derivative thereof, and generating base calls and/or sequencing reads using the flowgram.

As will be appreciated, in flow-based sequencing, the signal for any flow position in the sequencing data is flow order-dependent in that the same flow position for a same template nucleic acid may express different flow signals for different flow orders. Any useful predetermined flow cycles and/or flow orders may be designed to sequence a template nucleic acid and/or more accurately or precisely detect a particular type of sequence (e.g., single nucleotide polymorphisms (SNPs)) within the template nucleic acid (e.g., of a genome).

### Sequencing Data Sets and Cycle Skip Variant Detection

Flow sequencing data sets can be generated based on the detection of an incorporated nucleotide and the order of nucleotide introduction. Take, for example, the extended sequences (i.e., each reverse complement of a corresponding template sequence): CTG, CAG, CCG, CGT, and CAT, and a repeating flow cycle of T-A-C-G (that is, sequential addition of T, A, C, and G nucleotides in separate repeating cycles). A particular type of nucleotide in a flow would be incorporated into the primer only if a complementary base is present in the template polynucleotide. An exemplary resulting flowgram is shown in Table 1, where 1 or 2 indicates incorporation of introduced nucleotide(s) and 0 indicates no incorporation of an introduced nucleotide. The flowgram can be used to derive the sequence of the template strand. For example, the sequencing data (e.g., a flowgram) discussed herein represent the sequence of the extended primer strand, and the reverse complement of which can readily be determined to represent the sequence of the template strand. An asterisk (*) in Table 1 indicates that a signal may be present in the sequencing data if additional nucleotides are incorporated in the extended sequencing strand (e.g., a longer template strand).

TABLE 1

| | Cycle 1 | | | | Cycle 2 | | | | Cycle 3 | | | |
|---|---|---|---|---|---|---|---|---|---|---|---|---|
| Flow Position | 1 | 2 | 3 | 4 | 5 | 6 | 7 | 8 | 9 | 10 | 11 | 12 |
| Base in Flow | T | A | C | G | T | A | C | G | T | A | C | G |
| Extended sequence: CTG | 0 | 0 | 1 | 0 | 1 | 0 | 0 | 1 | * | * | * | * |
| Extended sequence: CAG | 0 | 0 | 1 | 0 | 0 | 1 | 0 | 1 | * | * | * | * |
| Extended sequence: CCG | 0 | 0 | 2 | 1 | * | * | * | * | * | * | * | * |
| Extended sequence: CGT | 0 | 0 | 1 | 1 | 1 | * | * | * | * | * | * | * |
| Extended sequence: CAT | 0 | 0 | 1 | 0 | 0 | 1 | 0 | 0 | 1 | * | * | * |

A flowgram may be binary or non-binary. A binary flowgram detects the presence (1) or absence (0) of an incorporated nucleotide. A non-binary flowgram, such as shown in FIG. 25B, can more quantitatively determine a number of incorporated nucleotides at each flow position. That is, flow signals may represent a base count indicative of the number of bases in the sequenced nucleic acid molecule that are incorporated at each flow position. Table 1 is a non-binary flowgram. For example, an extended sequence of CCG incorporates two C bases in the extending primer within the same C flow (e.g., at flow position 3), and signals emitted by the labeled base have an intensity greater than (i.e., approximately twice) an intensity level corresponding to a single base incorporation. The signal values do not need to be integers. In some cases, the values can be reflective of uncertainty and/or probabilities of a number of bases being incorporated at a given flow position (e.g., due to colony sequencing).

Flow signals may include one or more statistical parameters indicative of a likelihood or confidence interval for one or more base counts at each flow position. In some embodiments, the flow signal is determined from an analog signal that is detected during the sequencing process, such as a fluorescent signal of the one or more bases incorporated into the sequencing primer during sequencing. In some cases, the analog signal can be processed to generate the statistical parameter. For example, a machine learning algorithm can be used to correct for context effects of the analog sequencing signal as described in U.S. Pat. No. 11,107,554, which is incorporated by reference herein in its entirety. Although an integer number of zero or more bases are incorporated at any given flow position, a given detected analog signal may not perfectly match with the expected analog signal. Therefore, given the detected signal, a statistical parameter indicative of the likelihood of a number of bases incorporated at the flow position can be determined. Solely by way of example, for the CCG sequence in Table 1, the likelihood that the flow signal indicates 2 bases incorporated at flow position 3 may be 0.999, and the likelihood that the flow signal indicates 1 base incorporated at flow position 3 may be 0.001.

In some cases, a sequencing data set in flowspace can be readily converted to basespace (or vice versa, if the flow order used in sequencing is known), and any mapping to a reference sequence may be done in flowspace or basespace. Mapping in flowspace is typically less computationally expensive than mapping in basespace.

By way of example, FIG. 25C shows two extended primer sequences (e.g., reverse complements of respective template molecules) that differ at one locus. Sequence 1 contains TATGGTCATCGA (SEQ ID NO: 389) and Sequence 2 contains TATGGTCGTCGA (SEQ ID NO: 390). FIG. 25D illustrates the nucleotide incorporation of Sequence 1 and Sequence 2 respectively, using a same flow order (e.g., unterminated nucleotides T-A-C-G), starting at cycle n in cycle X. In cycle X+2 of the flow order, the two sequences begin to diverge (e.g., after incorporation of C). After flow n+10, in Sequence 2, a G is incorporated in flow n+11, while in Sequence 1 no additional nucleotides are incorporated until flow n+13. Sequence 2 is completed in cycle X+4 while Sequence 1 is not completed until cycle X+5. This illustrates that different sequences can, when extended using a same non-terminated nucleotide flow order, require a different number of flows (e.g., incorporation steps). Thus, Sequences 1 and 2 will be offset in all future flow steps. Such an offset can be used to identify a variant locus between two sequences.

This continued propagation of different incorporation pattern (e.g., differences in sequencing signals) across one or more flow cycles may be referred to as a “flow shift” or a “cycle shift,” or “cycle skip,” and is generally a very unlikely event if two sequences are homologous.

These sequencing offsets can be flow order dependent. FIG. 25E is an extension of the data in FIG. 25D (e.g., using the flow order T-A-C-G). FIG. 25F illustrates nucleotide incorporation for Sequences 1 and 2 using a different flow order (e.g., A-G-C-T). As discussed above, using the first flow order (T-A-C-G) reveals a difference between these two sequences. However, despite the A to G substitution, a same number of flow cycles (e.g., 29 flow steps across 8 flow cycles) are used to extend both Sequences 1 and 2. This illustrates that the use of different flow orders can be helpful in revealing variants. Preferentially, multiple different flow orders (e.g., at least 2) may be used to interrogate a template molecule.

A short genetic variant can be, for example, a variant or mutation found within a subpopulation of individuals or a variant or mutation unique to a single or specific individual. The short genetic variants may be germline variants or somatic variants. A sequencing data set arising from sequencing a nucleic acid molecule having the short genetic variant may differ from another sequencing data set arising from sequencing a nucleic acid molecule that does not have the short genetic variant at two or more flow positions, which may be two or more consecutive flow positions or two or more non-consecutive flow positions. In some cases, the variant-containing sequence may differ from the non-variant-containing sequence at 3 or more, 4 or more, 5 or more, 6 or more, 7 or more, 8 or more, 9 or more, or 10 or more flow positions. In some cases, the variant-containing sequence may differ from the non-variant-containing sequence across 1 or more, 2 or more, 3 or more, 4 or more, or 5 or more flow cycles. An increase in the number of different flow positions between the variant-containing sequence and the non-variant-containing sequence increases the confidence that the two sequences differ at least at the variant locus.

### Sequencing Duplicate Colonies

Various methods described herein comprise generating duplicate colonies in different clusters (e.g., optionally immobilized to separate supports) by performing amplification (e.g., ePCR, eRPA) on amplification products. Such duplication may further reduce sequencing errors by providing comparative data points within the sample (e.g., one duplicate may be compared to another duplicate for internal error correction, such as by discarding base calls or reads with discrepancies between duplicate colonies).

Additional duplicate colonies may be generated by introducing empty (template-free) supports during amplification, such as ePCR amplification or eRPA amplification, where the empty supports comprise surface primers capable of binding to the templates. If multiple supports are in the vicinity (e.g., in the same partition, droplet, well, or in the same local area on surface or in bulk solution, etc.) of a template that is being amplified, the multiple supports may be amplified to generate multiple amplified supports derived from the same template. The multiple amplified supports may each be sequenced to generate duplicate sequencing data for the same template. In some cases, the template may comprise a unique molecular identifier (UMI) prior to amplification such that the sequence reads comprising the same UMIs may be associated together. Otherwise, the identical insert sequences in the duplicate reads may be matched to associate the duplicate sequence reads. In some cases, the empty supports may be amplified to generate polyclonal supports (pertaining to two or more templates) during the amplification, in which case the signal noise from such polyclonal support locations at sequencing can be used to readily discard sequencing data from such polyclonal supports. Sequencing of the same template multiple times (i.e., duplication) may further reduce sequencing errors and noise attributed to a single template by providing comparative data points within the sample (e.g., one duplicate may be compared to another duplicate for internal error correction, such as by discarding base calls or reads with discrepancies between duplicate colonies).

### Re-Sequencing

In some cases, a method for sequencing may comprise sequencing a same template strand multiple times to generate robust sequencing data (e.g., a high-quality sequencing read) corresponding to the template strand. In some cases, a method for sequencing may comprise sequencing a same template strand multiple times and/or sequencing a same reverse complement strand of the template strand multiple times to generate robust sequencing data (e.g., a high-quality paired end read) corresponding to the template strand. A method for resequencing a template strand may comprise annealing a first sequencing primer to the template strand, extending the first sequencing primer through at least a first portion of the template strand via any combination of bright steps and/or dark steps to generate first sequencing data, denaturing the extended strand from the template strand, annealing a second sequencing primer to the template strand, and extending the second sequencing primer through at least a second portion of the template strand via any combination of bright steps and/or dark steps to generate second sequencing data, and processing (e.g., combining, comparing, matching, aligning, resolving, etc.) the first sequencing data and the second sequencing data to generate a sequencing read of the template strand. A template strand may be denatured and re-sequenced any number of times, such as about, at least about, and/or at most about 1, 2, 3, 4, 5, 6, 7, 8, 9, 10 or more times, such as by annealing an nth sequencing primer to the template strand and extending the nth sequencing primer through at least an nth portion of the template strand. The different n sequencing primers may comprise the same or different sequences which may bind to same or different primer binding sites on the template strand, respectively. The different nth portions on the template strand may refer to the same portions or different portions on the template strand. Two portions on the template strand (that are extended through) may be partially overlapping, completely overlapping (for one or both portions), or non-overlapping. The respective extensions through the template strand in the different sequencing runs may use the same or different sequencing methods (e.g., non-terminated sequencing during a first sequencing run, terminated during a second sequencing run; terminated sequencing during a second sequencing run; etc.). The respective extensions through the template strand in the different sequencing runs may use the same or different nucleotide reagents (e.g., non-terminated nucleotides during a first sequencing run, terminated during a second sequencing run; green dye-labeled nucleotides during a first sequencing run, red dye-labeled nucleotides during a second sequencing run; labeled A-, T-, G-bases and unlabeled C-base nucleotides during a first sequencing run, labeled A-, T-, C-bases and unlabeled G-base nucleotides during a second sequencing run; 5% labeled A bases during a first sequencing run; 100% labeled A bases during a second sequencing run; etc.). The respective extensions through the template strand in the different sequencing runs may have the same flow order or flow cycle of nucleotide reagents. The respective extensions through the template strand in the different sequencing runs may have different flow orders or flow cycles of nucleotide reagents (e.g., A->T->G->C single base flow cycle order during a first sequencing run, T->A->G->C single base flow cycle order during a second sequencing run; A/T/G/C 4-base flow cycle order during a first sequencing run; A/T/G->A/T/C 3-base flow cycle order during a second sequencing run, etc.). Denaturing may comprise contacting the double-stranded nucleic acid molecule with denaturing agents, such as sodium hydroxide (NaOH) or ethylene carbonate. An entire substrate may be subjected to resequencing by, after a first sequencing run, contacting the entire surface with a solution comprising a denaturing agent, contacting the entire surface with a solution comprising sequencing primers under conditions sufficient to anneal them to template nucleic acid strands immobilized to the substrate, and subjecting them to extension reactions. In some cases, denaturing may comprise applying heat to the double-stranded nucleic acid molecule.

Additional sequencing schemes are described in U.S. Pat. Pub. Nos. 2021/0017593A1, 2022/0064728A1, and 2022/0154272A1, each of which is entirely incorporated herein by reference for all purposes.

In some cases, after generating a first sequencing read with a first sequencing primer, and after denaturing a first extension product of the first sequencing primer, a second sequencing primer may hybridize to the portion corresponding to the 3′ loop of the adapter and be extended to generate a second sequencing read. In some cases, the second sequencing primer may also be extended through a second barcode region. Such separate read generation can relax the limitation on sheared read length in genomic DNA sequencing and allows reading mixed di-nucleosome cfDNA molecules.

### Strand Recognition Adaptor Sequences

Tables 2 and 3 illustrate examples of strand recognition adaptor sequences (5′ to 3′). N* indicates N is a phosphorothioated base. /5Phos/=5′ phosphorylation. /ideoxyU/=deoxyuridine. /3bioTEG/=3′ biotin-triethylene glycol (TEG) spacer arm. An adaptor molecule comprises a sense strand with sequence of SEQ ID NO: N selected from 1-96 or 193-288 and an antisense strand with SEQ ID NO: N+96. The Table 2 adaptor molecules each comprises the mismatch sequence pair: AAAAAA/AAAAAA in a looped mismatch portion in the middle of the adaptor molecule. The Table 3 adaptor molecules each comprises the mismatch sequence pair: AAGG (/AAGG in a looped mismatch portion in the middle of the adaptor molecule.

TABLE 2
Adaptor Molecules with AAAAAA (SEQ ID NO 385) Mismatch Sequence Pair

| ID | Sense Strand Sequence |
|---|---|
| SEQ ID NO: 1 | ATCTCATCCCTGCGTGTCTCCGACTGCACAGCTCGAATGCGATCAAAAAACATGAGCAGC*A*T |
| SEQ ID NO: 2 | ATCTCATCCCTGCGTGTCTCCGACTGCACATCACACATGAATGATCAAAAAACATGAGCAGC*A*T |
| SEQ ID NO: 3 | ATCTCATCCCTGCGTGTCTCCGACTGCACTGTGTAGGCATGATCAAAAAACATGAGCAGC*A*T |
| SEQ ID NO: 4 | ATCTCATCCCTGCGTGTCTCCGACTGCACATGTATCCTCTGATCAAAAAACATGAGCAGC*A*T |
| SEQ ID NO: 5 | ATCTCATCCCTGCGTGTCTCCGACTGCACATATAGCCTATGATCAAAAAACATGAGCAGC*A*T |
| SEQ ID NO. 6 | ATCTCATCCCTGCGTGTCTCCGACTGCACGATTCATGCTCGATCAAAAAACATGAGCAGC*A*T |
| SEQ ID NO: 7 | ATCTCATCCCTGCGTGTCTCCGACTGCACACATCCTGCATGTGATCAAAAAACATGAGCAGC*A*T |
| SEQ ID NO: 8 | ATCTCATCCCTGCGTGTCTCCGACTGCACACTGCACGAATGATCAAAAAACATGAGCAGC*A*T |
| SEQ ID NO. 9 | ATCTCATCCCTGCGTGTCTCCGACTGCACTGCCATAGCACGATCAAAAAACATGAGCAGC*A*T |
| SEQ ID NO: 10 | ATCTCATCCCTGCGTGTCTCCGACTGCACAGTTGTGCTGTGATCAAAAAACATGAGCAGC*A*T |
| SEQ ID NO: 11 | ATCTCATCCCTGCGTGTCTCCGACTGCACTTAGATATCATGATCAAAAAACATGAGCAGC*A*T |
| SEQ ID NO: 12 | ATCTCATCCCTGCGTGTCTCCGACTGCACATCCTGTGCGCATGATCAAAAAACATGAGCAGC*A*T |
| SEQ ID NO: 13 | ATCTCATCCCTGCGTGTCTCCGACTGCACATGCGTCCTGTGATCAAAAAACATGAGCAGC*A*T |
| SEQ ID NO: 14 | ATCTCATCCCTGCGTGTCTCCGACTGCACAATGCTCTGCATAGATCAAAAAACATGAGCAGC*A*T |
| SEQ ID NO: 15 | ATCTCATCCCTGCGTGTCTCCGACTGCACTACATTGCACAGATCAAAAAACATGAGCAGC*A*T |
| SEQ ID NO: 16 | ATCTCATCCCTGCGTGTCTCCGACTGCACATAGCGAGCCAGATCAAAAAACATGAGCAGC*A*T |
| SEQ ID NO: 17 | ATCTCATCCCTGCGTGTCTCCGACTGCACTCTGTATTGCAGATCAAAAAACATGAGCAGC*A*T |
| SEQ ID NO: 18 | ATCTCATCCCTGCGTGTCTCCGACTGCACATCATCGATTCGATCAAAAAACATGAGCAGC*A*T |
| SEQ ID NO: 19 | ATCTCATCCCTGCGTGTCTCCGACTGCACTGTGAATATGCGATCAAAAAACATGAGCAGC*A*T |
| SEQ ID NO: 20 | ATCTCATCCCTGCGTGTCTCCGACTGCACAGAAGCTGCATGAGATCAAAAAACATGAGCAGC*A*T |
| SEQ ID NO: 21 | ATCTCATCCCTGCGTGTCTCCGACTGCACGCATCCTCACAGATCAAAAAACATGAGCAGC*A*T |
| SEQ ID NO: 22 | ATCTCATCCCTGCGTGTCTCCGACTGCACACAACATATCAGATCAAAAAACATGAGCAGC*A*T |
| SEQ ID NO: 23 | ATCTCATCCCTGCGTGTCTCCGACTGCACACATCAGCTCAATGATCAAAAAACATGAGCAGC*A*T |
| SEQ ID NO: 24 | ATCTCATCCCTGCGTGTCTCCGACTGCACATGCATATAATGCGATCAAAAAACATGAGCAGC*A*T |
| SEQ ID NO: 25 | ATCTCATCCCTGCGTGTCTCCGACTGCACTCAATGCATCAGCGATCAAAAAACATGAGCAGC*A*T |
| SEQ ID NO: 26 | ATCTCATCCCTGCGTGTCTCCGACTGCACAATGTGTGCTAGATCAAAAAACATGAGCAGC*A*T |
| SEQ ID NO. 27 | ATCTCATCCCTGCGTGTCTCCGACTGCACTCTCGCATGCAATGATCAAAAAACATGAGCAGC*A*T |
| SEQ ID NO: 28 | ATCTCATCCCTGCGTGTCTCCGACTGCACTAGCAGCCAGCGATCAAAAAACATGAGCAGC*A*T |
| SEQ ID NO: 29 | ATCTCATCCCTGCGTGTCTCCGACTGCACTGCCAGACTGTGATCAAAAAACATGAGCAGC*A*T |
| SEQ ID NO: 30 | ATCTCATCCCTGCGTGTCTCCGACTGCACACATGGCAGCACAGATCAAAAAACATGAGCAGC*A*T |
| SEQ ID NO: 31 | ATCTCATCCCTGCGTGTCTCCGACTGCACACTTGCAGATCGATCAAAAAACATGAGCAGC*A*T |
| SEQ ID NO: 32 | ATCTCATCCCTGCGTGTCTCCGACTGCACTATGCCACAGCATGATCAAAAAACATGAGCAGC*A*T |
| SEQ ID NO: 33 | ATCTCATCCCTGCGTGTCTCCGACTGCACAACATCAGCATGAGATCAAAAAACATGAGCAGC*A*T |
| SEQ ID NO: 34 | ATCTCATCCCTGCGTGTCTCCGACTGCACTGCAGTGATTCATGATCAAAAAACATGAGCAGC*A*T |
| SEQ ID NO: 35 | ATCTCATCCCTGCGTGTCTCCGACTGCACAGCACCTGCATCAGATCAAAAAACATGAGCAGC*A*T |
| SEQ ID NO: 36 | ATCTCATCCCTGCGTGTCTCCGACTGCACTTATGCTATCAGATCAAAAAACATGAGCAGC*A*T |
| SEQ ID NO: 37 | ATCTCATCCCTGCGTGTCTCCGACTGCACATCTCAGTGCAATGATCAAAAAACATGAGCAGC*A*T |
| SEQ ID NO: 38 | ATCTCATCCCTGCGTGTCTCCGACTGCACACAGTCAATGTGATCAAAAAACATGAGCAGG*A*T |
| SEQ ID NO: 39 | ATCTCATCCCTGCGTGTCTCCGACTGCACATAGAGCCTCAGATCAAAAAACATGAGCAGC*A*T |
| SEQ ID NO: 40 | ATCTCATCCCTGCGTGTCTCCGACTGCACTTGTGTCATGAGATCAAAAAACATGAGCAGC*A*T |
| SEQ ID NO: 41 | ATCTCATCCCTGCGTGTCTCCGACTGCACAATGCACTCGAGATCAAAAAACATGAGCAGC*A*T |
| SEQ ID NO: 42 | ATCTCATCCCTGCGTGTCTCCGACTGCACGTCATTGCACAGATCAAAAAACATGAGCAGC*A*T |
| SEQ ID NO: 43 | ATCTCATCCCTGCGTGTCTCCGACTGCACATCACTGCAACGATGAAAAAACATGAGCAGC*A*T |
| SEQ ID NO: 44 | ATCTCATCCCTGCGTGTCTCCGACTGCACTTGCATGCGATGCGATCAAAAAACATGAGCAGC*A*T |
| SEQ ID NO: 45 | ATCTCATCCCTGCGTGTCTCCGACTGCACGTGCGCAAGCAGATCAAAAAACATGAGCAGC*A*T |
| SBQ ID NO: 46 | ATCTCATCCCTGCGTGTCTCCGACTGCACTATCTCATAATGATCAAAAAACATGAGCAGC*A*T |
| SEQ ID NO: 47 | ATCTCATCCCTGCGTGTGTCCGACTGCACATGGCTATGCACTGATCAAAAAACATGAGCAGC*A*T |
| SEQ ID NO: 48 | ATCTCATCCCTGCGTGTCTCCGACTGCACTATGAATGAGCGATCAAAAAACATGAGCAGC*A*T |
| SEQ ID NO: 49 | ATCTCATCCCTGCGTGTCTCCGACTGCACTATGCACCATCGATCAAAAAACATGAGCAGC*A*T |
| SEQ ID NO: 50 | ATCTCATCCCTGCGTGTCTCCGACTGCACACAATGTGCGCGATCAAAAAACATGAGCAGC*A*T |
| SEQ ID NO: 51 | ATCTCATCCCTGCGTGTCTCCGACTGCACAATGACTATCTGATCAAAAAACATGAGCAGC*A*T |
| SEQ ID NO: 52 | ATCTCATCCCTGCGTGTCTCCGACTGCACAGCCTCAGCGTGATCAAAAAACATGAGCAGC*A*T |
| SEQ ID NO: 53 | ATCTCATCCCTGCGTGTCTCCGACTGCACTCTGCTGTGCAATGATCAAAAAACATGAGCAGC*A*T |
| SEQ ID NO: 54 | ATCTCATCCCTGCGTGTCTCCGACTGCACTGTGCATCTGCCTGATCAAAAAACATGAGCAGC*A*T |
| SEQ ID NO: 55 | ATCTCATCCCTGCGTGTCTCCGACTGCACAACTATCTGCAGATCAAAAAACATGAGCAGC*A*T |
| SEQ ID NO: 56 | ATCTCATCCCTGCGTGTCTCCGACTGCACAGATCTCATGAATGATCAAAAAACATGAGCAGC*A*T |
| SEQ ID NO: 57 | ATCTCATCCCTGCGTGTCTCCGACTGCACTATCATCCAGTGATCAAAAAACATGAGCAGC*A*T |
| SEQ ID NO: 58 | ATCTCATCCCTGCGTGTCTCCGACTGCACTGCTACAAGCAGATCAAAAAACATGAGCAGC*A*T |
| SEQ ID NO: 59 | ATCTCATCCCTGCGTGTCTCCGACTGCACAATATGCACGTGATCAAAAAACATGAGCAGC*A*T |
| SEQ ID NO: 60 | ATCTCATCCCTGCGTGTCTCCGACTGCACACTTCTGCGATGATCAAAAAACATGAGCAGC*A*T |
| SEQ ID NO: 61 | ATCTCATCCCTGCGTGTCTCCGACTGCACAAGCATATCTAGATCAAAAAACATGAGCAGC*A*T |
| SEQ ID NO: 62 | ATCTCATCCCTGCGTGTCTCCGACTGCACAATGACAGCTCGATCAAAAAACATGAGCAGC*A*T |
| SEQ ID NO: 63 | ATCTCATCCCTGCGTGTCTCCGACTGCACATATGACCTGAGATCAAAAAACATGAGCAGC*A*T |
| SEQ ID NO: 64 | ATCTCATCCCTGCGTGTCTCCGACTGCACAGCCGATATCAGATCAAAAAACATGAGCAGC*A*T |
| SEQ ID NO: 65 | ATCTCATCCCTGCGTGTCTCCGACTGCACAGTCAGTTGCAGATCAAAAAACATGAGCAGC*A*T |
| SEQ ID NO: 66 | ATCTCATCCCTGCGTGTCTCCGACTGCACTCATCTGCGCAATGATCAAAAAACATGAGCAGC*A*T |
| SEQ ID NO: 67 | ATCTCATCCCTGCGTGTCTCCGACTGCACTATTGCATGCTCTGATCAAAAAACATGAGCAGC*A*T |
| SEQ ID NO: 68 | ATCTCATCCCTGCGTGTCTCCGACTGCACATATAATCACAGATCAAAAAACATGAGCAGC*A*T |
| SEQ ID NO: 69 | ATCTCATCCCTGCGTGTCTCCGACTGCACTGAGAATGTGTGATCAAAAAACATGAGCAGC*A*T |
| SEQ ID NO: 70 | ATCTCATCCCTGCGTGTCTCCGACTGCACATGCATGGTACGATCAAAAAACATGAGCAGC*A*T |
| SEQ ID NO: 71 | ATCTCATCCCTGCGTGTCTCCGACTGCACATGCATGCGAGGAGATCAAAAAACATGAGCAGC*A*T |
| SEQ ID NO: 72 | ATCTCATCCCTGCGTGTCTCCGACTGCACAATCTGCATACGATCAAAAAACATGAGCAGC*A*T |
| SEQ ID NO: 73 | ATCTCATCCCTGCGTGTCTCCGACTGCACAGCATGAGCGCCAGATCAAAAAACATGAGCAGC*A*T |
| SEQ ID NO: 74 | ATCTCATCCCTGCGTGTCTCCGACTGCACTTATCTGATCTGATCAAAAAACATGAGCAGC*A*T |
| SEQ ID NO: 75 | ATCTCATCCCTGCGTGTCTCCGACTGCACATCCAGCGCATGTGATCAAAAAACATGAGCAGC*A*T |
| SEQ ID NO: 76 | ATCTCATCCCTGCGTGTCTCCGACTGCACAGTTCATCTGTGATCAAAAAACATGAGCAGC*A*T |
| SEQ ID NO: 77 | ATCTCATCCCTGCGTGTCTCCGACTGCACAACATACATCAGATCAAAAAACATGAGCAGC*A*T |
| SEQ ID NO: 78 | ATCTCATCCCTGCGTGTCTCCGACTGCACGGCTAGATGCAGATCAAAAAACATGAGCAGC*A*T |
| SEQ ID NO: 79 | ATCTCATCCCTGCGTGTCTCCGACTGCACTGCCGAGCAGCATGATCAAAAAACATGAGCAGC*A*T |
| SEQ ID NO: 80 | ATCTCATCCCTGCGTGTCTCCGACTGCACTGCCTCAGATCATGATCAAAAAACATGAGCAGC*A*T |
| SEQ ID NO: 81 | ATCTCATCCCTGCGTGTCTCCGACTGCACTGATCAGTGGCGATCAAAAAACATGAGCAGC*A*T |
| SEQ ID NO: 82 | ATCTCATCCCTGCGTGTCTCCGACTGCACACATATGGCATATGATCAAAAAACATGAGCAGC*A*T |
| SEQ ID NO: 83 | ATCTCATCCCTGCGTGTCTCCGACTGCACAGATCGCCACAGATCAAAAAACATGAGCAGC*A*T |
| SEQ ID NO: 84 | ATCTCATCCCTGCGTGTCTCCGACTGCACTTGATGATAGTGATCAAAAAACATGAGCAGC*A*T |
| SEQ ID NO: 85 | ATCTCATCCCTGCGTGTCTCCGACTGCACATCTCTGGCTGCAGATCAAAAAACATGAGCAGC*A*T |
| SEQ ID NO: 86 | ATCTCATCCCTGCGTGTCTCCGACTGCACATCTGGTGCATGTGATCAAAAAACATGAGCAGC*A*T |
| SEQ ID NO: 87 | ATCTCATCCCTGCGTGTCTCCGACTGCACAGCAGCTTCGCGATCAAAAAACATGAGCAGC*A*T |
| SEQ ID NO: 88 | ATCTCATCCCTGCGTGTCTCCGACTGCACGCATATGGCAGCAGATCAAAAAACATGAGCAGC*A*T |
| SEQ ID NO: 89 | ATCTCATCCCTGCGTGTCTCCGACTGCACATGCAGATGGCGAGATCAAAAAACATGAGCAGC*A*T |
| SEQ ID NO: 90 | ATCTCATCCCTGCGTGTCTCCGACTGCACTTCATGCATCTCAGATCAAAAAACATGAGCAGC*A*T |
| SEQ ID NO: 91 | ATCTCATCCCTGCGTGTCTCCGACTGCACATGCAAGTGTGATGATCAAAAAACATGAGCAGC*A*T |
| SEQ ID NO: 92 | ATCTCATCCCTGCGTGTCTCCGACTGCACTGTTCGCTGCAGATCAAAAAACATGAGCAGC*A*T |
| SBQ ID NO: 93 | ATCTCATCCCTGCGTGTCTCCGACTGCACTCAGATCCTGCATGATCAAAAAACATGAGCAGC*A*T |
| SEQ ID NO: 94 | ATCTCATCCCTGCGTGTCTCCGACTGCACACACAGATAATGATCAAAAAACATGAGCAGC*A*T |
| SEQ ID NO: 95 | ATCTCATCCCTGCGTGTCTCCGACTGCACGATGCTCTGGCGATCAAAAAACATGAGCAGC*A*T |
| SEQ ID NO: 96 | ATCTCATCCCTGCGTGTCTCCGACTGCACAGATCCATCATCTGATCAAAAAACATGAGCAGC*A*T |
| ID | Antisense Strand Sequence |
| SEQ ID NO: 97 | /5Phos/TGCTGCTCATGAAAAAAGATCGCATTCGAGCTGTGCAGTCGGAGACACGCAGGGATGAGA/ideoxyU/ |
| | GG/ideoxyU//3BioTEG/ |
| SEQ ID NO: 98 | /5Phos/TGCTGCTCATGAAAAAAGATCATTCATGTGTGATGTGCAGTCGGAGACACGCAGGGATGAGA |
| | /ideoxyU/GG/ideoxyU//3BioTEG/ |
| SEQ ID NO: 99 | /5Phos/TGCTGCTCATGAAAAAAGATCATGCCTACACAGTGCAGTCGGAGACACGCAGGGATGAGA/ideoxyU/ |
| | GG/ideoxyU//3BioTEG/ |
| SEQ ID NO: 100 | /5Phos/TGCTGCTCATGAAAAAAGATCAGAGGATACATGTGCAGTCGGAGACACGCAGGGATGAGA/ideoxyU/ |
| | GG/ideoxyU//3BioTEG/ |
| SEQ ID NO: 101 | /5Phos/TGCTGCTCATGAAAAAAGATCATAGGCTATATGTGCAGTCGGAGACACGCAGGGATGAGA/ideoxyU/ |
| | GG/ideoxyU//3BioTEG/ |
| SEQ ID NO: 102 | /5Phos/TGCTGCTCATGAAAAAAGATCGAGCATGAATCGTGCAGTCGGAGACACGCAGGGATGAGA/ideoxyU/ |
| | GG/ideoxyU//3BioTEG/ |
| SEQ ID NO: 103 | /5Phos/TGCTGCTCATGAAAAAAGATCACATGCAGGATGTGTGCAGTCGGAGACACGCAGGGATGAGA/ |
| | /ideoxyU/GG/ideoxyU//3BioTEG/ |
| SEQ ID NO: 104 | /5Phos/TGCTGCTCATGAAAAAAGATCATTCGTGCAGTGTGCAGTCGGAGACACGCAGGGATGAGA/ |
| | ideoxyU/GG/ideoxyU//3BioTEG/ |
| SEQ ID NO: 105 | /5Phos/TGCTGCTCATGAAAAAAGATCGTGCTATGGCAGTGCAGTCGGAGACACGCAGGGATGAGA/ |
| | ideoxyU/GG/ideoxyU//3BioTEG/ |
| SEQ ID NO: 106 | /5Phos/TGCTGCTCATGAAAAAAGATCACAGCACAACTGTGCAGTCGGAGACACGCAGGGATGAGA/ |
| | ideoxyU/GG/ideoxyU//3BioTEG/ |
| SEQ ID NO: 107 | /5Phos/TGCTGCTCATGAAAAAAGATCATGATATCTAAGTGCAGTCGGAGACACGCAGGGATGAGA/ideoxyU/ |
| | GG/ideoxyU//3BioTEG/ |
| SEQ ID NO: 108 | /5Phos/TGCTGCTCATGAAAAAAGATCATGCGCACAGGATGTGCAGTCGGAGACACGCAGGGATGAGA/ |
| | ideoxyU/GG/ideoxyU//3BioTEG/ |
| SEQ ID NO: 109 | /5Phos/TGCTGCTCATGAAAAAAGATCACAGGACGCATGTGCAGTCGGAGACACGCAGGGATGAGA/ |
| | ideoxyU/GG/ideoxyU//3BioTEG/ |
| SEQ ID NO: 110 | /5Phos/TGCTGCTCATGAAAAAAGATCTATGCAGAGCATTGTGCAGTCGGAGACACGCAGGGATGAGA/ |
| | ideoxyU/GG/ideoxyU//3BioTEG/ |
| SEQ ID NO: 111 | /5Phos/TGCTGCTCATGAAAAAAGATCTGTGCAATGTAGTGCAGTCGGAGACACGCAGGGATGAGA/ |
| | ideoxyU/GG/ideoxyU//3BioTEG/ |
| SEQ ID NO: 112 | /5Phos/TGCTGCTCATGAAAAAAGATCTGGCTCGCTATGTGCAGTCGGAGACACGCAGGGATGAGA/ |
| | ideoxyU/GG/ideoxyU//3BioTEG/ |
| SEQ ID NO: 113 | /5Phos/TGCTGCTCATGAAAAAAGATCTGCAATACAGAGTGCAGTCGGAGACACGCAGGGATGAGA/ |
| | ideoxyU/GG/ideoxyU//3BioTEG/ |
| SEQ ID NO: 114 | /5Phos/TGCTGCTCATGAAAAAAGATCGAATCGATGATGTGCAGTCGGAGACACGCAGGGATGAGA/ |
| | ideoxyU/GG/ideoxyU//3BioTEG/ |
| SEQ ID NO: 115 | /5Phos/TGCTGCTCATGAAAAAAGATCGCATATTCACAGtGCAGTCGGAGACACGCAGGGATGAGA/ |
| | ideoxyU/GG/ideoxyU//3BioTEG/ |
| SEQ ID NO: 116 | /5Phos/TGCTGCTCATGAAAAAAGATCTCATGCAGCTTCTGTGCAGTCGGAGACACGCAGGGATGAGA/ |
| | ideoxyU/GG/ideoxyU//3BioTEG/ |
| SEQ ID NO: 117 | /5Phos/TGCTGCTCATGAAAAAAGATCTGTGAGGATGCGTGCAGTCGGAGACACGCAGGGATGAGA/ |
| | ideoxyU/GG/ideoxyU//3BioTEG/ |
| SEQ ID NO: 118 | /5Phos/TGCTGCTCATGAAAAAAGATCTGATATGTTGTGTGCAGTCGGAGACACGCAGGGATGAGA/ |
| | ideoxyU/GG/ideoxyU//3BioTEG/ |
| SEQ ID NO: 119 | /5Phos/TGCTGCTCATGAAAAAAGATCTGATATGTTGTGTGCAGTCGGAGACACGCAGGGATGAGA/ |
| | ideoxyU/GG/ideoxyU//3BioTEG/ |
| SEQ ID NO: 120 | /5Phos/TGCTGCTCATGAAAAAAGATCGCATTATATGCATGTGCAGTCGGAGACACGCAGGGATGAGA/ |
| | ideoxyU/GG/ideoxyU//3BioTEG/ |
| SEQ ID NO: 121 | /5Phos/TGCTGCTCATGAAAAAAGATCGCTGATGCATTGAGTGCAGTCGGAGACACGCAGGGATGAGA/ |
| | ideoxyU/GG/ideoxyU//3BioTEG/ |
| SEQ ID NO: 122 | /5Phos/TGCTGCTCATGAAAAAAGATCTAGCACACATTGTGCAGTCGGAGACACGCAGGGATGAGA/ |
| | ideoxyU/GG/ideoxyU//3BioTEG/ |
| SEQ ID NO: 123 | /5Phos/TGCTGCTCATGAAAAAAGATCATTGCATGCGAGAGTGCAGTCGGAGACACGCAGGGATGAGA/ |
| | ideoxyU/GG/ideoxyU//3BioTEG/ |
| SEQ ID NO: 124 | /5Phos/TGCTGCTCATGAAAAAAGATCGCTGGCTGCTAGTGCAGTCGGAGACACGCAGGGATGAGA/ |
| | ideoxyU/GG/ideoxyU//3BioTEG/ |
| SEQ ID NO: 125 | /5Phos/TGCTGCTCATGAAAAAAGATCACAGTCTGGCAGTGCAGTCGGAGACACGCAGGGATGAGA/ |
| | ideoxyU/GG/ideoxyU//3BioTEG/ |
| SEQ ID NO: 126 | /5Phos/TGCTGCTCATGAAAAAAGATCTGTGCTGCCATGTGTGCAGTCGGAGACACGCAGGGATGAGA/ |
| | ideoxyU/GG/ideoxyU//3BioTEG/ |
| SEQ ID NO: 127 | /5Phos/TGCTGCTCATGAAAAAAGATCGATCTGCAAGTGTGCAGTCGGAGACACGCAGGGATGAGA/ |
| | ideoxyU/GG/ideoxyU//3BioTEG/ |
| SEQ ID NO: 128 | /5Phos/TGCTGCTCATGAAAAAAGATCATGCTGTGGCATAGTGCAGTCGGAGACACGCAGGGATGAGA/ |
| | ideoxyU/GG/ideoxyU//3BioTEG/ |
| SEQ ID NO. 129 | /5Phos/TGCTGCTCATGAAAAAAGATCTCATGCTGATGTTGTGCAGTCGGAGACACGCAGGGATGAGA/ |
| | ideoxyU/GG/ideoxyU//3BioTEG/ |
| SEQ ID NO: 130 | /5Phos/TGCTGCTCATGAAAAAAGATCATGAATCACTGCAGTGCAGTCGGAGACACGCAGGGATGAGA/ |
| | ideoxyU/GG/ideoxyU//3BioTEG/ |
| SEQ ID NO: 131 | /5Phos/TGCTGCTCATGAAAAAAGATCTGATGCAGGTGCTGTGCAGTCGGAGACACGCAGGGATGAGA/ |
| | ideoxyU/GG/ideoxyU//3BioTEG/ |
| SEQ ID NO: 132 | /5Phos/TGCTGCTCATGAAAAAAGATCTGATAGCATAAGTGCAGTCGGAGACACGCAGGGATGAGA/ |
| | ideoxyU/GG/ideoxyU//3BioTEG/ |
| SEQ ID NO: 133 | /5Phos/TGCTGCTCATGAAAAAAGATCATTGCACTGAGATGTGCAGTCGGAGACACGCAGGGATGAGA/ |
| | ideoxyU/GG/ideoxyU//3BioTEG/ |
| SEQ ID NO: 134 | /5Phos/TGCTGCTCATGAAAAAAGATCACATTGACTGTGTGCAGTCGGAGACACGCAGGGATGAGA/ |
| | ideoxyU/GG/ideoxyU//3BioTEG/ |
| SEQ ID NO: 135 | /5Phos/TGCTGCTCATGAAAAAAGATCTGAGGCTCTATGTGCAGTCGGAGACACGCAGGGATGAGA/ |
| | ideoxyU/GG/ideoxyU//3BioTEG/ |
| SEQ ID NO: 136 | /5Phos/TGCTGCTCATGAAAAAAGATCTCATGACACAAGTGCAGTCGGAGACACGCAGGGATGAGA/ |
| | ideoxyU/GG/ideoxyU//3BioTEG/ |
| SEQ ID NO: 137 | /5Phos/TGCTGCTCATGAAAAAAGATCTCGAGTGCATTGTGCAGTCGGAGACACGCAGGGATGAGA/ |
| | ideoxyU/GG/ideoxyU//3BioTEG/ |
| SEQ ID NO: 138 | /5Phos/TGCTGCTCATGAAAAAAGATCTGTGCAATGACGTGCAGTCGGAGACACGCAGGGATGAGA/ |
| | ideoxyU/GG/ideoxyU//3BioTEG/ |
| SEQ ID NO: 139 | /5Phos/TGCTGCTCATGAAAAAAGATCGTTGCAGTGATGTGCAGTCGGAGACACGCAGGGATGAGA/ |
| | ideoxyU/GG/ideoxyU//3BioTEG/ |
| SEQ ID NO: 140 | /5Phos/TGCTGCTCATGAAAAAAGATCGCATCGCATGCAAGTGCAGTCGGAGACACGCAGGGATGAGA/ |
| | ideoxyU/GG/ideoxyU//3BioTEG/ |
| SEQ ID NO: 141 | /5Phos/TGCTGCTCATGAAAAAAGATCTGCTTGCGCACGTGCAGTCGGAGACACGCAGGGATGAGA/ |
| | ideoxyU/GG/ideoxyU//3BioTEG/ |
| SEQ ID NO: 142 | /5Phos/TGCTGCTCATGAAAAAAGATCATTATGAGATAGTGCAGTCGGAGACACGCAGGGATGAGA/ |
| | ideoxyU/GG/ideoxyU//3BioTEG/ |
| SEQ ID NO: 143 | /5Phos/TGCTGCTCATGAAAAAAGATCAGTGCATAGCCATGTGCAGTCGGAGACACGCAGGGATGAGA/ |
| | ideoxyU/GG/ideoxyU//3BioTEG/ |
| SEQ ID NO: 144 | /5Phos/TGCTGCTCATGAAAAAAGATCGCTCATTCATAGTGCAGTCGGAGACACGCAGGGATGAGA/ |
| | ideoxyU/GG/ideoxyU//3BioTEG/ |
| SEQ ID NO: 145 | /5Phos/TGCTGCTCATGAAAAAAGATCGATGGTGCATAGTGCAGTCGGAGACACGCAGGGATGAGA/ |
| | ideoxyU/GG/ideoxyU//3BioTEG/ |
| SEQ ID NO: 146 | /5Phos/TGCTGCTCATGAAAAAAGATCGCGCACATTGTGTGCAGTCGGAGACACGCAGGGATGAGA/ |
| | ideoxyU/GG/ideoxyU//3BioTEG/ |
| SEQ ID NO: 147 | /5Phos/TGCTGCTCATGAAAAAAGATCAGATAGTCATTGTGCAGTCGGAGACACGCAGGGATGAGA/ |
| | ideoxyU/GG/ideoxyU//3BioTEG/ |
| SEQ ID NO: 148 | /5Phos/TGCTGCTCATGAAAAAAGATCACGCTGAGGCTGTGCAGTCGGAGACACGCAGGGATGAGA/ |
| | ideoxyU/GG/ideoxyU//3BioTEG/ |
| SEQ ID NO: 149 | /5Phos/TGCTGCTCATGAAAAAAGATCATTGCACAGCAGAGTGCAGTCGGAGACACGCAGGGATGAGA/ |
| | ideoxyU/GG/ideoxyU//3BioTEG/ |
| SEQ ID NO: 150 | /5Phos/TGCTGCTCATGAAAAAAGATCAGGCAGATGCACAGTGCAGTCGGAGACACGCAGGGATGAGA/ |
| | ideoxyU/GG/ideoxyU//3BioTEG/ |
| SEQ ID NO: 151 | /5Phos/TGCTGCTCATGAAAAAAGATCTGCAGATAGTTGTGCAGTCGGAGACACGCAGGGATGAGA/ |
| | ideoxyU/GG/ideoxyU//3BioTEG/ |
| SEQ ID NO: 152 | /5Phos/TGCTGCTCATGAAAAAAGATCATTCATGAGATCTGTGCAGTCGGAGACACGCAGGGATGAGA/ |
| | ideoxyU/GG/ideoxyU//3BioTEG/ |
| SEQ ID NO: 153 | /5Phos/TGCTGCTCATGAAAAAAGATCACTGGATGATAGTGCAGTCGGAGACACGCAGGGATGAGA/ |
| | ideoxyU/GG/ideoxyU//3BioTEG/ |
| SEQ ID NO: 154 | /5Phos/TGCTGCTCATGAAAAAAGATCTGCTTGTAGCAGTGCAGTCGGAGACACGCAGGGATGAGA/ |
| | ideoxyU/GG/ideoxyU//3BioTEG/ |
| SEQ ID NO: 155 | /5Phos/TGCTGCTCATGAAAAAAGATCACGTGCATATTGTGCAGTCGGAGACACGCAGGGATGAGA/ |
| | ideoxyU/GG/ideoxyU//3BioTEG/ |
| SEQ ID NO: 156 | /5Phos/TGCTGCTCATGAAAAAAGATCATCGCAGAAGTGTGCAGTCGGAGACACGCAGGGATGAGA/ |
| | ideoxyU/GG/ideoxyU//3BioTEG/ |
| SEQ ID NO: 157 | /5Phos/TGCTGCTCATGAAAAAAGATCTAGATATGCTTGTGCAGTCGGAGACACGCAGGGATGAGA/ |
| | ideoxyU/GG/ideoxyU//3BioTEG/ |
| SEQ ID NO: 158 | /5Phos/TGCTGCTCATGAAAAAAGATCGAGCTGTCATTGTGCAGTCGGAGACACGCAGGGATGAGA/ |
| | ideoxyU/GG/ideoxyU//3BioTEG/ |
| SEQ ID NO: 159 | /5Phos/TGCTGCTCATGAAAAAAGATCTCAGGTCATATGTGCAGTCGGAGACACGCAGGGATGAGA/ |
| | ideoxyU/GG/ideoxyU//3BioTEG/ |
| SEQ ID NO: 160 | /5Phos/TGCTGCTCATGAAAAAAGATCTGATATCGGCTGTGCAGTCGGAGACACGCAGGGATGAGA/ |
| | ideoxyU/GG/ideoxyU//3BioTEG/ |
| SEQ ID NO: 161 | /5Phos/TGCTGCTCATGAAAAAAGATCTGCAACTGACTGTGCAGTCGGAGACACGCAGGGATGAGA/ |
| | ideoxyU/GG/ideoxyU//3BioTEG/ |
| SEQ ID NO: 162 | /5Phos/TGCTGCTCATGAAAAAAGATCATTGCGCAGATGAGTGCAGTCGGAGACACGCAGGGATGAGA/ |
| | ideoxyU/GG/ideoxyU//3BioTEG/ |
| SEQ ID NO: 163 | /5Phos/TGCTGCTCATGAAAAAAGATCAGAGCATGCAATAGTGCAGTCGGAGACACGCAGGGATGAGA/ |
| | ideoxyU/GG/ideoxyU//3BioTEG/ |
| SEQ ID NO: 164 | /5Phos/TGCTGCTCATGAAAAAAGATCTGTGATTATATGTGCAGTCGGAGACACGCAGGGATGAGA/ |
| | ideoxyU/GG/ideoxyU//3BioTEG/ |
| SEQ ID NO: 165 | /5Phos/TGCTGCTCATGAAAAAAGATCACACATTCTCAGTGCAGTCGGAGACACGCAGGGATGAGA/ |
| | ideoxyU/GG/ideoxyU//3BioTEG/ |
| SEQ ID NO: 166 | /5Phos/TGCTGCTCATGAAAAAAGATCGTACCATGCATGTGCAGTCGGAGACACGCAGGGATGAGA/ |
| | ideoxyU/GG/ideoxyU//3BioTEG/ |
| SEQ ID NO: 167 | /5Phos/TGCTGCTCATGAAAAAAGATCTCCTCGCATGCATGTGCAGTCGGAGACACGCAGGGATGAGA/ |
| | ideoxyU/GG/ideoxyU//3BioTEG/ |
| SEQ ID NO: 168 | /5Phos/TGCTGCTCATGAAAAAAGATCGTATGCAGATTGTGCAGTCGGAGACACGCAGGGATGAGAA/ |
| | ideoxyU/GG/ideoxyU//3BioTEG/ |
| SEQ ID NO: 169 | /5Phos/TGCTGCTCATGAAAAAAGATCTGGCGCTCATGCTGTGCAGTCGGAGACACGCAGGGATGAGA/ |
| | ideoxyU/GG/ideoxyU//3BioTEG/ |
| SEQ ID NO: 170 | /5Phos/TGCTGCTCATGAAAAAAGATCAGATCAGATAAGTGCAGTCGGAGACACGCAGGGATGAGA/ |
| | ideoxyU/GG/ideoxyU//3BioTEG/ |
| SEQ ID NO: 171 | /5Phos/TGCTGCTCATGAAAAAAGATCACATGCGCTGGATGTGCAGTCGGAGACACGCAGGGATGAGA/ |
| | ideoxyU/GG/ideoxyU//3BioTEG/ |
| SEQ ID NO: 172 | /5Phos/TGCTGCTCATGAAAAAAGATCACAGATGAACTGTGCAGTCGGAGACACGCAGCGATGAGA/ |
| | ideoxyU/GG/ideoxyU//3BioTEG/ |
| SEQ ID NO: 173 | /5Phos/TGCTGCTCATGAAAAAAGATCTGATGTATGTTGTGCAGTCGGAGACACGCAGGGATGAGA/ |
| | ideoxyU/GG/ideoxyU//3BioTEG/ |
| SEQ ID NO: 174 | /5Phos/TGCTGCTCATGAAAAAAGATCTGCATCTAGCCGTGCAGTCGGAGACACGCAGGGATGAGA/ |
| | ideoxyU/GG/ideoxyU//3BioTEG/ |
| SEQ ID NO: 175 | /5Phos/TGCTGCTCATGAAAAAAGATCATGCTGCTCGGCAGTGCAGTCGGAGACACGCAGGGATGAGA/ |
| | ideoxyU/GG/ideoxyU//3BioTEG/ |
| SEQ ID NO: 176 | /5Phos/TGCTGCTCATGAAAAAAGATCATGATCTGAGGCAGTGCAGTCGGAGACACGCAGGGATGAGA/ |
| | ideoxyU/GG/ideoxyU//3BioTEG/ |
| SEQ ID NO: 177 | /5Phos/TGCTGCTCATGAAAAAAGATCGCCACTGATCAGTGCAGTCGGAGACACGCAGGGATGAGA/ |
| | ideoxyU/GG/ideoxyU//3BioTEG/ |
| SEQ ID NO: 178 | /5Phos//TGCTGCTCATGAAAAAAGATCATATGCCATATGTGTGCAGTCGGAGACACGCAGGGATGAGA/ |
| | ideoxyU/GG/ideoxyU//3BioTEG/ |
| SEQ ID NO: 179 | /5Phos/TGCTGCTCATGAAAAAAGATCTGTGGCGATCTGTGCAGTCGGAGACACGCAGGGATGAGA/ |
| | ideoxyU/GG/ideoxyU//3BioTEG/ |
| SEQ ID NO: 180 | /5Phos/TGCTGCTCATGAAAAAAGATCACTATCATCAAGTGCAGTCGGAGACACGCAGGGATGAGA/ |
| | ideoxyU/GG/ideoxyU//3BioTEG/ |
| SEQ ID NO: 181 | /5Phos/TGCTGCTCATGAAAAAAGATCTGCAGCCAGAGATGTGCAGTCGGAGACACGCAGGGATGAGA/ |
| | ideoxyU/GG/ideoxyU//3BioTEG/ |
| SEQ ID NO: 182 | /5Phos/TGCTGCTCATGAAAAAAGATCACATGCACCAGATGTGCAGTCGGAGACACGCAGGGATGAGA/ |
| | ideoxyU/GG/ideoxyU//3BioTEG/ |
| SEQ ID NO: 183 | /5Phos/TGCTGCTCATGAAAAAAGATCGCGAAGCTGCTGTGCAGTCGGAGACACGCAGGGATGAGA/ |
| | ideoxyU/GG/ideoxyU//3BioTEG/ |
| SEQ ID NO: 184 | /5Phos/TGCTGCTCATGAAAAAAGATCTGCTGCCATATGCGTGCAGTCGGAGACACGCAGGGATGAGA/ |
| | ideoxyU/GG/ideoxyU//3BioTEG/ |
| SEQ ID NO: 185 | /5Phos/TGCTGCTCATGAAAAAAGATCTCGCCATCTGCATGTGCAGTCGGAGACACGCAGGGATGAGA/ |
| | ideoxyU/GG/ideoxyU//3BioTEG/ |
| SEQ ID NO: 186 | /5Phos/TGCTGCTCATGAAAAAAGATCTGAGATGCATGAAGTGCAGTCGGAGACACGCAGGGATGAGA/ |
| | ideoxyU/GG/ideoxyU//3BioTEG/ |
| SEQ ID NO: 187 | /5Phos/TGCTGCTCATGAAAAAAGATCATCACACTTGCATGTGCAGTCGGAGACACGCAGGGATGAGA/ |
| | ideoxyU/GG/ideoxyU//3BioTEG/ |
| SEQ ID NO: 188 | /5Phos/TGCTGCTCATGAAAAAAGATCTGCAGCGAACAGTGCAGTCGGAGACACGCAGGGATGAGA/ |
| | ideoxyU/GG/ideoxyU//3BioTEG/ |
| SEQ ID NO: 189 | /5Phos/TGCTGCTCATGAAAAAAGATCATGCAGGATCTGAGTGCAGTCGGAGACACGCAGGGATGAGA/ |
| | ideoxyU/GG/ideoxyU//3BioTEG/ |
| SEQ ID NO: 190 | /5Phos/TGCTGCTCATGAAAAAAGATCATTATCTGTGTGTGCAGTCGGAGACACGCAGGGGATGAGA/ |
| | ideoxyU/GG/ideoxyU//3BioTEG/ |
| SEQ ID NO: 191 | /5Phos/TGCTGCTCATGAAAAAAGATCGCCAGAGCATCGTGCAGTCGGAGACACGCAGGGATGAGA/ |
| | ideoxyU/GG/ideoxyU//3BioTEG/ |
| SEQ ID NO: 192 | /5Phos/TGCTOCTCATGAAAAAAGATCAGATGATGGATGTGTGCAGTCGGAGACACGCAGGGATGAGA/ |
| | ideoxyU/GG/ideoxyU//3BioTEG/ |

TABLE 3
Adaptor Molecules with AAGG (SEQ ID NO: 386) Mismatch Sequence Pair

| | Sense Strand Sequence |
|---|---|
| SEQ ID NO: 193 | ATCTCATCCCTGCGTGTCTCCGACTGCACTGCACCATCATATGATCAAGGACATGAGCAGC*A*T |
| SEQ ID NO: 194 | ATCTCATCCCTGCGTGTCTCCGACTGCACATGCTATAGCAATGATCAAGGACATGAGCAGC*A*T |
| SEQ ID NO: 195 | ATCTCATCCCTGCGTGTCTCCGACTGCACATCTGCACATGGCGATCAAGGACATGAGCAGC*A*T |
| SEQ ID NO: 196 | ATCTCATCCCTGCGTGTCTCCGACTGCACTATTCAGATGCATGATCAAGGACATGAGCAGC*A*T |
| SEQ ID NO: 197 | ATCTCATCCCTGCGTGTCTCCGACTGCACATGCAACACTAGATCAAGGACATGAGCAGC*A*T |
| SEQ ID NO: 198 | ATCTCATCCCTGCGTGTCTCCGACTGCACTATTATGATATGATCAAGGACATGAGCAGC*A*T |
| SEQ ID NO: 199 | ATCTCATCCCTGCGTGTCTCCGACTGCACTCAGAAGCATCGATCAAGGACATGAGCAGC*A*T |
| SEQ ID NO: 200 | ATCTCATCCCTGCGTGTCTCCGACTGCACTTCATATCTGAGATCAAGGACATGAGCAGC*A*T |
| SEQ ID NO: 201 | ATCTCATCCCTGCGTGTCTCCGACTGCACAATAGCTATGAGATCAAGGACATGAGCAGC*A*T |
| SEQ ID NO: 202 | ATCTCATCCCTGCGTGTCTCCGACTGCACATGGATAGCTGCAGATCAAGGACATGAGCAGC*A*T |
| SEQ ID NO: 203 | ATCTCATCCCTGCGTGTCTCCGACTGCACATAATATCTGCGATCAAGGACATGAGCAGC*A*T |
| SEQ ID NO: 204 | ATCTCATCCCTGCGTGTCTCCGACTGCACAGCACAGAGGAGATCAAGGACATGAGCAGC*A*T |
| SEQ ID NO: 205 | ATCTCATCCCTGCGTGTCTCCGACTGCACAACTGCGCTCTGATCAAGGACATGAGCAGC*A*T |
| SEQ ID NO: 206 | ATCTCATCTCTGTGTGTCTCCGACTGCACACAAGATGACAGATCAAGGACATGAGCAGC*A*T |
| SEQ ID NO: 207 | ATCTCATCCCTGCGTGTCTCCGACTGCACAGATGACATTAGATCAAGGACATGAGCAGC*A*T |
| SEQ ID NO: 208 | ATCTCATCCCTGCGTGTCTCCGACTGCACATGGCTCACATATGATCAAGGACATGAGCAGC*A*T |
| SEQ ID NO: 209 | ATCTCATCCCTGCGTGTCTCCGACTGCACAAGAGATGCATCTGATCAAGGACATGAGCAGC*A*T |
| SEQ ID NO: 210 | ATCTCATCCCTGCGTGTCTCCGACTGCACTTAGCTGTGATGATCAAGGACATGAGCAGC*A*T |
| SEQ ID NO: 211 | ATCTCATCCCTGCGTGTCTCCGACTGCACTCGCAATAGATGATCAAGGACATGAGCAGC*A*T |
| SEQ ID NO: 212 | ATCTCATCCCTGCGTGTCTCCGACTGCACACTCTCTCAATGATCAAGGACATGAGCAGC*A*T |
| SEQ ID NO: 213 | ATCTCATCCCTGCGTGTCTCCGACTGCACACTGCCTGATGATGATCAAGGACATGAGCAGC*A*T |
| SEQ ID NO: 214 | ATCTCATCCCTGCGTGTCTCCGACTGCACTCTATTCATATGATCAAGGACATGAGCAGC*A*T |
| SEQ ID NO: 215 | ATCTCATCCCTGCGTGTCTCCGACTGCACTTGAGCGCACTGATCAAGGACATGAGCAGC*A*T |
| SEQ ID NO: 216 | ATCTCATCCCTGCGTGTCTCCGACTGCACTCAACATATCTGATCAAGGACATGAGCAGC*A*T |
| SEQ ID NO: 217 | ATCTCATCCCTGCGTGTCTCCGACTGCACATGCGGCGCAGCAGATCAAGGACATGAGCAGC*A*T |
| SEQ ID NO: 218 | ATCTCATCCCTGCGTGTCTCCGACTGCACTTGCTGTGCGCGATCAAGGACATGAGCAGC*A*T |
| SEQ ID NO: 219 | ATCTCATCCCTGCGTGTCTCCGACTGCACATAGCGTTCATGATCAAGGACATGAGCAGC*A*T |
| SEQ ID NO: 220 | ATCTCATCCCTGCGTGTCTCCGACTGCACAGCCATCTGCTGAGATCAAGGACATGAGCAGC*A*T |
| SEQ ID NO: 221 | ATCTCATCCCTGCGTGTCTCCGACTGCACACTCAGCGCCAGATCAAGGACATGAGCAGC*A*T |
| SEQ ID NO: 222 | ATCTCATCCCTGCGTGTCTCCGACTGCACAACATCTGATAGATCAAGGACATGAGCAGC*A*T |
| SEQ ID NO: 223 | ATCTCATCCGTGCGTGTCTCCGACTGCACTGCTCCGCATCGATCAAGGACATGAGCAGC*A*T |
| SEQ ID NO: 224 | ATCTCATCCCTGCGTGTCTCCGACTGCACTATCGCTTGATGATCAAGGACATGAGCAGC*A*T |
| SEQ ID NO: 225 | ATCTCATCCCTGCGTGTCTCCGACTGCACTCTTCTCAGCAGATCAAGGACATGAGCAGC*A*T |
| SEQ ID NO: 226 | ATCTCATCCCTGCGTGTCTCCGACTGCACATGGTGTGCACGATCAAGGACATGAGCAGC*A*T |
| SEQ ID NO: 227 | ATCTCATCCCTGCGTGTCTCCGACTGCACATATCATCTTAGATCAAGGACATGAGCAGC*A*T |
| SEQ ID NO: 228 | ATCTCATCCCTGCGTGTCTCCGACTGCACATGATCACACAATGATCAAGGACATGAGCAGC*A*T |
| SEQ ID NO: 229 | ATCTCATCCCTGCGTGTCTCCGACTGCACTGCACATTGTAGATCAAGGACATGAGCAGC*A*T |
| SEQ ID NO: 230 | ATCTCATCCCTGCGTGTCTCCGACTGCACTCATGGTATATGATCAAGGACATGAGCAGC*A*T |
| SEQ ID NO: 231 | ATCTCATCCCTGCGTGTCTCCGACTGCACTGCATGCTGGCGTGATCAAGGACATGAGCAGC*A*T |
| SEQ ID NO: 232 | ATCTCATCCCTGCGTGTCTCCGACTGCACTCTTCATGAGCATGATCAAGGACATGAGCAGC*A*T |
| SEQ ID NO: 233 | ATCTCATCCCTGCGTGTCTCCGACTGCACTCATGGAGCATATGATCAAGGACATGAGCAGC*A*T |
| SEQ ID NO: 234 | ATCTCATCCCTGCGTGTCTCCGACTGCACATACTGCCTATGATCAAGGACATGAGCAGC*A*T |
| SEQ ID NO: 235 | ATCTCATCCCTGCGTGTCTCCGACTGCACAATGCATCTATATGATCAAGGACATGAGCAGC*A*T |
| SEQ ID NO: 236 | ATCTCATCCCTGCGTGTCTCCGACTGCACGAGCACAATGCATGATCAAGGACATGAGCAGC*A*T |
| SEQ ID NO: 237 | ATCTCATCCCTGCGTGTCTCCGACTGCACAACATGCACATCTGATCAAGGACATGAGCAGC*A*T |
| SEQ ID NO: 238 | ATCTCATCCCTGCGTGTCTCCGACTGCACGTGCATGGCATCTGATCAAGGACATGAGCAGC*A*T |
| SEQ ID NO: 239 | ATCTCATCCCTGCGTGTCTCCGACTGCACTACAGCAATGTGATCAAGGACATGAGCAGC*A*T |
| SEQ ID NO: 240 | ATCTCATCCCTGCGTGTCTCCGACTGCACATCAGTCCTGCGATCAAGGACATGAGCAGC*A*T |
| SEQ ID NO: 241 | ATCTCATCCCTGCGTGTCTCCGACTGCACAAGCGACATGCGATCAAGGACATGAGCAGC*A*T |
| SEQ ID NO: 242 | ATCTCATCCCTGCGTGTCTCCGACTGCACATGCTCAATATCTGATCAAGGACATGAGCAGC*A*T |
| SEQ ID NO: 243 | ATCTCATCCCTGCGTGTCTCCGACTGCACAATCGCATCGTGATCAAGGACATGAGCAGC*A*T |
| SEQ ID NO: 244 | ATCTCATCCCTGCGTGTCTCCGACTGCACGGCACTGCAGTGATCAAGGACATGAGCAGC*A*T |
| SEQ ID NO: 245 | ATCTCATCCCTGCGTGTCTCCGACTGCACTATTGCTCTGCATGATCAAGGACATGAGCAGC*A*T |
| SEQ ID NO: 246 | ATCTCATCCCTGCGTGTCTCCGACTGCACATCTGCGGCACATGATCAAGGACATGAGCAGC*A*T |
| SEQ ID NO: 247 | ATCTCATCCCTGCGTGTCTCCGACTGCACATCGATGCAGAATGATCAAGGACATGAGCAGC*A*T |
| SEQ ID NO: 248 | ATCTCATCCCTGCGTGTCTCCGACTGCACACATGACCAGCGATCAAGGACATGAGCAGC*A*T |
| SEQ ID NO: 249 | ATCTCATCCCTGCGTGTCTCCGACTGCACAATGCTGTAGTGATCAAGGACATGAGCAGC*A*T |
| SEQ ID NO: 250 | ATCTCATCCCTGCGTGTCTCCGACTGCACATCATTGATCATCGATCAAGGACATGAGCAGC*A*T |
| SEQ ID NO: 251 | ATCTCATCCCTGCGTGTCTCGGACTGCACACTCACAATGCATGATCAAGGACATGAGCAGC*A*T |
| SEQ ID NO: 252 | ATCTCATCCCTGCGTGTCTCCGACTGCACATGTGGCTATCATGATCAAGGACATGAGCAGC*A*T |
| SEQ ID NO: 253 | ATCTCATCCCTGCGTGTCTCCGACTGCACATGTATGCGCAATGATCAAGGACATGAGCAGC*A*T |
| SEQ ID NO: 254 | ATCTCATCCCTGCGTGTCTCCGACTGCACTTGCGCTCTCAGATCAAGGACATGAGCAGC*A*T |
| SEQ ID NO: 255 | ATCTCATCCCTGCGTGTCTCCGACTGCACGCGCACAATATGATCAAGGACATGAGCAGC*A*T |
| SEQ ID NO: 256 | ATCTCATCCCTGCGTGTCTCCGACTGCACAGCGCACCAGCATGATCAAGGACATGAGCAGC*A*T |
| SEQ ID NO: 257 | ATCTCATCCCTGCGTGTCTCCGACTGCACTGCTATCTGGTGATCAAGGACATGAGCAGC*A*T |
| SEQ ID NO: 258 | ATCTCATCCCTGCGTGTCTCCGACTGCACATGCATGTGGATAGATCAAGGACATGAGCAGC*A*T |
| SEQ ID NO: 259 | ATCTCATCCCTGCGTGTCTCCGACTGCACATCGCACCAGAGATCAAGGACATGAGCAGC*A*T |
| SEQ ID NO: 260 | ATCTCATCCCTGCGTGTCTCCGACTGCACAATCTGATATGATGATCAAGGACATGAGCAGC*A*T |
| SEQ ID NO: 261 | ATCTCATCCCTGCGTGTCTCCGACTGCACATACTGCAGGAGATCAAGGACATGAGCAGC*A*T |
| SEQ ID NO: 262 | ATCTCATCCCTGCGTGTCTCCGACTGCACTTCATGACAGAGATCAAGGACATGAGCAGC*A*T |
| SEQ ID NO: 263 | ATCTCATCCCTGCGTGTCTCCGACTGCACTACATCATGGCATGATCAAGGACATGAGCAGC*A*T |
| SEQ ID NO: 264 | ATCTCATCCCTGCGTGTCTCCGACTGCACTTCACTCATGAGATCAAGGACATGAGCAGC*A*T |
| SEQ ID NO: 265 | ATCTCATCCCTGCGTGTCTCCGACTGCACATGCGCTTACAGATCAAGGACATGAGCAGC*A*T |
| SEQ ID NO: 266 | ATCTCATCCCTGCGTGTCTCCGACTGCACAGCATCTCTGCCTGATCAAGGACATGAGCAGC*A*T |
| SEQ ID NO: 267 | ATCTCATCCCTGCGTGTCTCCGACTGCACACTGCCATATCATGATCAAGGACATGAGCAGC*A*T |
| SEQ ID NO: 268 | ATCTCATCCCTGCGTGTCTCCGACTGCACTCAGCTGCGGOGATCAAGGACATGAGCAGC*A*T |
| SEQ ID NO: 269 | ATCTCATCCCTGCGTGTCTCCGACTGCACAATGCTATCGAGATCAAGGACATGAGCAGC*A*T |
| SEQ ID NO: 270 | ATCTCATCCCTGCGTGTCTCCGACTGCACAGATAAGAGCAGATCAAGGACATGAGCAGC*A*T |
| SEQ ID NO: 271 | ATCTCATCCCTGCGTGTCTCCGACTGCACAGAGCCACTCAGATCAAGGACATGAGCAGC*A*T |
| SEQ ID NO: 272 | ATCTCATCCCTGCGTGTCTCCGACTGCACATGTAATATCAGATCAAGGACATGAGCAGC*A*T |
| SEQ ID NO: 273 | ATCTCATCCCTGCGTGTCTCCGACTGCACATAGATAGCCAGATCAAGGACATGAGCAGC*A*T |
| SEQ ID NO: 274 | ATCTCATCCGTGCGTGTCTCCGACTGCACACTAGCATGGCGATCAAGGACATGAGCAGC*A*T |
| SEQ ID NO: 275 | ATCTCATCCCTGCGTGTCTCCGACTGCACAATAGCATCGAGATCAAGGACATGAGCAGC*A*T |
| SEQ ID NO: 276 | ATCTCATCCCTGCGTGTCTCCGACTGCACTTCTGTGAGATGATCAAGGACATGAGCAGC*A*T |
| SEQ ID NO: 277 | ATCTCATCCCTGCGTGTCTCCGACTGCACACAATAGCGATGATCAAGGACATGAGCAGC*A*T |
| SEQ ID NO: 278 | ATCTCATCCCTGCGTGTCTCCGACTGCACTGTAGGCATCTGATCAAGGACATGAGCAGC*A*T |
| SEQ ID NO: 279 | ATCTCATCCCTGCGTGTCTCCGACTGCACAACAGCTCTCTGATCAAGGACATGAGCAGC*A*T |
| SEQ ID NO: 280 | ATCTCATCCCTGCGTGTCTCCGACTGCACAGACACAATATGATCAAGGACATGAGCAGC*A*T |
| SEQ ID NO: 281 | ATCTCATCCCTGCGTGTCTCCGACTGCACTAGCAAGATCAGATCAAGGACATGAGCAGC*A*T |
| SEQ ID NO: 282 | ATCTCATCCCTGCGTGTCTCCGACTGCACTAGAGACATGAGATCAAGGACATGAGCAGC*A*T |
| SEQ ID NO: 283 | ATCTCATCCCTGCGTGTCTCCGACTGCACAGCCGCATGCACAGATCAAGGACATGAGCAGC*A*T |
| SEQ ID NO: 284 | ATCTCATCCCTGCGTGTCTCCGACTGCACTCGCATGTGGAGATCAAGGACATGAGCAGC*A*T |
| SEQ ID NO: 285 | ATCTCATCCCTGCGTGTCTCCGACTGCACATGCGAGCATGGTGATCAAGGACATGAGCAGC*A*T |
| SEQ ID NO: 286 | ATCTCATCCCTGCGTGTCTCCGACTGCACAATACATGATCGATCAAGGACATGAGCAGC*A*T |
| SEQ ID NO: 287 | ATCTCATCCCTGCGTGTCTCCGACTGCACATGGACATATGCAGATCAAGGACATGAGCAGC*A*T |
| SEQ ID NO: 288 | ATCTCATCCCTGCGTGTCTCCGACTGCACATGCGTCACCTGATCAAGGACATGAGCAGC*A*T |
| ID | Antisense Strand Sequence |
| SEQ ID NO: 289 | /5Phos/TGCTGCTCATGTGGAAGATCATATGATGGTGCAGTGCAGTCGGAGACACGCAGGGATGAGA/ |
| | ideoxyU/GG/ideoxyU//3BioTEG/ |
| SEQ ID NO: 290 | /5Phos/TGCTGCTCATGTGGAAGATCATTGCTATAGCATGTGCAGTCGGAGACACGCAGGGATGAGA/ |
| | ideoxyU/GG/ideoxyU//3BioTEG/ |
| SEQ ID NO: 291 | /5Phos/TGCTGCTCATGTGGAAGATCGCCATGTGCAGATGTGCAGTCGGAGACACGCAGGGATGAGA/ |
| | ideoxyU/GG/ideoxyU//3BioTEG/ |
| SEQ ID NO: 292 | /5Phos/TGCTGCTCATGTGGAAGATCATGCATCTGAATAGTGCAGTCGGAGACACGCAGGGATGAGA/ |
| | ideoxyU/GG/ideoxyU//3BioTEG/ |
| SEQ ID NO: 293 | /5Phos/TGCTGCTCATGTGGAAGATCTAGTGTTGCATGTGCAGTCGGAGACACGCAGGGATGAGA/ |
| | ideoxyU/GG/ideoxyU//3BioTEG/ |
| SEQ ID NO: 294 | /5Phos/TGCTGCTCATGTGGAAGATCATATCATAATAGTGCAGTCGGAGACACGCAGGGATGAGA/ |
| | ideoxyU/GG/ideoxyU//3BioTEG/ |
| SEQ ID NO: 295 | /5Phos/TGCTGCTCATGTGGAAGATCGATGCTTCTGAGTGCAGTCGGAGACACGCAGGGATGAGA/ |
| | ideoxyU/GG/ideoxyU//3BioTEG/ |
| SEQ ID NO: 296 | /5Phos/TGCTGCTCATGTGGAAGATCTCAGATATGAAGTGCAGTCGGAGACACGCAGGGATGAGA/ |
| | ideoxyU/GG/ideoxyU//3BioTEG/ |
| SEQ ID NO: 297 | /5Phos/TGCTGCTCATGTGGAAGATCTCATAGCTATTGTGCAGTCGGAGACACGCAGGGATGAGA/ |
| | ideoxyU/GG/ideoxyU//3BioTEG/ |
| SEQ ID NO: 298 | /5Phos/TGCTGCTCATGTGGAAGATCTGCAGCTATCCATGTGCAGTCGGAGACACGCAGGGATGAGA/ |
| | ideoxyU/GG/ideoxyU//3BioTEG/ |
| SEQ ID NO: 299 | /5Phos/TGCTGCTCATGTGGAAGATCGCAGATATTATGTGCAGTCGGAGACACGCAGGGATGAGA/ |
| | ideoxyU/GG/ideoxyU//3BioTEG/ |
| SEQ ID NO: 300 | /5Phos/TGCTGCTCATGTGGAAGATCTCCTCTGTGCTGTGCAGTCGGAGACACGCAGGGATGAGA/ |
| | ideoxyU/GG/ideoxyU//3BioTEG/ |
| SEQ ID NO: 301 | /5Phos/TGCTGCTCATGTGGAAGATCAGAGCGCAGTTGTGCAGTCGGAGACACGCAGGGATGAGA/ |
| | ideoxyU/GG/ideoxyU//3BioTEG/ |
| SEQ ID NO: 302 | /5Phos/TGCTGCTCATGTGGAAGATCTGTCATCTTGTGTGCAGTCGGAGACACGCAGGGATGAGA/ |
| | ideoxyU/GG/ideoxyU//3BioTEG/ |
| SEQ ID NO: 303 | /5Phos/TGCTGCTCATGTGGAAGATCTAATGTCATCTGTGCAGTCGGAGACACGCAGGGATGAGA/ |
| | ideoxyU/GG/ideoxyU//3BioTEG/ |
| SEQ ID NO: 304 | /5Phos/TGCTGCTCATGTGGAAGATCATATGTGAGCCATGTGCAGTCGGAGACACGCAGGGATGAGA/ |
| | ideoxyU/GG/ideoxyU//3BioTEG/ |
| SEQ ID NO: 305 | /5Phos/TGCTGCTCATGTGGAAGATCAGATGCATCTCTTGTGCAGTCGGAGACACGCAGGGATGAGA/ |
| | ideoxyU/GG/ideoxyU//3BioTEG/ |
| SEQ ID NO: 306 | /5Phos/TGCTGCTCATGTGGAAGATCATCACAGCTAAGTGCAGTCGGAGACACGCAGGGATGAGA/ |
| | ideoxyU/GG/ideoxyU//3BioTEG/ |
| SEQ ID NO: 307 | /5Phos/TGCTGCTCATGTGGAAGATCATCTATTGCGAGTGCAGTCGGAGACACGCAGGGATGAGA/ |
| | ideoxyU/GG/ideoxyU//3BioTEG/ |
| SEQ ID NO: 308 | /5Phos/TGCTGCTCATGTGGAAGATCATTGAGAGAGTGTGCAGTCGGAGACACGCAGGGATGAGA/ |
| | ideoxyU/GG/ideoxyU//3BioTEG/ |
| SEQ ID NO: 309 | /5Phos/TGCTGCTCATGTGGAAGATCATCATCAGGCAGTGTGCAGTCGGAGACACGCAGGGATGAGA/ |
| | ideoxyU/GG/ideoxyU//3BioTEG/ |
| SEQ ID NO: 310 | /5Phos/TGCTGCTCATGTGGAAGATCATATGAATAGAGTGCAGTCGGAGACACGCAGGGATGAGA/ |
| | ideoxyU/GG/ideoxyU//3BioTEG/ |
| SEQ ID NO: 311 | /5Phos/TGCTGCTCATGTGGAAGATCAGTGCGCTCAAGTGCAGTOGGAGACACGCAGGGATGAGA/ |
| | ideoxyU/GG/ideoxyU//3BioTEG/ |
| SEQ ID NO: 312 | /5Phos/TGCTGCTCATGTGGAAGATCAGATATGTTGAGTGCAGTCGGAGACACGCAGGGATGAGA/ |
| | ideoxyU/GG/ideoxyU//3BioTEG/ |
| SEQ ID NO: 313 | /5Phos/TGCTGCTCATGTGGAAGATCTGCTGCGCCGCATGTGCAGTCGGAGACACGCAGGGATGAGA/ |
| | ideoxyU/GG/ideoxyU//3BioTEG/ |
| SEQ ID NO: 314 | /5Phos/TGCTGCTCATGTGGAAGATCGCGCACAGCAAGTGCAGTCGGAGACACGCAGGGATGAGA/ |
| | ideoxyU/GG/ideoxyU//3BioTEG/ |
| SEQ ID NO: 315 | /5Phos/TGCTGCTCATGTGGAAGATCATGAACGCTATGTGCAGTCGGAGACACGCAGGGATGAGA/ |
| | ideoxyU/GG/ideoxyU//3BioTEG/ |
| SEQ ID NO: 316 | /5Phos/TGCTGCTCATGTGGAAGATCTCAGCAGATGGCTGTGCAGTCGGAGACACGCAGGGATGAGA/ |
| | ideoxyU/GG/ideoxyU//3BioTEG/ |
| SEQ ID NO: 317 | /5Phos/TGCTGCTCATGTGGAAGATCTGGCGCTGAGTGTGCAGTCGGAGACACGCAGGGATGAGA/ |
| | ideoxyU/GG/ideoxyU//3BioTEG/ |
| SEQ ID NO: 318 | /5Phos/TGCTGCTCATGTGGAAGATCTATCAGATGTTGTGCAGTCGGAGACACGCAGGGATGAGA/ |
| | ideoxyU/GG/ideoxyU//3BioTEG/ |
| SEQ ID NO: 319 | /5Phos/TGCTGCTCATGTGGAAGATCGATGCGGAGCAGTGCAGTCGGAGACACGCAGGGATGAGA/ |
| | ideoxyU/GG/ideoxyU//3BioTEG/ |
| SEQ ID NO: 320 | /5Phos/TGCTGCTCATGTGGAAGATCATCAAGCGATAGTGCAGTCGGAGACACGCAGGGATGAGA/ |
| | ideoxyU/GG/ideoxyU//3BioTEG/ |
| SEQ ID NO: 321 | /5Phos/TGCTGCTCATGTGGAAGATCTGCTGAGAAGAGTGCAGTCGGAGACACGCAGGGATGAGA/ |
| | ideoxyU/GG/ideoxyU//3BioTEG/ |
| SEQ ID NO: 322 | /5Phos/TGCTGCTCATGTGGAAGATCGTGCACACCATGTGCAGTCGGAGACACGCAGGGTGAGA/ |
| | ideoxyU/GG/ideoxyU//3BioTEG/ |
| SEQ ID NO: 323 | /5Phos/TGCTGCTCATGTGGAAGATCTAAGATGATATGTGCAGTCGGAGACACGCAGGGATGAGA/ |
| | ideoxyU/GG/ideoxyU//3BioTEG/ |
| SEQ ID NO: 324 | /5Phos/TGCTGCTCATGTGGAAGATCATTGTGTGATCATGTGCAGTCGGAGACACGCAGGGATGAGA/ |
| | ideoxyU/GG/ideoxyU//3BioTEG/ |
| SEQ ID NO: 325 | /5Phos/TGCTGCTCATGTGGAAGATCTACAATGTGCAGTGCAGTCGGAGACACGCAGGGATGAGA/ |
| | ideoxyU/GG/ideoxyU//3BioTEG/ |
| SEQ ID NO: 326 | /5Phos/TGCTGCTCATGTGGAAGATCATATACCATGAGTGCAGTCGGAGACACGCAGGGATGAGA/ |
| | ideoxyU/GG/ideoxyU//3BioTEG/ |
| SEQ ID NO: 327 | /5Phos/TGCTGCTCATGTGGAAGATCACGCCAGCATGCAGTGCAGTCGGAGACACGCAGGGATGAGA/ |
| | ideoxyU/GG/ideoxyU//3BioTEG/ |
| SEQ ID NO: 328 | /5Phos/TGCTGCTCATGTGGAAGATCATGCTCATGAAGAGTGCAGTCGGAGACACGCAGGGATGAGA/ |
| | ideoxyU/GG/ideoxyU//3BioTEG/ |
| SEQ ID NO: 329 | /5Phos/TGCTGCTCATGTGGAAGATCATATGCTCCATGAGTGCAGTCGGAGACACGCAGGGATGAGA/ |
| | ideoxyU/GG/ideoxyU//3BioTEG/ |
| SEQ ID NO: 330 | /5Phos/TGCTGCTCATGTGGAAGATCATAGGCAGTATGTGCAGTCGGAGACACGCAGGGATGAGA/ |
| | ideoxyU/GG/ideoxyU//3BioTEG/ |
| SEQ ID NO: 331 | /5Phos/TGCTGCTCATGTGGAAGATCATATAGATGCATTGTGCAGTCGGAGACACGCAGGGATGAGA/ |
| | ideoxyU/GG/ideoxyU//3BioTEG/ |
| SEQ ID NO: 332 | /5Phos/TGCTGCTCATGTGGAAGATCATGCATTGTGCTCGTGCAGTCGGAGACACGCAGGGATGAGA/ |
| | ideoxyU/GG/ideoxyU//3BioTEG/ |
| SEQ ID NO: 333 | /5Phos/TGCTGCTCATGTGGAAGATCAGATGTGCATGTTGTGCAGTCGGAGACACGCAGGGATGAGA/ |
| | ideoxyU/GG/ideoxyU//3BioTEG/ |
| SEQ ID NO: 334 | /5Phos/TGCTGCTCATGTGGAAGATCAGATGCCATGCACGTGCAGTCGGAGACACGCAGGGATGAGA/ |
| | ideoxyU/GG/ideoxyU//3BioTEG/ |
| SEQ ID NO: 335 | /5Phos/TGCTGCTCATGTGGAAGATCACATTGCTGTAGTGCAGTCGGAGACACGCAGGGATGAGA/ |
| | ideoxyU/GG/ideoxyU//3BioTEG/ |
| SEQ ID NO: 336 | /5Phos/TGCTGCTCATGTGGAAGATCGCAGGACTGATGTGCAGTCGGAGACACGCAGGGATGAGA/ |
| | ideoxyU/GG/ideoxyU//3BioTEG/ |
| SEQ ID NO: 337 | /5Phos/TGCTGCTCATGTGGAAGATCGCATGTCGCTTGTGCAGTCGGAGACACGCAGGGATGAGA/ |
| | ideoxyU/GG/ideoxyU//3BioTEG/ |
| SEQ ID NO: 338 | /5Phos/TGCTGCTCATGTGGAAGATCAGATATTGAGCATGTGCAGTCGGAGACACGCAGGGATGAGA/ |
| | ideoxyU/GG/ideoxyU//3BioTEG/ |
| SEQ ID NO: 339 | /5Phos/TGCTGCTCATGTGGAAGATCACGATGCGATTGTGCAGTCGGAGACACGCAGGGATGAGA/ |
| | ideoxyU/GG/ideoxyU//3BioTEG/ |
| SEQ ID NO: 340 | /5Phos/TGCTGCTCATGTGGAAGATCACTGCAGTGCCGTGCAGTCGGAGACACGCAGGGATGAGA/ |
| | ideoxyU/GG/ideoxyU//3BioTEG/ |
| SEQ ID NO: 341 | /5Phos/TGCTGCTCATGTGGAAGATCATGCAGAGCAATAGTGCAGTCGGAGACACGCAGGGATGAGA/ |
| | ideoxyU/GG/ideoxyU//3BioTEG/ |
| SEQ ID NO: 342 | /5Phos/TGCTGCTCATGTGGAAGATCATGTGCCGCAGATGTGCAGTCGGAGACACGCAOGGATGAGA/ |
| | ideoxyU/GG/ideoxyU//3BioTEG/ |
| SEQ ID NO: 343 | /5Phos/TGCTGCTCATGTGGAAGATCATTCTGCATCGATGTGCAGTCGGAGACACGCAGGGATGAGA/ |
| | ideoxyU/GG/ideoxyU//3BioTEG/ |
| SEQ ID NO: 344 | /5Phos/TGCTGCTCATGTGGAAGATCGCTGGTCATGTGTGCAGTCGGAGACACGCAGGGATGAGA/ |
| | ideoxyU/GG/ideoxyU//3BioTEG/ |
| SEQ ID NO: 345 | /5Phos/TGCTGCTCATGTGGAAGATCACTACAGCATTGTGCAGTCGGAGACACGCAGGGATGAGA/ |
| | ideoxyU/GG/ideoxyU//3BioTEG/ |
| SEQ ID NO: 346 | /5Phos/TGCTGCTCATGTGGAAGATCGATGATCAATGATGTGCAGTCGGAGACACGCAGGGATGAGA/ |
| | ideoxyU/GG/ideoxyU//3BioTEG/ |
| SEQ ID NO: 347 | /5Phos/TGCTGCTCATGTGGAAGATCATGCATTGTGAGTGTGCAGTCGGAGACACGCAGGGATGAGA/ |
| | ideoxyU/GG/ideoxyU//3BioTEG/ |
| SEQ ID NO: 348 | /5Phos/TGCTGCTCATGTGGAAGATCATGATAGCCACATGTGCAGTCGGAGACACGCAGGGATGAGA/ |
| | ideoxyU/GG/ideoxyU//3BioTEG/ |
| SEQ ID NO: 349 | /5Phos/TGCTGCTCATGTGGAAGATCATTGCGCATACATGTGCAGTCGGAGACACGCAGGGATGAGA/ |
| | ideoxyU/GG/ideoxyU//3BioTEG/ |
| SEQ ID NO: 350 | /5Phos/TGCTGCTCATGTGGAAGATCTGAGAGCGCAAGTGCAGTCGGAGACACGCAGGGATGAGA/ |
| | ideoxyU/GG/ideoxyU//3BioTEG/ |
| SEQ ID NO: 351 | /5Phos/TGCTGCTCATGTGGAAGATCATATTGTGCGCGTGCAGTCGGAGACACGCAGGGATGAGA/ |
| | ideoxyU/GG/ideoxyU//3BioTEG/ |
| SEQ ID NO: 352 | /5Phos/TGCTGCTCATGTGGAAGATCATGCTGGTGCGCTGTGCAGTCGGAGACACGCAGGGATGAGA/ |
| | ideoxyU/GG/ideoxyU//3BioTEG/ |
| SEQ ID NO: 353 | /5Phos/TGCTGCTCATGTGGAAGATCACCAGATAGCAGTGCAGTCGGAGACACGCAGGGATGAGA/ |
| | ideoxyU/GG/ideoxyU//3BioTEG/ |
| SEQ ID NO: 354 | /5Phos/TGCTGCTCATGTGGAAGATCTATCCACATGCATGTGCAGTCGGAGACACGCAGGGATGAGA/ |
| | ideoxyU/GG/ideoxyU//3BioTEG/ |
| SEQ ID NO: 355 | /5Phos/TGCTGCTCATGTGGAAGATCTCTGGTGCGATGTGCAGTCGGAGACACGCAGGGATGAGA/ |
| | ideoxyU/GG/ideoxyU//3BioTEG/ |
| SEQ ID NO: 356 | /5Phos/TGCTGCTCATGTGGAAGATCATCATATCAGATTGTGCAGTCGGAGACACGCAGGGATGAGA/ |
| | ideoxyU/GG/ideoxyU//3BioTEG/ |
| SEQ ID NO: 357 | /5Phos/TGCTGCTCATGTGGAAGATCTCCTGCAGTATGTGCAGTCGGAGACACGCAGGGATGAGA/ |
| | ideoxyU/GG/ideoxyU//3BioTEG/ |
| SEQ ID NO: 358 | /5Phos/TGCTGCTCATGTGGAAGATCTCTGTCATGAAGTGCAGTCGGAGACACGCAGGGATGAGA/ |
| | ideoxyU/GG/ideoxyU//3BioTEG/ |
| SEQ ID NO: 359 | /5Phos/TGCTGCTCATGTGGAAGATCATGCCATGATGTAGTGCAGTCGGAGACACGCAGGGATGAGA/ |
| | ideoxyU/GG/ideoxyU//3BioTEG/ |
| SEQ ID NO: 360 | /5Phos/TGCTGCTCATGTGGAAGATCTCATGAGTGAAGTGCAGTCGGAGACACGCAGGGATGAGA/ |
| | ideoxyU/GG/ideoxyU//3BioTEG/ |
| SEQ ID NO: 361 | /5Phos/TGCTGCTCATGTGGAAGATCTGTAAGCGCATGTGCAGTCGGAGACACGCAGGGATGAGA/ |
| | ideoxyU/GG/ideoxyU//3BioTEG/ |
| SEQ ID NO: 362 | /5Phos/TGCTGCTCATGTGGAAGATCAGGCAGAGATGCTGTGCAGTCGGAGACACGCAGGGATGAGA/ |
| | ideoxyU/GG/ideoxyU//3BioTEG/ |
| SEQ ID NO: 363 | /5Phos/TGCTGCTCATGTGGAAGATCATGATATGGCAGTGTGCAGTCGGAGACACGCAGGGATGAGA/ |
| | ideoxyU/GG/ideoxyU//3BioTEG/ |
| SEQ ID NO: 364 | /5Phos/TGCTGCTCATGTGGAAGATCGCCGCAGCTGAGTGCAGTCGGAGACACGCAGGGATGAGA/ |
| | ideoxyU/GG/ideoxyU//3BioTEG/ |
| SEQ ID NO: 365 | /5Phos/TGCTGCTCATGTGGAAGATCTCGATAGCATTGTGCAGTCGGAGACACGCAGGGATGAGA/ |
| | ideoxyU/GG/ideoxyU//3BioTEG/ |
| SEQ ID NO: 366 | /5Phos/TGCTGCTCATGTGGAAGATCTGCTCTTATCTGTGCAGTCGGAGACACGCAGGGATGAGA/ |
| | ideoxyU/GG/ideoxyU//3BioTEG/ |
| SEQ ID NO: 367 | /5Phos/TGCTGCTCATGTGGAAGATCTGAGTGGCTCTGTGCAGTCGGAGACACGCAGGGATGAGA/ |
| | ideoxyU/GG/ideoxyU//3BioTEG/ |
| SEQ ID NO: 368 | /5Phos/TGCTGCTCATGTGGAAGATCTGATATTACATGTGCAGTCGGAGACACGCAGGGATGAGA/ |
| | ideoxyU/GG/ideoxyU//3BioTEG/ |
| SEQ ID NO: 369 | /5Phos/TGCTGCTCATGTGGAAGATCTGGCTATCTATGTGCAGTCGGAGACACGCAGGGATGAGA/ |
| | ideoxyU/GG/ideoxyU//3BioTEG/ |
| SEQ ID NO: 370 | /5Phos/TGCTGCTCATGTGGAAGATCGCCATGCTAGTGTGCAGTCGGAGACACGCAGGGATGAGA/ |
| | ideoxyU/GG/ideoxyU//3BioTEG/ |
| SEQ ID NO: 371 | /5Phos/TGCTGCTCATGTGGAAGATCTCGATGCTATTGTGCAGTCGGAGACACGCAGGGATGAGA/ |
| | ideoxyU/GG/ideoxyU//3BioTEG/ |
| SEQ ID NO: 372 | /5Phos/TGCTGCTCATGTGGAAGATCATCTCACAGAAGTGCAGTCGGAGACACGCAGGGATGAGA/ |
| | ideoxyU/GG/ideoxyU//3BioTEG/ |
| SEQ ID NO: 373 | /5Phos/TGCTGCTCATGTGGAAGATCATCGCTATTGTGTGCAGTCGGAGACACGCAGGGATGAGA/ |
| | ideoxyU/GG/ideoxyU//3BioTEG/ |
| SEQ ID NO: 374 | /5Phos/TGCTGCTCATGTGGAAGATCAGATGCCTACAGTGCAGTCGGAGACACGCAGGGATGAGA/ |
| | ideoxyU/GG/ideoxyU//3BioTEG/ |
| SEQ ID NO: 375 | /5Phos/TGCTGCTCATGTGGAAGATCAGAGAGCTGTTGTGCAGTCGGAGACACGCAGGGATGAGA/ |
| | ideoxyU/GG/ideoxyU//3BioTEG/ |
| SEQ ID NO: 376 | /5Phos/TGCTGCTCATGTGGAAGATCATATTGTGTCTGTGCAGTCGGAGACACGCAGGGATGAGA/ |
| | ideoxyU/GG/ideoxyU//3BioTEG/ |
| SEQ ID NO: 377 | /5Phos/TGCTGCTCATGTGGAAGATCTGATCTTGCTAGTGCAGTCGGAGACACGCAGGGATGAGA/ |
| | ideoxyU/GG/ideoxyU//3BioTEG/ |
| SEQ ID NO: 378 | /5Phos/TGCTGCTCATGTGGAAGATCTCATGTCTCAAGTGCAGTCGGAGACACGCAGGGATGAGA/ |
| | ideoxyU/GG/ideoxyU//3BioTEG/ |
| SBQ ID NO: 379 | /5Phos/TGCTGCTCATGTGGAAGATCTGTGCATGCGGCTGTGCAGTCGGAGACACGCAGGGATGAGA/ |
| | ideoxyU/GG/ideoxyU//3BioTEG/ |
| SEQ ID NO: 380 | /5Phos/TGCTGCTCATGTGGAAGATCTCCACATGCGAGTGCAGTCGGAGACACGCAGGGATGAGA/ |
| | ideoxyU/GG/ideoxyU//3BioTEG/ |
| SEQ ID NO: 381 | /5Phos/TGCTGCTCATGTGGAAGATCACCATGCTCGCATGTGCAGTCGGAGACACGCAGGGATGAGA/ |
| | ideoxyU/GG/ideoxyU//3BioTEG/ |
| SEQ ID NO: 382 | /5Phos/TGCTGCTCATGTGGAAGATCGATCATGTATTGTGCAGTCGGAGACACGCAGGGATGAGA/ |
| | ideoxyU/GG/ideoxyU//3BioTEG/ |
| SEQ ID NO: 383 | /5Phos/TGCTGCTCATGTGGAAGATCTGCATATGTCCATGTGCAGTCGGAGACACGCAGGGATGAGA/ |
| | ideoxyU/GG/ideoxyU//3BioTEG/ |
| SEQ ID NO: 384 | /5Phos/TGCTGCTCATGTGGAAGATCAGGTGACGCATGTGCAGTCGGAGACACGCAGGGATGAGA/ |
| | ideoxyU/GG/ideoxyU//3BioTEG/ |

The adaptor molecules from Tables 2 and 3 comprise in the sense strand the following functional sequences in direction of 5′ to 3′: [adaptor1][barcode][mismatch][adaptor2].

It will be appreciated that the different functional sequences may be reordered to any order (e.g., for [mismatch] to come before [barcode] in the 5′ to 3′ direction), that any of the example functional sequences other than the [mismatch] sequence may be omitted (e.g., [adaptor1], [adaptor2], [barcode], etc.) for the purposes of strand recognition workflows described herein, and/or that any additional functional sequence(s) may be included (e.g., [UMI], [barcode2], [adaptor3], [index], [sample], [preamble], [calibration], [resynching], etc.). The example adaptor molecules from Tables 2 and 3 each comprises the sequence ATCTCATCCCTGCGTGTCTCCGACTGCAC (SEQ ID NO: 387) in the sense strand and its reverse complement sequence in the antisense strand in the [adaptor1] sequence. The example adaptor molecules from Tables 2 and 3 each comprises the sequence CATGAGCAGCAT (SEQ ID NO: 388) in the sense strand and its reverse complement sequence (excluding the reverse complement base for the last T base) in the antisense strand in the [adaptor2], which [adaptor2] portion is configured to attach to a double-stranded insert molecule (e.g., A-tailed insert molecule). Each adaptor molecule in Table 2 comprises a different [barcode] sequence between the [adaptor1] and [mismatch] sequence. Each adaptor molecule in Table 3 comprises a different [barcode] sequence. It will be appreciated that any of the exemplified sequence portions may be different, shorter, and/or longer than the ones listed in the two tables above. Additional examples of barcode sequences and other functional sequences, which can supplement and/or replace any of the listed functional sequences, are provided in Int. Pat. Pub. Nos. WO2023288018A2 and WO2023122104A2, each of which is entirely incorporated by reference for all purposes.

As described elsewhere herein, a strand recognition adaptor may comprise any pair of mismatch sequences in the mismatch portion. In some cases, the pair of mismatch sequences may be designed to optimize adaptor stability (e.g., decreasing size of the loop) and sequencing quality. For example, the mismatch sequence pair of AAGG/AAGG may in some instances obviate nucleotide depletion problems that could arise from the mismatch sequence pair of AAAAAA/AAAAAA, which could lead to phasing problems.

Provided herein are kits comprising a plurality of strand recognition adaptor molecules. A kit may comprise a plurality of strand recognition adaptor molecules, a strand recognition adaptor molecule of the plurality comprising a first strand with a sequence of SEQ ID NO: N selected from 1-96 or 193-288 and a second strand with a sequence of SEQ ID NO: N+96. A kit may comprise a plurality of strand recognition adaptor molecules, comprising first strands with SEQ ID NO: N selected from 1-96 and second strands with SEQ ID NO: N+96. A kit may comprise a plurality of strand recognition adaptor molecules, comprising first strands with SEQ ID NO: N selected from 193-288 and second strands with SEQ ID NO: N+96. A kit may comprise a plurality of strand recognition adaptor molecules, each comprising a mismatch sequence pair: AAAAAA/AAAAAA in a looped mismatch portion. A kit may comprise a plurality of strand recognition adaptor molecules, each comprising a mismatch sequence pair: AAGG/AAGG in a looped mismatch portion. A kit may comprise a synthesized strand recognition molecule comprising a first strand and a second strand, the first strand comprising a first sequence of at least a contiguous 4 base length selected from a sequence of SEQ ID NO: N selected from 1-96 or 193-288. A kit may comprise a synthesized molecule comprising a sequence of any one of SEQ ID NOs: 1-384. A kit may comprise a synthesized molecule comprising a sequence that is at least 80% identical to any one of SEQ ID NOs: 1-384.

### Computer Systems

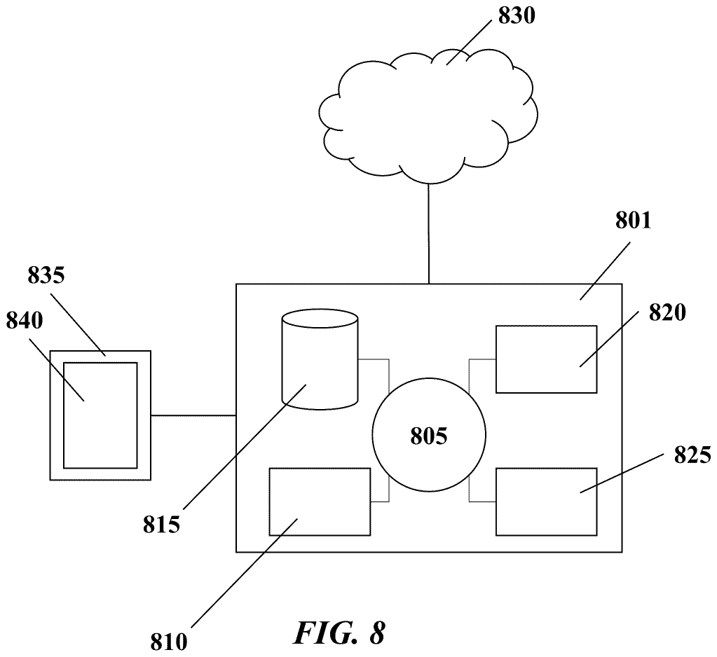

The present disclosure provides computer control systems that are programmed to implement methods of the disclosure. FIG. 8 shows a computer system 801 that is programmed or otherwise configured to implement methods of the disclosure, such as to control the systems described herein (e.g., reagent dispensing, detecting, etc.) and collect, receive, and/or analyze sequencing information. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adaptor) for communicating with one or more other systems, and peripheral devices 825, such as cache, other memory, data storage and/or electronic display adaptors. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an isolated or substantially isolated internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine-readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, results of a nucleic acid sequencing run (e.g., sequence reads, base calls, etc.). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, perform photometry, base calling, and determine forward-reverse % ratio of amplified beads as described elsewhere herein.

### EXAMPLES

These examples are provided for illustrative purposes only and are not intended to limit the scope of the claims provided herein.

### Example 1: Measuring Forward and Reverse Strand Ratio Distributions

PCR-free samples were prepared for sequencing according to the general workflow described with respect to FIGS. 10A-10B. During preparation, double-stranded template molecules each comprising an adaptor comprising the same mismatch portion were attached to beads, to provide a population of single template-attached beads (i.e., beads which each comprise a single template molecule attached thereto). The population of single template-attached beads may be a subset of a larger population of pre-enriched beads (e.g., including multiple template-attached beads and single template-attached beads). The mismatch portion of the adaptor used in this experiment comprises a poly-T/poly-C pair. The template-attached beads were amplified via ePCR, in which the double-stranded template molecules were amplified on the beads. The resulting amplified beads were sequenced, according to methods described elsewhere herein. Sequencing reads for the beads were generated from the sequencing run. The following data analyzes two of the samples, identified by barcode “BC5” and barcode “BC6”, for forward and reverse strand ratios on the amplified beads comprising these samples. The forward strands correspond to amplified strands derived from a top strand of the double-stranded template (expected to contain the poly-T sequence of this mismatch portion) and the reverse strands correspond to amplified strands derived from a bottom strand of the double-stranded template (expected to contain a poly-G sequence, complementary to the poly-C sequence of this mismatch portion). One or more base caller or photometry algorithms were used to process sequencing reads corresponding to the mismatch portion of the adaptor, to determine a distribution of the forward and reverse strands on each bead.

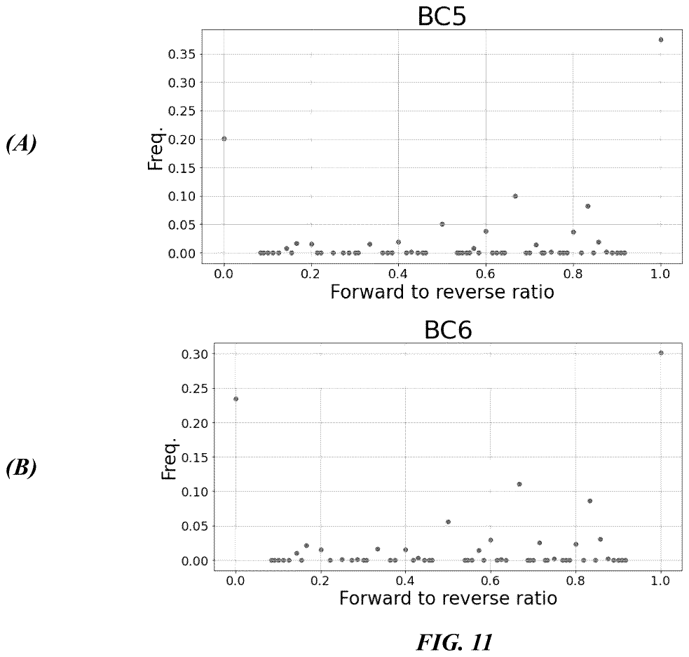

FIG. 11 illustrates a frequency distribution chart of beads having different forward-to-reverse strand ratios for the BC5 and BC6 samples in panels (A) and (B), respectively. The plots are Frequency vs. Forward-to-Reverse Ratio. Panel (A) shows a frequency of about 0.2 for 0.0 forward-to-reverse ratio (20% of beads have only reverse strands) and a frequency of about 0.37 for 1.0 forward-to-reverse ratio (37% of beads have only forward strands). Thus, approximately 43% of the beads have mixed forward and reverse strands, i.e., approximately 43% of the beads (detectably) amplified both strands of the template onto the beads. Panel (B) shows a frequency of about 0.24 for 0.0 forward-to-reverse ratio (24% of beads have only reverse strands) and a frequency of about 0.30 for 1.0 forward-to-reverse ratio (30% of beads have only forward strands). Thus, approximately 46% of the beads have mixed forward and reverse strands, i.e., approximately 46% of the beads amplified both strands of the template onto the beads. In both samples, approximately 42%-46% of the reads are mixed in different ratios of forward-to-reverse strands.

The data demonstrates that the methods of the present disclosure can generate a good distribution of amplified beads comprising mixed amplified strands derived from both template strands, and further that it is possible to detect and determine the forward-to-reverse ratio of an amplified bead based on the sequencing read corresponding to the mismatched portion of the adaptors used for library preparation.

### Example 2: Cycle Skip Substitution Error Rate Analysis

Cycle skip substitution errors for beads of differing forward-to-reverse ratios were examined for the two samples, identified by barcode “BC5” and barcode “BC6”, using the sequencing data obtained from the sequencing run described in Example 1.

The suspected cycle skip substitution errors were examined with some basic filtering to avoid artifacts (edit distance <=3, coverage >=5 to avoid germline variants). The forward only (100%-0% forward-reverse %) and reverse only (0%-100% forward-reverse %) reads were found to account for 57% of the data and yield approximately 25,000 substitutions, and the 50%-50% forward-reverse %% reads were found to account for 5% of the data and yield approximately 200 substitutions—surprisingly, there was at least a 10-fold decrease in error rates for reads of mixed cases than reads of unmixed cases. The term ‘unmixed case’ as used herein generally refers to the case of a bead comprising substantially forward only or substantially reverse only strands, or beads having 100%-0% or 0%-100% forward-reverse % strands. The term ‘mixed case’ as used herein generally refers to the case of a bead comprising a mix of forward and reverse strands, or beads having non-0% or non-100% forward % or reverse % strands. The 66%-33% forward-reverse % reads yielded data that was 2-fold worse than the 50%-50% case with about 4× as many errors but about 2× higher number of reads.

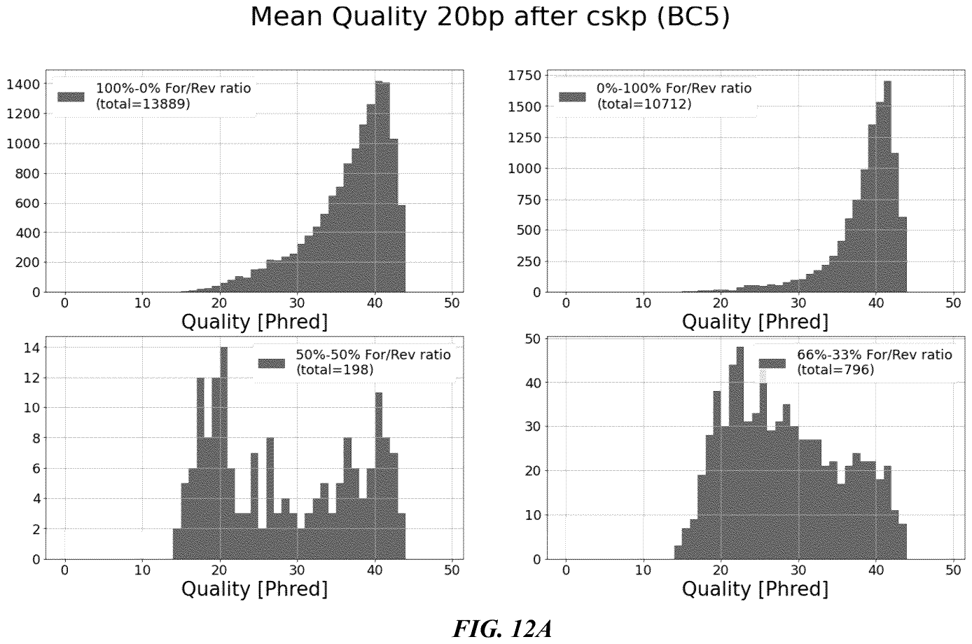

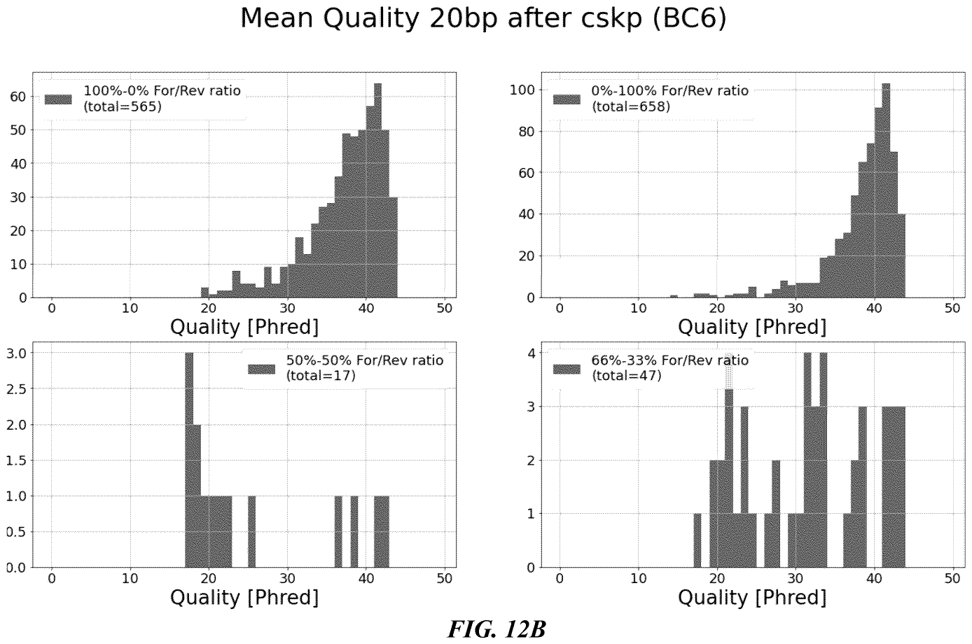

Additionally, the quality of the substitutions in 4 different forward-to-reverse cases (100%-0%, 0%-100%, 50%-50% and 66%-33% forward-reverse %) in the beads were examined and it was found that the quality behavior is markedly different. FIG. 12A shows a chart plotting (#of reads) vs (mean quality of the 20 bp read following cycle skip substitution) for the 100%-0%, 0%-100%, 50%-50%, and 66%-33% forward-reverse % beads in the top left, top right, bottom left, and bottom right panels, respectively, for the BC5 sample. FIG. 12B shows a chart plotting (#of reads) vs (mean quality of the 20 bp read following cycle skip substitution) for the 100%-0%, 0%-100%, 50%-50%, and 66%-33% forward-reverse % beads in the top left, top right, bottom left, and bottom right panels, respectively, for the BC6 sample.

In the BC5 sample data, shown in FIG. 12A, the mean quality of the 20 bp read following the substitution was found to be lower in the mixed forward-reverse % cases than in the unmixed (100%-0% or 0%-100% forward-reverse %) cases. Also, a stronger trend in the 50%-50% forward-reverse % case was observed compared to the 66%-33% forward-reverse % case. The BC6 sample data, shown in FIG. 12B, demonstrated similar results but with lower coverage compared to the BC5 sample data. It is noted that it is undetermined from this data whether the remaining substitutions are real substitutions.

This analysis demonstrates unexpected results of at least a 10-fold, and likely higher than 10-fold, lower error rate for mixed cases than unmixed cases.

### Example 3: Library Prep Error Correction

Preparing a library (“library prep”) from a nucleic acid sample may introduce artificial errors during various operations (e.g., excision, nicking, cleaving, gap filling, ligating, extending, etc.), which oftentimes manifest in only one strand of a double-stranded molecule. The methods described herein, such as sequencing material derived from both strands of a sample (paired plus-and-minus or paired forward-and-reverse), balanced strand confirmation or detection, and/or any other method, may be applied to library-prepped templates to beneficially generate error-corrected sequencing reads, reduce sequencing error rates (e.g., ruling out the single-strand artifactual mutations from being called as a true SNV), increase sequencing accuracy, obviate the need for correction steps, and save resources.

### Example 4: FFPE Artifact Correction

DNA extracted from formalin-fixed paraffin-embedded (FFPE) samples (e.g., tissue specimens) typically has many artifactual mutations, which are mostly manifested on only one strand of the extracted DNA. Various protocols used to improve DNA quality, such as excising and filling nicks, can introduce additional errors. The methods described herein, such as sequencing material derived from both strands of a sample (paired plus-and-minus or paired forward-and-reverse), balanced strand confirmation or detection, and/or any other method, may be applied to assaying DNA extracted from FFPE samples to beneficially generate error-corrected sequencing reads, reduce sequencing error rates (e.g., ruling out the single-strand artifactual mutations from being called as a true SNV), increase sequencing accuracy, obviate the need for correction steps, and save resources.

### Example 5: Single Cell WGS Error Correction

Reducing the error rates for whole genome sequencing (WGS) using the methods described herein, such as sequencing material derived from both strands of a sample (paired plus-and-minus or paired forward-and-reverse), balanced strand confirmation or detection, and/or any other method, to generate error-corrected WGS reads can permit the detection of somatic mutations from individual reads without requiring a high sequencing coverage (or without requiring a high sequencing depth) per cell, enabling the collection of more cells per experiment.

### Example 6: Error Correction of Environmental DNA (eDNA) and Ancient DNA (aDNA) Sequencing Data

Reducing the error rates for sequencing of eDNA and/or aDNA using the methods described herein, such as sequencing material derived from both strands of a sample (paired plus-and-minus or paired forward-and-reverse), balanced strand confirmation or detection, and/or any other method, can beneficially generate error-corrected sequencing reads, reduce sequencing error rates (e.g., ruling out the single-strand artifactual mutations from being called as a true SNV), increase sequencing accuracy, obviate the need for correction steps, and save resources.

### Example 7: Detection of Rare SNVs in MRD with Error Correction

Detection of circulating tumor DNA (ctDNA) in blood can help identify patients whose cancers are more likely to recur and monitor treatment efficacy based on detection and quantification of residual ctDNA before, during, and after treatment. Currently available clinical assays for residual disease or minimal residual disease (MRD) detection employ deep sequencing of a limited number of targeted mutations or methylation markers. An alternative approach, overcoming limited sample complexity and simplifying the clinical workflow, employs whole genome sequencing (WGS) to detect somatic mutations in cell-free DNA (cfDNA) across the genome. Proof of concept studies and statistical models suggest a limit of detection (LoD) of 10⁻⁵ could potentially be achieved using this approach, limited only by the affordable depth of WGS and the background error rate of the sequencing data—originating from both sample preparation and sequencing noise (See A. Zviran et al., Genome-wide cell-free DNA mutational integration enables ultra-sensitive cancer monitoring, Nat. Med., 2020). WGS characterization of tumor tissue also provides additional benefits, enabling both better biomarker characterization of structural and copy number variants and improved tumor burden estimations.

Novel mostly natural sequencing by synthesis and ppmSeq™ were found to enable detection of rare single nucleotide variants (SNVs) with high sensitivity and background error rates under 10⁻⁶. To demonstrate this, data from the following matched samples were analyzed: tumor tissue (i.e., fresh-frozen (FF) and formalin-fixed paraffin-embedded (FFPE)), normal tissue (i.e., peripheral blood mononuclear cells (PBMCs)), and plasma from 8 cancer patients and 2 negative plasma samples. All samples were sourced from the MIDGAM biobank (Israel National Biobank for Research; MID-116-2019). As used herein, the term ppmSeq™ may generally refer to sequencing of nucleic acid molecules that were prepared with any strand recognition adaptors described herein. As used herein, the term “ppmSeq™ library” may generally refer to a sample library prepared with any strand recognition adaptors described herein. As used herein, the term “ppmSeq™ reads” may generally refer to sequencing reads generated using ppmSeq™ sequencing.

### Methods

Genomic DNA extracted from patient-matched tumor and normal samples was sequenced for tumor tissue profiling. A somatic variant calling pipeline was used to determine patient-specific whole genome tumor profiles. These whole genome tumor profiles typically comprise on the order of 1,000s to 10,000s of individual SNV mutations (See M. S. Lawrence et al., Mutational heterogeneity in cancer and the search for new cancer-associated genes, Nature, 2013). To quantify circulating tumor fraction (CTF), cell-free DNA (cfDNA) was extracted from a patient-matched plasma sample and whole genome sequencing to a target depth of 30-100× was performed. CTF quantification was performed by counting the total number of reads at each relevant genomic location containing the tumor-specific mutations (SNVs) in the cfDNA data. By way of example, when CTF is low (e.g., <1%) each mutation is expected to appear in the cfDNA at most once, hence requiring single read SNV calling accuracy.

Mixed reads (e.g., ppmSeq reads) are derived from native PCR free library preparation, and pre-sequencing amplification on beads using ePCR. Thus, DNA from the matching forward and reverse strands are concurrently amplified on the same bead. Reads that are confirmed to contain both strands are called mixed reads (e.g., >40% of reads in the dataset). Using this method, mixed reads with sample errors that appear only in one strand are identified and rejected by the base-calling algorithm.

Both standard reads and mixed reads are generated by assaying with flow-based sequencing.

Samplepreparation and sequencing: FFPE DNA was extracted using RecoverAll Total Nucleic Acid Isolation Kit for FFPE (AB-AM1975; Invitrogen); FFPE was sheared using Covaris and treated by NEBNext FFPE DNA Repair (M6630S; NEB). Sequencing libraries were generated from 95-250 ng DNA using xGen Prism DNA Library Prep Kit (IDT). Fresh Frozen samples were extracted using AllPrep DNA/RNA Mini Kit (20-80204; Qiagen). PBMC DNA was extracted using DNeasy Blood & Tissue Kit (20-69504; Qiagen). PCR free sequencing libraries for FF and PBMC samples were generated from 200 ng DNA using KAPA HYPER-PLUS PCR-FREE (KK8513; Roche) after Covaris shearing.

The 16 tissue sample libraries (FF and FFPE) were sequenced to an average WGS coverage depth of 104× and the PBMC samples were sequenced to an average 79×. PBMC data was then downsampled to 40×.

SNV detection: Somatic SNVs were called by comparing each pair of matched tumor/normal samples using a UG-optimized DeepVariant pipeline. Detected variants were filtered with high stringency to generate a highly reliable profile for MRD detection (filtering criteria include: the variant is an SNV, is not biallelic, allele fraction is greater than 10%, SNVQ is greater than 10, is not in dbSNP or in GNOMAD with a frequency greater than 0.1%, and is not in empirical blacklist including regions with known low complexity). Summary statistics of tissue samples are listed in Table 4.

TABLE 4
Estimation of circulating tumor fraction in cfDNA samples.

| | | Tissue sample | | | | Normal sample (PBMC) | | | | |
|---|---|---|---|---|---|---|---|---|---|---|
| | | | | | Mean | | | Mean | Est. | |
| Sample | Tissue | Sample | | Mean | read | | Mean | read | Tumor | SNV |
| ID | type | type | # reads | coverage | length | # reads | coverage | length | fraction | count |
| PA_46 | Lung | FFPE | 3.2E+9 | 128.5 | 131.7 | 94.E+8 | 76.6 | 277.1 | 25% | 44,067 |
| | | FF | 1.3E+9 | 104.8 | 279.2 | | | | 35% | 41,565 |
| PA_47 | Lung | FFPE | 5.5E+9 | 236.5 | 144.2 | 8.5E+8 | 70.0 | 277.8 | 10% | 4,427 |
| | | FF | 1.4E+9 | 108.9 | 265.5 | | | | 5% | — |
| PA_67 | Bladder | FFPE | 2.9E+9 | 110.6 | 129.1 | 1.0E+9 | 85.0 | 280.5 | 10% | 6,647 |
| | | FF | 9.9E+8 | 81.7 | 281.0 | | | | 15% | 5,803 |
| PA_68 | Colorectal | FFPE | 2.9E+9 | 125.9 | 143.5 | 8.9E+8 | 73.0 | 277.5 | 45% | 4,401 |
| | cancer | FF | 1.1E+9 | 89.4 | 280.5 | | | | 4% | — |
| | (CRC) | | | | | | | | | |
| PA_69 | CRC | FFPE | 4.6E+9 | 33.5 | 170.4 | 8.7E+8 | 71.2 | 278.9 | 21% | 3,195 |
| | | FF | 9.1E+8 | 74.8 | 277.6 | | | | 24% | 4,982 |
| PA_70 | CRC | FFPE | 3.4E+9 | 137.8 | 138.9 | 9.0E+8 | 74.3 | 279.5 | 28% | 4,717 |
| | | FF | 1.5E+9 | 123.7 | 279.1 | | | | 11% | 3,916 |
| PA_71 | Lung | FFPE | 2.6E+9 | 90.8 | 119.7 | 8.8E+8 | 72.4 | 280.5 | 9% | 8,989 |
| | | FF | 9.4E+8 | 78.2 | 281.4 | | | | 17% | 17,568 |
| PA_75 | CRC | FFPE | 2.6E+9 | 95.5 | 124.8 | 8.9E+8 | 72.3 | 276.0 | 23% | 15,925 |
| | | FF | 1.7E+9 | 133.0 | 266.4 | | | | 14% | 12,937 |

Samplepreparation and sequencing: cfDNA was extracted from 4 mL of plasma using cfPure® V2 Cell-Free DNA Extraction Kit (PN: K5011610-V2, BioChain). For standard sequencing, libraries were generated from a range of 4.2 to 10 ng per sample of cfDNA using xGen Prism DNA Library Prep Kit (IDT) and sequenced to an average coverage depth of 138×. Between 3.7 and 20 ng of cfDNA were used to prepare ppmSeq libraries by KAPA HYPER-PLUS PCR-FREE (KK8513; Roche) that were then sequenced to an average coverage depth of 70×. Summary statistics of cfDNA samples are listed in Table 5.

TABLE 5
Summary of plasma sample sequencing

| | | Standard | | | Mixed | | |
|---|---|---|---|---|---|---|---|
| Sample | Cancer | | Mean | Mean read | | Mean | Mean read |
| ID | type | # reads | coverage | length | # reads | coverage | length |
| PA_46 | Lung | 2.9E+9 | 149.8 | 167.9 | 1.4E+9 | 67.1 | 168.7 |
| PA_47 | Lung | 3.2E+9 | 165.8 | 166.5 | 1.5E+9 | 70.3 | 168.6 |
| PA_67 | Bladder | 2.7E+9 | 146.4 | 172.2 | 5.3E+9 | 27.6 | 174.6 |
| PA_68 | CRC | 2.9E+9 | 153.7 | 170.3 | 1.7E+9 | 81.8 | 172.7 |
| PA_69 | CRC | 3.0E+9 | 150.3 | 162.7 | 1.4E+9 | 63.7 | 165.3 |
| PA_70 | CRC | 2.6E+9 | 130.9 | 159.9 | 1.4E+9 | 77.4 | 163.3 |
| PA_71 | Lung | 2.9E+9 | 149.7 | 167.9 | 1.1E+9 | 65.3 | 172.6 |
| PA_75 | CRC | 2.3E+9 | 117.2 | 161.8 | 1.9E+9 | 102.8 | 165.4 |
| PA_393 | Healthy | 3.7E+9 | 196.7 | 173.4 | 1.2E+9 | 71.5 | 181.6 |
| PA_396 | Healthy | 3.8E+9 | 199.2 | 171.5 | 4.3E+8 | 26.5 | 178.7 |

### SNV Quality Calculation

Circulating tumor fraction estimation: To estimate tumor fraction in each of the plasma samples, the cfDNA data was compared with the corresponding tumor tissue profile and reads covering each of the SNV positions were analyzed to determine whether it contained a detectable mutation.

First, cfDNA reads were filtered based on mapping quality (e.g., mapq greater than or equal to 60). Reads with high edit distance from the reference (e.g., distance greater than or equal to 10) and low quality (e.g., score less than or equal to 4) were also removed.

Then, each candidate SNV in each relevant read was scored for its predicted SNV quality (SNVQ) based on multiple sequence parameters (the specific base change, flow sequencing order, flow quality information, position in the read). For ppmSeq, additional information regarding estimated strand balance ratio was also incorporated into the score. The resulting SNVQ scores were recalibrated for the specific dataset to produce a score indicating the probability of the specific base change being incorrect. Note that the SNVQ score is different from a standard base quality (BQ) score since it represents the error probability of the specific base substitution (e.g. A>G) rather than the error probability of any substitution (A>C/G/T).

Finally, circulating tumor fraction (CTF) was calculated by totaling the number of detected tumor profile SNVs passing a quality threshold. Median SNVQ was selected for each sample. On average, SNVQ was greater than 56.2 for standard sequencing and greater than 62.2 for ppmSeq. The CTF value for each sample was then normalized for the assessed coverage at each of the profile positions:

$$CTF = ( total \# of detected SNV s ) / ( total coverage at candidate SNV positions ) .$$

### Results

Somatic SNV detection: Somatic SNV profiles were generated by comparing the data from matched tumor/normal samples using DeepVariant optimized for flow sequencing. To assess the tumor fraction in each sample, SNV profiles of FF and FFPE were compared, and the bin with the highest number of common variants was identified. According to this estimation, two of the FF samples had insufficient tumor content (i.e., less than 5% tumor reads) and were discarded from the analysis. For the remaining 14 out of the 16 tissue samples, patient-specific mutation profiles with between 3,000 and 45,000 SNVs were detected.

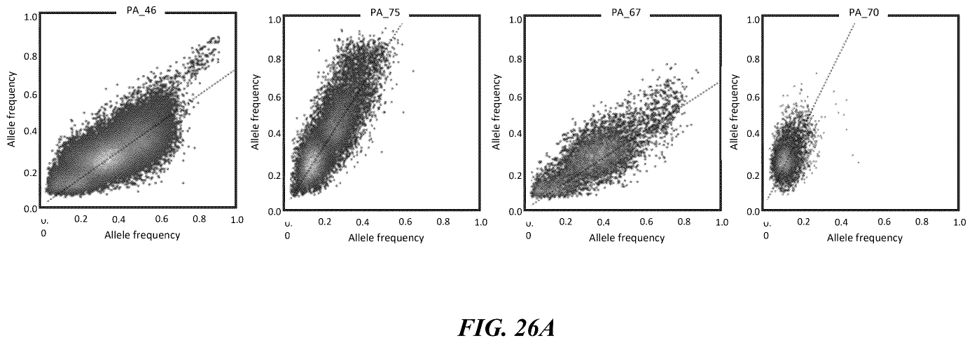

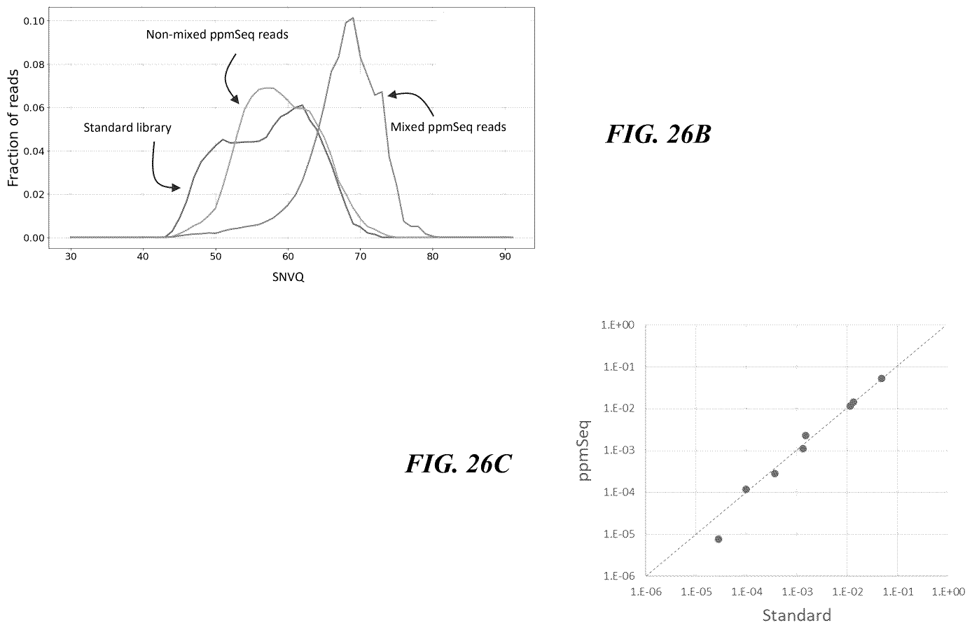

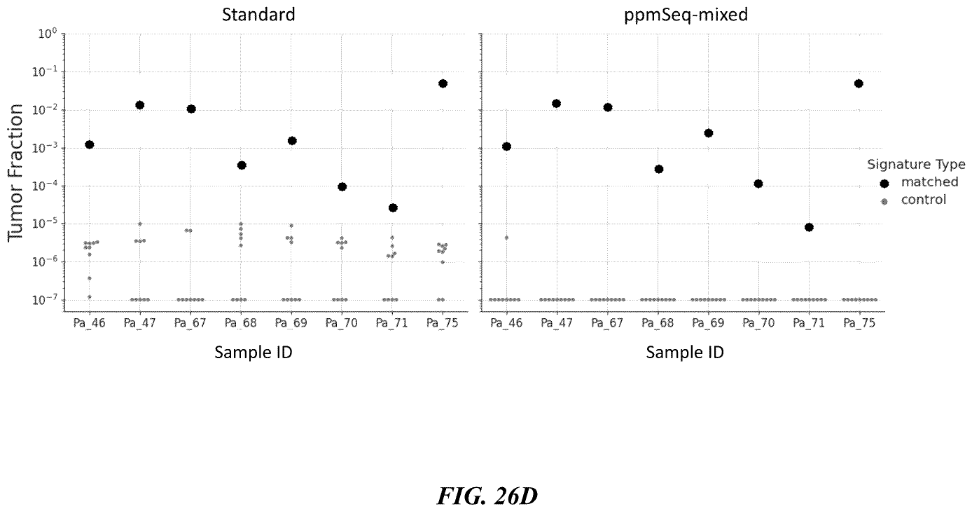

FIG. 26A depicts several examples of the comparison of SNV profiles detected in matched FF and FFPE samples.

Estimation of circulating tumor fraction (CTF): To assess WGS-based tissue informed MRD detection, the CTF was quantified using the 8 patient-specific profiles in the matched plasma samples. As a negative control, the 7 unmatched plasma samples and 2 negative controls samples were assayed for the same SNV profiles, yielding a total of 72 negative control tests. The full assessment was repeated for both standard cfDNA sample preparation and ppmSeq.

For each cfDNA/tissue profile combination, SNVQ quality values were calculated for all SNVs (single read substitution events) detected in the dataset. As shown in FIG. 26B, standard libraries exhibited a similar SNVQ distribution to that of non-mixed ppmSeq library reads. Reads obtained from mixed reads in ppmSeq libraries had, overall, a higher SNVQ and a narrower SNVQ distribution.

CTF values assessed in the 8 cfDNA values were highly correlated between the standard cfDNA and ppmSeq assays, as shown in FIG. 26C (plotting ppmSeq vs. Standard assay CTF values).

Results for estimation of CTF for the standard cfDNA assay and for ppmSeq are depicted in FIG. 26D: estimated tumor fraction in matched and control cfDNA samples, for standard cfDNA prep (left) and ppmSeq (right). Each column in each graph of FIG. 26D corresponds to one patient SNV profile, with one matched cfDNA sample and 9 negative control samples. A positive CTF in the range of 0.001%-5% was detected in each of the samples, with all negative control samples showing CTF below 0.001% (i.e., 10⁻⁵). Remarkably and surprisingly, for ppmSeq only a single positive read was detected in all of the negative control samples assessed (˜10M reads) demonstrating the capability of this method to reach background error rates below 10⁻⁶.

Summary: As demonstrated above, flow sequencing methods can generate broad, patient-specific tumor profiles by WGS of DNA from fresh-frozen and FFPE tumor tissue samples. These flow sequencing results accurately quantify circulating tumor fraction by WGS in cell-free DNA from matched plasma samples. While the background SNV error rate of raw flow sequencing data is estimated to be less than 10⁻⁵, the source of this error has been shown to be dominated by errors introduced during sample preparation (e.g., PCR errors). The novel ppmSeq method can reduce the observed error rate to less than 10⁻⁶. This is due to the concurrent amplification of DNA from matching forward and reverse strands on the same sequencing bead (e.g., within the same sequencing cluster), which can be used to eliminate errors that do not appear on both strands.

Due to its unique performance, in cases where tumor input and SNV profile are sufficient, PCR-free WGS with ppmSeq may allow detection of CTF with a limit of detection of less than 10⁻⁵. Advantageously, ppmSeq further does not require the high sequence redundancy needed for duplex sequencing. Standard cfDNA whole genome sequencing at high coverage allows detection of CTF at a range of 10⁻⁴ to 10⁻⁵ in cases where cfDNA input is a limiting factor. The methodology described above provides a path to develop simple and affordable cancer monitoring assays with the high analytical sensitivity required for clinical applications such as early detection and MRD.

### Example 8: Error Rates with Strand Recognition Error Correction

Samples were prepared for sequencing according to the general workflow described with respect to FIGS. 10A-10B. The resulting amplified beads were sequenced, according to methods described elsewhere herein. Sequencing reads for the beads were generated from the sequencing run. Table 6 summarizes the error rates calculated for each of beads with mixed strands that have both forward and reverse strands vs beads with non-mixed strands.

TABLE 6
Error Rate Comparison

| | Mixed | Non-mixed |
|---|---|---|
| | strands | strands |
| Median error rate [cycle-shift SNVs] | 1.8 E−6 | 1.7 E−5 |
| Median error rate [all SNVs] | 2.7 E−6 | 1.8 E−5 |

As seen in Table 4, compared to non-mixed strand reads, mixed strand reads have a significantly lower error rate for any metric, including an improvement by an order of magnitude for median error rate for cycle-shift SNV errors and median error rate for all SNV errors, confirming that the methods described herein for retaining double strand information for sequencing improves sequencing accuracy.

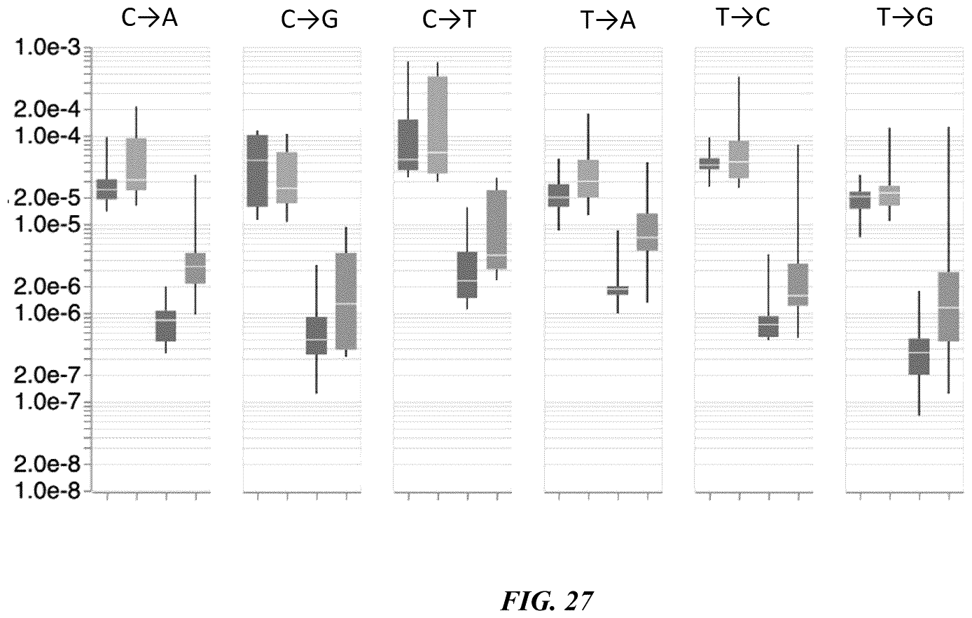

Separately, FIG. 27 shows a residual error rate distribution comparison between mixed strand (or balanced strand) and non-mixed strand (or non-balanced strand) sequencing for different mutations, where the y-axis shows the error rates. The sequencing assay was conducted on different samples than the ones used to generate the data for Table 4. In each graph, from left to right, the four bars represent (1) non-mixed strand & cycle skip error, (2) non-mixed strand & non-cycle skip error, (3) mixed strand & cycle skip error, and (4) mixed strand & non-cycle skip errors. Across all types mutations plotted (C->A; C->G; C->T; T->A; T->C; T->G), it can be seen that consistently the error rates for cases (3) and (4), mixed strand cycle skip and non-cycle skip errors, respectively, have lower error rates (by an order of magnitude), than for cases (1) and (2), non-mixed strand cycle skip and non-cycle skip errors, respectively.

The methods provided herein demonstrated a surprising order of magnitude reduction in sequencing error rates.

### Example 9: Strand Recognition Adaptor-Ligated Templates

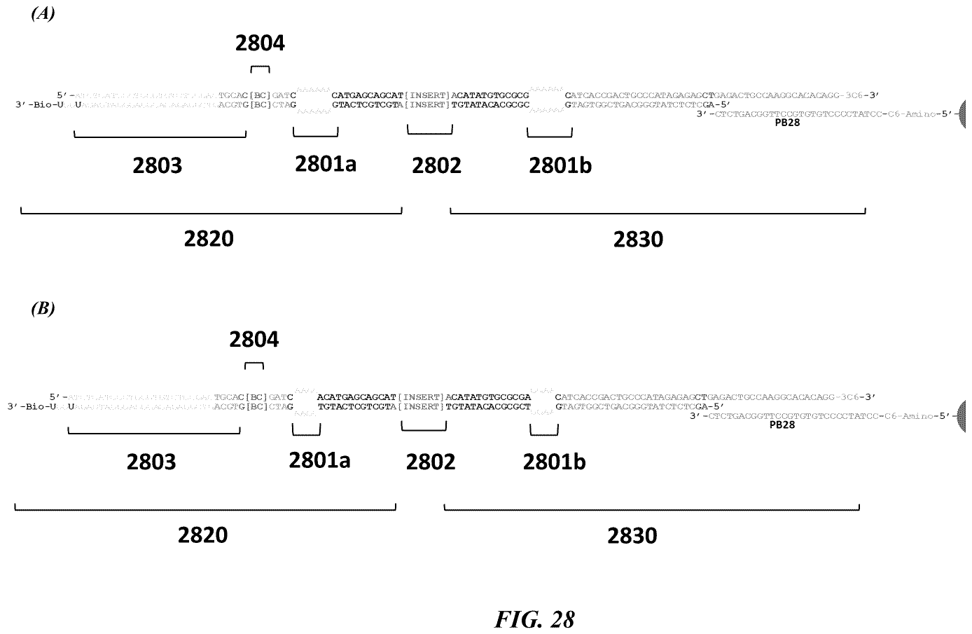

FIG. 28 illustrates two examples, (A) and (B), of strand recognition adaptor-ligated template insert molecules. For example, the strand recognition adaptor in (A) exemplifies Table 1 adaptor molecules and the strand recognition adaptor in (B) exemplifies Table 1 adaptor molecules, with a [BC] barcode sequence in the sense strand.

In both (A) and (B), a template insert molecule 2802 is ligated to two strand recognition adaptors, a first adaptor 2820 and a second adaptor 2830, each of the first and second adaptors comprising a mismatch portion 2801a-b. The first adaptor 2820 may comprise, among other sequence portions, an adaptor sequence portion 2803, a barcode sequence portion 2804, a mismatch portion 2801a, and a second adaptor 2830 may comprise, among other sequence portions, a mismatch portion 2801b. The second adaptor 2830 may comprise an overhang sequence configured to hybridize to a support primer sequence (illustrated). In (A), the first adaptor 2820 and the second adaptor 2830 comprises a looped mismatch portion 2801a-b, respectively, each comprising mismatch sequence pair: AAAAAA/AAAAAA. In (B), the first adaptor 2820 and the second adaptor 2830 comprises a looped mismatch portion 2801a-b, respectively, each comprising mismatch sequence pair: AAGG/AAGG.

While this example illustrates that a template insert molecule (e.g., 2802) is ligated to two strand recognition adaptors, one on each end, it will be appreciated that a template insert molecule may be ligated to only one strand recognition adaptor, as described in various methods and workflows described herein. In some cases, attaching a strand recognition adaptor to both ends of the template insert molecule may allow for strand recognition and/or quantification when reading from either end, which may be helpful for paired end sequencing workflows. In some cases, one or both strand recognition sequences may be identified when an entire strand sequence is sequenced through to the end with sufficient sequencing quality for additional verification.

### NUMBERED EMBODIMENTS

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

1. A method for high accuracy sequencing, comprising: (a) providing an amplified cluster of a plurality of nucleic acid molecules derived from a double-stranded nucleic acid molecule which comprises a first strand and a second strand, wherein a first subset of the plurality of nucleic acid molecules each comprises a first sequence that is a copy of at least a portion of the first strand sequence and wherein a second subset of the plurality of nucleic acid molecules each comprises a second sequence that is a reverse complement copy of at least a portion of the second strand sequence; (b) collecting sequencing signals from the amplified cluster to determine a disagreement between the first sequence and the second sequence at a locus; and (c) exclemvuding the locus from single nucleotide variant (SNV) or single nucleotide polymorphism (SNP) calling based at least in part on the disagreement. 2. The method of embodiment 1, wherein the sequencing signals from the amplified cluster are collected in (b) by hybridizing sequencing primers to the plurality of nucleic acid molecules and simultaneously interrogating both the first subset and the second subset with a same set of nucleotide mixtures. 3. The method of embodiment 2, wherein the sequencing primers hybridized to the first subset and the second subset comprise a same sequence. 4. The method of embodiment 2, wherein the sequencing primers hybridized to the first subset and the second subset comprise different sequences. 5. The method of any one of embodiments 1-4, wherein in (b) a same nucleotide mixture interrogates a same locus between the first sequence and the second sequence. 6. The method of embodiment 5, wherein the same locus has a length of 1 base position. 7. The method of embodiment 5, wherein the same locus has a length of at least 2 base positions. 8. The method of any one of embodiments 1-7, wherein the same set of nucleotide mixtures interrogates a same locus between the first sequence and the second sequence. 9. The method of embodiment 8, wherein the same locus has a length of 1 base position. 10. The method of embodiment 8, wherein the same locus has a length of at least 2 base positions. 11. The method of any one of embodiments 1-10, wherein (b) comprises simultaneously generating sequencing data from the first subset and the second subset. 12. The method of embodiment 1, wherein the sequencing signals from the amplified cluster are collected in (b) by (i) hybridizing a first set of sequencing primers to the first subset of the plurality of nucleic acid molecules and interrogating the first subset with a first set of nucleotide mixtures, and (ii) hybridizing a second set of sequencing primers to the second subset of the plurality of nucleic acid molecules and interrogating the second subset with a second set of nucleotide mixtures that is different from the first set of nucleotide mixtures. 13. The method of embodiment 11, wherein the first set of sequencing primers and the second set of sequencing primers comprise different sequences. 14. The method of embodiment 11, wherein the first set of sequencing primers and the second set of sequencing primers have the same sequence. 15. The method of embodiments 11-14, wherein (b) comprises generating sequencing data from the first subset and the second subset at different timepoints. 16. The method of any one of embodiments 1-15, further comprising generating a single sequencing read from the amplified cluster, wherein the single sequencing read is generated by interrogating both the first subset and the second subset of the plurality of nucleic acid molecules. 17. The method of any one of embodiments 1-15, further comprising generating two sequencing reads from the amplified cluster, a first sequencing read generated by interrogating the first subset of the plurality of nucleic acid molecules and a second sequencing read generated by interrogating the second subset of the plurality of nucleic acid molecules. 18. The method of any one of embodiments 1-17, further comprising generating at least two candidate base calls for the locus at which the disagreement is determined. 19. The method of embodiment 18, further comprising comparing the at least two candidate base calls to the locus in a reference sequence and selecting one of the at least two candidate base calls or a base at the locus in the reference sequence for a consensus read. 20. The method of any one of embodiments 1-19, further comprising generating a sequencing read for the amplified cluster using the sequencing signals. 21. The method of embodiment 19, further comprising aligning the sequencing read to the reference sequence. 22. The method of embodiment 21, further comprising calling one or more SNVs or SNPs for a sample or subject that the double-stranded nucleic acid molecule is derived from. 23. The method of any one of embodiments 1-22, wherein the first subset each further comprises a first strand recognition element and wherein the second subset each further comprises a second strand recognition element is different from the first strand recognition element. 24. The method of embodiment 23, wherein the first strand recognition element and the second strand recognition element are different sequences. 25. The method of embodiment 24, wherein the different sequences are different homopolymer sequences. 26. The method of embodiment 24, wherein the different sequences are different non-homopolymer sequences. 27. The method of any one of embodiments 24-26, wherein the different sequences have different lengths. 28. The method of any one of embodiments 24-26, wherein the different sequences have the same length. 29. The method of embodiment 28, wherein the different sequences each have a single base length. 30. The method of any one of embodiments 24-28, wherein the different sequences each have at least 1 base lengths. 31. The method of any one of embodiments 24-30, wherein the different sequences each have at least 3 base lengths. 32. The method of any one of embodiments 24-31, wherein the different sequences each have at least 5 base lengths. 33. The method of embodiment 23, wherein the first strand recognition element and the second strand recognition element are not nucleic acid sequences. 34. The method of any one of embodiments 23-33, further comprising detecting a presence of one or both of the first strand recognition element and the second strand recognition element in the amplified cluster. 35. The method of embodiment 34, wherein the detecting comprises sequencing the plurality of nucleic acid molecules to identify a sequence of the first strand recognition element, the second strand recognition element, or both. 36. The method of embodiment 34, wherein the detecting comprises sequencing the plurality of nucleic acid molecules to identify a disagreement in sequence in a common portion of the plurality of nucleic acid molecules that comprises the first strand recognition element or the second strand recognition element. 37. The method of embodiment 34, wherein the detecting comprises hybridizing labeled oligonucleotide probes to the first strand recognition element, the second strand recognition element, or both, and detecting signals from the labeled oligonucleotide probes. 38. The method of embodiment 34, further comprising determining a ratio of the first subset and the second subset in the plurality of nucleic acid molecules. 39. The method of embodiment 38, wherein the ratio is determined by processing signal intensities collected from interrogating the first strand recognition element, the second strand recognition element, or both. 40. The method of any one of embodiment 38-39, further comprising generating a sequencing read for the amplified cluster, wherein the sequencing read is generated based at least in part on the sequencing signals and the ratio. 41. The method of any one of embodiments 1-39, wherein the amplified cluster is immobilized to an individually addressable location on a substrate. 42. The method of embodiment 41, wherein the substrate comprises at least 1,000,000 individually addressable locations. 43. The method of embodiment 42, wherein the substrate comprises at least 1,000,000,000 individually addressable locations. 44. The method of embodiment 43, wherein the substrate comprises at least 5,000,000,000 individually addressable locations. 45. The method of embodiment 44, wherein the substrate comprises at least 10,000,000,000 individually addressable locations. 46. The method of embodiment 45, wherein the substrate comprises at least 20,000,000,000 individually addressable locations. 47. The method of any one of embodiments 41-46, wherein the substrate is substantially planar. 48. The method of any one of embodiments 41-47, wherein the substrate is textured or patterned. 49. The method of any one of embodiments 41-47, wherein the substrate is unpatterned. 50. The method of any one of embodiments 41-49, wherein the substrate comprises a layer of aminosilane that immobilizes the amplified cluster.

51. The method of any one of embodiments 41-49, wherein the substrate comprises a layer of surface primers that immobilizes the amplified cluster. 52. The method of any one of embodiments 41-51, wherein the plurality of nucleic acid molecules are coupled to a bead, which bead is immobilized to the individually addressable location on the substrate. 53. The method of any one of embodiments 41-52, wherein the substrate is rotated during sequencing of the plurality of nucleic acid molecules. 54. The method of any one of embodiments 1-53, wherein the plurality of nucleic acid molecules are single-stranded molecules.

55. A method for high accuracy sequencing, comprising: (a) providing an amplified cluster of a plurality of nucleic acid molecules derived from a double-stranded nucleic acid molecule which comprises a first strand and a second strand, wherein a first subset of the plurality of nucleic acid molecules each comprises a first strand recognition element and a first sequence that is a copy of at least a portion of the first strand sequence, and wherein a second subset of the plurality of nucleic acid molecules each comprises a second strand recognition element and a second sequence that is a reverse complement copy of at least a portion of the second strand sequence, wherein the first strand recognition element and the second strand recognition elements are different; and (b) detecting a presence of the first strand recognition element, the second strand recognition element, or both in the amplified cluster. 56. The method of embodiment 55, wherein the plurality of nucleic acid molecules are immobilized to a support. 57. The method of any one of embodiments 55-56, further comprising subjecting the double-stranded nucleic acid molecule to amplification to generate the plurality of nucleic acid molecules. 58. The method of embodiment 57, wherein the amplification comprises PCR, emulsion PCR (ePCR), recombinase polymerase amplification (RPA), emulsion RPA (eRPA), rolling circle amplification (RCA), multiple displacement amplification (MDA), bridge amplification, or a combination thereof. 59. The method of any one of embodiments 57-58, further comprising, prior to the amplification, ligating a strand recognition adapter comprising a pair of mismatch sequences to the double-stranded nucleic acid molecule. 60. The method of any one of embodiments 57-58, wherein double-stranded nucleic acid molecule is amplified by (1) ligating a hairpin adapter to each end to generate a dumbbell molecule, and subjecting the dumbbell molecule to rolling circle amplification (RCA) to generate a first amplification product, (2) cleaving or digesting portions of the first amplification product corresponding to the hairpin adapter to generate a plurality of copy molecules each comprising a copy of the first strand the second strand. 61. The method of any one of embodiments 57-58, wherein double-stranded nucleic acid molecule is amplified by (1) ligating a hairpin adapter to each end to generate a dumbbell molecule, and subjecting the dumbbell molecule to rolling circle amplification (RCA) by contacting the dumbbell molecule with a plurality of random primers and dNTPs comprising dUTPs but not dTTPs to generate first amplification products, (2) hybridizing second primers to the first amplification products in the presence of dNTPs comprising dTTPs but not dUTPs to generate second amplification products, (3) degrading the first amplification products based on uracil residues, to isolate second amplification products, and (4) cleaving or digesting portions of the second amplification product corresponding to the hairpin adapter to generate a plurality of copy molecules each comprising a copy of the first strand the second strand. 62. The method of any one of embodiments 57-58, wherein double-stranded nucleic acid molecule is amplified by (1) ligating a hairpin adapter to each end to generate a dumbbell molecule, and subjecting the dumbbell molecule to rolling circle amplification (RCA) to generate a first amplification product, (2) hybridizing second primers to the first amplification product under suppressed strand displacement conditions to generate a plurality of second amplification products each comprising a single copy of the first strand and the second strand. 63. The method of any one of embodiments 60-61, further comprising ligating a strand recognition adapter comprising a pair of mismatch sequences to each of the plurality of copy molecules to generate the plurality of nucleic acid molecules. 64. The method of any one of embodiments 60-62, wherein at least one of the hairpin adapter comprises a strand recognition adapter comprising a pair of mismatch sequences, and wherein the plurality of copy molecules each comprises a copy of the pair of mismatch sequences.

65. A method for detecting amplified strands on a support, comprising: (a) attaching a double-stranded template molecule to a support to generate a template-attached support, wherein the double-stranded template molecule comprises a first strand and a second strand, wherein the double-stranded template molecule comprises an adaptor comprising a mismatch portion, wherein the mismatch portion comprises in the first strand a first mismatch sequence and in the second strand a second mismatch sequence that is not complementary to the first mismatch sequence; (b) subjecting the double-stranded template molecule of the template-attached support to amplification to generate an amplified support comprising a plurality of amplified strands attached thereto; (c) sequencing the amplified support to generate a sequencing read; and (d) based at least in part on a portion of the sequencing read that corresponds to the mismatch portion, determining a percentage of amplified strands in the plurality of amplified strands in the amplified support that derives from the first strand.

66. A method for detecting amplified strands on a support, comprising: (a) attaching a double-stranded template molecule to a support to generate a template-attached support, wherein the double-stranded template molecule comprises a first strand and a second strand, wherein the double-stranded template molecule comprises an adaptor comprising a mismatch portion, wherein the mismatch portion comprises in the first strand a first homopolymer sequence and in the second strand a second homopolymer sequence that is not complementary to the first homopolymer sequence; (b) subjecting the double-stranded template molecule of the template-attached support to amplification to generate an amplified support comprising a plurality of amplified strands attached thereto; (c) sequencing the amplified support to generate a sequencing read; and (d) based at least in part on a portion of the sequencing read that corresponds to the mismatch portion, determining a percentage of amplified strands in the plurality of amplified strands in the amplified support that derives from the first strand.

67. A kit, comprising: a double-stranded adaptor comprising a mismatch portion, wherein the double-stranded adaptor comprises a first strand and a second strand, wherein the mismatch portion comprises in the first strand a first mismatch sequence and in the second strand a second mismatch sequence that is not complementary to the first mismatch sequence. 68. The kit of embodiment 67, further comprising a plurality of double-stranded adaptor comprising the mismatch portion, wherein the mismatch portion of the plurality of double-stranded adaptor is identical.

69. A kit, comprising: a double-stranded adaptor comprising a mismatch portion, wherein the double-stranded adaptor comprises a first strand and a second strand, wherein the mismatch portion comprises in the first strand a first homopolymer sequence and in the second strand a second homopolymer sequence that is not complementary to the first homopolymer sequence. 70. The kit of embodiment 69, further comprising a plurality of double-stranded adaptor comprising the mismatch portion, wherein the mismatch portion of the plurality of double-stranded adaptor is identical.

71. A composition, comprising: a double-stranded adaptor comprising a mismatch portion, wherein the double-stranded adaptor comprises a first strand and a second strand, wherein the mismatch portion comprises in the first strand a first mismatch sequence and in the second strand a second mismatch sequence that is not complementary to the first mismatch sequence. 72. The composition of embodiment 71, further comprising a plurality of double-stranded adaptors each comprising the mismatch portion, wherein the mismatch portion of the plurality of double-stranded adaptor is identical. 73. The composition of embodiment 72, further comprising a plurality of template molecules, wherein the plurality of template molecules comprises a plurality of double-stranded template insert molecules ligated to the plurality of double-stranded adaptors. 74. The composition of embodiment 71, further comprising a template molecule, wherein the template molecule comprises a double-stranded template insert molecule ligated to the double-stranded adaptor. 75. The composition of embodiment 74, further comprising a support. 76. The composition of embodiment 75, wherein the support is attached to the template molecule. 77. The composition of embodiment 71, further comprising a support.

78. A composition, comprising: a double-stranded adaptor comprising a mismatch portion, wherein the double-stranded adaptor comprises a first strand and a second strand, wherein the mismatch portion comprises in the first strand a first homopolymer sequence and in the second strand a second homopolymer sequence that is not complementary to the first homopolymer sequence. 79. The composition of embodiment 78, further comprising a plurality of double-stranded adaptors each comprising the mismatch portion, wherein the mismatch portion of the plurality of double-stranded adaptor is identical.

80. A method for enrichment, comprising: (a) providing a plurality of balanced supports which comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first enrichment molecules to the plurality of amplified strands, wherein the plurality of first enrichment molecules comprises a first capture sequence complementary to the first mismatch sequence, to generate a first set of enriched balanced supports; and (c) contacting a plurality of second enrichment molecules to the first set of enriched balanced supports, wherein the plurality of second enrichment molecules comprises a second capture sequence comprising the second mismatch sequence, to generate a second set of enriched balanced supports.

81. A method for enrichment, comprising: (a) providing a plurality of balanced supports which comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first enrichment molecules to the plurality of amplified strands, wherein the plurality of first enrichment molecules comprises a first capture sequence comprising the second mismatch sequence, to generate a first set of enriched balanced supports comprising the second mismatch sequence; and (c) contacting a plurality of second enrichment molecules to the first set of enriched balanced supports, wherein the plurality of second enrichment molecules comprises a second capture sequence complementary to the first mismatch sequence, to generate a second set of enriched balanced supports.

82. A method for enrichment, comprising: (a) providing a plurality of balanced supports which comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first enrichment molecules to the plurality of amplified strands, wherein the plurality of first enrichment molecules comprises a first capture sequence complementary to the first mismatch sequence, to capture a subset of balanced supports; and (c) removing at least a subset of the subset of balanced supports from the plurality of plurality of balanced supports to generate a first set of enriched, balanced supports.

83. A method for enrichment, comprising: (a) providing a plurality of balanced supports which comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first enrichment molecules to the plurality of amplified strands, wherein the plurality of first enrichment molecules comprises a first capture sequence comprising the second mismatch sequence, to capture a subset of balanced supports; and (c) removing at least a subset of the subset of balanced supports from the plurality of plurality of balanced supports to generate a first set of enriched, balanced supports.

84. A method for generating an amplified support with a predetermined forward-reverse strand ratio, comprising: contacting (i) a template-attached support, wherein the template-attached support comprise a support attached to a double-stranded template molecule, wherein the double-stranded template molecule comprises a first strand and a second strand, wherein the double-stranded template molecule comprises an adaptor comprising a mismatch portion, wherein the mismatch portion comprises in the first strand a first mismatch sequence and in the second strand a second mismatch sequence that is not complementary to the first mismatch sequence, (ii) a plurality of forward amplification primers at a first predetermined concentration or comprising a first annealing temperature with the first mismatch sequence, wherein each forward amplification primer of the plurality of forward amplification primers comprises a reverse complement of the first mismatch sequence to hybridize to first amplified strands derived from the first strand, and (iii) a plurality of reverse amplification primers at a second predetermined concentration or comprising a second annealing temperature with the reverse complement of the second mismatch sequence, wherein each reverse amplification primer of the plurality of reverse amplification primers comprises the second mismatch sequence to hybridize to second amplified strands derived from the second strand, to generate the amplified support comprising a plurality of amplified strands with the predetermined forward-reverse strand ratio.

85. A method for detecting strands on a support, comprising: (a) providing a plurality of balanced supports immobilized to a substrate, wherein the plurality of balanced supports comprises a plurality of amplified strands, wherein the plurality of amplified strands are derived from a plurality of double-stranded template nucleic acid molecules each comprising a mismatch portion, wherein the mismatch portion comprises a first strand comprising a first mismatch sequence and a second strand comprising a second mismatch sequence that is not complementary to the first mismatch sequence, such that each of the plurality of amplified strands comprises at a respective portion corresponding to the mismatch portion either the first mismatch sequence or a reverse complement of the second mismatch sequence; (b) contacting a plurality of first type of probes to the plurality of amplified strands, wherein the plurality of first type of probes each comprises a first capture sequence complementary to the first mismatch sequence and a first detectable moiety; and (c) detecting first signals from the first detectable moiety of a subset of the plurality of first type of probes bound to a subset of the plurality of amplified strands. 86. The method of embodiment 85, further comprising: (d) contacting a plurality of second type of probes to the plurality of amplified strands, wherein the plurality of second type of probes each comprises a second capture sequence comprising the second mismatch sequence and a second detectable moiety; and (e) detecting second signals from the second detectable moiety of a subset of the plurality of second type of probes bound to a second subset of the plurality of amplified strands.

87. A method of amplification, comprising: (a) providing a double-stranded target molecule and a plurality of adaptors, wherein the double-stranded target molecule comprises a first strand and a second strand and at least one adaptor of the plurality of adaptors comprises a hairpin sequence; (b) exposing the double-stranded target molecule and the plurality of adaptors to conditions sufficient to attach an adaptor to each end of the double-stranded target molecule, thereby generating a double-stranded template-adaptor molecule, wherein at least one attached adaptor comprises a hairpin sequence; and (c) subjecting the double-stranded template-adaptor molecule to amplification to generate a plurality of copies of the double-stranded template-adaptor molecule, wherein each copy of the double-stranded template-adaptor molecule comprises a copy of the sequence of the first strand and a copy of the sequence of the second strand. 88. The method of embodiment 87, wherein the amplification comprises rolling circle amplification (RCA), and the plurality of copies of the double-stranded template-adaptor molecule is attached to each other. 89. The method of embodiment 87, wherein the amplification comprises PCR. 90. The method of embodiment 87, wherein the amplification comprises loop-mediated isothermal amplification (LAMP). 91. The method of embodiment 87, wherein, the at least one adaptor further comprises a first region and a second region that do not have sequence complementarity, wherein the first and second regions are distal from the double-stranded template molecule. 92. The method of embodiment 91, wherein the double-stranded template-adapter molecule is attached to a support, wherein the support comprises a plurality of primers, wherein a first subset of the primers has sequence complementarity to the first region and a second subset of the primers have sequence complementarity to the second region.

93. A method of sequencing, comprising: (a) providing a double-stranded template molecule comprising, a first strand and a second strand with sequence complementarity to each other and at least one adaptor region comprising a single-stranded hairpin region; (b) annealing a primer to the single-stranded hairpin region; (c) extending the primer to generate a partially single-stranded template molecule, comprising a double-stranded region and a single-stranded region; (d) processing the partially single-stranded template molecule to generate a single-stranded template molecule; and (e) sequencing the single-stranded template molecule. 94. The method of sequencing of embodiment 93, wherein: processing (d) the partially single-stranded template molecule comprises filtering based on the sequence of the single-stranded region; and sequencing (e) comprises targeted sequencing. 95. The method of sequencing of embodiment 93, wherein: processing (d) the partially single-stranded template molecule comprises methylation conversion of the single-stranded region; and sequencing (e) comprises methylation sequencing.

96. A method for sequencing, comprising: (a) providing a balanced construct comprising a mixture of a forward strand and a reverse strand, wherein (i) the forward strand comprises a first sequence that is identical to or a reverse complement of a first strand of a double-stranded template molecule of a sample, and (ii) the reverse strand comprises a second sequence that is a reverse complement of or identical to a methylation-converted sequence of a second strand of the double-stranded template molecule of the sample, respectively; and (b) sequencing the forward strand and the reverse strand by: (i) hybridizing primers to the forward strand and the reverse strand, respectively, (ii) extending the primers with nucleotides in nucleotide flows provided according to a repeating flow order, wherein a nucleotide flow comprises nucleotides of a single canonical base type, wherein the repeating flow order comprises a consecutive three flow order of a thymine-base flow, cytosine-base flow, and thymine-base flow, and (iii) detecting flow signals indicative of incorporation of nucleotides, or lack thereof, by the primers subsequent to each respective nucleotide flows. 97. The method of embodiment 96, further comprising using the flow signals detected in (b)(iii) to determine a methylation status of the double-stranded template molecule. 98. The method of embodiment 96, wherein the forward strand comprises a first strand recognition element comprising a first homopolymer sequence and wherein the reverse strand comprises a second strand recognition element comprising a second homopolymer sequence, wherein the first homopolymer sequence and the second homopolymer sequence comprise different bases. 99. The method of embodiment 98, further comprising using a subset of the flow signals detected in (b)(iii) which corresponds to the first strand recognition element and the second strand recognition element to determine a forward-reverse ratio of a number of copies of the forward strand and a number of copies of the reverse strand on the balance construct. 100. The method of embodiment 99, further comprising determining a methylation status of the double-stranded template molecule based at least in part on the forward-reverse ratio. 101. The method of embodiment 22, further comprising detecting a minimal residual disease (MRD), tumor fraction, or circulating tumor fraction in the sample or the subject based on the one or more SNVs or SNPs called.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for high accuracy sequencing, comprising:
(a) providing an amplified cluster of a plurality of nucleic acid molecules derived from a double-stranded nucleic acid molecule, which comprises a first strand and a second strand, wherein a first subset of the plurality of nucleic acid molecules each comprises a first sequence that is a copy of at least a portion of the first strand sequence and wherein a second subset of the plurality of nucleic acid molecules each comprises a second sequence that is a reverse complement copy of at least a portion of the second strand sequence;
(b) collecting sequencing signals from the amplified cluster to determine a disagreement between the first sequence and the second sequence at a locus; and
(c) excluding the locus from single nucleotide variant (SNV) or single nucleotide polymorphism (SNP) calling based at least in part on the disagreement.

2. The method of claim 1, wherein the sequencing signals from the amplified cluster are collected in (b) by hybridizing sequencing primers to the plurality of nucleic acid molecules and simultaneously interrogating both the first subset and the second subset with a same set of nucleotide mixtures.

3. The method of claim 2, wherein the sequencing primers hybridized to the first subset and the second subset comprise a same sequence.

4. The method of claim 2, wherein the sequencing primers hybridized to the first subset and the second subset comprise different sequences.

5. The method of claim 1, wherein in (b) a same nucleotide mixture interrogates a same locus between the first sequence and the second sequence.

6. The method of claim 5, wherein the same locus has a length of 1 base position.

7. The method of claim 5, wherein the same locus has a length of at least 2 base positions.

8.-10. (canceled)

11. The method of claim 1, wherein (b) comprises simultaneously generating sequencing data from the first subset and the second subset.

12.-15. (canceled)

16. The method of claim 1, further comprising generating a single sequencing read from the amplified cluster, wherein the single sequencing read is generated by interrogating both the first subset and the second subset of the plurality of nucleic acid molecules.

17. (canceled)

18. The method of claim 1, further comprising generating at least two candidate base calls for the locus at which the disagreement is determined.

19. The method of claim 18, further comprising comparing the at least two candidate base calls to the locus in a reference sequence and selecting one of the at least two candidate base calls or a base at the locus in the reference sequence for a consensus read.

20. The method of claim 1, further comprising generating a sequencing read for the amplified cluster using the sequencing signals.

21. The method of claim 19, further comprising aligning the sequencing read to the reference sequence.

22. The method of claim 21, further comprising calling one or more SNVs or SNPs for a sample or subject that the double-stranded nucleic acid molecule is derived from.

23. The method of claim 1, wherein nucleic acid molecules in the first subset further comprise a first strand recognition element and wherein nucleic acid molecules the second subset further comprise a second strand recognition element that is different from the first strand recognition element.

24. The method of claim 23, wherein the first strand recognition element and the second strand recognition element are different sequences.

25.-33. (canceled)

34. The method of claim 23, further comprising detecting a presence of one or both of the first strand recognition element and the second strand recognition element in the amplified cluster by sequencing the plurality of nucleic acid molecules to identify a sequence of the first strand recognition element, the second strand recognition element, or both.

35.-37. (canceled)

38. The method of claim 34, further comprising determining a ratio of the first subset and the second subset in the plurality of nucleic acid molecules.

39. The method of claim 38, wherein the ratio is determined by processing signal intensities collected from interrogating the first strand recognition element, the second strand recognition element, or both.

40. The method of claim 38 further comprising generating a sequencing read for the amplified cluster, wherein the sequencing read is generated based at least in part on the sequencing signals and the ratio.

41.-100. (canceled)

101. The method of claim 22, further comprising detecting a minimal residual disease (MRD), tumor fraction, or circulating tumor fraction in the sample or the subject based on the one or more SNVs or SNPs called.
